SEQUENCE LISTING (46 sequences)

SEQ ID NO: 1 - protein, 15 aa, synthetic construct
  VARIANT 1..3: note = GLN, LN, N, or absent
  VARIANT 13..15: note = WHE, WH, W, or absent
XXXDIFEAQKIEXXX

SEQ ID NO: 2 - protein, 12 aa, synthetic construct
GLNDIFEAQKIE

SEQ ID NO: 3 - protein, 13 aa, synthetic construct
MGLNDIFEAQKIE

SEQ ID NO: 4 - protein, 5 aa, synthetic construct
GSGGS

SEQ ID NO: 5 - protein, 4 aa, synthetic construct
GSGG

SEQ ID NO: 6 - protein, 4 aa, synthetic construct
SGGS

SEQ ID NO: 7 - protein, 15 aa, synthetic construct
GLNDIFEAQKIEWHE

SEQ ID NO: 8 - protein, 14 aa, synthetic construct
GLNDIFEAQKIEWH

SEQ ID NO: 9 - protein, 13 aa, synthetic construct
GLNDIFEAQKIEW

SEQ ID NO: 10 - protein, 14 aa, synthetic construct
LNDIFEAQKIEWHE

SEQ ID NO: 11 - protein, 13 aa, synthetic construct
LNDIFEAQKIEWH

SEQ ID NO: 12 - protein, 12 aa, synthetic construct
LNDIFEAQKIEW

SEQ ID NO: 13 - protein, 11 aa, synthetic construct
LNDIFEAQKIE

SEQ ID NO: 14 - protein, 13 aa, synthetic construct
NDIFEAQKIEWHE

SEQ ID NO: 15 - protein, 12 aa, synthetic construct
NDIFEAQKIEWH

SEQ ID NO: 16 - protein, 11 aa, synthetic construct
NDIFEAQKIEW

SEQ ID NO: 17 - protein, 10 aa, synthetic construct
NDIFEAQKIE

SEQ ID NO: 18 - protein, 12 aa, synthetic construct
DIFEAQKIEWHE

SEQ ID NO: 19 - protein, 11 aa, synthetic construct
DIFEAQKIEWH

SEQ ID NO: 20 - protein, 10 aa, synthetic construct
DIFEAQKIEW

SEQ ID NO: 21 - protein, 9 aa, synthetic construct
DIFEAQKIE

SEQ ID NO: 22 - protein, 15 aa, synthetic construct
GLNDIFEAQKIEGSG

SEQ ID NO: 23 - protein, 18 aa, synthetic construct
MGLNDIFEAQKIEGSGGS

SEQ ID NO: 24 - protein, 17 aa, synthetic construct
MGLNDIFEAQKIEGSGG

SEQ ID NO: 25 - protein, 16 aa, synthetic construct
MGLNDIFEAQKIEGSG

SEQ ID NO: 26 - protein, 17 aa, synthetic construct
MGLNDIFEAQKIESGGS

SEQ ID NO: 27 - protein, 16 aa, synthetic construct
MGLNDIFEAQKIEGGS

SEQ ID NO: 28 - protein, 15 aa, synthetic construct
MGLNDIFEAQKIEGS

SEQ ID NO: 29 - protein, 321 aa, Escherichia coli
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 30 - protein, 441 aa, Homo sapiens
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQRRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 31 - protein, 441 aa, Homo sapiens
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 32 - DNA, 2213 bp, Homo sapiens
  source 1..2213: mol_type = genomic DNA
gcgtgtaaacagacatggccggcgaaggagatcagcaggacgctgcgcacaacatgggcaaccacctgccgctcctgcctgagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaggagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaaacagatgtgatagagataaagttagttatctaacaaattggttatattctaagatctgctttggaaattattgcctctgatacatacctaagtaaacataacattaatacctaagtaaacataacattacttggagggttgcagtttctaagtgaaactgtatttgaaacttttaagtatactttaggaaacaagcatgaacggcagtctagaataccagaaacatctacttgggtagcttggtgccattatcctgtggaatctgatatgtctggtagcgtgtcattgatgggacatgaagacatctttggaaatgatgagattatttcctgtgttaaaaaaaaaaaaaaaatcttaaattcctacaatgtgaaactgaaactaataatttgatcctgatgtatgggacagcgtatctgtaccagtgctctaaataacaaaagctagggtgacaagtacatgttccttttggaaagaagcaaggcaatgtatattaattattctaaaagggctttgttcctttccattttctttaacttctctgagatactgatttgtaaattttgaaaattagttaaaatatgcagttttttgagcccacgaatagttgtcatttcctttatgtgcctgttagtaaaaagtagtattgtgtatttgctcagtatctgaactataagcccatttatactgttccatacaaaagctatttttcaaaaattaatttgaaccaaaactactactatagggaaaagatgccaaaacatgtcccctcacccaggctaaacttgatactgtattattttgttcaatgtaaattgaagaaaatctgtaagtaagtaaaccttaagtgtgaaactaaaaaaaaaaaaaaaa

SEQ ID NO: 33 - protein, 1140 aa, Homo sapiens [sequence omitted]

SEQ ID NO: 34 - DNA, 4372 bp, Homo sapiens
  source 1..4372: mol_type = genomic DNA
ggtgcctccgggggcggggcctccttcggttggcggcctcgggcttcgggagtcctccaagaggccaggtgaggccgtcccgtgatgccccgcgccccggccgctctggcctgcaacgtgtctctggggcggaggcagcggcagtggagttcgctgcgcgctgttgggggccacctgtcttttcgcttgtgtccctctttctagtgtcgcgctcgagtcccgacgggccgctccaagcctcgacatgtcgtacaactacgtggtaacggcccagaagcccaccgccgtgaacggctgcgtgaccggacactttacttcggccgaagacttaaacctgttgattgccaaaaacacgagattagagatctatgtggtcaccgccgaggggcttcggcccgtcaaagaggtgggcatgtatgggaagattgcggtcatggagcttttcaggcccaagggggagagcaaggacctgctgtttatcttgacagcgaagtacaatgcctgcatcctggagtataaacagagtggcgagagcattgacatcattacgcgagcccatggcaatgtccaggaccgcattggccgcccctcagagaccggcattattggcatcattgaccctgagtgccggatgattggcctgcgtctctatgatggccttttcaaggttattccactagatcgcgataataaagaactcaaggccttcaacatccgcctggaggagctgcatgtcattgatgtcaagttcctatatggttgccaagcacctactatttgctttgtctaccaggaccctcaggggcggcacgtaaaaacctatgaggtgtctctccgagaaaaggaattcaataagggcccttggaaacaggaaaatgtcgaagctgaagcttccatggtgatcgcagtcccagagccctttgggggggccatcatcattggacaggagtcaatcacctatcacaatggtgacaaatacctggctattgcccctcctatcatcaagcaaagcacgattgtgtgccacaatcgagtggaccctaatggctcaagatacctgctgggagacatggaaggccggctcttcatgctgcttttggagaaggaggaacagatggatggcaccgtcactctcaaggatctccgtgtagaactccttggagagacctctattgctgagtgcttgacataccttgataatggtgttgtgtttgtcgggtctcgcctgggtgactcccagcttgtgaagctcaacgttgacagtaatgaacaaggctcctatgtagtggccatggaaacctttaccaacttaggacccattgtcgatatgtgcgtggtggacctggagaggcaggggcaggggcagctggtcacttgctctggggctttcaaggaaggttctttgcggatcatccggaatggaattggaatccacgagcatgccagcattgacttaccaggcatcaaaggattatggccactgcggtctgaccctaatcgtgagactgatgacactttggtgctctcttttgtgggccagacaagagttctcatgttaaatggagaggaggtagaagaaaccgaactgatgggtttcgtggatgatcagcagactttcttctgtggcaacgtggctcatcagcagcttatccagatcacttcagcatcggtgaggttggtctctcaagaacccaaagctctggtcagtgaatggaaggagcctcaggccaagaacatcagtgtggcctcctgcaatagcagccaggtggtggtggctgtaggcagggccctctactatctgcagatccatcctcaggagctccggcagatcagccacacagagatggaacatgaagtggcttgcttggacatcaccccattaggagacagcaatggactgtcccctctttgtgccattggcctctggacggacatctcggctcgtatcttgaagttgccctcttttgaactactgcacaaggagatgctgggtggagagatcattcctcgctccatcctgatgaccacctttgagagtagccattacctcctttgtgccttgggagatggagcgcttttctactttgggctcaacattgagacaggtctgttgagcgaccgtaagaaggtgactttgggcacccagcccaccgtattgaggacttttcgttctctttctaccaccaacgtctttgcttgttctgaccgccccactgtcatctatagcagcaaccacaaattggtcttctcaaatgtcaacctcaaggaagtgaactacatgtgtcccctcaattcagatggctatcctgacagcctggcgctggccaacaatagcaccctcaccattggcaccatcgatgagatccagaagctgcacattcgcacagttcccctctatgagtctccaaggaagatctgctaccaggaagtgtcccagtgtttcggggtcctctccagccgcattgaagtccaagacacgagtgggggcacgacagccttgaggcccagcgctagcacccaggctctgtccagcagtgtaagctccagcaagctgttctccagcagcactgctcctcatgagacctcctttggagaagaggtggaggtgcacaacctacttatcattgaccaacacacctttgaagtgcttcatgcccaccagtttctgcagaatgaatatgccctcagtctggtttcctgcaagctgggcaaagaccccaacacttacttcattgtgggcacagcaatggtgtatcctgaagaggcagagcccaagcagggtcgcattgtggtctttcagtattcggatggaaaactacagactgtggctgaaaaggaagtgaaaggggccgtgtactctatggtggaatttaacgggaagctgttagccagcatcaatagcacggtgcggctctatgagtggacaacagagaaggagctgcgcactgagtgcaaccactacaacaacatcatggccctctacctgaagaccaagggcgacttcatcctggtgggcgaccttatgcgctcagtgctgctgcttgcctacaagcccatggaaggaaactttgaagagattgctcgagactttaatcccaactggatgagtgctgtggaaatcttggatgatgacaattttctgggggctgaaaatgcctttaacttgtttgtgtgtcaaaaggatagcgctgccaccactgacgaggagcggcagcacctccaggaggttggtcttttccacctgggcgagtttgtcaatgtcttttgccacggctctctggtaatgcagaatctgggtgagacttccacccccacacaaggctcggtgctcttcggcacggtcaacggcatgatagggctggtgacctcactgtcagagagctggtacaacctcctgctggacatgcagaatcgactcaataaagtcatcaaaagtgtggggaagatcgagcactccttctggagatcctttcacaccgagcggaagacagaaccagccacaggtttcatcgacggtgacttgattgagagtttcctggatattagccgccccaagatgcaggaggtggtggcaaacctacagtatgacgatggcagcggtatgaagcgagaggccactgcagacgacctcatcaaggttgtggaggagctaactcggatccattagccaagggcagggggcccctttgctgaccctccccaaaggctttgccctgctgccctccccctcctctccaccatcgtcttcttggccatgggaggcctttccctaagccagctgcccccagagccacagttcccctatgtggaagtggggcgggcttcatagagacttgggaatgagctgaaggtgaaacattttctccctggatttttaccagtctcacatgattccagccatcaccttagaccaccaagccttgattggtgttgccagttgtcctccttccggggaaggattttgcagttctttggctgaaaggaagctgtgcgtgtgtgtgtgtgtatgtgtgtgtgtgtatgtgtatctcacactcatgcattgtcctctttttatttagattggcagtgtagggagttgtgggtagtggggaagagggttaggagggtttcattgtctgtgaagtgagaccttccttttacttttcttctattgcctctgagagcatcaggcctagaggcctgactgccaagccatgggtagcctgggtgtaaaacctggagatggtggatgatccccacgccacagcccttttgtctctgcaaactgccttcttcggaaagaagaaggtgggaggatgtgaattgttagtttctgagttttaccaaataaagtagaatataagaagaaaggtaaaaaaaaaaaaaaaaa

SEQ ID NO: 35 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 36 - DNA, 4414 bp, Homo sapiens
  source 1..4414: mol_type = genomic DNA
gcagctccgccccgcgtccgacccgcggatcccgcggcgtccggcccgggtggtctggatcgcggagggaatgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattgaagatttctgttgaaacttacactgtttcatctcagcttttgatggtactgatgagtcttgatctagatacaggactggttccttccttagtttcaaagtgtctcattctcagagtaaaataggcaccattgcttaaaagaaagttaactgacttcactaggcattgtgatgtttaggggcaaacatcacaaaatgtaatttaatgcctgcccattagagaagtatttatcaggagaaggtggtggcatttttgcttcctagtaagtcaggacagcttgtatgtaaggaggtttgtataagtaattcagtgggaattgcagcatatcgtttaattttaagaaggcattggcatctgcttttaatggatgtataatacatccattctacatccgtagcggttggtgacttgtctgcctcctgctttgggaagactgaggcatccgtgaggcagggacaagtctttctcctctttgagaccccagtgcctgcacatcatgagccttcagtcagggtttgtcagaggaacaaaccaggggacactttgttagaaagtgcttagaggttctgcctctatttttgttggggggtgggagaggggaccttaaaatgtgtacagtgaacaaatgtcttaaagggaatcatttttgtaggaagcattttttataattttctaagtcgtgcactttctcggtccactcttgttgaagtgctgttttattactgtttctaaactaggattgacattctacagttgtgataatagcatttttgtaacttgccatccgcacagaaaatacgagaaaatctgcatgtttgattatagtattaatggacaaataagtttttgctaaatgtgagtatttctgttcctttttgtaaatatgtgacattcctgattgatttgggtttttttgttgttgttgttttgttttgttttgtttttttgagatggagtctcactcttgtcacccaggctggagtgcagtggcgccatctcggctcactgcaacctctgcctcctgggttcacgtaatcctcctgagtagctgggattacaggcgcctgccaccacgctggccaatttttgtacttttagtagagacagtgtttcgtcatgttggccaggctggtttcaaactcctgacctcaggtgatccgcccacctcagcctcccaaaatggtgggattacaggtgtgtgggccaccgtgcctggctgattcagcattttttatcaggcaggaccaggtggcacttccacctccagcctctggtcctaccaatggattcatggagtagcctggactgtttcatagttttctaaatgtacaaattcttataggctagacttagattcattaactcaaattcaatgcttctatcagactcagttttttgtaactaatagatttttttttccacttttgttctactccttccctaatagctttttaaaaaaatctccccagtagagaaacatttggaaaagacagaaaactaaaaaggaagaaaaaagatccctattagatacacttcttaaatacaatcacattaacattttgagctatttccttccagcctttttagggcagattttggttggtttttacatagttgagattgtactgttcatacagttttataccctttttcatttaactttataacttaaatattgctctatgttagtataagcttttcacaaacattagtatagtctcccttttataattaatgtttgtgggtatttcttggcatgcatctttaattccttatcctagcctttgggcacaattcctgtgctcaaaaatgagagtgacggctggcatggtggctcccgcctgtaatcccagtactttggaaagccaaggtaagaggattgcttgagcccagaacttcaagatgagcctgggctcatagtgagaacccatctatacaaaaaatttttaaaaattagcatggcggcacacatctgtaatcctagctacttggcaggctgaggtgagaagatcattggagtttaggaattggaggctgcagtgagccatgagtatgccactgcactccagcctgggggacagagcaagaccctgcctcaaaaaaaaaaaaaaaaaaaaaatcaggccgggcatggtggctcacgcctgtaatcccagcactttgggaggtcgaggtgggcagatcacctgaggtcaggagttcgagaccagcctggccaacatggtaaaaccccatttctactaaaaaatacaagaattagctgggtgtggtggcgcatgcctgtaatcctagctactcaggaggctgaggcaggagaatcacttgaacccaggaggcgaagattgcagtgagctgatatcgcaccattgtactccagcctgtgtgacagagcaatactcttgtctcaaaaaaaaaaaaaaattcaaatcagagtgaagtgaatgagacactccagttttccttctactccgaatttcaactgattttagctcctcctttcaacattcaacaaatagtctttttttttttttttttttttttttttttgagatggagtctcactctgttgcccaggctggagtgcagtggtgcgatctctgctcactacaagctctgcctcccgagttcaagtgattctcctggctcaccctcctgagtagctgggattacaggcgcctgccaccatgcctggctaattttgtgtttttagtggagacggggtttcaccatgttgtccaggatggtcttgatctcctgaccttgtgatccacccacctcagcctcccaaagtgctgggattacaggtgtgagccaccgcgtccagccagctttattattttttttaagctgtctttgtgtcaaaatgatagttcatgctcctcttgttaaaacctgcaggccgagcacagtggctcatgcctgtaatcccagcattttgggagaccaaggcggatggatcacctgaggtcaggagctgaagaccagcctggctaacatggtgaaacctcatctccacttaaaatacaaaaattgccggccgcggcggctcatgcctgtaatcccagcactttgggaggcctaggcgggtggatcacgaggtcaggaaatcgagaccatcctggctaacacgggtgaaaccccgtctctattaaaaaatagaaaaaattaggcgggcgtggtggtgagcgcctgtagtcccagctactcgagagcctgaggcaggagaatggcatgaacctggaaggcggagcttgcagtgagctgagatggtgccactgcactctaacctgggcgacagagtgagacaccgtctcaaaaaaaaaaacaaaaaacaaaaattatccaggtgtggcggtgggcgcctgtgaggcaggcgaatctcttgaacccgggaggcggaggttgcagtgagccaagatcacaccattgcactccagcctgggcaacaagagtgaaattccatctcaaaaagaaaccaaaaaaacaaaaaaaaaacatgccgtttgagtactgtgtttttggtgttgtccaaggaaaattaaaaacctgtagcatgaataatgtttgtttttcatttcgaatcttgtgaatgtattaaatatatcgctcttaagagacggtgaagttcctatttcaagtttttttttttttttttttttttaaagctgttttttaatacattaaatggtgctgagtaaaggaaa

SEQ ID NO: 37 - protein, 4 aa, synthetic construct
GGSG

SEQ ID NO: 38 - protein, 76 aa, unidentified [sequence omitted]

SEQ ID NO: 39 - protein, 18 aa, synthetic construct
MGLNDIFEAQKIEWHEGS

SEQ ID NO: 40 - protein, 14 aa, synthetic construct
GKPIPNPLLGLDST

SEQ ID NO: 41 - protein, 9 aa, synthetic construct
IPNPLLGLD

SEQ ID NO: 42 - DNA, 42 bp, synthetic construct
  source 1..42: mol_type = other DNA
ggtaagcctatccctaaccctctcctcggtctcgattctacg

SEQ ID NO: 43 - DNA, 25 bp, synthetic construct
  source 1..25: mol_type = other DNA
caccgtaaacagacatggccggcga

SEQ ID NO: 44 - DNA, 25 bp, synthetic construct
  source 1..25: mol_type = other DNA
aaactcgccggccatgtctgtttac

SEQ ID NO: 45 - DNA, 18 bp, synthetic construct
  source 1..18: mol_type = other DNA
ggcctgtaattgtccctc

SEQ ID NO: 46 - DNA, 18 bp, synthetic construct
  source 1..18: mol_type = other DNA
gtaaccgctgtgaatctg

(19) United States
(12) Patent Application Publication
(10) Pub. No.: US 2025/0084453 A1
(43) Pub. Date: Mar. 13, 2025

(54) SYSTEMS, COMPOSITIONS AND METHODS FOR IDENTIFYING E3 LIGASE SUBSTRATES BY UBIQUITIN BIOTINYLATION

(71) Applicants: The Broad Institute, Inc., Cambridge, MA (US); Dana-Farber Cancer Institute, Inc., Boston, MA (US)

(72) Inventors: William Sellers, Cambridge, MA (US); Hai-Tsang Huang, Cambridge, MA (US)

(73) Assignees: The Broad Institute, Inc., Cambridge, MA (US); Dana-Farber Cancer Institute, Inc., Boston, MA (US)

(21) Appl. No.: 18/960,980

(22) Filed: Nov. 26, 2024

Related U.S. Application Data
(63) Continuation of application No. PCT/US2023/023863, filed on May 30, 2023.
(60) Provisional application No. 63/347,802, filed on Jun. 1, 2022.

(51) Int. Cl. C12Q 1/48; C12N 9/00

(52) U.S. Cl. CPC C12Q 1/48; C12N 9/93; C12Y 603/04015; G01N 2333/9015

(57) ABSTRACT

Described and featured are compositions, a system and methods for identifying and selecting substrates of E3 ligases by ubiquitin biotinylation. The components of the compositions, system and methods are ubiquitin- and interaction-specific, thereby providing the enrichment and identification of endogenous or exogenous E3 ligase substrate molecules that are proximally ubiquitinated and biotinylated by components designed to interact both physically and functionally in the compositions, system and methods. The compositions, system and methods are useful and advantageous for identifying and selecting E3 ligase substrates (and/or associated molecules) that are modulated or induced by other agents, such as immunomodulatory imide agents (IMiDs), molecular glues and bifunctional proteolysis targeting chimeras (PROTAC®s).

### CROSS-REFERENCE TO RELATED APPLICATION

This application is a continuation under 35 U.S.C. § 111(a) of PCT International Patent Application No. PCT/US2023/023863, filed May 30, 2023, designating the United States and published in English, which claims priority to and benefit of U.S. Provisional Application No. 63/347,802, filed on Jun. 1, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Jul. 5, 2023, is named “167741-042302US_PCT_SL.xml” and is 55,368 bytes in size.

### BACKGROUND

Ubiquitination is a prevalent post-translational modification of proteins that involves the addition of ubiquitin molecules to lysine residues of the protein. Following ubiquitination, most proteins are targeted to the 26S proteosome for degradation. Ubiquitination plays a significant role in controlling protein levels and signal transduction in cells. Post-translational modification by ubiquitin and ubiquitin-like proteins is fundamental for maintaining protein homeostasis, which contributes to the natural equilibrium within the cells. Disruption of protein homeostasis is frequently the cause or consequence of multiple diseases, including neurological diseases and cancers. The intricate balance is typically achieved through post-translational modifications by ubiquitin and ubiquitin-like proteins, which control the function, localization, or stability of target proteins.

In the ubiquitination system, ubiquitin is attached to target proteins by a three-step mechanism involving the sequential actions of the E1, E2 and E3 enzymes, which activate, conjugate, and ligate, respectively, the ubiquitin protein to a substrate protein. In humans, two E1-activating enzymes transfer ubiquitin to over thirty E2 enzymes, which function together with over six hundred different E3 ubiquitin ligase enzymes to modify substrate proteins by ubiquitination. The specificity of the ubiquitination system depends on the specific E3 ubiquitin ligase enzyme that catalyzes the transfer of a ubiquitin molecule onto the correct target protein or substrate.

Determining the direct substrates of individual ubiquitin ligases is important in understanding how ubiquitination exerts its effect on cellular functions. However, methods and processes to determine the linkage between individual E3 ligases and their specific substrates are lacking. It is also challenging to identify the substrates of a specific E3 ligase (“E3”), largely because of the weak interaction that exists between E3 and substrates, the heterogeneity of modifications, and the rapid degradation of ubiquitinated substrates. Existing approaches used to identify ubiquitin substrates involve hypothesis-driven candidate approaches, which are slow and heavily biased, as well as low-throughput. In addition, the E3 and substrate interaction may be transient and, therefore, difficult to detect by known methods, such as affinity binding; moreover, binding of E3 to the substrate does not always result in productive ubiquitination or degradation of protein. Ubiquitinated substrates may be labile due to proteasomal degradation or hydrolyzed by de-ubiquitinating enzymes. Specific and reliable chemical tools are lacking to modulate the activities of E3 ligases, and E3 ligases often have redundant and pleiotropic substrate profiles, which hamper the identification of specific E3 substrates.

In view of the foregoing, new, robust, effective, and unbiased methods for identifying substrates of E3 ligases are needed.

### SUMMARY

In an aspect, a method of identifying a substrate of an E3 ligase is provided, in which the method involves contacting a ubiquitinated substrate of an E3 ligase with a biotin ligase fused to the E3 ligase, wherein the E3 ligase substrate is ubiquitinated with one or more ubiquitin or ubiquitin-like molecules, each of which is fused to a biotin ligase peptide substrate comprising a biotinylation site of the biotin ligase, wherein the biotin ligase fused to the E3 ligase biotinylates the tagged ubiquitinated E3 ligase substrate when in proximity to the tag; and identifying the substrate of the E3 ligase by detecting and/or selecting the biotinylated ubiquitinated E3 ligase substrate. In an embodiment, the contacting is within a cell. In an embodiment, the E3 ligase is fused at its amino (NH₂)- or carboxy (COOH)-terminus to the biotin ligase.

In an aspect, a method of identifying a substrate of an E3 ligase is provided, in which the method involves (i) expressing in a cell one or more ubiquitin or ubiquitin-like molecules fused to a biotin ligase peptide substrate which comprises a biotinylation site of the biotin ligase; (ii) expressing in the cell an E3 ligase fused at its amino (NH₂)- or carboxy (COOH)-terminus to a non-promiscuous biotin ligase, wherein the E3 ligase catalyzes the ubiquitination of an E3 ligase substrate bound thereto with the one or more ubiquitin or ubiquitin-like molecules of (i), which are biotinylated by the non-promiscuous biotin ligase fused to the E3 ligase; and (iii) identifying the substrate of the E3 ligase by detecting and/or selecting the biotinylated and ubiquitinated substrate bound to the E3 ligase.

In an embodiment of the above-delineated methods and embodiments thereof, the biotin ligase fused to the E3 ligase comprises a non-promiscuous biotin ligase. In an embodiment, the non-promiscuous biotin ligase comprises a wild-type biotin ligase derived from E. coli. In an embodiment, the E. coli wild-type biotin ligase is BirA. In an embodiment of the above-delineated methods and embodiments thereof, the biotin ligase peptide substrate comprises an amino acid sequence (X)DIFEAQKIE(Y) (SEQ ID NO: 1), wherein X is selected from no amino acid; amino acids G, L, and N; amino acids L and N; or amino acid N; and Y is selected from no amino acid; amino acids W, H, and E; amino acids W and H; or amino acid W. In an embodiment, the biotin ligase peptide substrate comprises an amino terminus initial methionine (M) residue. In an embodiment of the above-delineated methods and embodiments thereof, the biotin ligase peptide substrate comprises an amino acid sequence having at least 80% sequence identity to amino acid sequence GLNDIFEAQKIE (SEQ ID NO: 2) or MGLNDIFEAQKIE (SEQ ID NO: 3). In an embodiment of the above-delineated methods and embodiments thereof, the biotin ligase peptide substrate comprises an amino acid sequence having at least 90% sequence identity to amino acid sequence GLNDIFEAQKIE (SEQ ID NO: 2) or MGLNDIFEAQKIE (SEQ ID NO: 3). In an embodiment of the above-delineated methods and embodiments thereof, the biotin ligase peptide substrate comprises an amino acid sequence having at least 95% sequence identity to amino acid sequence GLNDIFEAQKIE (SEQ ID NO: 2) or MGLNDIFEAQKIE (SEQ ID NO: 3). In an embodiment of the above-delineated methods and embodiments thereof, the biotin ligase peptide substrate comprises or consists of an amino acid sequence having amino acid sequence GLNDIFEAQKIE (SEQ ID NO: 2) or MGLNDIFEAQKIE (SEQ ID NO: 3). In an embodiment of the above-delineated methods and embodiments thereof, the biotin ligase peptide substrate comprises a carboxy terminal linker sequence. In an embodiment, the linker sequence is selected from the group consisting of GSGGS (SEQ ID NO: 4), GSGG (SEQ ID NO: 5), GSG, SGGS (SEQ ID NO: 6), GGS and GS.

In an embodiment of the above-delineated methods and embodiments thereof, the biotin ligase fused to the E3 ligase is a biotin ligase enzyme genetically or recombinantly substituted with an unnatural amino acid residue. In an embodiment, the unnatural amino acid residue comprises a photocaged lysine (K) analog at position 183 (K183) in a non-promiscuous BirA biotin ligase enzyme derived from wild-type E. coli, or a photocaged K analog at an analogous position in another biotin ligase enzyme. In an embodiment, the photocaged lysine analog comprises N^{ε}-[1-(6-Nitrobenzo[d][1,3]dioxol-5yl)ethoxy)carbonyl]-L-lysine (ONPK) at position 183 in the BirA biotin ligase enzyme or at an analogous position in another biotin ligase enzyme. In an embodiment, the method further involves expressing in the cells pyrrolysine-amino acyl tRNA synthase (PylRS) for ONPK incorporation in the BirA lysine analog; and subjecting the cell to photo-illumination prior to step (iii) to restore activity of the photocaged BirA biotin ligase enzyme.

In an embodiment of the above-delineated methods and embodiments thereof, the ubiquitin-like molecule is selected from the group consisting of NEDD8, SUMO, ISG15, ATG8, ATG12, FAT10 and functional equivalents thereof. In an embodiment of the above-delineated methods and embodiments thereof, the E3 ligase is selected from an RBR, HECT, RING, or cullin-RING, class of E3 ubiquitin ligases, or multimeric complexes of the foregoing. In an embodiment of the above-delineated methods and embodiments thereof, the E3 ligase is selected from the group consisting of E3A, MDM2, Anaphase-promoting complex (APC), UBR5 (EDD1), SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HERC5, HERC6, HUWE1, ITCH, NEDD4, NEDD4L, PPIL2, PRPF19, PIAS1, PIAS2, PIAS3, PIAS4, RANBP2, RNF4, RNF13, RNF38, RNF139, RNFx2, RNF126, RBX1, SMURF1, SMURF2, STUB1, TEB4, TOPORS, TRIP12, UBE3A, UBE3B, UBE3C, UBE3D, UBE4A, UBE4B, UBOX5, UBR5, VHL, WWP1, WWP2, Parkin, MKRN1, CRBN, CRL4-CRBN, XIAP, cIAPl, gp78, DoalO, Hrdl, MARCH1, MARCH5, DCAF15, and multimeric complexes thereof. In an embodiment, the E3 ligase is CRBN or CRL4-CRBN fused to the biotin ligase. In another embodiment, the E3 ligase is von Hippel-Lindau disease tumor suppressor (VHL).

In an embodiment of the above-delineated methods and embodiments thereof, the method further involves contacting the E3 ligase with an immunomodulatory imide drug (IMiD). In an embodiment of the above-delineated methods and embodiments thereof, the method further involves introducing an immunomodulatory imide drug (IMiD) into the cell, wherein the IMiD contacts the E3 ligase. In an embodiment, the IMiD promotes binding of CRBN or CRL4-CRBN E3 ligase to an E3 ligase substrate; and wherein the E3 ligase substrate is ubiquitinated with the one or more ubiquitin or ubiquitin-like molecules fused to the non-promiscuous biotin ligase peptide substrate comprising a biotinylation site. In embodiments, the IMiD is selected from lenalidomide, thalidomide, pomalidomide, or CC-885. In an embodiment, the IMiD is CC-885.

In an embodiment of the above-delineated methods and embodiments thereof, the E3 ligase substrate is an endogenous substrate or an exogenous substrate. In embodiments, the E3 ligase substrate is selected from Ikaros (IKZF1), Aiolos (IKZF3), or Casein kinase 1 Alpha (CSNK1a1). In embodiments, the E3 ligase substrate is Ikaros (IKZF1) or Aiolos (IKZF3). In an embodiment, the E3 ligase substrate is translation termination factor GSPT1 or GSPT2. In an embodiment, the E3 ligase substrate is HIF1A and/or HIF2A.

In an embodiment of the above-delineated methods and embodiments thereof, an agent selected from a molecular glue, reprogramming agent, or proteolysis targeting chimera (PROTAC®) molecule is provided or introduced into the cell. In an embodiment, the proteolysis targeting chimera (PROTAC®) comprises a first component which binds to the E3 ligase and a second component which binds to a target molecule or target protein. In an embodiment, the target molecule or target protein is ubiquitinated and biotinylated. In an embodiment, the second component comprises a protein kinase inhibitor or protein kinase degrader, which binds to a target protein kinase. In an embodiment, the protein kinase inhibitor or kinase degrader is a multikinase degrader. In an embodiment, the multikinase degrader is selected from SK-3-91, DB0646, SB1-G-187, or WH-10417-099. In an embodiment of the methods, the proteolysis targeting chimera (PROTAC®) comprises a Bromo- and Extra-terminal (BET) bromodomain degrader or a PTK2 (Protein Tyrosine Kinase 2) degrader. In an embodiment, the E3 ligase is CRBN or VHL. In an embodiment of the methods, the agent comprises a molecular glue. In an embodiment, the molecular glue comprises a sulfonamide molecular glue. In an embodiment, the sulfonamide molecular glue is E7820. In an embodiment, the E3 ligase is DCAF15. In an embodiment of the methods, the proteolysis targeting chimera (PROTAC®) comprises a Histone Deacetylase (HDAC) inhibitor (degrader). In an embodiment, the E3 ligase is CRBN or VHL.

In an embodiment of any of the above-delineated methods and embodiments thereof, the method further involves enriching for and/or isolating the biotinylated and ubiquitinated E3 ligase substrate or target molecule or protein associated with the E3 ligase. In an embodiment of the above-delineated methods and embodiments thereof, the expressing comprises introducing into the cell one or more polynucleotides encoding the one or more ubiquitin or ubiquitin-like molecules fused to the peptide substrate of the biotin ligase and/or one or more polynucleotides encoding the E3 ligase fused to the biotin ligase. In an embodiment, the one or more polynucleotides are contained in an expression vector. In an embodiment, the one or more polynucleotides is operably linked to one or more promoters and/or regulatory sequences. In an embodiment, the expression vector is a plasmid vector or a viral vector. In an embodiment, the viral vector is a lentivirus vector, an adenovirus vector (AAV), a recombinant adenovirus vector (rAAV), or a retrovirus vector.

In an embodiment of any of the above-delineated methods and embodiments thereof, selecting the biotinylated E3 ligase substrate, target molecule, or target protein comprises use of an assay comprising streptavidin, avidin, or an analog thereof, attached to a solid support. In an embodiment, the assay is a precipitation or pulldown assay. In an embodiment, the streptavidin or avidin is attached to a solid support selected from beads, particles, microparticles, nanoparticles, an array, a microarray, or an affinity column.

In an embodiment of the above-delineated methods and embodiments thereof, the cell is a mammalian, non-mammalian, human, non-human, invertebrate, insect, prokaryotic, eukaryotic, algal, yeast, fungal, plant, nematode, or protoplast cell. In an embodiment, the cell is a human cell. In embodiments, the cell is selected from a primary cell, a cultured cell, a cancer cell, a tumor cell, a neoplastic cell, an HEK293T cell, a MOLT4 cell, an NIH 3T3 cell, a HeLa cell or a COS cell, a cell derived or obtained from an organism at different stages of development, a cell derived or obtained from a subject with a genetic disorder, a cell derived or obtained from a subject at different stages of disease, or a cell derived or obtained from a subject with disease, at different stages of treatment or therapy for the disease. In embodiments, the cell is in vitro, in vivo, or ex vivo. In an embodiment, the cell is grown or cultured in a medium devoid of biotin prior to the step of identifying the substrate of the E3 ligase or a target molecule or target protein associated with the E3 ligase.

In an embodiment of any of the above-delineated methods and embodiments thereof, the selected biotinylated E3 ligase substrate or target molecule or target protein is identified using Western blot or mass spectrometry. In an embodiment, the mass spectrometry is liquid chromatography-tandem mass spectrometry (LC/MS/MS).

In another aspect, a system or composition is provided, in which the system or composition includes: (i) one or more ubiquitin or ubiquitin-like molecules fused to a biotin ligase peptide substrate which comprises a biotinylation site of the biotin ligase, or one or more polynucleotides encoding said one or more ubiquitin or ubiquitin-like molecules; and (ii) an E3 ligase fused to a biotin ligase, or a polynucleotide encoding said E3 ligase; wherein the E3 ligase catalyzes the ubiquitination of an E3 ligase substrate bound thereto with the one or more ubiquitin or ubiquitin-like molecules of (i), and wherein the one or more ubiquitin or ubiquitin-like molecules are biotinylated by the biotin ligase fused to the E3 ligase. In an embodiment, the E3 ligase is fused at its amino (NH₂)- or carboxy (COOH)-terminus to the biotin ligase. In an embodiment, the biotin ligase fused to the E3 ligase comprises a non-promiscuous biotin ligase. In an embodiment, the non-promiscuous biotin ligase comprises a wild-type biotin ligase derived from E. coli. In an embodiment, the E. coli wild-type biotin ligase is BirA.

In an embodiment of the above-delineated system or composition and embodiments thereof, the biotin ligase peptide substrate comprises an amino acid sequence (X)DIFEAQKIE(Y) (SEQ ID NO: 1), wherein X is selected from no amino acid; amino acids G, L, and N; amino acids L and N; or amino acid N; and Y is selected from no amino acid; amino acids W, H, and E; amino acids W and H; or amino acid W. In an embodiment, the biotin ligase peptide substrate comprises an amino terminus initial methionine (M) residue. In an embodiment of the above-delineated system or composition and embodiments thereof, the biotin ligase peptide substrate comprises an amino acid sequence having at least 80% sequence identity to amino acid sequence GLNDIFEAQKIE (SEQ ID NO: 2) or MGLNDIFEAQKIE (SEQ ID NO: 3). In an embodiment of the above-delineated system or composition and embodiments thereof, the biotin ligase peptide substrate comprises an amino acid sequence having at least 90% sequence identity to amino acid sequence GLNDIFEAQKIE (SEQ ID NO: 2) or MGLNDIFEAQKIE (SEQ ID NO: 3). In an embodiment of the above-delineated system or composition and embodiments thereof, the biotin ligase peptide substrate comprises an amino acid sequence having at least 95% sequence identity to amino acid sequence GLNDIFEAQKIE (SEQ ID NO: 2) or MGLNDIFEAQKIE (SEQ ID NO: 3). In an embodiment of the above-delineated system or composition and embodiments thereof, the biotin ligase peptide substrate comprises or consists of an amino acid sequence having amino acid sequence GLNDIFEAQKIE (SEQ ID NO: 2) or MGLNDIFEAQKIE (SEQ ID NO: 3).

In an embodiment of the above-delineated system or composition and embodiments thereof, the biotin ligase peptide substrate comprises a carboxy terminal linker sequence. In an embodiment, the linker sequence is selected from the group consisting of GSGGS (SEQ ID NO: 4), GSGG (SEQ ID NO: 5), GSG, SGGS (SEQ ID NO: 6), GGS and GS.

In an embodiment of the above-delineated system or composition and embodiments thereof, the biotin ligase fused to the E3 ligase is a biotin ligase enzyme genetically or recombinantly substituted with an unnatural amino acid residue. In an embodiment, the unnatural amino acid residue comprises a photocaged lysine (K) analog at position 183 (K183) in a non-promiscuous BirA biotin ligase enzyme derived from wild-type E. coli, or a photocaged K analog at an analogous position in another biotin ligase enzyme. In an embodiment, the photocaged lysine analog comprises N^{ε}-[1-(6-Nitrobenzo[d][1,3]dioxol-5yl)ethoxy)carbonyl]-L-lysine (ONPK) at position 183 in the BirA biotin ligase enzyme or at an analogous position in another biotin ligase enzyme.

In an embodiment of the above-delineated system or composition and embodiments thereof, the ubiquitin-like molecule is selected from the group consisting of NEDD8, SUMO, ISG15, ATG8, ATG12, FAT10 and functional equivalents thereof. In an embodiment of the above-delineated system or composition and embodiments thereof, the E3 ligase is selected from an RBR, HECT, RING, or cullin-RING, class of E3 ubiquitin ligases, or multimeric complexes of the foregoing. In an embodiment of the above-delineated system or composition and embodiments thereof, the E3 ligase is selected from the group consisting of E3A, MDM2, Anaphase-promoting complex (APC), UBR5 (EDD1), SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HERC5, HERC6, HUWE1, ITCH, NEDD4, NEDD4L, PPIL2, PRPF19, PIAS1, PIAS2, PIAS3, PIAS4, RANBP2, RNF4, RNF13, RNF38, RNF139, RNFx2, RNF126, RBX1, SMURF1, SMURF2, STUB1, TEB4, TOPORS, TRIP12, UBE3A, UBE3B, UBE3C, UBE3D, UBE4A, UBE4B, UBOX5, UBR5, VHL, WWP1, WWP2, Parkin, MKRN1, CRBN, CRL4-CRBN, CRBN XIAP, cIAPl, gp78, DoalO, Hrdl, MARCH1 MARCH5, DCAF15, and multimeric complexes thereof. In embodiments, the E3 ligase is CRBN or CRL4-CRBN fused to the biotin ligase. In an embodiment, the E3 ligase is von Hippel-Lindau disease tumor suppressor (VHL).

In an embodiment of the above-delineated system or composition and embodiments thereof, the system or composition further includes an immunomodulatory imide drug (IMiD), wherein the IMiD contacts the E3 ligase. In an embodiment, the IMiD promotes binding of CRBN or CRL4-CRBN E3 ligase to an E3 ligase substrate; and wherein the E3 ligase substrate is ubiquitinated with the one or more ubiquitin or ubiquitin-like molecules fused to the non-promiscuous biotin ligase peptide substrate comprising a biotinylation site. In an embodiment, the IMiD is selected from lenalidomide, thalidomide, pomalidomide, or CC-885. In an embodiment, the IMiD is CC-885.

In an embodiment of the above-delineated system or composition and embodiments thereof, the E3 ligase substrate is an endogenous substrate or an exogenous substrate. In an embodiment, the E3 ligase substrate is selected from Ikaros (IKZF1), Aiolos (IKZF3), or Casein kinase 1 Alpha (CSNK1a1). In an embodiment, the E3 ligase substrate is Ikaros (IKZF1) or Aiolos (IKZF3). In an embodiment, the E3 ligase substrate is translation termination factor GSPT1 or GSPT2. In an embodiment, the E3 ligase substrate is HIF1A and/or HIF2A.

In an embodiment of the above-delineated system or composition and embodiments thereof, the system or composition further includes an agent selected from a molecular glue, reprogramming agent, or proteolysis targeting chimera (PROTAC®) molecule. In an embodiment, the proteolysis targeting chimera (PROTAC®) comprises a first component which binds to the E3 ligase and a second component which binds to a target molecule or target protein. In an embodiment, the target molecule or target protein is ubiquitinated and biotinylated. In an embodiment, the second component comprises a protein kinase inhibitor or protein kinase degrader, which binds to a target protein kinase. In an embodiment, the protein kinase inhibitor of kinase degrader is a multikinase degrader. In an embodiment, the multikinase degrader is selected from SK-3-91, DB0646, SB1-G-187, or WH10417-099. In an embodiment of the system or composition, the proteolysis targeting chimera (PROTAC®) comprises a Bromo- and Extra-terminal (BET) bromodomain degrader or a PTK2 (Protein Tyrosine Kinase 2) degrader. In an embodiment, the E3 ligase is CRBN or VHL. In an embodiment of the system or composition, the agent comprises a molecular glue. In an embodiment, the molecular glue comprises a sulfonamide molecular glue. In an embodiment, the sulfonamide molecular glue is E7820. In an embodiment, the E3 ligase is DCAF15. In an embodiment of the system or composition, the proteolysis targeting chimera (PROTAC®) comprises a Histone Deacetylase (HDAC) inhibitor (degrader). In an embodiment, the E3 ligase is CRBN or VHL

In another aspect, a cell including the system or composition of the above-delineated aspect and embodiments thereof is provided. In embodiments, the cell is selected from a primary cell, a cultured cell, a cell line, a cancer cell, a tumor cell, a neoplastic cell, a cell obtained from a subject with a disease or pathology, an HEK293T cell, a MOLT4 cell, an NIH 3T3 cell, a HeLa cell or a COS cell, a cell derived or obtained from an organism at different stages of development, a cell derived or obtained from a subject with a genetic disorder, a cell derived or obtained from a subject at different stages of disease, or a cell derived or obtained from a subject with disease, at different stages of treatment or therapy for the disease. In embodiments, the cell is derived from or is located within a prokaryotic, eukaryotic, non-mammalian, mammalian, invertebrate, vertebrate, insect, nematode, yeast, fungal, algal, protozoan or plant organism. In embodiments, the cell is or is located within a human, an insect, or a nematode organism.

In another aspect, a kit including the system or composition of the above-delineated aspect and embodiments thereof is provided.

In another aspect, a kit including the cell of the above-delineated aspect and embodiments thereof is provided.

In another aspect, an in vitro or ex vivo method is provided, in which the method involves: contacting a cell with the system or composition of any one of claims 57-92, wherein the contacting results in the expression in the cell of the one or more ubiquitin or ubiquitin-like molecules fused to a biotin ligase peptide substrate or one or more polynucleotides encoding said one or more ubiquitin or ubiquitin-like molecules; and the E3 ligase fused at its amino (NH₂)- or carboxy (COOH)-terminus to a non-promiscuous biotin ligase, or a polynucleotide encoding said E3 ligase. In embodiments, the cell is selected from a primary cell, a cultured cell, a cell line, a cancer cell, a tumor cell, a neoplastic cell, a cell obtained from a subject with a disease or pathology, an HEK293T cell, a MOLT4 cell, an NIH 3T3 cell, a HeLa cell or a COS cell, a cell derived or obtained from an organism at different stages of development, a cell derived or obtained from a subject with a genetic disorder, a cell derived or obtained from a subject at different stages of disease, or a cell derived or obtained from a subject with disease, at different stages of treatment or therapy for the disease. In embodiments, the cell is derived from or is located within a prokaryotic, eukaryotic, non-mammalian, mammalian, invertebrate, vertebrate, insect, nematode, yeast, fungal, algal, protozoan or plant organism. In an embodiment, the non-promiscuous biotin ligase fused to the E3 ligase is a wild-type biotin ligase derived from E. coli. In an embodiment, the E. coli wild-type biotin ligase is BirA. In an embodiment, the non-promiscuous biotin ligase fused to the E3 ligase is a biotin ligase enzyme genetically or recombinantly substituted with an unnatural amino acid residue. In an embodiment of the method and embodiments thereof, the unnatural amino acid residue comprises a photocaged lysine (K) analog at position 183 (K183) in a non-promiscuous BirA biotin ligase enzyme derived from wild-type E. coli, or a K analog at an analogous position in another BirA biotin ligase enzyme. In an embodiment, the photocaged lysine analog comprises N^{ε}-[1-(6-Nitrobenzo[d][1,3]dioxol-5yl)ethoxy)carbonyl]-L-lysine (ONPK) at position 183 in the BirA biotin ligase enzyme or at an analogous position another BirA biotin ligase enzyme. In an embodiment, the method further involves expressing in the cells pyrrolysine-amino acyl tRNA synthase (PylRS) for ONPK incorporation in the BirA lysine analog. In an embodiment, the method further involves subjecting the cell to photo-illumination to restore activity of the photocaged BirA biotin ligase. In an embodiment, the photo-illumination comprises 365 nm for at least 5 minutes. In an embodiment of the above-delineated method or the above-delineated system or composition, and embodiments thereof, the unnatural amino acid residue comprises a chemical-caged lysine analog N-((E)-cyclooct-2-en-1-yl)-oxy)carbonyl-L-lysine (TCOK) is incorporated at position 183 in the BirA biotin ligase enzyme or at an analogous position another BirA biotin ligase enzyme. In an embodiment of the in vitro or ex vivo method and embodiments thereof, the method further involves subjecting the cell, or an organism comprising the cell, to chemical activator dimethyl tetrazine (DM-Tz) to restore activity of the chemically-caged BirA biotin ligase.

In another aspect, a system or composition is provided, which include (i) one or more ubiquitin proteins fused to a biotin ligase peptide substrate tag which comprises a biotinylation site of the biotin ligase; or one or more polynucleotides encoding said one or more ubiquitin proteins; (ii) an E3 ligase fused at its amino (NH₂)- or carboxy (COOH)-terminus to a biotin ligase, or a polynucleotide encoding said E3 ligase; and (iii) an agent selected from an IMiD, a molecular glue, a reprogramming molecule, or a proteolysis targeting chimera (PROTAC®), that facilitates an association of a target protein or a target molecule with the E3 ligase, whereby the target protein or target molecule is ubiquitinated with the one or more tagged ubiquitin proteins of (i), which are biotinylated by the biotin ligase fused to the E3 ligase.

In another aspect, a method for identifying a target protein is provided, in which the method involves (i) providing one or more ubiquitin proteins fused to a biotin ligase peptide substrate tag which comprises a biotinylation site of the biotin ligase; or one or more polynucleotides encoding said one or more ubiquitin proteins; (ii) providing an E3 ligase fused at its amino (NH₂)- or carboxy (COOH)-terminus to a biotin ligase, or a polynucleotide encoding said E3 ligase; (iii) providing an agent selected from an IMiD, a molecular glue, a reprogramming molecule, or a proteolysis targeting chimera (PROTAC®), that facilitates an association of the target protein with the E3 ligase, whereby the target protein is ubiquitinated with the one or more tagged ubiquitin proteins of (i), which are biotinylated by the biotin ligase fused to the E3 ligase; and (iv) detecting and/or selecting the biotinylated and ubiquitinated target protein associated with the E3 ligase, thereby identifying the target protein.

In an embodiment of the above-delineated system or composition, or the above-delineated method, which include an agent selected from an IMiD, a molecular glue, a reprogramming molecule, or a proteolysis targeting chimera (PROTAC®), the biotin ligase fused to the E3 ligase comprises a non-promiscuous biotin ligase. In an embodiment, the non-promiscuous biotin ligase comprises a wild-type biotin ligase derived from E. coli. In an embodiment, the E. coli wild-type biotin ligase is BirA. In an embodiment of the above-delineated system or composition, or the above-delineated method, which include an agent selected from an IMiD, a molecular glue, a reprogramming molecule, or a proteolysis targeting chimera (PROTAC®), the biotin ligase peptide substrate comprises an amino acid sequence (X)DIFEAQKIE(Y) (SEQ ID NO: 1), wherein X is selected from no amino acid; amino acids G, L, and N; amino acids L and N; or amino acid N; and Y is selected from no amino acid; amino acids W, H, and E; amino acids W and H; or amino acid W. In an embodiment, the biotin ligase peptide substrate comprises an amino terminus initial methionine (M) residue.

In an embodiment of the above-delineated system or composition, or the above-delineated method, which include an agent selected from an IMiD, a molecular glue, a reprogramming molecule, or a proteolysis targeting chimera (PROTAC®), and embodiments thereof, the biotin ligase peptide substrate comprises an amino acid sequence having at least 80% sequence identity to amino acid sequence GLNDIFEAQKIE (SEQ ID NO: 2) or MGLNDIFEAQKIE (SEQ ID NO: 3). In an embodiment of the above-delineated system or composition, or the above-delineated method, which include an agent selected from an IMiD, a molecular glue, a reprogramming molecule, or a proteolysis targeting chimera (PROTAC®), and embodiments thereof, the biotin ligase peptide substrate comprises an amino acid sequence having at least 90% sequence identity to amino acid sequence GLNDIFEAQKIE (SEQ ID NO: 2) or MGLNDIFEAQKIE (SEQ ID NO: 3). In an embodiment of the above-delineated system or composition, or the above-delineated method, which include an agent selected from an IMiD, a molecular glue, a reprogramming molecule, or a proteolysis targeting chimera (PROTAC®), and embodiments thereof, the biotin ligase peptide substrate comprises an amino acid sequence having at least 95% sequence identity to amino acid sequence GLNDIFEAQKIE (SEQ ID NO: 2) or MGLNDIFEAQKIE (SEQ ID NO: 3). In an embodiment of the above-delineated system or composition, or the above-delineated method, which include an agent selected from an IMiD, a molecular glue, a reprogramming molecule, or a proteolysis targeting chimera (PROTAC®), and embodiments thereof, the biotin ligase peptide substrate comprises or consists of an amino acid sequence having amino acid sequence GLNDIFEAQKIE (SEQ ID NO: 2) or MGLNDIFEAQKIE (SEQ ID NO: 3). In an embodiment of the above-delineated system or composition, or the above-delineated method, which include an agent selected from an IMiD, a molecular glue, a reprogramming molecule, or a proteolysis targeting chimera (PROTAC®), and embodiments thereof, the biotin ligase peptide substrate comprises a carboxy terminal linker sequence. In an embodiment, the linker sequence is selected from the group consisting of GSGGS (SEQ ID NO: 4), GSGG (SEQ ID NO: 5), GSG, SGGS (SEQ ID NO: 6), GGS and GS. In an embodiment of the above-delineated system or composition, or the above-delineated method, which include an agent selected from an IMiD, a molecular glue, a reprogramming molecule, or a proteolysis targeting chimera (PROTAC®), and embodiments thereof, the biotin ligase fused to the E3 ligase is a biotin ligase enzyme genetically or recombinantly substituted with an unnatural amino acid residue. In an embodiment, the unnatural amino acid residue comprises a photocaged lysine (K) analog at position 183 (K183) in a non-promiscuous BirA biotin ligase enzyme derived from wild-type E. coli, or a photocaged K analog at an analogous position in another biotin ligase enzyme. In an embodiment, the photocaged lysine analog comprises N^{ε}-[1-(6-Nitrobenzo[d][1,3]dioxol-5yl)ethoxy)carbonyl]-L-lysine (ONPK) at position 183 in the BirA biotin ligase enzyme or at an analogous position in another biotin ligase enzyme. In an embodiment, photo-illumination exposure restores activity of the photocaged BirA biotin ligase enzyme.

In an embodiment of any of the above-delineated methods and embodiments thereof, wherein the method is performed in a high-throughput screening (HTS) assay format. In an embodiment, the HTS assay, levels of biotinylated substrate, target protein, or target molecule are quantified. In an embodiment, the levels of biotinylated substrate, target protein, or target molecule are quantified by a proximity-based detection assay. In an embodiment, the proximity-based detection assay comprises donor beads conjugated to streptavidin and acceptor beads conjugated to substrate-specific antibody. In an embodiment, the proximity-based detection assay is a bead-based Amplified Luminescent Proximity Homogeneous Assay (ALPHASCREEN™) assay.

In another aspect, a high-throughput screening method for screening for target proteins is provided, in which the method involves: (a) contacting a cell with the system or composition of any one of the above-delineated aspects and embodiments thereof, wherein the contacting results in the delivery into the cell of the one or more ubiquitin or ubiquitin-like molecules fused to a biotin ligase peptide substrate, or one or more polynucleotides encoding said one or more ubiquitin or ubiquitin-like molecules; and the E3 ligase fused at its amino (NH₂)- or carboxy (COOH)-terminus to a non-promiscuous biotin ligase, or a polynucleotide encoding said E3 ligase; (b) contacting a cell lysate or extract produced from the cell of step (a) with donor beads conjugated to streptavidin and acceptor beads conjugated to an antibody specifically directed to the substrate of the E3 ligase; and (c) measuring levels of biotinylated and ubiquitinated substrate produced following ubiquitin biotinylation by the non-promiscuous biotin ligase fused to the E3 ligase. In an embodiment, the levels of biotinylated and ubiquitinated substrate are measured by an Amplified Luminescent Proximity Homogenous Assay (Alpha) Screening assay. In an embodiment, the cells are contacted in wells of a multi-well microtiter plate, which may contain 96, 192, 384, 1536, 3456, or 6144 wells.

In another aspect, a method of identifying a substrate of an E3 ubiquitin ligase is provided, in which the method involves introducing into a cell a polynucleotide encoding a mutant E3 ubiquitin ligase fused to a biotin ligase and expressing the mutant E3 ubiquitin ligase in the cell; wherein the cell expresses one or more ubiquitin or ubiquitin-like molecules fused to a biotin ligase peptide substrate which comprises a biotinylation site of the biotin ligase; culturing the cell in biotin-containing medium for a time sufficient for biotinylation of ubiquitinated protein substrates of the E3 ubiquitin ligase; enriching the biotinylated protein substrates with streptavidin; and detecting the levels of biotinylated protein substrates ubiquitinated by the mutant E3 ubiquitin ligase in lysates of the cells compared with the levels of biotinylated protein substrates ubiquitinated by a wildtype E3 ubiquitin ligase to identify substrate proteins differentially enriched by the mutant E3 ubiquitin ligase versus the wildtype E3 ubiquitin ligase. In an embodiment of the method, the cell is a cell according to any one of the above delineated aspects and/or embodiments thereof. In an embodiment of the method, the mutant E3 ubiquitin ligase comprises a loss-of-function mutation. In an embodiment of the method, the E3 ubiquitin ligase mutant comprising a loss-of-function mutation has a reduced, diminished, or loss of ubiquitinating activity compared with the wildtype E3 ubiquitin ligase. In an embodiment, the E3 ubiquitin ligase is CRBN or VHL E3 ubiquitin ligase. In an embodiment of the method, the E3 ubiquitin ligase mutant comprising a loss-of-function mutation has a reduced, diminished, or decreased association with a cullin RING complex. In an embodiment, the E3 ubiquitin ligase mutant comprising a loss-of-function mutation comprises CRBN comprising amino acid D249Y. In an embodiment, the E3 ubiquitin ligase mutant comprising a loss-of-function mutation has decreased association with CRL4 complex. In an embodiment, the E3 ubiquitin ligase mutant comprising a loss-of-function mutation has a reduced, diminished, or decreased affinity for an immunomodulatory molecule (IMiD). In an embodiment, the E3 ubiquitin ligase mutant comprising a loss-of-function mutation comprises CRBN comprising amino acid W386A. In an embodiment, the E3 ubiquitin ligase mutant comprising a loss-of-function mutation has decreased affinity for CC-885. In an embodiment of the method, the biotin ligase fused to the E3 ubiquitin ligase comprises a non-promiscuous biotin ligase. In an embodiment, the non-promiscuous biotin ligase comprises a BirA wild-type biotin ligase derived from E. coli. In an embodiment of the method, the biotin ligase peptide substrate comprises a A3-Ubiquitin tag.

In yet another aspect, a method of identifying a substrate of an E3 ligase is provided, in which the method involves contacting a cell, which is a cell according to any one of the above delineated aspects and/or embodiments thereof, with an inhibitor of all cullin-RING ligase (CRL) activity or an inhibitor of all ubiquitin ligase activity in a cell for a time sufficient to block E3 ligase activity and accumulate E3 ligase substrate in the cell; wherein the cell is cultured in biotin-free medium; removing the inhibitor from the culture to restore E3 ligase activity and culturing the cell in biotin-free medium prior to supplementing the culture medium with biotin, wherein ubiquitinated substrates of the E3 ligase are biotinylated in the cell; enriching the biotinylated protein substrates with streptavidin following cell lysis; and detecting and identifying the ubiquitinated substrate of the E3 ligase in the cell lysate. In an embodiment of the method, the inhibitor blocks all cullin-RING ligase (CRL) activity in the cell. In an embodiment, the inhibitor is MLN4924. In an embodiment, the E3 ligase is a cullin-RING ligase (CRL). In an embodiment, the E3 ligase is VHL. In an embodiment, the inhibitor blocks all ubiquitin ligase activity in the cell. In an embodiment, the inhibitor blocks non-cullin-RING E3 ligase activity in the cell. In an embodiment, the inhibitor is MLN7243. In an embodiment, the E3 ligase is a non-cullin-RING E3 ligase. In an embodiment, the biotin labeling of the ubiquitinated substrates is carried out by the method according to any one of the above-delineated methods and/or embodiments thereof. In an embodiment, the cell is contacted with E3 ligase inhibitor for a time period of at least 15 minutes to 2 hours, at least 30 minutes to 1.5 hours, at least 30 minutes to 1 hour, or at least 30 minutes to 2 hours.

In another aspect, a method of identifying a substrate of an E3 ligase is provided, in which the method involves (a) contacting a cell, which is a cell according to any one of the above-delineated aspects and/or embodiments thereof, with a first E3 ligase inhibitor for a time sufficient to block E3 ligase and cullin ring ligase (CRL) activity in the cell; wherein the cell is cultured in biotin-free medium; (b) removing the first inhibitor from the culture to restore E3 ligase and CRL activity and culturing the cell in biotin-free medium prior to supplementing the culture medium with biotin and a second inhibitor that blocks or inhibits E3 ligase activity; (c) lysing the cell and enriching the biotinylated ubiquitinated protein substrates of the E3 ligase from the cell lysate with streptavidin; and (d) detecting and identifying the enriched biotinylated and ubiquitinated protein substrates having decreased biotinylation due to inhibition of E3 ligase activity by the first and second E3 ligase inhibitors. In an embodiment of the method, the first E3 ligase inhibitor comprises a compound or ligand that blocks or inhibits all cullin RING ligase activity and/or E3 ligase activity in the cell. In an embodiment of the method, the first inhibitor is an enzymatic inhibitor of E3 ligase and/or CRL. In embodiments, the inhibitor is MLN4924 or MLN7243. In an embodiment of the method, the second E3 ligase inhibitor comprises a compound or ligand that competes with the E3 ligase for binding endogenous substrates of the E3 ligase. In an embodiment, the E3 ligase is VHL E3 ligase. In an embodiment, the VHL E3 ligase inhibitor comprises a compound or ligand that competes with VHL for binding endogenous substrates of VHL. In an embodiment, the inhibitor is VH298. In an embodiment of the method, the cell is contacted with the first inhibitor for at least 15 minutes to 2 hours and the cell is contacted with the second inhibitor for at least 20 to 30 minutes. In an embodiment of any one of the above-delineated methods and/or embodiments thereof, the streptavidin is attached to a support or solid substrate. In an embodiment, the support or solid substrate comprises beads, particles, nanoparticles, or microparticles. In an embodiment of any one of the above-delineated methods and/or embodiments thereof, the detection and/or identification of biotinylated substrates is performed by mass spectrophotometry.

### Definitions

Unless defined otherwise, all technical and scientific terms used herein have the meaning according to conventional usage and as commonly understood by a person of skill in the pertinent art. The following references provide one of skill with a general definition of many of the terms used and referred to herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). Definitions of common terms in molecular biology may be found in standard texts (e.g. Benjamin Lewin, Genes V, published by Oxford University Press, 1994 (ISBN 0-19854287-9); Kendrew et ah. (eds.), The Encyclopedia of Molecular Biology, published by Blackwell Science Ltd, 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression or activity levels, preferably a 25% change, more preferably a 40% change, and most preferably a 50% or greater change in expression or activity levels.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine modifications. Amino acid analogs refers to compounds that have the same basic chemical structure as a naturally occurring amino acid, e.g., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid. Amino acids may be referred to by either their commonly-known and understood three letter symbols or by the one-letter symbols recommended by the IUPAC-IUB Biochemical Nomenclature Commission. Similarly, nucleotides may be referred to by their commonly accepted single-letter codes.

The term “antibody” refers to a polypeptide comprising a framework region from an immunoglobulin gene, or fragments thereof, that specifically binds and recognizes an antigen through complementarity determining regions (CDRs) in the variable regions of the heavy and light chains of the immunoglobulin polypeptide. The recognized immunoglobulin genes include kappa and lambda light chain constant region genes, and alpha, gamma, delta, epsilon, and mu constant region genes, as well as a vast number of immunoglobulin variable region genes. Heavy chains are classified as gamma, mu, alpha, delta, or epsilon, which, in turn, define the immunoglobulin classes, IgG, IgM, IgA, IgD and IgE, respectively. Typically, the antigen-binding region (variable region) of an antibody will be most critical in specificity and affinity of binding. Antibodies can be polyclonal or monoclonal, derived from serum, a hybridoma or recombinantly cloned, and can also be chimeric, primatized, or humanized. Antibodies exist. e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially a Fab with part of the hinge region (see Fundamental Immunology (Paul ed., 3d ed. 1993). While various antibody fragments are defined in terms of the digestion of an intact antibody, one of skill in the art will appreciate that such fragments may be synthesized de novo either chemically or by using well-known recombinant DNA methodology and techniques. Thus, the term antibody, as used herein, also includes antibody fragments either produced by the modification of whole antibodies, or those synthesized de novo using recombinant DNA methodologies (e.g., single chain antibodies, diabodies, VHHs, single chain Fv, etc.) or those identified using phage display libraries (e.g., McCafferty et al., Nature 348:552-554 (1990)).

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. Lenalidomide analogs include, but are not limited to, thalidomide or pomalidomide.

As used herein, the term “Avi-tag” (such as AVI-TAG™) refers, in general, to a 15-amino acid peptide tag comprising the sequence GLNDIFEAQKIEWHE (SEQ ID NO: 7). (See. e.g., U.S. Pat. Nos. 5,723,584, 5,874,239 and 5,932,433). This peptide serves as a substrate (or substrate mimic) for a biotin ligase, such as the E. coli biotin ligase BirA, and is specifically biotinylated with a biotin molecule at a single site (i.e., lysine (K)) within the amino acid sequence of the peptide substrate by BirA. The BirA biotin ligase specifically and covalently attaches biotin to the 15 amino acid AVI-TAG™ peptide, producing a homogeneous product with high yield. (M. Fairhead et al., 2015, Methods Mol Biol., 1266:171-184. doi:10.1007/978-1-4939-2272-2277), incorporated herein by reference in its entirety. Proteins that are tagged with a biotin ligase peptide substrate, such as Avi-tag, and that are biotinylated can be detected, selected, immobilized, visualized, or purified by avidin or streptavidin, e.g., streptavidin attached to a solid support or surface, due to the specific and reversible binding of avidin or streptavidin to biotin. Avi-tag peptides are commercially available, e.g., Avidity, LLC (Aurora, CO), GeneCopoeia, Inc. (Rockville, MD), Cusabiotechnology, LLC (Houston, TX). Antibodies directed against the Avi-tag peptide may be produced using methods known in the art and are also commercially available, allowing for immunological detection or investigation of Avi-tagged recombinant proteins.

As used herein, a biotin ligase peptide substrate, such as an “A3-tag,” refers to a peptide comprising the following amino acid sequence GLNDIFEAQKIE (SEQ ID NO: 2), shown in “NH₂” (amino) to “COOH” (carboxy) terminal orientation. In various embodiments, the biotin ligase peptide substrate tag, or A3-tag peptide, and variants thereof, include the following amino acid sequences:

| | | | |
|---|---|---|---|
| GLNDI | GLNDI | GLNDI | GLNDI |
| FEAQK | FEAQK | FEAQK | FEAQK |
| IEWHE | IEWH | IEW | IE |
| (SEQ ID | (SEQ ID | (SEQ ID | (SEQ ID |
| NO: 7) | NO: 8) | NO: 9) | NO: 2) |
| LNDIF | LNDIF | LNDIF | LNDIF |
| EAQKI | EAQKI | EAQKI | EAQKI |
| EWHE | EWH | EW | E |
| (SEQ ID | (SEQ ID | (SEQ ID | (SEQ ID |
| NO: 10) | NO: 11) | NO: 12) | NO: 13) |
| NDIFE | NDIFE | NDIFE | NDIFE |
| AQKIE | AQKIE | AQKIE | AQKIE |
| WHE | WH | W | (SEQ ID |
| (SEQ ID | (SEQ ID | (SEQ ID | NO: 17) |
| NO: 14) | NO: 15) | NO: 16) | |
| DIFEA | DIFEA | DIFEA | DIFEA |
| QKIEW | QKIEW | QKIEW | QKIE |
| HE | H | (SEQ ID | (SEQ ID |
| (SEQ ID | (SEQ ID | NO: 20) | NO: 21) |
| NO: 18) | NO: 19) | | |

A biotin ligase peptide substrate (also called a biotin ligase peptide substrate tag herein), such as an A3-tag or a variant thereof, or an Avi-tag, may also include an initiating methionine (M or M1) residue at the amino terminus. In addition or alternatively, a biotin ligase peptide substrate tag, such as an Avi-tag or an A3-tag peptide, or a variant thereof, can be connected or fused to ubiquitin by a linker sequence. In embodiments, the linker sequences encompass variable GS linkers. By way of nonlimiting example, the linker sequences can include GSGGS (SEQ ID NO: 4), GSGG (SEQ ID NO: 5), GSG, SGGS (SEQ ID NO: 6), GGS, or GS. In an embodiment, the linker sequence is connected or fused to the biotin ligase peptide substrate tag, such as an Avi-tag or an A3-tag, at the carboxy terminus of the peptide. In an embodiment, the linker sequence is connected or fused to the biotin ligase peptide substrate tag, Avi-tag, or the A3-tag, at the amino terminus of the peptide. Accordingly, biotin ligase peptide substrate tags, such as A3 tag variants, may differ at their amino and/or carboxy termini, and also provide specificity for the system and methods described herein.

In an embodiment, the biotin ligase peptide substrate tag, such as an A3-tag, comprises the amino acid sequence GLNDIFEAQKIE (SEQ ID NO: 2). In an embodiment, the A3-tag comprises the amino acid sequence MGLNDIFEAQKIE (SEQ ID NO: 3), which includes an M1 residue. In an embodiment, the A3-tag comprises the amino acid sequence GLNDIFEAQKIE (SEQ ID NO: 2) and a carboxy terminal linker sequence, such as GSGGS (SEQ ID NO: 4), GSGG (SEQ ID NO: 5), GSG, SGGS (SEQ ID NO: 6), GGS, or GS. In an embodiment, the A3-tag peptide comprises the amino acid sequence GLNDIFEAQKIEGSG (SEQ ID NO: 22). In an embodiment, the A3-tag comprises the amino acid sequence MGLNDIFEAQKIE (SEQ ID NO: 3) and a carboxy terminal linker sequence, such as GSGGS (SEQ ID NO: 4), GSGG (SEQ ID NO: 5), GSG, SGGS (SEQ ID NO: 6), GGS, or GS, e.g., MGLNDIFEAQKIEGSGGS (SEQ ID NO: 23), MGLNDIFEAQKIEGSGG (SEQ ID NO: 24), MGLNDIFEAQKIEGSG (SEQ ID NO: 25), MGLNDIFEAQKIESGGS (SEQ ID NO: 26), MGLNDIFEAQKIEGGS (SEQ ID NO: 27), or MGLNDIFEAQKIEGS (SEQ ID NO: 28)), for example, for use in the transient transfection of cells. In embodiments, a biotin ligase peptide substrate tag, e.g., an A3-tag, may be linked, joined, or fused to another biotin ligase peptide substrate tag, e.g., an A3-tag, such that 1, 2, 3, 4 or more biotin ligase peptide substrate tags, e.g., A3-tags, are used in combination, for example, tandem or triplicate biotin ligase peptide substrate tags, or A3 tags. In embodiments, the biotin ligase peptide substrate tags, such as A3-tags or variants thereof, used in combination may be the same or different biotin ligase peptide substrate tags, or A3-tags. In embodiments, the biotin ligase peptide substrate tag, A3-tag, or a variant thereof, comprises or consists of any of the foregoing amino acid sequences.

The biotin ligase peptide substrate tag, or A3-tag peptide, contains a target site (i.e., lysine (K)) that is specifically biotinylated with biotin by biotin ligase, e.g., the E. coli biotin ligase, BirA. The biotin ligase peptide substrate tag, or A3-tag peptide, serves as a substrate (or substrate mimic) for the E. coli biotin ligase BirA and is specifically biotinylated with a biotin molecule at a single site within the amino acid sequence of the biotin ligase peptide substrate tag, or A3-tag peptide substrate, by BirA biotin ligase, which specifically and covalently attaches biotin to the peptide substrate tag. In an embodiment, a biotin ligase peptide substrate tag, such as an A3-tag, fused to ubiquitin can substitute for endogenous ubiquitin and can be biotinylated.

In embodiments, the biotin ligase peptide substrate tag, such as an A3-tag, comprises an amino acid sequence having at least 80% sequence identity, at least 85% sequence identity, at least 88% sequence identity, at least 9(0% sequence identity, at least 93% sequence identity, at least 95% sequence identity, at least 97%, 98%, 99%, or greater sequence identity with the above-noted biotin ligase peptide substrate tag, or A3-tag, amino acid sequences or variants thereof. The A3-tag exhibits a slightly weaker affinity for BirA biotin ligase compared with Avi-tag or AVI-TAG™. In an embodiment, the BirA biotin ligase utilized with the biotin ligase peptide substrate tag, or A3-tag, is anon-promiscuous BirA biotin ligase, such as the non-promiscuous E. coli wild type BirA. Notwithstanding its binding affinity for BirA, the A3-tag is highly useful and advantageous, as it ultimately provides for greater specificity in the interaction-specific methods and systems described herein, which can involve a non-promiscuous biotin-ligase, e.g., non-promiscuous BirA, fused to an E3 ligase, which functions in close proximity to the biotin ligase peptide substrate tag, or A3-tag peptide substrate, fused to ubiquitin or a ubiquitin-like protein, which is attached to a substrate of an E3 ligase, or to a target protein or target molecule associated with an E3 ligase.

In other embodiments, the system and methods herein may also be used with one or more biotin ligase substrate peptide tags that can be biotinylated by a biotin ligase such as BirA. By way of example, the one or more alternative tags comprises an amino acid sequence having at least 80% sequence identity, at least 85% sequence identity, at least 88% sequence identity, at least 90% sequence identity, at least 93% sequence identity, at least 95% sequence identity, at least 97%, 98%, 99%, or greater sequence identity with the above-noted A3-tag amino acid sequences or variants thereof.

By “biological sample” is meant any liquid, cell, or tissue obtained from a subject.

By “biomarker” or “marker” is meant any protein or polynucleotide having an alteration in expression level or activity that is associated with a disease or disorder. As used herein, “BirA” is an E. coli biotin ligase (35 kDa MW) having an amino acid sequence at least 85%, at least 90%, at least 93%, at least 95%, or greater, amino acid sequence identity with the BirA biotin ligase amino acid sequence (Accession No. UniprotKB-P06709) as follows:

```
(SEQ ID NO: 29)
                10 20 30 40 50
MKDNTVPLKL IALLANGEFH SGEQLGETLG MSRAAINKHI QTLRDWGVDV
                60 70 80 90 100
FTVPGKGYSL PEPIQLLNAK QILGQLDGGS VAVLPVIDST NQYLLDRIGE
              110 120 130 140 150
LKSGDACIAE YQQAGRGRRG RKWFSPFGAN LYLSMFWRLE QGPAAAIGLS
              160 170 180 190 200
LVIGIVMAEV LRKLGADKVR VKWPNDLYLQ DRKLAGILVE LTGKTGDAAQ
              210 220 230 240 250
IVIGAGINMA MRRVEESVVN QGWITLQEAG INLDRNTLAA MLIRELRAAL
              260 270 280 290 300
ELFEQEGLAP YLSRWEKLDN FINRPVKLII GDKEIFGISR GIDKQGALLL
              310 320
EQDGIIKPWM GGEISLRSAE K
```

The BirA biotin ligase enzyme site-specifically biotinylates a lysine side chain within a 15-amino acid acceptor peptide, e.g., A3-tag or Avi-tag, as described supra. In an embodiment, the BirA protein is a wild-type E. coli BirA biotin ligase, i.e., a biotin ligase that contains no mutations or amino acid variations. In an embodiment, the BirA protein is a recombinant, wild-type E. coli BirA biotin ligase. Recombinant E. coli BirA protein is commercially available, e.g., Abcam (Waltham, MA).

Biotin is a small organic molecule that binds avidin and streptavidin with extremely high affinity. Proteins can be covalently labeled with biotin, a process known as biotinylation, through chemical or enzymatic means. Biotinylation allows for the detection, immobilization, and purification of biotin-labeled proteins. The BirA enzyme catalyzes the covalent attachment of biotin to the lysine side chain within the 15-amino acid A3-tag or Avi-tag peptide. (Y. Li and R. Sousa, 2012, Protein Erpr Purif, 82(1):162-167).

A BirA biotin ligase may be a variant Bir A in which a catalytic amino acid residue in the BirA enzyme is substituted with an unnatural amino acid. In an embodiment, a catalytic lysine (K) amino acid residue in BirA, namely, K183, is genetically replaced with a photocaged lysine analog to produce a photocaged BirA variant. The K183 residue is required for the adenylation of biotin with ATP to produce the biotinyl-5′-AMP reactive intermediate. In an embodiment, the photocaged lysine analog N^{ε}-[1-(6-Nitrobenzo[d][1,3]dioxol-5yl)ethoxy)carbonyl]-L-lysine (ONPK) is genetically incorporated into BirA, for example, as described in Y. Liu et al., 2021, PNAS USA, Vol. 118, No. 25, which is incorporated by reference herein. The resulting BirA-K183(ONPK), termed a “photocaged BirA biotin ligase” is inactive in the presence of biotin substrate in culture medium under normal conditions. Upon light illumination or photolysis (e.g., 365 nm light for 5 minutes), ONPK liberates the lysine side-chain and restores the biotin ligase activity of the enzyme, e.g., to generate biotin-AMP from biotin and ATP. Such a photocaged BirA-K183(ONPK) may be fused to an E3 ligase in the system and methods described herein. Following photo-illumination, the biotinylated and ubiquitinated E3 ligase substrate attached to tagged ubiquitins can be selected, identified and/or isolated from the cells via streptavidin bead pulldown, for example, and analyzed via Western blot and mass spectrometry. In another embodiment, a chemically activatable biotin ligase enzyme, e.g., a non-promiscuous BirA, or a non-promiscuous BirA derived from wild-type E. coli, for use of the systems and methods herein in deep tissue or intact organisms (animals). Accordingly, a chemical-caged lysine analog N-((E)-cyclooct-2-en-1-yl)-oxy)carbonyl-L-lysine (TCOK) is incorporated in BirA ligase at amino acid residue K183 (or an analogous K residue in another biotin ligase enzyme), e.g., as described in Y. Liu et al., 2021, PNAS USA, Vol. 118, No. 25.

As used herein, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited are not changed by the presence of more than that which is recited, but excludes prior art embodiments.

By “CRBN polypeptide” or “Cereblon” is meant a polypeptide or fragment thereof having at least 85% amino acid sequence identity to human CRBN of NCBI Accession No. AAH67811.1 or of NP_001166953.1 and having IKZF1 and/or IKZF3 binding activity. Exemplary human CRBN polypeptide sequences are provided below:

```
AAH67811.1:
(SEQ ID NO: 30)
    1 MAGEGDQQDA AHNMGNHLPL LPESEEEDEM EVEDQDSKEA KKPNIINFDT SLPTSHTYLG
  61 ADMEEFHGRT LHDDDSCQVI PVLPQVMMIL IPGQTLPLQL FHPQEVSMVR NLIQKDRTFA
121 VLAYSNVQER EAQFGTTAEI YAYREEQDFG IEIVKVKAIG RQRFKVLELR TQSDGIQQAK
181 VQILPECVLP STMSAVQLES LNKCQIFPSK PVSREDQCSY KWWQKYQRRK FHCANLTSWP
241 RWLYSLYDAE TLMDRIKKQL REWDENLKDD SLPSNPIDFS YRVAACLPID DVLRIQLLKI
301 GSAIQRLRCE LDIMNKCTSL CCKQCQETEI TTKNEIFSLS LCGPMAAYVN PHGYVHETLT
361 VYKACNLNLI GRPSTEHSWF PGYAWTVAQC KICASHIGWK FTATKKDMSP QKFWGLTRSA
421 LLPTIPDTED EISPDKVILC L
NP_001166953.1:
(SEQ ID NO: 31)
    1 MAGEGDQQDA AHNMGNHLPL LPESEEEDEM EVEDQDSKEA KKPNIINFDT SLPTSHTYLG
  61 ADMEEFHGRT LHDDDSCQVI PVLPQVMMIL IPGQTLPLQL FHPQEVSMVR NLIQKDRTFA
121 VLAYSNVQER EAQFGTTAEI YAYREEQDFG IEIVKVKAIG RQRFKVLELR TQSDGIQQAK
181 VQILPECVLP STMSAVQLES LNKCQIFPSK PVSREDQCSY KWWQKYQKRK FHCANLTSWP
241 RWLYSLYDAE TLMDRIKKQL REWDENLKDD SLPSNPIDFS YRVAACLPID DVLRIQLLKI
301 GSAIQRLRCE LDIMNKCTSL CCKQCQETEI TTKNEIFSLS LCGPMAAYVN PHGYVHETLT
361 VYKACNLNLI GRPSTEHSWF PGYAWTVAQC KICASHIGWK FTATKKDMSP QKFWGLTRSA
421 LLPTIPDTED EISPDKVILC L
```

By “CRBN polynucleotide” is meant a nucleic acid molecule encoding a human CRBN polypeptide. An exemplary human CRBN polynucleotide sequence is provided at NCBI Accession No. BC067811, which is reproduced below:

| (SEQ ID NO: 32) | |
|---|---|
| 1 | gcgtgtaaac agacatggcc ggcgaaggag atcagcagga cgctgcgcac aacatgggca |
| 61 | accacctgcc gctcctgcct gagagtgagg aagaagatga aatggaagtt gaagaccagg |
| 121 | atagtaaaga agccaaaaaa ccaaacatca taaattttga caccagtctg ccgacatcac |
| 181 | atacatacct aggtgctgat atggaagaat ttcatggcag gactttgcac gatgacgaca |
| 241 | gctgtcaggt gattccagtt cttccacaag tgatgatgat cctgattccc ggacagacat |
| 301 | tacctcttca gctttttcac cctcaagaag tcagtatggt gcggaattta attcagaaag |
| 361 | atagaacctt tgctgttctt gcatacagca atgtacagga aagggaagca cagtttggaa |
| 421 | caacagcaga gatatatgcc tatcgagaag aacaggattt tggaattgag atagtgaaag |
| 481 | tgaaagcaat tggaagacaa aggttcaaag tccttgagct aagaacacag tcagatggaa |
| 541 | tccagcaagc taaagtgcaa attcttcccg aatgtgtgtt gccttcaacc atgtctgcag |
| 601 | ttcaattaga atccctcaat aagtgccaga tatttccttc aaaacctgtc tcaagagaag |
| 661 | accaatgttc atataaatgg tggcagaaat accagaggag aaagtttcat tgtgcaaatc |
| 721 | taacttcatg gcctcgctgg ctgtattcct tatatgatgc tgagacctta atggacagaa |
| 781 | tcaagaaaca gctacgtgaa tgggatgaaa atctaaaaga tgattctctt ccttcaaatc |
| 841 | caatagattt ttcttacaga gtagctgctt gtcttcctat tgatgatgta ttgagaattc |
| 901 | agctccttaa aattggcagt gctatccagc gacttcgctg tgaattagac attatgaata |
| 961 | aatgtacttc cctttgctgt aaacaatgtc aagaaacaga aataacaacc aaaaatgaaa |
| 1021 | tattcagttt atccttatgt gggccgatgg cagcttatgt gaatcctcat ggatatgtgc |
| 1081 | atgagacact tactgtgtat aaggcttgca acttgaatct gataggccgg ccttctacag |
| 1141 | aacacagctg gtttcctggg tatgcctgga ctgttgccca gtgtaagatc tgtgcaagcc |
| 1201 | atattggatg gaagtttacg gccaccaaaa aagacatgtc acctcaaaaa ttttggggct |
| 1261 | taacgcgatc tgctctgttg cccacgatcc cagacactga agatgaaata agtccagaca |
| 1321 | aagtaatact ttgcttgtaa acagatgtga tagagataaa gttagttatc taacaaattg |
| 1381 | gttatattct aagatctgct ttggaaatta ttgcctctga tacataccta agtaaacata |
| 1441 | acattaatac ctaagtaaac ataacattac ttggagggtt gcagtttcta agtgaaactg |
| 1501 | tatttgaaac ttttaagtat actttaggaa acaagcatga acggcagtct agaataccag |
| 1561 | aaacatctac ttgggtagct tggtgccatt atcctgtgga atctgatatg tctggtagcg |
| 1621 | tgtcattgat gggacatgaa gacatctttg gaaatgatga gattatttcc tgtgttaaaa |
| 1681 | aaaaaaaaaa aatcttaaat tcctacaatg tgaaactgaa actaataatt tgatcctgat |
| 1741 | gtatgggaca gcgtatctgt accagtgctc taaataacaa aagctagggt gacaagtaca |
| 1801 | tgttcctttt ggaaagaagc aaggcaatgt atattaatta ttctaaaagg gctttgttcc |
| 1861 | tttccatttt ctttaacttc tctgagatac tgatttgtaa attttgaaaa ttagttaaaa |
| 1921 | tatgcagttt tttgagccca cgaatagttg tcatttcctt tatgtgcctg ttagtaaaaa |
| 1981 | gtagtattgt gtatttgctc agtatctgaa ctataagccc atttatactg ttccatacaa |
| 2041 | aagctatttt tcaaaaatta atttgaacca aaactactac tatagggaaa agatgccaaa |
| 2101 | acatgtcccc tcacccaggc taaacttgat actgtattat tttgttcaat gtaaattgaa |
| 2161 | gaaaatctgt aagtaagtaa accttaagtg tgaaactaaa aaaaaaaaaa aaa |

By “DDB1 (DNA damage-binding protein 1) polypeptide” is meant a polypeptide or fragment thereof having at least 85% amino acid sequence identity to human DDB1 polypeptide NCBI Accession No. NP_001914.3 and having regulatory activity of CUL4A- and CUL4B-based E3 ubiquitin ligase complexes and nucleotide excision repair activity. An exemplary human DDB1 polypeptide sequence is provided below:

| (SEQ ID NO: 33) | |
|---|---|
| 1 | MSYNYVVTAQ KPTAVNGCVT GHFTSAEDLN LLIAKNTRLE IYVVTAEGLR PVKEVGMYGK |
| 61 | IAVMELFRPK GESKDLLFIL TAKYNACILE YKQSGESIDI ITRAHGNVQD RIGRPSETGI |
| 121 | IGIIDPECRM IGLRLYDGLF KVIPLDRDNK ELKAFNIRLE ELHVIDVKFL YGCQAPTICF |
| 181 | VYQDPQGRHV KTYEVSLREK EFNKGPWKQE NVEAEASMVI AVPEPFGGAI IIGQESITYH |
| 241 | NGDKYLAIAP PIIKQSTIVC HNRVDPNGSR YLLGDMEGRL FMLLLEKEEQ MDGTVTLKDL |
| 301 | RVELLGETSI AECLTYLDNG VVFVGSRLGD SQLVKLNVDS NEQGSYVVAM ETFTNLGPIV |
| 361 | DMCVVDLERQ GQGQLVTCSG AFKEGSLRII RNGIGIHEHA SIDLPGIKGL WPLRSDPNRE |
| 421 | TDDTLVLSFV GQTRVLMLNG EEVEETELMG FVDDQQTFFC GNVAHQQLIQ ITSASVRLVS |
| 481 | QEPKALVSEW KEPQAKNISV ASCNSSQVVV AVGRALYYLQ IHPQELRQIS HTEMEHEVAC |
| 541 | LDITPLGDSN GLSPLCAIGL WTDISARILK LPSFELLHKE MLGGEIIPRS ILMTTFESSH |
| 601 | YLLCALGDGA LFYFGLNIET GLLSDRKKVT LGTQPTVLRT FRSLSTTNVF ACSDRPTVIY |
| 661 | SSNHKLVFSN VNLKEVNYMC PLNSDGYPDS LALANNSTLT IGTIDEIQKL HIRTVPLYES |
| 721 | PRKICYQEVS QCFGVLSSRI EVQDTSGGTT ALRPSASTQA LSSSVSSSKL FSSSTAPHET |
| 781 | SFGEEVEVHN LLIIDQHTFE VLHAHQFLQN EYALSLVSCK LGKDPNTYFI VGTAMVYPEE |
| 841 | AEPKQGRIVV FQYSDGKLQT VAEKEVKGAV YSMVEFNGKL LASINSTVRL YEWTTEKELR |
| 901 | TECNHYNNIM ALYLKTKGDF ILVGDLMRSV LLLAYKPMEG NFEEIARDFN PNWMSAVEIL |
| 961 | DDDNFLGAEN AFNLFVCQKD SAATTDEERQ HLQEVGLFHL GEFVNVFCHG SLVMQNLGET |
| 1021 | STPTQGSVLF GTVNGMIGLV TSLSESWYNL LLDMQNRLNK VIKSVGKIEH SFWRSFHTER |
| 1081 | KTEPATGFID GDLIESFLDI SRPKMQEVVA NLQYDDGSGM KREATADDLI KVVEELTRIH |

By “DDB1 polynucleotide” is meant a nucleic acid molecule encoding a human DDB1 polypeptide. An exemplary human DDB1 polynucleotide sequence is provided at NCBI Accession No. NM-001923.4, which is reproduced below:

| (SEQ ID NO: 34) | |
|---|---|
| 1 | ggtgcctccg ggggcggggc ctccttcggt tggcggcctc gggcttcggg agtcctccaa |
| 61 | gaggccaggt gaggccgtcc cgtgatgccc cgcgccccgg ccgctctggc ctgcaacgtg |
| 121 | tctctggggc ggaggcagcg gcagtggagt tcgctgcgcg ctgttggggg ccacctgtct |
| 181 | tttcgcttgt gtccctcttt ctagtgtcgc gctcgagtcc cgacgggccg ctccaagcct |
| 241 | cgacatgtcg tacaactacg tggtaacggc ccagaagccc accgccgtga acggctgcgt |
| 301 | gaccggacac tttacttcgg ccgaagactt aaacctgttg attgccaaaa acacgagatt |
| 361 | agagatctat gtggtcaccg ccgaggggct tcggcccgtc aaagaggtgg gcatgtatgg |
| 421 | gaagattgcg gtcatggagc ttttcaggcc caagggggag agcaaggacc tgctgtttat |
| 481 | cttgacagcg aagtacaatg cctgcatcct ggagtataaa cagagtggcg agagcattga |
| 541 | catcattacg cgagcccatg gcaatgtcca ggaccgcatt ggccgcccct cagagaccgg |
| 601 | cattattggc atcattgacc ctgagtgccg gatgattggc ctgcgtctct atgatggcct |
| 661 | tttcaaggtt attccactag atcgcgataa taaagaactc aaggccttca acatccgcct |
| 721 | ggaggagctg catgtcattg atgtcaagtt cctatatggt tgccaagcac ctactatttg |
| 781 | ctttgtctac caggaccctc aggggcggca cgtaaaaacc tatgaggtgt ctctccgaga |
| 841 | aaaggaattc aataagggcc cttggaaaca ggaaaatgtc gaagctgaag cttccatggt |
| 901 | gatcgcagtc ccagagccct ttgggggggc catcatcatt ggacaggagt caatcaccta |
| 961 | tcacaatggt gacaaatacc tggctattgc ccctcctatc atcaagcaaa gcacgattgt |
| 1021 | gtgccacaat cgagtggacc ctaatggctc aagatacctg ctgggagaca tggaaggccg |
| 1081 | gctcttcatg ctgcttttgg agaaggagga acagatggat ggcaccgtca ctctcaagga |
| 1141 | tctccgtgta gaactccttg gagagacctc tattgctgag tgcttgacat accttgataa |
| 1201 | tggtgttgtg tttgtcgggt ctcgcctggg tgactcccag cttgtgaagc tcaacgttga |
| 1261 | cagtaatgaa caaggctcct atgtagtggc catggaaacc tttaccaact taggacccat |
| 1321 | tgtcgatatg tgcgtggtgg acctggagag gcaggggcag gggcagctgg tcacttgctc |
| 1381 | tggggctttc aaggaaggtt ctttgcggat catccggaat ggaattggaa tccacgagca |
| 1441 | tgccagcatt gacttaccag gcatcaaagg attatggcca ctgcggtctg accctaatcg |
| 1501 | tgagactgat gacactttgg tgctctcttt tgtgggccag acaagagttc tcatgttaaa |
| 1561 | tggagaggag gtagaagaaa ccgaactgat gggtttcgtg gatgatcagc agactttctt |
| 1621 | ctgtggcaac gtggctcatc agcagcttat ccagatcact tcagcatcgg tgaggttggt |
| 1681 | ctctcaagaa cccaaagctc tggtcagtga atggaaggag cctcaggcca agaacatcag |
| 1741 | tgtggcctcc tgcaatagca gccaggtggt ggtggctgta ggcagggccc tctactatct |
| 1801 | gcagatccat cctcaggagc tccggcagat cagccacaca gagatggaac atgaagtggc |
| 1861 | ttgcttggac atcaccccat taggagacag caatggactg tcccctcttt gtgccattgg |
| 1921 | cctctggacg gacatctcgg ctcgtatctt gaagttgccc tcttttgaac tactgcacaa |
| 1981 | ggagatgctg ggtggagaga tcattcctcg ctccatcctg atgaccacct ttgagagtag |
| 2041 | ccattacctc ctttgtgcct tgggagatgg agcgcttttc tactttgggc tcaacattga |
| 2101 | gacaggtctg ttgagcgacc gtaagaaggt gactttgggc acccagccca ccgtattgag |
| 2161 | gacttttcgt tctctttcta ccaccaacgt ctttgcttgt tctgaccgcc ccactgtcat |
| 2221 | ctatagcagc aaccacaaat tggtcttctc aaatgtcaac ctcaaggaag tgaactacat |
| 2281 | gtgtcccctc aattcagatg gctatcctga cagcctggcg ctggccaaca atagcaccct |
| 2341 | caccattggc accatcgatg agatccagaa gctgcacatt cgcacagttc ccctctatga |
| 2401 | gtctccaagg aagatctgct accaggaagt gtcccagtgt ttcggggtcc tctccagccg |
| 2461 | cattgaagtc caagacacga gtgggggcac gacagccttg aggcccagcg ctagcaccca |
| 2521 | ggctctgtcc agcagtgtaa gctccagcaa gctgttctcc agcagcactg ctcctcatga |
| 2581 | gacctccttt ggagaagagg tggaggtgca caacctactt atcattgacc aacacacctt |
| 2641 | tgaagtgctt catgcccacc agtttctgca gaatgaatat gccctcagtc tggtttcctg |
| 2701 | caagctgggc aaagacccca acacttactt cattgtgggc acagcaatgg tgtatcctga |
| 2761 | agaggcagag cccaagcagg gtcgcattgt ggtctttcag tattcggatg gaaaactaca |
| 2821 | gactgtggct gaaaaggaag tgaaaggggc cgtgtactct atggtggaat ttaacgggaa |
| 2881 | gctgttagcc agcatcaata gcacggtgcg gctctatgag tggacaacag agaaggagct |
| 2941 | gcgcactgag tgcaaccact acaacaacat catggccctc tacctgaaga ccaagggcga |
| 3001 | cttcatcctg gtgggcgacc ttatgcgctc agtgctgctg cttgcctaca agcccatgga |
| 3061 | aggaaacttt gaagagattg ctcgagactt taatcccaac tggatgagtg ctgtggaaat |
| 3121 | cttggatgat gacaattttc tgggggctga aaatgccttt aacttgtttg tgtgtcaaaa |
| 3181 | ggatagcgct gccaccactg acgaggagcg gcagcacctc caggaggttg gtcttttcca |
| 3241 | cctgggcgag tttgtcaatg tcttttgcca cggctctctg gtaatgcaga atctgggtga |
| 3301 | gacttccacc cccacacaag gctcggtgct cttcggcacg gtcaacggca tgatagggct |
| 3361 | ggtgacctca ctgtcagaga gctggtacaa cctcctgctg gacatgcaga atcgactcaa |
| 3421 | taaagtcatc aaaagtgtgg ggaagatcga gcactccttc tggagatcct ttcacaccga |
| 3481 | gcggaagaca gaaccagcca caggtttcat cgacggtgac ttgattgaga gtttcctgga |
| 3541 | tattagccgc cccaagatgc aggaggtggt ggcaaaccta cagtatgacg atggcagcgg |
| 3601 | tatgaagcga gaggccactg cagacgacct catcaaggtt gtggaggagc taactcggat |
| 3661 | ccattagcca agggcagggg gcccctttgc tgaccctccc caaaggcttt gccctgctgc |
| 3721 | cctccccctc ctctccacca tcgtcttctt ggccatggga ggcctttccc taagccagct |
| 3781 | gcccccagag ccacagttcc cctatgtgga agtggggcgg gcttcataga gacttgggaa |
| 3841 | tgagctgaag gtgaaacatt ttctccctgg atttttacca gtctcacatg attccagcca |
| 3901 | tcaccttaga ccaccaagcc ttgattggtg ttgccagttg tcctccttcc ggggaaggat |
| 3961 | tttgcagttc tttggctgaa aggaagctgt gcgtgtgtgt gtgtgtatgt gtgtgtgtgt |
| 4021 | atgtgtatct cacactcatg cattgtcctc tttttattta gattggcagt gtagggagtt |
| 4081 | gtgggtagtg gggaagaggg ttaggagggt ttcattgtct gtgaagtgag accttccttt |
| 4141 | tacttttctt ctattgcctc tgagagcatc aggcctagag gcctgactgc caagccatgg |
| 4201 | gtagcctggg tgtaaaacct ggagatggtg gatgatcccc acgccacagc ccttttgtct |
| 4261 | ctgcaaactg ccttcttcgg aaagaagaag gtgggaggat gtgaattgtt agtttctgag |
| 4321 | ttttaccaaa taaagtagaa tataagaaga aaggtaaaaa aaaaaaaaaa aa |

By “VHL polypeptide” or “von Hippel-Lindau” is meant a polypeptide or fragment thereof having at least 85% amino acid sequence identity to human VHL polypeptide of NCBI Accession No. NP_000542.1 and having E3 ligase activity. An exemplary human VHL polypeptide sequence is provided below:

(SEQ ID NO: 35)
MPRRAENWDE AEVGAEEAGV EEYGPEEDGG
EESGAEESGP EESGPEELGA EEEMEAGRPR
PVLRSVNSRE PSQVIFCNRS PRVVLPVWLN
FDGEPQPYPT LPPGTGRRIH SYRGHLWLFR
DAGTHDGLLV NQTELFVPSL NVDGQPIFAN
ITLPVYTLKE RCLQVVRSLV KPENYRRLDI
VRSLYEDLED HPNVQKDLER LTQERIAHQR
MGD

By “VHL polynucleotide” is meant a nucleic acid molecule encoding a human VHL polypeptide. An exemplary human VHL polynucleotide sequence is provided at NCBI Accession No. NM 000551.4, which is reproduced below:

| (SEQ ID NO: 36) | |
|---|---|
| 1 | gcagctccgc cccgcgtccg acccgcggat cccgcggcgt ccggcccggg tggtctggat |
| 61 | cgcggaggga atgccccgga gggcggagaa ctgggacgag gccgaggtag gcgcggagga |
| 121 | ggcaggcgtc gaagagtacg gccctgaaga agacggcggg gaggagtcgg gcgccgagga |
| 181 | gtccggcccg gaagagtccg gcccggagga actgggcgcc gaggaggaga tggaggccgg |
| 241 | gcggccgcgg cccgtgctgc gctcggtgaa ctcgcgcgag ccctcccagg tcatcttctg |
| 301 | caatcgcagt ccgcgcgtcg tgctgcccgt atggctcaac ttcgacggcg agccgcagcc |
| 361 | ctacccaacg ctgccgcctg gcacgggccg ccgcatccac agctaccgag gtcacctttg |
| 421 | gctcttcaga gatgcaggga cacacgatgg gcttctggtt aaccaaactg aattatttgt |
| 481 | gccatctctc aatgttgacg gacagcctat ttttgccaat atcacactgc cagtgtatac |
| 541 | tctgaaagag cgatgcctcc aggttgtccg gagcctagtc aagcctgaga attacaggag |
| 601 | actggacatc gtcaggtcgc tctacgaaga tctggaagac cacccaaatg tgcagaaaga |
| 661 | cctggagcgg ctgacacagg agcgcattgc acatcaacgg atgggagatt gaagatttct |
| 721 | gttgaaactt acactgtttc atctcagctt ttgatggtac tgatgagtct tgatctagat |
| 781 | acaggactgg ttccttcctt agtttcaaag tgtctcattc tcagagtaaa ataggcacca |
| 841 | ttgcttaaaa gaaagttaac tgacttcact aggcattgtg atgtttaggg gcaaacatca |
| 901 | caaaatgtaa tttaatgcct gcccattaga gaagtattta tcaggagaag gtggtggcat |
| 961 | ttttgcttcc tagtaagtca ggacagcttg tatgtaagga ggtttgtata agtaattcag |
| 1021 | tgggaattgc agcatatcgt ttaattttaa gaaggcattg gcatctgctt ttaatggatg |
| 1081 | tataatacat ccattctaca tccgtagcgg ttggtgactt gtctgcctcc tgctttggga |
| 1141 | agactgaggc atccgtgagg cagggacaag totttctcct ctttgagacc ccagtgcctg |
| 1201 | cacatcatga gccttcagtc agggtttgtc agaggaacaa accaggggac actttgttag |
| 1261 | aaagtgctta gaggttctgc ctctattttt gttggggggt gggagagggg accttaaaat |
| 1321 | gtgtacagtg aacaaatgtc ttaaagggaa tcatttttgt aggaagcatt ttttataatt |
| 1381 | ttctaagtcg tgcactttct cggtccactc ttgttgaagt gctgttttat tactgtttct |
| 1441 | aaactaggat tgacattcta cagttgtgat aatagcattt ttgtaacttg ccatccgcac |
| 1501 | agaaaatacg agaaaatctg catgtttgat tatagtatta atggacaaat aagtttttgc |
| 1561 | taaatgtgag tatttctgtt cctttttgta aatatgtgac attcctgatt gatttgggtt |
| 1621 | tttttgttgt tgttgttttg ttttgttttg tttttttgag atggagtctc actcttgtca |
| 1681 | cccaggctgg agtgcagtgg cgccatctcg gctcactgca acctctgcct cctgggttca |
| 1741 | cgtaatcctc ctgagtagct gggattacag gcgcctgcca ccacgctggc caatttttgt |
| 1801 | acttttagta gagacagtgt ttcgtcatgt tggccaggct ggtttcaaac tcctgacctc |
| 1861 | aggtgatccg cccacctcag cctcccaaaa tggtgggatt acaggtgtgt gggccaccgt |
| 1921 | gcctggctga ttcagcattt tttatcaggc aggaccaggt ggcacttcca cctccagcct |
| 1981 | ctggtcctac caatggattc atggagtagc ctggactgtt tcatagtttt ctaaatgtac |
| 2041 | aaattcttat aggctagact tagattcatt aactcaaatt caatgcttct atcagactca |
| 2101 | gttttttgta actaatagat ttttttttcc acttttgttc tactccttcc ctaatagctt |
| 2161 | tttaaaaaaa tctccccagt agagaaacat ttggaaaaga cagaaaacta aaaaggaaga |
| 2221 | aaaaagatcc ctattagata cacttcttaa atacaatcac attaacattt tgagctattt |
| 2281 | ccttccagcc tttttagggc agattttggt tggtttttac atagttgaga ttgtactgtt |
| 2341 | catacagttt tatacccttt ttcatttaac tttataactt aaatattgct ctatgttagt |
| 2401 | ataagctttt cacaaacatt agtatagtct cccttttata attaatgttt gtgggtattt |
| 2461 | cttggcatgc atctttaatt ccttatccta gcctttgggc acaattcctg tgctcaaaaa |
| 2521 | tgagagtgac ggctggcatg gtggctcccg cctgtaatcc cagtactttg gaaagccaag |
| 2581 | gtaagaggat tgcttgagcc cagaacttca agatgagcct gggctcatag tgagaaccca |
| 2641 | tctatacaaa aaatttttaa aaattagcat ggcggcacac atctgtaatc ctagctactt |
| 2701 | ggcaggctga ggtgagaaga tcattggagt ttaggaattg gaggctgcag tgagccatga |
| 2761 | gtatgccact gcactccagc ctgggggaca gagcaagacc ctgcctcaaa aaaaaaaaaa |
| 2821 | aaaaaaaaat caggccgggc atggtggctc acgcctgtaa tcccagcact ttgggaggtc |
| 2881 | gaggtgggca gatcacctga ggtcaggagt tcgagaccag cctggccaac atggtaaaac |
| 2941 | cccatttcta ctaaaaaata caagaattag ctgggtgtgg tggcgcatgc ctgtaatcct |
| 3001 | agctactcag gaggctgagg caggagaatc acttgaaccc aggaggcgaa gattgcagtg |
| 3061 | agctgatatc gcaccattgt actccagcct gtgtgacaga gcaatactct tgtctcaaaa |
| 3121 | aaaaaaaaaa attcaaatca gagtgaagtg aatgagacac tccagttttc cttctactcc |
| 3181 | gaatttcaac tgattttagc tcctcctttc aacattcaac aaatagtctt tttttttttt |
| 3241 | tttttttttt tttttttgag atggagtctc actctgttgc ccaggctgga gtgcagtggt |
| 3301 | gcgatctctg ctcactacaa gctctgcctc ccgagttcaa gtgattctcc tggctcaccc |
| 3361 | tcctgagtag ctgggattac aggcgcctgc caccatgcct ggctaatttt gtgtttttag |
| 3421 | tggagacggg gtttcaccat gttgtccagg atggtcttga tctcctgacc ttgtgatcca |
| 3481 | cccacctcag cctcccaaag tgctgggatt acaggtgtga gccaccgcgt ccagccagct |
| 3541 | ttattatttt ttttaagctg tctttgtgtc aaaatgatag ttcatgctcc tottgttaaa |
| 3601 | acctgcaggc cgagcacagt ggctcatgcc tgtaatccca gcattttggg agaccaaggc |
| 3661 | ggatggatca cctgaggtca ggagctgaag accagcctgg ctaacatggt gaaacctcat |
| 3721 | ctccacttaa aatacaaaaa ttgccggccg cggcggctca tgcctgtaat cccagcactt |
| 3781 | tgggaggcct aggcgggtgg atcacgaggt caggaaatcg agaccatcct ggctaacacg |
| 3841 | ggtgaaaccc cgtctctatt aaaaaataga aaaaattagg cgggcgtggt ggtgagcgcc |
| 3901 | tgtagtccca gctactcgag agcctgaggc aggagaatgg catgaacctg gaaggcggag |
| 3961 | cttgcagtga gctgagatgg tgccactgca ctctaacctg ggcgacagag tgagacaccg |
| 4021 | tctcaaaaaa aaaaacaaaa aacaaaaatt atccaggtgt ggcggtgggc gcctgtgagg |
| 4081 | caggcgaatc tcttgaaccc gggaggcgga ggttgcagtg agccaagatc acaccattgc |
| 4141 | actccagcct gggcaacaag agtgaaattc catctcaaaa agaaaccaaa aaaacaaaaa |
| 4201 | aaaaacatgc cgtttgagta ctgtgttttt ggtgttgtcc aaggaaaatt aaaaacctgt |
| 4261 | agcatgaata atgtttgttt ttcatttcga atcttgtgaa tgtattaaat atatcgctct |
| 4321 | taagagacgg tgaagttcct atttcaagtt tttttttttt tttttttttt taaagctgtt |
| 4381 | ttttaataca ttaaatggtg ctgagtaaag gaaa |

The terms “conjugating” or “to conjugate” or “conjugation” refer to the process of linking, connecting, coupling, fusing, associating, or any combination thereof, two or more entities, such as protein or protein fragments, to form a larger entity. In an 50 embodiment, conjugating is via a non-covalent interaction. In an embodiment, conjugating is via a covalent interaction.

“Detect” refers to identifying the presence, absence or amount of the analyte to be detected. In particular embodiments, the sequence of a polynucleotide of a gene in Table 1 is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, pathology, or disorder that damages or interferes with the normal function of a cell, tissue, organ, or organism. Examples of diseases include autoimmune diseases and cancers, such as a B cell neoplasia or malignancies, for example, plasma cell malignancy, multiple myeloma or a myelodysplastic syndrome, erythema nodosum leparum, 5q-myelodysplastic syndrome. Cancers, tumors, or neoplasias may include, without limitation, a breast, ovarian, cervical, prostate, testis, lung, liver, bladder, lymphocytic, brain, nervous system, or immune system, cardiac, colon, kidney, gall bladder, pancreas cancer, tumor, or neoplasia.

By “effective amount” is meant the amount of a required to ameliorate the symptoms of a disease relative to an untreated patient. The effective amount of active compound(s) used to practice aspects and embodiments for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “E2 ubiquitin conjugating enzyme,” “E2 ubiquitin-like conjugating enzyme,” “E2 protein,” “ubiquitin-like conjugating enzyme,” and “ubiquitin conjugating enzyme,” which may be used interchangeably, to refer to enzymes that interact with ubiquitin or a ubiquitin-like protein and an E3 protein in the processes of ubiquitination or neddylation. These terms also refer to naturally-occurring variants of a given E2 protein and recombinantly prepared variants, or functional fragments thereof. Non-limiting examples of ubiquitin-like proteins include NEDD8 (NCBI Reference Sequence: NP_006147.1), NEDD8 E2 (e.g., Ubcl2), SUMO-activating enzyme subunit 1 (UniProtKB/Swiss-Prot Reference No.: Q9UBE0.1), SUMO-activating enzyme subunit 2 (NCBI Reference Sequence: NP_005490.1), SUMO E2, ISG15 (Ubiquitin-like protein ISG15, UniProtKB/Swiss-Prot Reference No.: P05161.5), ISG15 E2, ATG8 (ubiquitin-like-conjugating enzyme ATG3 isoform 1, NCBI Reference Sequence: NP_071933.2), ATG8 (ubiquitin-like-conjugating enzyme ATG3 isoform 2, NCBI Reference Sequence: NP_001265641.1), ATG8 E2, ATG12 (Ubiquitin-like protein ATG12 isoform 1, NCBI Reference Sequence: NP_004698.3), ATG12 (Ubiquitin-like protein ATG12 isoform 2, NCBI Reference Sequence: NP_001264712.1), ATG12 E2, and FAT10 (Ubiquitin D or Ubiquitin-like protein FAT10, UniProtKB/Swiss-Prot Reference No.: 015205.2 and GenBank Reference No.: AAD52982.1), FAT10 E2.

The terms “E3 ubiquitin ligase,” “E3 ligase,” “E3 protein”, “ubiquitin-protein ligase” and “ubiquitin ligase” are used interchangeably herein to refer to ubiquitin E3 ligases, which are enzymes that mediate the covalent attachment of ubiquitin or a ubiquitin-like protein to a substrate (e.g., a substrate protein) to produce a ubiquitinated substrate. These terms also refer to naturally-occurring variants of a given E3 ubiquitin ligase. Non-limiting examples of E3 proteins include RING ligase; HECT; RBR; XIAP; cIAPl, human ubiquitin-protein ligase gp78, also known as autocrine motility factor receptor, isoform 2; yeast ubiquitin-protein ligase DoalO; human ubiquitin-protein ligase RNF13; human ubiquitin-protein ligase RNF38; human ubiquitin-protein ligase TEB4; human ubiquitin-protein ligase RNF139 also known as trc8; human ubiquitin-protein ligase RNFx2; human ubiquitin-protein ligase RNF126; human ubiquitin-protein ligase Hrdl; and human ubiquitin-protein ligase MARCH 1.

A “modified E3 ligase” is a ligase modified from its naturally occurring form, for example, to prevent the E3 ligase from binding to a ubiquitin E2 ligase. A non-limiting example of a modified E3 ligase is an E3 ligase comprising a substrate binding domain and flexible linker, e.g., a Gly-Gly-Ser-Gly linker (SEQ ID NO: 37), and absent any portion of a RING domain, that is able to bind to an E2 ubiquitin-like conjugating enzyme.

A “cullin-RING ligase” (CRL) refers to a multisubunit complex composed of a cullin, a RING H2 finger protein, a variable substrate-recognition subunit (SRS), and for most CRLs, an adaptor that links the SRS to the complex. CRLs comprise the largest known category of ubiquitin ligases and are activated by the covalent attachment of the ubiquitin-like protein Nedd8 to the cullin; CRLs are inhibited by binding to the CANDI inhibitor. (Bosu, D. R. et al., 2008, Cell Division, 3:7; doi:10.1186/1747-1028-3-7). CRLs regulate diverse cellular processes, including multiple aspects of the cell cycle, transcription, signal transduction, and development.

An expression control sequence refers to a nucleotide sequence that regulates the transcription or translation of a polynucleotide or the localization of a polypeptide to which it is operatively linked. Expression control sequences are “operatively linked” when the expression control sequence controls or regulates the transcription and, as appropriate, the translation of the nucleotide sequence (e.g., a transcription or translation regulatory element, respectively), or localization of an encoded polypeptide to a specific compartment of a cell. Thus, an expression control sequence can be, without wishing to be limiting, a promoter, enhancer, transcription terminator, a start codon (ATG), a splicing signal for intron excision and maintenance of the correct reading frame, a STOP codon, or a ribosome binding site. An expression control sequence can also be a sequence that targets a polypeptide to a particular location, for example, a cell compartmentalization signal, which can target a polypeptide to the cytosol, nucleus, plasma membrane, endoplasmic reticulum, mitochondrial membrane or matrix, chloroplast membrane or lumen, medial trans-Golgi cistemae, or a lysosome or endosome. Cell compartmentalization domains are well known in the art and include, for example, a peptide containing amino acid residues 1 to 81 of human type II membrane-anchored protein galactosyltransferase, or amino acid residues 1 to 12 of the presequence of subunit IV of cytochrome c oxidase (Hancock et al., 1991, EMBO J., 10:4033-4039; Buss et al., 1988, Mol. Cell. Biol., 8:3960-3963; and U.S. Pat. No. 5,776,689; incorporated by reference herein.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “fusion” or “fusing” and grammatical variants thereof, when used in reference to a composition such as a protein or protein fragment, refers to the assembly, joining, or coupling of two or more proteins, protein regions, domains, or fragments thereof, to generate a fused molecule, e.g., one having discrete components. Fusions can be created by several means, e.g., chemical fusions, achieved by coupling, conjugation or cross-linking, either directly or through an intermediate structure; physical fusions, achieved by coupling through capture in or on a macromolecular structure; or molecular biological (recombinantly produced) fusion, achieved through the molecular combination of recombinant nucleic acid molecules that comprise nucleic acid molecules or fragments thereof capable of encoding the fusion components, such that a single continuous expression product is produced. An exemplary fusion includes, but is not limited to, a component of the system described herein in which an E3 ligase or the substrate binding domain of an E3 ligase is fused to an E. coli biotin ligase BirA. In an embodiment, the E3 ligase is fused to a non-promiscuous, E. coli wild type BirA biotin ligase. In some embodiments, one or more spacer or linker sequences can be used to couple or join the components of the fusion. The term “fused” means to create a fusion protein as mentioned above. The term “fusion protein” used herein relates to a protein construct, e.g., a non-naturally occurring or artificial construct. A fusion protein may comprise at least two heterologous amino acid sequences that are defined by their origin and/or by special functions. In embodiments of the system described herein, a ubiquitin protein fused to a peptide substrate of BirA biotin ligase (e.g., A3-tag or AVI-TAG™ and the like), and an E3 ligase fused to a BirA biotin ligase (e.g., non-promiscuous, E. coli wild type BirA biotin ligase) are fusion proteins. In addition, a fusion protein can refer to fusion proteins that also contain non-protein molecule components, such as nucleic acids, sugars, or markers for radioactive, chemiluminescent, electro-chemiluminescent, enzyme, or fluorescent labeling and/or detection.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid (polynucleotide, e.g., DNA, RNA, mRNA), polypeptide, or peptide as described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA or RNA) that is free of the genes which, in the naturally-occurring genome of the organism from which the nucleic acid molecule as described herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA (e.g., mRNA) molecule that is transcribed from a DNA molecule, as well as a recombinant DNA that is part of a hybrid gene encoding additional polypeptide sequence.

By an “isolated polypeptide” is meant a polypeptide that has been separated from components that naturally accompany it. Typically, the polypeptide is isolated when it is at least 60%, by weight, free from the proteins and naturally-occurring organic molecules with which it is naturally associated. Alternatively, the preparation is at least 75%, or at least 90%, or at least 99%, by weight, a polypeptide as described herein. An isolated polypeptide such as described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “mass spectrometry” or “MS” refers to an analytical technique to identify compounds by their mass. MS refers to methods of filtering, detecting, and measuring charged particles (ions) based on their mass-to-charge ratio, or “m/z”. Although there are many different kinds of mass spectrometers, all of them make use of electric or magnetic fields to manipulate the motion of ions produced from an analyte of interest and determine their m/z. The basic components of a mass spectrometer are the ion source, the mass analyzer, the detector, and the data and vacuum systems. The ion source is where the components of a sample introduced in a MS system are ionized by means of electron beams, photon beams (UV lights), laser beams, or corona discharge. The ion source converts and fragments the neutral sample molecules into gas-phase ions that are sent to the mass analyzer. The mass analyzer applies the electric and magnetic fields to sort the ions by their masses, and the detector measures and amplifies the ion current to calculate the abundances of each mass-resolved ion. To generate a mass spectrum that a human eye can easily recognize, the data system records, processes, stores, and displays data in a computer.

In general, one or more molecules of interest is ionized, and the ions are subsequently introduced into a mass spectrographic instrument where, using a combination of magnetic and electric fields, the ions follow a path in space that is dependent upon mass (“m”) and charge (“z”). See, e.g., U.S. Pat. Nos. 6.204,500, entitled “Mass Spectrometry From Surfaces;” 6,107,623, entitled “Methods and Apparatus for Tandem Mass Spectrometry;” 6,268,144, entitled “DNA Diagnostics Based On Mass Spectrometry;” 6,124,137, entitled “Surface-Enhanced Photolabile Attachment And Release For Desorption And Detection Of Analytes;” Wright et al, 1999, Prostate Cancer and Prostatic Diseases 2:264-76; and Merchant and Weinberger, 2000, Electrophoresis 21(1) 164-67.

The mass spectrum can be used to determine the mass of the analytes, their elemental and isotopic composition, or to elucidate the chemical structure of the sample. MS involves a process that occurs in gas phase and under vacuum (e.g., 1.33*10⁻² to 1.33*10⁻⁶ pascal). Devices have been developed and are used to facilitate the transition from samples at higher pressure and in condensed phase (solid or liquid) into a vacuum system, thus providing MS as a useful tool for identification and quantification of organic compounds like peptides. MS is routinely used in analytical laboratories to study physical, chemical, or biological properties of a great variety of compounds. Among the various kinds of mass analyzers, those that find application in LC-MS systems are the quadrupole, time-of-flight (TOF), ion traps, and hybrid quadrupole-TOF (QTOF) analyzers. Other non-limiting examples of types of mass spectrometry include SILAC mass spectrometry and liquid chromatography-tandem mass spectrometry (LC/MS/MS). SILAC (“Stable Isotope Labeling by/with Amino acids in Cell culture”) mass spectrometry detects differences in protein abundance among samples using non-radioactive isotopic labeling, especially for performing quantitative proteomics (G. Zhang et al., 2009, Methods Mol. Biol., 527:79-89. SILAC MS uses in vivo metabolic incorporation of “heavy” 13C- or 15N-labeled amino acids into proteins followed by mass spectrometry (MS) analysis for accelerated identification, characterization and quantification of proteins.

Liquid chromatography-mass spectrometry (LC-MS) is an analytical chemistry technique that combines the physical separation capabilities of liquid chromatography (or HPLC) with the mass analysis capabilities of mass spectrometry (MS). Coupling chromatography and MS systems is useful in chemical analyses because the individual capabilities of each technique are enhanced synergistically. While liquid chromatography separates mixtures with multiple components, mass spectrometry provides structural identity of the individual components with high molecular specificity and detection sensitivity. The tandem technique of LC-MS can be used to analyze biochemical, organic, and inorganic compounds commonly found in complex environmental and biological samples. In addition to the liquid chromatography and mass spectrometry devices, an LC-MS system contains an interface that efficiently transfers the separated components from the LC column into the MS ion source. The interface allows for compatibility between the LC and MS devices. While the mobile phase in a LC system is a pressurized liquid, the MS analyzers commonly operate under high vacuum (around 10⁻⁶ Torr/10⁻⁷ Hg). Therefore, it is not possible to directly pump the eluate from the LC column into the MS source. Overall, the interface is a mechanically simple part of the LC-MS system that transfers the maximum amount of analyte, removes a significant portion of the mobile phase used in LC, and preserves the chemical identity of the chromatography products (chemically inert). As a requirement, the interface should not interfere with the ionizing efficiency and vacuum conditions of the MS system. At present, the most extensively applied LC-MS interfaces are based on atmospheric pressure ionization (API) strategies, such as electrospray ionization (ESI), atmospheric-pressure chemical ionization (APCI), and atmospheric pressure photoionization (APPI).

LC-MS provides fast molecular weight confirmation and structure identification.

The term “modify” or “modifying” and grammatical variations thereof, when used in reference to a protein (polypeptide) or fragment thereof. e.g., a peptide, refers to a protein (polypeptide) or fragment thereof that deviates from a reference, such as an unmodified or wildtype protein (polypeptide) or fragment thereof. Modifying can include the addition or removal of protein domains to assist in the construction of a catalytic tagging system. e.g., removing the RING domain of an E3 ligase that under wild-type conditions would be necessary to bind to a ubiquitin E2 ligase to permit the remaining portions of the E3 ligase, absent the RING domain, to bind to a ubiquitin-like E2 ligase other than a ubiquitin-like E2 ligase, for example, a NEDD8 E2 ligase.

By “modulator of CRBN” or “modulator of Cereblon” is meant any agent which binds Cereblon (CRBN), an E3 ligase, and alters an activity of CRBN. In some embodiments, an activity of CRBN includes binding with and/or mediating degradation of one or more substrate proteins, such as Ikaros (IKZF1), Aiolos (IKZF3), Casein kinase 1 Alpha (CSNK1a1), GSPT1, or GSPT2. Thus, a modulator of CRBN includes agents that alter binding of CRBN with IKZF1, IKZF3, CSNK1a1, GSPT1, or GSPT2 and agents that alter CRBN's mediation of IKZF1, IKZF3, CSNK1a1, GSPT1, or GSPT2 degradation. In certain embodiments, a modulator of CRBN is lenalidomide or an analog thereof, e.g., pomalidomide or thalidomide. In another embodiment, the modulator of CRBN is CC-885, which recruits the E3 ligase substrate GSPT1 or GSPT2, a translation termination factor. (M. Matyskiela et al., 2016, Nature, 535(7611):252-257. doi: 10.1038/nature18611). In some embodiments, a CRBN substrate, e.g., IKZF1, may be fused or conjugated to a tag or marker protein, such as V5, as described herein.

The term “kinase” refers to any enzyme that catalyzes the addition of phosphate groups to an amino acid residue in a protein, polypeptide, or peptide; for example, serine and threonine kinases catalyze the addition of phosphate groups to serine and threonine amino acid residues in proteins, polypeptides, or peptides.

A “mutant” or “variant” polypeptide or protein refers to a polypeptide or protein that differs from the wild type (e.g., normal, nonmutated) polypeptide or protein by one or more new or different characteristics or functions caused by any change or alteration in the polynucleotide sequence or gene (DNA) encoding the polypeptide or protein. A change or alteration may be a substitution, addition (insertion), duplication, inversion, or deletion of a nucleotide base or sequence of bases, particularly, in the coding sequence of a polynucleotide or gene, which may result from a number of different causes. e.g., errors in DNA replication, exposure to DNA-damaging agents, environmental factors, e.g., ultraviolet radiation or x-ray exposure, mutagens, chemical exposure, genetic insults, and the like. Such a change or alteration in the encoding polynucleotide or gene can result in a change in function or activity of the encoded protein or polypeptide, and/or a disruption in normal gene or protein activity. By way of example, a loss-of-function mutation refers to a change or alteration in a protein or gene product and/or its encoding amino acid sequence that causes the protein to have reduced, decreased, or diminished activity or function. In an embodiment, the loss of function mutant or variant protein or product does not have the function or activity, or the level of function or activity, of the wild-type, nonmutated protein.

By a “non-promiscuous,” enzyme, such as a non-promiscuous E. coli wild type BirA biotin ligase, is meant that the enzyme exhibits specific recognition and activity for a given substrate. The non-promiscuous, wild-type BirA biotin ligase has a high specificity for its substrate.

Nucleic acid (polynucleotide) molecules useful in the methods described herein include any nucleic acid molecule that encodes a polypeptide as described herein, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those of ordinary skill in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those of ordinary skill in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those of ordinary skill in the art. Hybridization techniques are well known to those of ordinary skill in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The terms “nucleic acid” and “polynucleotide” refer to deoxyribonucleotides or ribonucleotides and polymers thereof in either single- or double-stranded form, as well as complements thereof. The terms embrace various forms of nucleic acids, for example, gene, pre-mRNA, mRNA, and their polymorphic variants, alleles, mutants, and interspecies homologs. The term nucleic acid is used interchangeably with gene, DNA, cDNA, RNA, mRNA, oligonucleotide, and polynucleotide, and encompasses nucleic acids that are naturally occurring or recombinant.

The term “operatively linked” or “operably linked” or “operatively coupled” or “operatively joined” and the like, when used to describe chimeric (e.g., fusion) proteins, refers to polypeptide sequences that are placed in a physical and functional relationship to each other. In an embodiment, the functions of the polypeptide components of the chimeric or fusion protein are unchanged compared to the functional activities of the parts in isolation. In embodiments, fusion proteins can be monomeric or multimeric (e.g., dimeric).

A polynucleotide encoding a polypeptide means that, upon transcription of the polynucleotide and translation of the mRNA resulting from transcription, a polypeptide is produced. The encoding polynucleotide is considered to include both the coding strand, whose nucleotide sequence is identical to an mRNA, as well as its complementary strand. An encoding polynucleotide may include degenerate nucleotide sequences or codons, which encode the same amino acid residues. Nucleotide sequences encoding a polypeptide can include polynucleotides containing introns (noncoding) as well as the encoding exons.

As used herein, “obtaining” as in “obtaining an agent” includes synthesizing, deriving, producing, purchasing, or otherwise acquiring the agent.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “phosphatase” refers to an enzyme that is capable of removing a phosphate group from a protein, polypeptide, or peptide, e.g., by hydrolysis.

As used herein, the terms “prevent,” “preventing,” “prevention,” “prophylactic treatment” and the like refer to reducing the probability of developing a disorder or condition in a subject who does not have, but is at risk of having, or is susceptible to developing, a disorder, disease, pathology, or condition.

The term “recombinant” used in reference to a cell, nucleic acid (polynucleotide), protein (polypeptide), or vector, for example, indicates that the cell, nucleic acid (polynucleotide), protein (polypeptide), or vector has been modified by the introduction of a heterologous nucleic acid (polynucleotide) or protein (polypeptide), or the alteration of a native nucleic acid (polynucleotide) or protein (polypeptide), or that the cell is derived from a cell so modified. By way of example, recombinant cells express polynucleotides (genes) that are not found within the native (non-recombinant) form of the cell, or they express native polynucleotides (genes) that are otherwise abnormally expressed, or are under-expressed or not expressed at all. A polynucleotide can be, without limitation, DNA, cDNA, RNA, mRNA, and the like).

The term “recombinant nucleic acid molecule” refers to a non-naturally occurring nucleic acid molecule containing two or more linked polynucleotide sequences. A recombinant nucleic acid molecule can be produced by known recombination methods, particularly genetic engineering techniques, or can be produced by a chemical synthesis method. A recombinant nucleic acid molecule can encode a fusion protein, for example, an E3 ligase fused to (tagged with) a non-promiscuous and/or E. coli wild-type BirA biotin ligase as described herein. The term “recombinant host cell” refers to a cell that contains a recombinant nucleic acid molecule. As such, a recombinant host cell can express a polypeptide from a “gene” that is not found within the native (non-recombinant) form of the cell.

The term “heterologous” when used with reference to portions of a nucleic acid molecule indicates that the nucleic acid molecule comprises two or more sequences or subsequences that are not found in the same relationship to each other in nature. For instance, the nucleic acid molecule is typically recombinantly produced and has two or more polynucleotide sequences (e.g., from unrelated genes) arranged to generate a new functional nucleic acid, e.g., a promoter from one source and a coding region from another source. Similarly, a heterologous protein indicates that the protein comprises two or more amino acid sequences or subsequences that are not found in the same relationship to each other in nature (e.g., a fusion protein). Similarly, a heterologous protein may refer to a protein which comprises two or more amino acid sequences or subsequences, e.g., comprising portions, components, fragments or sections of the polypeptide or protein, that are not found in the same relationship with each other in nature. By way of example, a chimeric protein contains heterologous components, which together, generate a new functional polypeptide or protein.

“Molecular glues” refer to small molecules that interact with two protein surfaces to induce or enhance affinity of the two proteins to each other. (See, e.g., T. M. Geiger et al., 2022, Curr. Res. Chem. Biol., Vol. 2, 1000018, and examples of molecular glues therein). As a general feature, molecular glues act on a protein-of-interest (POI or target protein) together with an accessory protein (AP), substrate adaptor protein or presenter protein or effector protein. If the accessory protein forms favorable direct contacts with the POI or target protein (cooperative binding), the enlarged composite contact surface allows binding to shallow protein surfaces in target proteins that would otherwise be inaccessible by classical small molecule drugs. Molecular glue degraders are small, drug-like compounds that induce interactions between an E3 ubiquitin ligase and a target (e.g., target protein); the interactions result in ubiquitination and subsequent degradation of the recruited protein. Modifications of the protein surface by a bound small molecule can change the interactome of the target protein. By inducing interactions between a ligase and a substrate, molecular glues stabilize novel protein-protein interactions between a ubiquitin ligase and proteins not otherwise targeted by the ligase, thereby leading to neo-substrate degradation. While molecular glues generally display low binding affinities for either the E3 ligase or the POI or target protein, they enhance such protein-protein interactions enabling engagement of the POI or target protein with the E3 ligase and the subsequent polyubiquitination and degradation of the POI or target molecule. Molecular glues are unidirectional molecules that act to bridge interactions between the target protein and the ligase substrate receptor (See. e.g., T. B. Faust et al., 2021, Annu. Rev. Cancer Biol., 5:181-201; S. Rana et al., 2021, Cancers (Basel), 13(21):5506). A nonlimiting example of a biological molecular glue includes the plant hormone auxin, which binds in a hydrophobic pocket of the TIR1 receptor. In some cases, a molecular glue comprises an IMiD. A nonlimiting example of a synthetic molecular glue includes lenalidomide and analogs thereof, which bind the substrate receptor CRBN to recruit CK1α through its 0-hairpin loop (PDB ID 5fqd). CK1α Gly40 packs between lenalidomide and CRBN Val388.

A “PROTAC®” (i.e., “proteolysis targeting chimera”) is a heterobifunctional small molecule or compound composed of a ligand that binds to a target protein or protein of interest (POI) to be degraded and a ligand that binds an E3 ubiquitin ligase (and its associated proteins). A proteolysis targeting chimera (PROTAC®) can also be referred to as a protein or polypeptide “degrader.” The two elements are joined by a linker molecule. Upon engagement of the POI with the E3 ligase complex to form a ternary complex, the target molecule or POI may be polyubiquitinated if it is appropriately positioned relative to the E3 ligase complex. The target molecule or POI is then degraded by the proteasome complex. Since the PROTAC® can be recycled to repeat this process, it is therefore “event driven” with the PROTAC® having a catalytic mechanism of action (MOA). (See. e.g., S. B. Alabi et al., 2021, J. Biol. Chem. Reviews; 296:100467; X. Sun et al., 2019, Nature, Signal Transduction and Targeted Therapy Review, 4:64). A PROTAC® protein degrader acts, in essence, to hijack the intrinsic catalytic activity of the E3 ubiquitin ligase and direct it toward the protein of interest (POI) as a neo-substrate, thus triggering its poly-ubiquitination and subsequent proteasome-dependent degradation. In embodiments, PROTAC®s that recruit a number of different types of E3 ligases, including, for example, CRBN, VHL, and those involved in essential pathways, may aid in bypassing potential resistance mechanisms and expand the range of viable target proteins. As will be appreciated by the skilled practitioner in the art, while PROTAC®s are bifunctional molecules, namely, chimeras, comprised of two (functional) moieties connected via a linker, molecular glues are small molecules that act as adhesives which enhance binding between two proteins that typically would not interact, e.g., by creating a new molecular surface by first binding to one of the proteins, such that the new molecular surface is complementary to the surface on the second protein.

A “pulldown” assay refers to an in vitro technique used to detect or determine a physical interaction between two or more proteins. In the case of the natural ligand-ligand (or protein binding partner) interaction between biotin and avidin/streptavidin, pulldown assays utilize protein (such as avidin or streptavidin) bound to beads, often in a column, that specifically bind to the cognate, protein-binding partner (such as biotin, which is stable and small and rarely interferes with the function of labeled molecules). The technique can be used to determine, identify, or verify a protein interaction via Western blot or to identify protein interactions using a total protein stain. In brief, pulldown assays are a form of affinity purification or chromatography. In a pulldown assay, a biotinylated substrate protein (in a cell lysate) is captured on the streptavidin/avidin affinity ligand immobilized on the support and specific for biotin, Avidin, streptavidin, or NeutrAvidin proteins can bind up to four biotin molecules, which are normally conjugated to an enzyme or target protein to form an Avidin-biotin complex. The avidin-biotin complex is the strongest known non-covalent interaction (K_{d}=10⁻¹⁵ M) between a protein and ligand. The bond formation between biotin and avidin is very rapid, and once formed, is unaffected by extremes of pH, temperature, organic solvents and other denaturing agents. The method of protein elution depends on the affinity ligand and ranges from using competitive analytes to low pH or reducing buffers.

By “reduces” is meant a negative alteration of at least 5%, 10%, 25%, 50%, 75%, or 100%.

By “reference” is meant a standard or control condition. A reference may be a normal or healthy, or non-diseased sample, subject, or condition against which a test or experimental sample and the like is compared, evaluated, measured/quantified, or assessed. A reference is generally the same type of biological entity, e.g., a cell, sample, subject, or condition, but that has not undergone an experimental treatment, procedure, and the like, or has not been affected in the same way, e.g., a placebo or normal physiological condition, as a test sample, subject, condition, and the like. In some cases, a reference may be a sample obtained from another subject having the same condition or a worse condition to serve as a comparison.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about or equal to a given number of amino acids, for example, at least about 16 amino acids, or at least about 20 amino acids, or at least about 25 amino acids, or about 35 amino acids, about 50 amino acids, or about 100 amino acids. For nucleic acids, the length of the reference nucleic acid sequence will generally be at least about or equal to a given number of nucleotides, for example, at least about 50 nucleotides, or at least about 60 nucleotides, or at least about 75 nucleotides, or about 100 nucleotides or about 300 nucleotides or any integer thereabout or therebetween.

By “specifically binds” is meant an agent, compound, or antibody that recognizes and binds a polypeptide (or polynucleotide), but which does not substantially recognize and bind other molecules in a sample, for example, a biological sample, which naturally includes a polypeptide (or polynucleotide) such as described herein.

The term “small molecule” refers to a non-peptidic, non-oligomeric, organic (chemical) compound either synthesized in the laboratory or found in nature. Small molecules can refer to compounds that are “natural product-like;” however, the term “small molecule” is not limited to “natural product-like” compounds. Without intending to be limiting, a small molecule is typically characterized by containing several carbon-carbon bonds and having a molecular weight of less than 1500. Non limiting examples of small molecules that can be used in accordance with the embodiment described herein are dasatinib, imatinib, nilotinib, thalidomide, lenalidomide, and pomalidomide.

The term “substrate” refers to any molecule, e.g., a protein, polypeptide, or peptide substrate, that is capable of being conjugated or associated with ubiquitin or a ubiquitin-like protein. The substrate can be any naturally occurring or synthetic substrate.

The term “substrate binding domain” refers to an E3 ligase domain that is capable of binding to a substrate that can be bound to ubiquitin or ubiquitin-like proteins (See, e.g., Liu et al., 2010, J. Mol. Biol., 395: 1508-23).

The term “stable transfection” refers to the permanent expression of a gene of interest, e.g., introduced into a cell by a plasmid or vector, through the integration of the transfected polynucleotide (e.g., DNA) into the genomic DNA of a cell, or to the maintenance of transfected plasmid as an extra chromosomal replicating episome within a cell. In stable transfection, introduced genetic material that is integrated into the cellular genome will be passed on to future generations of the cell.

The term “transient transfection” refers to the introduction of a polynucleotide (e.g., DNA or gene) into a cell for a limited period of time, as the polynucleotide is not integrated into the cellular genome. In some cases, the high copy number of the transfected polynucleotide leads to high levels of expressed protein within the period of time that the polynucleotide is present in the cell.

As used herein, the terms “treat,” “treating,” “treatment,” and the like refer to reducing, alleviating, diminishing, abating, ameliorating, or eradicating a disease, disorder, pathology, or condition, and/or symptoms associated therewith. It will be appreciated that, although not precluded, treating a disease, disorder, pathology, or condition does not require that the disease, disorder, pathology, or condition, or the symptoms associated therewith, be completely eliminated.

Ubiquitin is a small (8.6 kDa; 76 amino acids) regulatory protein found in most tissues of eukaryotic organisms. It is a posttranslational protein modifier, which forms an isopeptide bond between a lysine residue on a protein and the carboxyl terminus of ubiquitin. Four genes in the human genome code for ubiquitin: UBB and UBC, which code for polyubiquitin precursor proteins, and UBA52 and RPS27A, which code for a single copy of ubiquitin fused to the ribosomal proteins L40 and S27a, respectively. The amino acid sequence of ubiquitin is as follows:

(SEQ ID NO: 38)
MQIFVKTLTGKTITLEVEPSDTIENVKAKIQ
DKEGIPPDQQRLIFAGKQLEDGRTLSDYNIQ
KESTLHLVLRLRGG

The ubiquitin protein includes seven lysine (K) residues as shown in bold in the above sequence. In embodiments, a sequence having at least 85%, at least 90%, at least 93%, at least 95%, at least 98%, or greater, amino acid sequence identity to the above ubiquitin sequence is encompassed.

“Ubiquitination” or “Ubiquitylation” or “Ubiquitinylation” (all of which are interchangeable terms) refers to the addition of ubiquitin to a substrate protein. Ubiquitination can mark proteins for degradation via the proteasome, alter their cellular location, affect their activity, and promote or prevent protein interactions. Ubiquitination via the “ubiquitin-proteosome system” involves three main steps: activation, conjugation, and ligation, performed by ubiquitin-activating enzymes (E1s), ubiquitin-conjugating enzymes (E2s), and ubiquitin ligases (E3s), respectively. In brief, the ubiquitin-proteosome system includes an E1 activating enzyme that conjugates to an E2 enzyme, transferring a ubiquitin molecule to E2, E2 then binds to an E3 ubiquitin ligase in a complex that can then recognize target proteins (substrates) for subsequent ubiquitin tagging and degradation by the 26S proteosome in the cell. The result of the function of this sequential enzyme cascade is to bind ubiquitin to lysine (K) residues on a protein substrate via an iso-peptide bond, or cysteine residues through a thioester bond, or serine and threonine residues through an ester bond, or the amino group of the protein's N-terminus via a peptide bond.

https://en.wikipedia.org/wiki/Ubiquitin-cite_note-pmid15571809-7 Ubiquitin modification(s) of the substrate protein can be by the attachment of a single ubiquitin protein (mono-ubiquitination), by single ubiquitins on various lysines scattered over the substrate (multiubiquitination) or by ubiquitin chains on one or several lysines (polyubiquitination). Different ubiquitin chains (lysine 6, 11, 27, 29, 33, 48, 63 of ubiquitin) form various conformations). (K. Bielskiene et al., 2015, Medicina, Vol. 51(1):1-9). Secondary ubiquitin molecules can be linked to one of the seven lysine (K) residues or the N-terminal methionine (M) of the previous ubiquitin molecule. These ‘linking’ residues are represented by “K” or “M” and a number that refers to its position in the ubiquitin molecule, as in K48, K29 or M1. The first ubiquitin molecule is covalently bound through its C-terminal carboxylate group to a particular lysine, cysteine, serine, threonine or N-terminus of the target protein. Polyubiquitination occurs when the C-terminus of another ubiquitin is linked to one of the seven lysine residues or the first methionine on the previously-added ubiquitin molecule, thus creating a chain of attached ubiquitin molecules. This process repeats several times, leading to the addition of several ubiquitin proteins. Polyubiquitination on defined lysine residues, primarily on K48 and K29, is related to degradation by the proteasome, while other polyubiquitinations (e.g., on K63, K11, K6 and M1) and mono-ubiquitinations may regulate processes such as endocytic trafficking, inflammation, translation and DNA repair.

The term “ubiquitin-like protein” (UBL) refers to a protein that has a high degree of similarity, identity, or homology to ubiquitin and that can be covalently conjugated to its substrate proteins through lysines. By way of non-limiting example, ubiquitin-like proteins include NEDD8, SUMO, ISG15, ATG8, ATG12, and FAT10, or functional analogs thereof. In embodiments, a ubiquitin-like protein has an amino acid sequence with at least 80%, at least 85%, at least 90%, at least 93%, at least 95%, at least 98%, or greater, sequence identity to the ubiquitin amino acid sequence.

The term “wild-type conditions” as used herein refers to conditions under which a naturally occurring E3 ligase is able to carry out its normal ligase functions, such as binding an E2 ubiquitin conjugating enzyme. Non wild-type conditions may include abnormal or aberrant conditions, including, but not limited to, mutation(s) in the E3 ligase that (1) abrogate substrate recognition; (2) abrogate recruitment of E2 ligases; and (3) omit catalytic residues to carny out the ubiquitin transfer reaction.

The term “wild-type biotin ligase enzyme” refers to a biotin ligase enzyme derived or obtained from a bacterial microorganism, e.g., the BirA biotin protein ligase of Escherichia coli (E. coli) (Y. Li and R. Sousa, 2012, Protein Expr Purif. 82(1):162-167) or Staphylococcus aureus (S. aureus), that does not contain mutations or variations in sequence, e.g., a naturally-occurring biotin ligase enzyme. The BirA biotin-protein ligase (EC 6.3.4.15) activates biotin to form biotinyl 5′-adenylate and transfers the biotin to biotin-accepting proteins, such as the A3-tag peptide described herein. BirA also functions as a biotin operon repressor. The protein is encoded by the birA gene. Other names for this enzyme include biotin ligase; biotin operon repressor protein; birA; biotin holoenzyme synthetase; biotin-[acetyl-CoA carboxylase] synthetase; biotin-[acetyl-CoA-carboxylase]ligase; biotin-[acetyl-CoA carboxylase] synthetase; acetyl CoA holocarboxylase synthetase; acetyl CoA holocarboxylase synthetase; biotin:apocarboxylase ligase; biotin holoenzyme synthetase; and HCS. In an embodiment, biotinylation of a recombinant protein, e.g., ubiquitin or a ubiquitin-like protein, which expresses an A3-tag peptide, or the AVI-TAG™ peptide, as described herein, is highly specific. In the presence of biotin and ATP, the biotin ligase catalyzes an amide linkage between the biotin and the specific lysine of the A3-tag peptide, or AVI-TAG™ peptide, as described herein.

The term “identical” or “identity” or “percent identity,” or “sequence identity” in the context of two or more nucleic acid or polypeptide (amino acid) sequences that correspond to each other refer to two or more sequences or subsequences that are the same or have a specified percentage of amino acid residues or nucleotides that are the same (e.g., about or equal to 60% identity, or about or equal to 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured, for example, using a BLAST or BLAST 2.0 sequence comparison algorithms with default parameters described below, or by manual alignment and visual inspection. Such sequences are then said to be “substantially identical” and are embraced by the term “substantially identical.” This definition also refers to, or can be applied to, the complement of a test sequence. The definition also includes sequences that have deletions and/or additions, as well as those that have substitutions. Preferred algorithms can account for gaps and the like. Preferably, identity exists for a specified entire sequence or a specified portion thereof or over a region of the sequence that is at least about 20-25 amino acids or nucleotides in length, or over a region that is 50-100 amino acids or nucleotides in length. A corresponding region is any region within the reference sequence.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

For sequence comparisons, one sequence typically serves as a reference sequence to which test sequences are compared. When using a sequence comparison algorithm, test and reference sequences are entered into a computer, subsequence coordinates are designated, if necessary, and sequence algorithm program parameters are designated.

Preferably, default program parameters can be used, or alternative parameters can be designated. The sequence comparison algorithm then calculates the percent sequence identities for the test sequences relative to the reference sequence based on the program parameters. A comparison window includes reference to a segment of any one of the number of contiguous positions selected from, e.g., 20 to 600, usually about 50 to about 200, more usually about 100 to about 150 in which a sequence can be compared to a reference sequence of the same number of contiguous positions after the two sequences are optimally aligned.

Methods of alignment of sequences for comparison are well-known in the art. Optimal alignment of sequences for comparison can be conducted (e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA (PNAS USA) 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection, e.g., Current Protocols in Molecular Biology (Ausubel et al., eds. 1995 (or later) supplement)). Examples of algorithms that are suitable for determining percent sequence identity and sequence similarity are the BLAST and BLAST 2.0 algorithms, which are described in Altschul et al., Nucleic Acids Res. 25:3389-3402 (1977) and Altschul et al., J Mol. Biol., 215:403-410 (1990), respectively. BLAST and BLAST 2.0 can be used to determine percent sequence identity for nucleic acids and proteins. Software for performing BLAST analyses is publicly available through the National Center for Biotechnology Information.

By “subject” is meant a mammal, including, but not limited to, a human or non-human primate mammal, such as a bovine, equine, canine, ovine, camelid, rodent, or feline mammal. A subject may be a human or a human patient. A subject may also embrace a vertebrate or non-vertebrate organism, e.g., an insect or a nematode. Cells, tissues, organs and the like, for use in the described system, compositions and methods may be derived or obtained from a subject.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof, as described herein.

Any compositions or methods provided herein can be combined with one or more of any of the other compositions and methods provided herein.

### BRIEF DESCRIPTION OF THE DRAWINGS

FIGS. 1A and 1B present schematic illustrations of the methods and system described herein. The system described herein may also be considered to embrace a platform comprising the described components that can serve as a base or foundation for the selection, identification and/or isolation of other interacting molecules, substrates, and/or targets involved in the ubiquitination process and its proteins, such as the E1, E2 and E3 proteins, in cells, for example, for the development of therapeutics and therapeutic applications. FIG. 1A presents a schematic depiction of a method of specific E3 ligase (“E3” or “E3 ubiquitin ligase”) substrate tagging by ubiquitin biotinylation, where the E3 ligase is directly fused to (tagged with) a biotin ligase enzyme (e.g., a non-promiscuous, E. coli wild type BirA biotin ligase, BirA) and ubiquitin is fused to (tagged with) a peptide substrate of the BirA biotin ligase (e.g., A3-tag and variants thereof, or AVI-TAG™), which allows for the proximal and interaction-specific biotinylation of the ubiquitinated substrate of E3 via biotinylation of the peptide tagged ubiquitin(s) on the ubiquitinated E3 ligase substrate. FIG. 1B presents a schematic depiction of a method or system involving the biotinylation of ubiquitinated IKZF1 substrate by the CRBN E3 ligase fused to (tagged with) the biotin ligase enzyme BirA (CRBN-BirA) and includes the addition of one or more IMiD agents. The system is designed to bring into proximity, such as in a cellular environment, via the addition of IMiDs, the biotin ligase (e.g., BirA) tagged E3 ligase and a ubiquitinated E3 ligase substrate (e.g., IKZF1), which is attached to ubiquitins tagged with the peptide substrate of the biotin ligase (e.g., A3-tagged- or A3 variant-tagged ubiquitins or AVI-TAG™-tagged ubiquitins). The tagged ubiquitins attached to the E3 ligase substrate are then biotinylated by the biotin ligase attached to the E3 ligase in an interaction-specific manner. In the system and methods as described, the biotinylation of E3 substrate(s) is directed, interaction-specific, and non-promiscuous, thus providing improved and more particular detection and identification of substrate(s) of E3. In embodiments, the addition of IMiDs modulate the binding of the E3 ligase (e.g., CRBN) to tagged and ubiquitinated substrate molecules (e.g., IKZF1). In embodiments, the BirA biotin ligase fused to E3 ligase is non-promiscuous, E. coli wild type BirA biotin ligase, and the BirA biotin ligase peptide substrate is an A3-tag or an A3-tag variant fused to ubiquitin (or a UBL).

FIGS. 2A and 2B illustrate Western blots of proteins in cell (HEK293T) lysates. FIG. 2A shows Western blots of proteins in lysates of cells (HEK293T cells) in response to treatment with the IMiD analog CC-885. FIG. 2B shows Western blots of proteins in lysates of cells (HEK293T cells) expressing BirA-CRBN (BirA biotin ligase linked to the amino (NH₂) terminus of the protein) or CRBN-BirA (BirA biotin ligase linked to the carboxy (COOH) terminus of the protein) into which the IMiD CC885 was introduced to assess the ability of the BirA-tagged CRBN E3 ligase to ubiquitinate the CC-885-induced substrate GSPT1 and to detect the resulting CRBN-dependent degradation of the GSPT1 translation termination factor. The results indicate that CRBN linked to BirA at either the amino or carboxy terminus exhibited E3 ligase functions.

FIGS. 3A-3C illustrate Western blot analyses. FIGS. 3A and 3B show Western blotting analyses of lysates from HEK293T cells expressing the components of the system and methods described herein, namely, ubiquitin fused to AVI-TAG™ and attached to a hemagglutinin marker (“Avi-HA-Ub”), CRBN E3 ligase fused to E. coli wild type BirA, and V5-tagged IKZF1 protein. FIG. 3C shows Western blotting analysis of streptavidin affinity enrichment of biotinylated and ubiquitinated V5-IKZF1 from the same lysates. To prepare the lysates, HEK293T cells were treated with proteasome inhibitor carfilzomib (0.4 μM, 1 hour), followed by pomalidomide (1 μM, 1 hour), followed by biotin (50 μM, 15 minutes). Filled circles represent the above-described treatment, while empty circles represent lack of the treatment, where the same volume of vehicle control (DMSO) was instead added to the cells. Based on quantification of the Western blot signals, approximately 3.9-fold more ubiquitinated V5-IKZF1 substrate was captured by the streptavidin beads when the cells were treated with the IMiD agent pomalidomide (“pom”) compared with control cells treated with DMSO, consistent with the fact that IKZF1 is a pom-dependent substrate of E3 ligase CRBN.

FIGS. 4A-4C show Western blot analyses. FIGS. 4A and 4B show Western blotting analysis of lysates from HEK293T cells expressing the components of the system and methods described herein, namely, ubiquitin fused to AVI-TAG™ and attached to a hemagglutinin marker (“Avi-HA-Ub,” represented by “Avi” in the figures) or ubiquitin fused to A3-tag and attached to a hemagglutinin marker (“A3-HA-Ub,” represented by “A3” in the figures), CRBN E3 ligase fused to E. coli wild type BirA, and V5-tagged IKZF1 protein. FIG. 4C shows Western blotting analysis of streptavidin affinity enrichment of biotinylated and ubiquitinated V5-IKZF1 from the same lysates. The A3-tagged or Avi-tagged ubiquitin attached to the ubiquitinated V5-IKZF1 substrate was biotinylated and captured by the streptavidin beads in the cell lysates treated with the IMiD agent pomalidomide (“pom”) compared with control cells treated with DMSO. (FIGS. 4A-4C). To prepare the lysates, the cells were subject to the same treatment scheme as described in FIG. 3. The use of A3-tagged ubiquitin (“A3”) provided considerably less background compared with the use of the Avi-tagged ubiquitin (FIGS. 4A and 4C). This led to an increased sensitivity in detecting pomalidomide-induced ubiquitination of V5-IKZF1 (17.76 to 5.58 when comparing A3 to Avi) despite some loss of the specific signal (2.03 to 5.58 when comparing the normalized V5/biotin ratios (FIG. 4C).

FIGS. 5A-5C illustrate Western blot analyses. FIGS. 5A and 5B show Western blotting analyses of lysates from HEK293T cells expressing the components of the system and methods described herein, namely, ubiquitin fused to AVI-TAG™ and attached to a hemagglutinin marker (“Avi-HA-Ub”), and CRBN E3 ligase fused to E. coli wild type BirA. FIG. 5C shows Western blotting analysis of streptavidin affinity enrichment of biotinylated and ubiquitinated GSPT1 from the same lysates. To prepare the lysates, HEK293T cells were treated with proteasome inhibitor carfilzomib (0.4 μM, 1 hour), followed by CC-885 (1 μM, 2 hours), followed by biotin (50 μM, 15 minutes). Filled circles represent the described treatment, while empty circles represent the lack of this treatment where the same volume of vehicle control (DMSO) was instead added to cells. Based on quantification of the Western blot signals, 6-12 fold more ubiquitinated GSPT1 was captured by the streptavidin beads when the cells were treated with the IMiD analogue CC-885 compared with control cells treated with DMSO, consistent with the fact that GSPT1 is a CC-885-induced substrate of the E3 ligase CRBN (FIG. 5C).

FIGS. 6A-6C illustrate Western blot analyses. FIGS. 6A and 6B show Western blotting analyses of lysates from HEK293T cells expressing the components of the system and methods described herein, namely, ubiquitin fused to AVI-TAG™ and attached to a hemagglutinin marker (“Avi-HA-Ub,” represented by “Avi” in the figures) or ubiquitin fused to A3-tag and attached to a hemagglutinin marker (“A3-HA-Ub,” represented by “A3” in the figures), and CRBN E3 ligase fused to E. coli wild type BirA. FIG. 6C shows Western blotting analysis of streptavidin affinity enrichment of biotinylated and ubiquitinated GSPT1 from the same lysates. The A3-tagged or Avi-tagged ubiquitin attached to the ubiquitinated GSPT1 was biotinylated and captured by the streptavidin beads in the cell lysates treated with the IMiD analogue CC-88 compared with control cells treated with DMSO. (FIGS. 6A-6C). To prepare the lysates, the cells were subject to the same treatment scheme as described in FIG. 5. The use of A3-tagged ubiquitin (“A3”) provided considerably less background compared with the use of the Avi-tagged ubiquitin (FIGS. 6A and 6C). This led to an increased sensitivity in detecting CC-885-induced ubiquitination of GSPT1 by CRBN when comparing A3-tag to Avi-tag (36.39 to 15.46, respectively) and also an increase in specific signal when comparing the normalized GSPT1/biotin ratios (FIG. 6C).

FIG. 7 provides a schematic depiction of LC-MS/MS-based proteomics analysis as known and practiced in the art, for example, as described by F. Xie et al. in J. Biological Chemistry, 286(29):25443-25449 (2011).

FIG. 8 provides a schematic depiction of quantitative mass spectrometry proteomic analysis as known and practiced in the art, for example, as described by W. Zhu et al. in J. Biomed Biotechnol, 2010(9), 2010, Article ID 840518. Shown on the left is an isotope labeling method. After labeling by light and heavy stable isotopes, the control and sample are combined and analyzed by LC-MS/MS. Quantification is calculated based on the intensity ratio of isotope-labeled peptide pairs. Shown on the right is a label-free method for quantitative proteomics in which the control and sample are subjected to individual LC-MS/MS analysis. The quantification is based on the comparison of peak intensity of the same peptide or the spectral count of the same protein. (Ibid, at page 2).

FIG. 9 depicts a volcano dot plot showing the log 2-fold change in scaled abundance versus statistical significance comparing the biotinylated ubiquitinated proteins in lysates of 293T cells expressing ubiquitin linked to the peptide substrate of BirA in which the peptide substrate constitutes an A3-tag (“A3-Ub”); the E3 ligase CRBN linked to BirA biotin ligase (E. coli wild type BirA); and treated with either DMSO or the IMiD analog CC-885, which induces the ubiquitination of the endogenous neo-substrates of CRBN (e.g., GSPT1 and GSPT2). The ubiquitinated GSPT1 and GSPT2 substrates are observed in the upper right quadrant of FIG. 9. In the figure, proteins that were significantly enriched (log 2 FC>1; p-value<0.001) in treated (drug-treated) versus DMSO-treated conditions are designated as “up,” while proteins that were significantly depleted (log 2 FC<−1; p-value<0.001) are designated as “down.” The remaining proteins are designated as “non-significant.”

FIG. 10 provides volcano dot plots showing the results of mass spectrometry experiments performed to characterize protein degraders (PROTAC®s) that recruit CRBN E3 ubiquitin ligase and other E3 ubiquitin ligases, such as Von Hippel-Lindau (VHL) E3 ligase using the methods and systems described herein. The plot shows the log 2-fold change in scaled abundance versus statistical significance comparing the biotinylated ubiquitinated proteins in lysates of 293T cells expressing ubiquitin linked to the peptide substrate of BirA in which the peptide substrate constitutes an A3-tag (“A3-Ub”). As described in Example 6 herein, Bromo- and Extra-terminal (BET) bromodomain degraders and PTK2 (Protein Tyrosine Kinase 2) degraders were used as PROTAC®s in cell lines expressing CRBN E3 ligase-BirA or VHL E3 ligase-BirA and the appropriate protein substrates susceptible to induced ubiquitination were identified. In the figure, proteins that were significantly enriched (log 2 FC>1; p-value<0.001) in treated (drug-treated) versus DMSO-treated conditions are designated as “up,” while proteins that were significantly depleted (log 2 FC<−1; p-value<0.001) are designated as “down.” The remaining proteins are designated as “non-significant.”

FIG. 11 provides a volcano dot plot showing the results of mass spectrometry experiments performed to characterize a molecular glue, e.g., E7820, an aromatic sulfonamide molecular glue that degrades RBM39 (RNA-Binding Motif Protein 39), a target of sulfonamides that is upregulated in many cancers and regulates transcription of several tumor-related genes, in conjunction with DCAF15 E3 ubiquiun ligase, using the methods and systems described herein. (Example 7). Increased production of the ubiquitinated RBM39 product is observed ((“up” designation) in the upper right quadrant of FIG. 11. In the figure, proteins that were significantly enriched (log 2 FC>1; p-value<0.001) in treated (drug-treated) versus DMSO-treated conditions are designated as “up,” while proteins that were significantly depleted (log 2 FC<−1; p-value<0.001) are designated as “down.” The remaining proteins are designated as “non-significant.” FIG. 12 presents a schematic illustration, similar to FIG. 1B, of the methods and system described herein in which one or more PROTAC®s are included as components in the system and methods to aid in the identification/selection of and/or to functionally affect a target molecule that is ubiquitinated and tagged with a biotinylatable peptide substrate of the biotin ligase enzyme (BirA) linked to the E3 ligase. The illustrated PROTAC® contains an IMiD molecule that binds CRBN (represented by the star) linked to a target binding molecule (represented by the pentagon), which, by way of example, are kinase inhibitors or degraders (e.g., multikinase degraders). Schematically depicted is a system and method involving the biotinylation of a ubiquitinated target molecule associated via the added PROTAC® component with the CRBN E3 ligase fused to (tagged with) the biotin ligase enzyme BirA (CRBN-BirA). The biotin ligase (e.g., BirA) attached to the E3 ligase is brought into close proximity to the tagged ubiquitins on the target molecule (e.g., A3-tagged or A3-variant-tagged ubiquitin or AVI-TAG™-tagged ubiquitin), which is linked to the E3 substrate via the PROTAC®(s) and is biotinylated by the biotin ligase in an interaction-specific manner.

FIGS. 13A-13D present volcano dot plots showing the results of experiments conducted based on the system and method described above in FIG. 12. The plots show the log 2-fold change (FC) in scaled abundance versus statistical significance comparing the biotinylated ubiquitinated target proteins of the PROTAC® multikinase degrader molecules in 293T cells expressing ubiquitin linked to the peptide substrate of BirA in which the peptide substrate constitutes an A3-tag (“A3-Ub”); the E3 ligase CRBN linked to BirA biotin ligase (E. coli wild type BirA); and treated with either DMSO or with a PROTAC® kinase degrader or kinase inhibitor, or a PROTAC® multikinase degrader, e.g., SK-3-91, DB0646, SB1-G-187, WH10417-099, which induces the ubiquitination of the kinase degrader targets that bind CRBN via the presence of the PROTAC® component. FIG. 13A shows the target proteins ubiquitinated by CRBN E3 ligase in the system when the multikinase degrader SK-3-91 is used as a component of the PROTAC® (upper right quadrant). FIG. 13B shows the target proteins ubiquitinated by CRBN E3 ligase in the system when the multikinase degrader DB0646 is used as a component of the PROTAC® (upper right quadrant). FIG. 13C shows the target proteins ubiquitinated by CRBN E3 ligase in the system when the multikinase degrader SB1-G-187 is used as a component of the PROTAC® (upper right quadrant). FIG. 13D shows the target proteins ubiquitinated by CRBN E3 ligase in the system when the multikinase degrader WH10417-099 is used as a component of the PROTAC® (upper right quadrant). In FIGS. 13A-13D, proteins that were significantly enriched (log 2 FC>1; p-value<0.001) in treated (drug-treated) versus DMSO-treated conditions are designated as “up,” while proteins that were significantly depleted (log 2 FC<−1; p-value<0.001) are designated as “down.” The remaining proteins are designated as “non-significant.”

FIGS. 14A and 14B present plots show the log 2-fold change (FC) in scaled abundance versus statistical significance comparing the biotinylated ubiquitinated target proteins of the PROTAC® HDAC inhibitor (degrader) molecules in 293T-CRBN^{-/-}-CRBN-BirA cells (FIG. 14A; (XY-07-097 HDAC degrader)) or 293T-VHL-BirA cells (FIG. 14B; (XY-07-187 HDAC degrader)) expressing ubiquitin linked to the peptide substrate of BirA in which the peptide substrate constitutes an A3-tag (“A3-Ub”); the E3 ligase CRBN or VHL linked to BirA biotin ligase (E. coli wild type BirA); and treated with either DMSO or with a PROTAC® HDAC degrader, which induces the ubiquitination of the HDAC degrader targets (HDAC proteins and corepressor complex members associated with the HDAC proteins) that bind the CRBN E3 ligase or the VHL E3 ligase via the presence of the respective ligands for these E3 ligase components in the respective PROTAC®s. In the figures, proteins that were significantly enriched (log 2 FC>1; p-value<0.001) in treated (drug-treated) versus DMSO-treated conditions are designated as “up,” while proteins that were significantly depleted (log 2 FC<−1; p-value<0.001) are designated as “down.” The remaining proteins are designated as “non-significant.”

FIG. 15 provides a volcanic dot plot graphic representation showing log 2-fold change (FC) versus significance of ubiquitinated substrates identified using A3-tagged ubiquitin in conjunction with either the E3 ligase VHL fused to non-promiscuous wild type E. coli BirA or the E3 ligase CRBN fused to non-promiscuous wild type E. coli BirA in methods as described herein. In the figure, relative enrichment of the substrate proteins for the CRBN and VHL E3 ligases is shown. Proteins that were significantly enriched (log 2 FC>1; p-value<0.001) in VHL-BirA versus CRBN-BirA conditions are designated as “VHL,” while proteins that are significantly enriched (log 2 FC<−1; p-value<0.001) in CRBN-BirA versus VHL-BirA conditions are designated as “CRBN.” The remaining proteins are designated as “non-significant.”

FIGS. 16A and 16B provide volcano plots showing the log 2-fold change (FC) in scaled abundance versus statistical significance comparing the activity of loss-of-function mutants of a CRBN E3 ubiquitin ligase (FIG. 16A: CRBN D249Y mutant; FIG. 16B: CRBN W386A mutant) to the wild-type (WT) CRBN E3 ligase in experiments performed to identify substrates of the E3 ubiquitin ligase, in particular, CC-885 (CRBN) E3 ubiquitin ligase. As described in Example 9, a mass spectrometry experiment was performed to identify protein substrates that were susceptible to mutant and WT CC-885 E3 ubiquitin ligase-induced ubiquitination. Differentially enriched proteins were identified as candidate substrates. Relative enrichment of the substrate proteins for the mutant E3 ligases versus wild type ligase is shown. In FIG. 16A, proteins that were significantly enriched (log 2 FC>1; p-value<0.001) in CRBN(D249Y)-BirA versus CRBN(WT)-BirA conditions are designated as “D249Y,” while proteins that were significantly enriched (log 2 FC<−1; p-value<0.001) in CRBN(WT)-BirA versus CRBN(D249Y)-BirA conditions are designated as “WT.” The remaining proteins are designated as “non-significant.” In FIG. 16B, proteins that were significantly enriched (log 2 FC>1; p-value<0.001) in CRBN(W386A)-BirA versus CRBN(WT)-BirA conditions are designated as “W386A,” while proteins that were significantly enriched (log 2 FC<−1; p-value<0.001) in CRBN(WT)-BirA versus CRBN(W386A)-BirA conditions are designated as “WT.” The remaining proteins are designated as “non-significant.”

FIGS. 17A and 17B provide volcano dot plots showing the log 2-fold change (FC) in scaled abundance versus statistical significance comparing the activity of loss-of-function mutants of a VHL E3 ubiquitin ligase (FIG. 17A: VHL Y98H mutant; FIG. 17B: VHL C162F mutant) to the wild-type (WT) VHL E3 ubiquitin ligase in experiments performed to identify substrates of an E3 ubiquitin ligase, in particular, a VHL E3 ubiquitin ligase. As described in Example 10, a mass spectrometry experiment was performed to identify protein substrates that were susceptible to mutant and WT VHL E3 ubiquitin ligase-induced ubiquitination. Differentially enriched proteins were identified as candidate substrates. Relative enrichment of the substrate proteins for the mutant E3 ligases versus wild type ligase is shown. In FIG. 17A, proteins that were significantly enriched (log 2 FC>1; p-value<0.001) in VHL(Y98H)-BirA versus VHL(WT)-BirA conditions are designated as “Y98H,” while proteins that were significantly enriched (log 2 FC<−1; p-value<0.001) in VHL(WT)-BirA versus VHL(Y98H)-BirA conditions are designated as “WT.” The remaining proteins are designated as “non-significant.” In FIG. 17B, proteins that were significantly enriched (log 2 FC>1; p-value<0.001) in VHL(C162F)-BirA versus VHL(WT)-BirA conditions are designated as “C162F,” while proteins that are significantly enriched (log 2 FC<−1; p-value<0.001) in VHL(WT)-BirA versus VHL(C162F)-BirA conditions are designated as “WT.” The remaining proteins are designated as “non-significant.”

FIG. 18 provides a volcano dot plot showing the ubiquitinated substrate of VHL E3 ubiquitin ligase in the lower left quadrant, as revealed by a decrease in biotinylation due to the presence of an inhibitor of the VHL E3 ubiquitin ligase (VH298) in the cells used in the experiment as described in Example 11. HIF1A was identified as the ubiquitinated substrate (upper left quadrant), which is consistent with the expected activity of the VHL E3 ubiquitin ligase. The results suggest that E3 ubiquitin ligase substrates can be detected and identified by including an inhibitor of the particular E3 ligase that is of interest and/or used in the methods and systems described herein. In the figure, proteins that were significantly enriched (log 2 FC>1; p-value<0.001) in treated (drug-treated) versus DMSO-treated conditions are designated as “up,” while proteins that were significantly depleted (log 2 FC<−1; p-value<0.001) are designated as “down.” The remaining proteins are designated as “non-significant.”

### DETAILED DESCRIPTION OF THE EMBODIMENTS

Described and featured herein are a system, composition, and improved methods for selecting and identifying substrates of E3 ligases (E3 ubiquitin ligases) and for assessing E3 ligase-substrate interactions. The system and methods described herein provide a robust and unbiased approach for detecting, selecting, and identifying E3 ligase substrates, for example, in cells, both in vitro and in vivo. The identification of E3 ligase substrates is critical to understanding a number of human diseases, because the de- or dysregulation of E3 ligase-substrate interactions is often implicated as a major factor in such diseases. Because E3 ligases harbor substrate interaction domains, these E3 ligases and their substrates provide optimal sources for beneficial therapeutic targeting. In addition, the system and methods allow for the evaluation of E3 substrate specificity and the identification of E3 substrates in the presence or absence of modulating agents or drugs, immunomodulatory agents, such as immunomodulatory imide drugs (IMiDs), reprogramming molecules, and bifunctional molecules that artificially induce or modulate substrate-E3 ligase interactions in cells and cellular environments in which the components of the system and method are present and/or introduced. Such molecules include, without limitation, molecular glues and proteolysis targeting chimeras or proteolysis targeting chimeras (PROTAC®s). Drug screening and identification are further provided by the system and methods described herein.

In one aspect, the approach for identifying E3 substrates as described herein constitutes a system (or platform), which is composed of at least two, specifically interactive, component elements. The first component is ubiquitin (or a ubiquitin-like protein) that can be biotinylated by virtue of its being fused to (also referred to as “tagged with” herein) a peptide substrate for biotin ligase, such as a non-promiscuous biotin ligase, e.g., E. coli wild-type biotin ligase BirA. In an embodiment, the peptide substrate tag for BirA is an “A3-tag”-ubiquitin” (also called “A3, “A3-tagged-ubiquitin,” or “A3-Ub” herein), which contains an amino acid sequence that is specifically biotinylated by the biotin ligase fused to E3 ligase in the system. In embodiments, the amino acid sequence of the substrate peptide tag comprises GLNDIFEAQKIEGSG (SEQ ID NO: 22) or MGLNDIFEAQKIEGSGGS (SEQ ID NO: 23). In embodiments, the amino acid sequence of the substrate peptide tag comprises GLNDIFEAQKIEGSG (SEQ ID NO: 22), MGLNDIFEAQKIEGSGGS (SEQ ID NO: 23), or variants thereof, as described herein. In an embodiment, the peptide substrate tag for BirA is “AVI-TAG™”-ubiquitin” (also called “Avi-tag” or “AVI-TAG™” herein). In embodiments, the amino acid sequence of the “Avi-tag” or “AVI-TAG™” peptide comprises GLNDIFEAQKIEWHE (SEQ ID NO: 7) or MGLNDIFEAQKIEWHEGS (SEQ ID NO: 39). A3-tagged, A3-variant tagged, or Avi-tagged ubiquitin can substitute for endogenous ubiquitin and can be biotinylated, especially, when the A3-tagged, A3-variant tagged, or Avi-tagged ubiquitins are attached to substrates of E3 ligases.

Compared to the Avi-Tag, the truncated A3-Tag, or A3-Tag variants, derivatives and analogs thereof, has a lower binding affinity and reactivity toward biotin ligase BirA. For the system described herein, A3-Tag provides advantages over Avi-Tag because the former reduces background biotinylation of ubiquitin that is independent of E3 ligase-mediated interactions. The use of A3-tag results in improved specificity of the system where biotinylation of ubiquitin is driven by proximity to the E3 ligase to which the BirA enzyme is fused. It will be appreciated that throughout the disclosure, use of the term “A3-tag” also encompasses sequence variants of the A3-tag, as described herein.

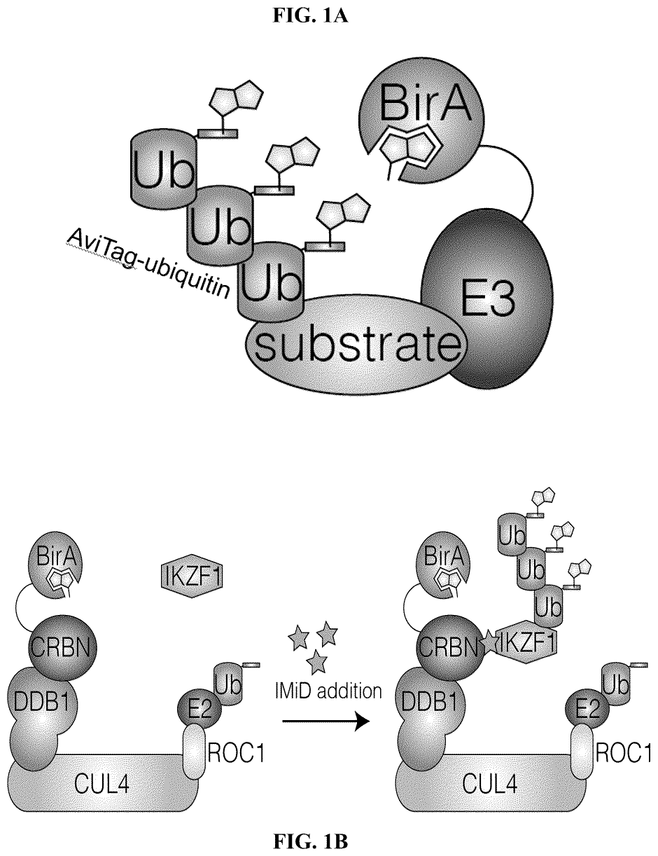

The second component of the system (platform) is an E3 ligase (or E3 ubiquitin ligase) protein that is fused to (also referred to as “tagged with” herein) a non-promiscuous biotin ligase, for example, E. coli wild-type BirA biotin ligase. Optimally, the biotin ligase (BirA) utilized in the system and methods described herein is non-promiscuous and, as such, is unlike and distinct from a promiscuous biotin ligase, such as BirA*, which non-specifically biotinylates proteins and detrimentally leaks activated biotin, as used, for example, in BioID, and as reported by, for example, R. Sears et al. (2019, Meths. Mol. Biol., 2012:299-313). In an embodiment, the E3 ligase protein is fused to a non-promiscuous E. coli wild-type BirA biotin ligase. In the system and methods described herein, the use of an E3 ligase fused to a non-promiscuous, wild-type BirA biotin ligase allows only for the biotinylation of specifically tagged and proximally-located proteins or substrates by E3 ligase-BirA fusion protein, namely, those proteins or substrates that are tagged with ubiquitin (or UBLs) that is fused to a peptide substrate for biotinylation, such as A3-Tag, as described herein. This system and approach enable the biotinylation of peptide tagged-ubiquitin E3 ligase substrates in a directed, ubiquitin-specific, and E3-substrate interaction-specific manner. (FIG. 1A). In an embodiment, the peptide substrate for biotinylation that is fused to ubiquitin (or a UBL) in the system and method herein is the A3-tag peptide substrate of the non-promiscuous BirA, or a sequence variant of the A3-tag peptide substrate. In embodiments, methods that identify or evolve peptide substrates that can be biotinylated by BirA (e.g., non-promiscuous BirA) or other biotinylation enzymes are encompassed. (See, e.g., D. Beckett et al., 1999, Protein Sci; 8(4):921-929; P. J. Schatz, 1993, Biotechnology (NY), 11(10):1138-1143), the contents of which are incorporated by reference herein. In an embodiment, the E3 ligase substrate is exogenously introduced into a cell. In an embodiment, the E3 ligase substrate is endogenously produced in a cell. In an embodiment, the cell is cultured in a cell culture medium that is depleted of biotin.

In another aspect, a variant BirA biotin ligase enzyme is used in the system and method, in which a catalytic amino acid residue in the biotin ligase enzyme (e.g., a non-promiscuous BirA or a non-promiscuous BirA from wild-type E. coli) is substituted with an unnatural amino acid. In an embodiment, a catalytic lysine (K) amino acid residue in BirA, namely, K183, is genetically replaced with a photocaged lysine analog. This residue is required for the adenylation of biotin with ATP to produce the biotinyl-5′-AMP reactive intermediate. In an embodiment, the photocaged lysine analog N^{ε}-[1-(6-Nitrobenzo[d][1,3]dioxol-5yl)ethoxy)carbonyl]-L-lysine (ONPK) is genetically incorporated into BirA, as described in Y. Liu et al., 2021, PNAS USA, Vol. 118, No. 25, which is incorporated by reference herein. The resulting BirA-K183(ONPK), termed a “photocaged BirA biotin ligase,” is inactive in the presence of biotin substrate in culture medium. Upon photolysis (e.g., 365 nm light for 5 minutes), ONPK liberates the lysine side-chain and restores the biotin ligase activity of the enzyme. Such a photocaged BirA-K183(ONPK) is fused to an E3 ligase in the system and methods described herein. In an embodiment, in the system and method involving the use of photocaged BirA-K183(ONPK) biotin ligase, an HEK293T cell line that is engineered to stably express pyrrolysine-amino acyl tRNA synthase (PylRS) for ONPK incorporation is also used. BirA-K183(ONPK) fused to an E3 ligase is expressed in PylRS-expressing HEK293T cells, along with a E3 ligase substrate, to which, in accordance with the described methods, tagged ubiquitins (e.g., A3-tagged ubiquitins) become attached. No biotinylation of the ubiquitin substrate occurs prior to subjecting the cells to light illumination due to the inactivation of the photocaged biotin ligase enzyme in the system. Upon photolysis, e.g., at 365 nm for at least 5 minutes, BirA-K183(ONPK) is decaged, and biotinylation of the E3 ligase substrate-bound, tagged ubiquitins by the decaged biotin ligase enzyme is detectable. In an embodiment, the photocaged BirA biotin ligase is a non-promiscuous BirA biotin ligase. In an embodiment, the photocaged BirA biotin ligase is a wild-type E. coli BirA biotin ligase. In an embodiment, the photocaged BirA biotin ligase is a non-promiscuous, wild-type E. coli BirA biotin ligase.

In another aspect, a chemically activatable biotin ligase enzyme, e.g., a non-promiscuous BirA, or a non-promiscuous BirA derived from wild-type E. coli, for use of the systems and methods herein in deep tissue or intact organisms (animals). Accordingly, a chemical-caged lysine analog N-((E)-cyclooct-2-en-1-yl)-oxy)carbonyl-L-lysine (TCOK) is incorporated in BirA ligase at amino acid residue K183 (or an analogous K residue in another biotin ligase enzyme), e.g., as described in Y. Liu et al., 2021, PNAS USA, Vol. 118, No. 25, to generate a chemoBirA, for example, in which TCOK is incorporated at position K183. The enzyme activity of the chemically-caged biotin ligase can be temporarily blocked, and then rescued by the addition of a chemical activator, such as dimethyl tetrazine (DM-Tz). Because UV illumination is not required, in situ activation of the biotin ligase enzyme fused to an E3 ligase can be carried out in tissue or in animals. The DM-Tz activator can be administered via intravenous (IV) injection, or another suitable route of administration. In an embodiment, biotin may be supplemented in the system and methods. Following the introduction of the DM-Tz (and, optionally, supplemental biotin), the chemBirA becomes functionally active and can biotinylate the tagged ubiquitins attached to the ubiquitinated E3 ligase substrate.

In an embodiment, the controlled biotinylation and ubiquitination system and approach described herein, the short N-terminal biotin epitope fused to ubiquitin, namely, the specific BirA recognition sequence added at the N-terminus of each ubiquitin protein or ubiquitin protein chain in the system, is efficiently biotinylated in cells by using the non-promiscuous, E. coli wild type BirA enzyme fused to an E3 ligase, and biotinylated ubiquitin molecules are incorporated efficiently into a substrate of the E3 ligase. In an embodiment, the BirA biotin ligase fused to an E3 ligase is a photocaged BirA as described supra. In an embodiment, the photocaged BirA biotin ligase is a non-promiscuous BirA biotin ligase. In an embodiment, the photocaged BirA biotin ligase is a wild-type E. coli BirA biotin ligase. In an embodiment, the photocaged BirA biotin ligase is a non-promiscuous, wild-type E. coli BirA biotin ligase. In an embodiment, the photocaged BirA biotin ligase is BirA-K183(ONPK). In an embodiment, the BirA biotin ligase fused to an E3 ligase is a chemically-caged BirA as described supra. In an embodiment, the chemically-caged BirA biotin ligase is a non-promiscuous BirA biotin ligase. In an embodiment, the chemically-caged BirA biotin ligase is a wild-type E. coli BirA biotin ligase. In an embodiment, the chemically-caged BirA biotin ligase is a non-promiscuous, wild-type E. coli BirA biotin ligase. In an embodiment, the chemically-caged BirA biotin ligase is BirA-(TCOK). The components of the compositions, system and methods described herein are designed so that they interact both physically and functionally in proximity within a cell and/or within the cellular environment. The proximity of the components that is induced by the described compositions, system and methods provides specificity of the interactions that occur among the components and allow for enrichment, selection and identification of substrates of E3 ligases that are ubiquitinated by the specifically tagged ubiquitins (or ubiquitin-like molecules), which are ultimately biotinylated by the non-promiscuous biotin ligase fused to the E3 ligase in the system.

In an embodiment, lysis of cells under denaturing conditions allows for the inactivation of UBL isopeptidases (and DUBs) and the high affinity biotin-streptavidin interaction allows for stringent washes, to yield pure biotinylated, ubiquitin-modified E3 ligase substrates with minimal background noise from non-covalent interactors and non-specific contaminants. In an embodiment, the biotinylation of A3-tagged or of Avi-tagged ubiquitinated substrates of E3 ligase using the system described herein may also be carried out in living organisms.

The system, compositions and method described herein provide improvements and advantages over previous biotin labeling techniques used to identify transient protein-protein interactions, such as BioID. BioID is a biotin-labeling method that utilizes a mutant version of the E. coli biotin ligase BirA (“hot” BirA) that promiscuously labels proteins that come into proximity to the mutant biotin ligase, which is linked to a protein of interest (“bait”). In particular, “hot” BirA is linked to a protein of interest (bait), and after a period of incubation with biotin, streptavidin beads are used to enrich for the biotinylated species, thereby revealing its interacting partners. However, use of promiscuous hot BirA is not advantageous for identifying specific cytosolic target proteins and substrates, because stochastic labeling makes it difficult to distinguish the true protein-protein interactions (true ‘hits’) from the false positives. Such problems are alleviated by the system and methods described herein, as a non-promiscuous, wild-type biotin ligase enzyme, and not a hot BirA, is fused to an E3 ligase, ultimately providing biotinylation of an tagged E3 ligase substrate in a ubiquitin- and interaction-specific manner among the components of the system and methods, which are brought into physical and functional proximity in a cell.

In addition, the potent and specific nature of the avidin-biotin interaction allows for the isolation and enrichment of biotinylated and ubiquitinated E3 ligase substrate proteins from cells at significant levels. In an embodiment, such biotinylated and ubiquitinated substrates can be isolated from cells or from a multicellular organism using the system and approaches described herein. In embodiments, biotinylation of the tagged ubiquitins (e.g., A3-tagged ubiquitins or a UBL) on an E3 ligase substrate occurs through the action of a photocaged, non-promiscuous BirA biotin ligase fused to the E3 ligase, following subjecting the photocaged BirA to light illumination (photolysis) as described herein. In an embodiment, the photocaged BirA biotin ligase is BirA-K183(ONPK). In embodiments, biotinylation of the tagged ubiquitins (e.g., A3-tagged ubiquitins or a UBL) on an E3 ligase substrate occurs through the action of a chemically-caged, non-promiscuous BirA biotin ligase fused to the E3 ligase, e.g., BirA-K183(TCOK), following activation of the chemically-caged BirA by a chemical trigger such as dimethyl tetrazine (DM-Tz) as described herein. Biotinylated and tagged, ubiquitinated E3 substrate proteins were able to be resolved by Western blotting and identified by mass spectrometry as described herein. In an embodiment, the mono- or polyubiquitinated-status of the E3 ligase substrates may be determined, for example, by omitting proteasome inhibitors, which allows for the determination of physiological levels of ubiquitination.

In the system described herein, ubiquitin is fused to (“tagged” with) a specific peptide substrate (e.g., A3-tag) of a non-promiscuous biotin ligase (e.g., E. coli wild type BirA), and the biotin ligase (e.g., BirA) is specifically fused to an E3 ligase. Thus, the E3 ligase-biotin ligase (e.g., non-promiscuous BirA) fusion protein transfers a peptide-tagged (e.g., A3-tagged) ubiquitin protein to an E3 ligase substrate. Given their interactive proximity in a cell environment, the particular peptide-tagged, ubiquitinated substrate of the E3 ligase is then specifically biotinylated by the biotin ligase fused to the E3 ligase. As a result, the biotinylated ubiquitins on the E3 ligase substrate allow for the selection, enrichment, identification, and/or isolation of the biotinylated, ubiquitinated E3 ligase substrate by a suitable detection method, such as, without limitation, pulldown/precipitation assays, affinity chromatography and/or mass spectrometry. The system and approach facilitate the identification of substrates of E3 ligases and provides a system and methods to select, enrich, screen for, identify, and/or isolate substrates, as well as various types of modulators, of E3 ligases. In embodiments, biotinylation of the tagged ubiquitins (e.g., A3-tagged ubiquitins or a UBL) on an E3 ligase substrate occurs through the action of a photocaged, non-promiscuous BirA biotin ligase, e.g., BirA-K183(ONPK), fused to the E3 ligase, following subjecting the photocaged BirA to light illumination (photolysis) as described herein. In embodiments, biotinylation of the tagged ubiquitins (e.g., A3-tagged ubiquitins or a UBL) on an E3 ligase substrate occurs through the action of a chemically-caged, non-promiscuous BirA biotin ligase, e.g., BirA-K183(TCOK), fused to the E3 ligase, following activation of the chemo-caged BirA by a chemical trigger, such as DM-Tz, as described herein. In addition, the approach is applicable for studying and identifying the components of other protein-protein or macro-molecular interactions that are involved in the cellular ubiquitination system.

In embodiments, the system and approaches described herein are suitable for use in diverse cell types and organisms. By way of nonlimiting example, prokaryotic, eukaryotic, mammalian and non-mammalian, and human and non-human cells, cell lines and organisms are suitable for use with the described systems and methods. The system and methods may be applied across species, e.g., in human and non-human species, in vitro, ex vivo, or in vivo. Nonlimiting examples of cells and cell lines include those derived or isolated from, or located within, bacteria, fungi, yeast (Saccharomyces sp., Schizosaccharomyces sp., Candida sp., Rhodotorula sp., Debaryomyces (Schwanniomyces) occidentalis, Lipomyces sp., Schizoblastosporion starkeyihenricii, Cryptococcus, etc.), insects (e.g., Drosophila melanogaster), algae, protoplasts, plants, and the like. The systems and methods described herein are applicable for use in human and non-human organisms, e.g., in vivo, for example, in Drosophila sp, and nematodes (e.g., Caenorhabditis elegans).

### Ubiquitin and Ubiquitin-Like Proteins (UBLs)

Ubiquitin is a 76-aa polypeptide that can modify target proteins through the process of ubiquitination, which involves the attachment of an activated ubiquitin moiety through a C-terminal glycine to a lysine or selected other residues in the target substrate. The process involves the activation of ubiquitin by an E1 enzyme, the transfer of the active moiety to an E2 conjugating enzyme and, in many instances, the cooperation of an E3 ligase that binds both the E2-bound ubiquitin and the substrate. De-ubiquitinase enzymes (deubiquitinases; DUBs) can reverse the modification, conferring flexibility and regulation to the process. (L. Pirone et al., 2017, Scientific Reports, 7, 40756; doi: 10.1038/srep40756). Ubiquitination has been most commonly associated with protein degradation by the proteasome; however, the process has more recently been related to a number of other cellular processes, including protein trafficking and DNA repair among others. Ubiquitin itself can be ubiquitinated at any of its seven lysine residues, or the initiating methionine, to form chains that can adopt different conformations, thus resulting in a complex system having the ability to lead modified proteins to different outcomes.

While ubiquitin is the most conserved protein found in all eukaryotes, approximately 20 proteins have been identified that are related to ubiquitin. These related proteins are known as ubiquitin-like proteins (UBLs). While some UBLs have recognizable sequence homology with ubiquitin, others are more divergent and share similar structural or three-dimensional features. All of the UBLs share the beta-grasp fold characteristic of ubiquitin and all participate in processes similar to ubiquitination, which suggests that this family of proteins share a common ancestry. The UBL that shares the highest homology with ubiquitin is NEDD8 (NEural precursor cell-expressed, Developmentally Downregulated 8). While thousands of ubiquitin targets have been identified, the reported number of NEDDylated proteins is lower. Among those, the cullins are RING E3 ligases that link NEDDylation to the ubiquitination of a wide spectrum of targets that participate in many cellular processes.

Other known UBLs include, for example, small ubiquitin-like modifier (SUMO); ubiquitin cross-reactive protein (UCRP), also known as Interferon-Stimulated Gene 15, (ISG15); ubiquitin-related modifier-1 (URM1); neuronal-precursor-cell-expressed developmentally downregulated protein-8 (NEDD8, also called Rub1 in S. cerevisiae); human HLA-F Adjacent Transcript 10, (FAT10), also called UBD; Autophagy-8 (ATG8); and -12 (ATG12); Few ubiquitin-like protein (FUB1); MUB (membrane-anchored UBL); Ubiquitin Fold-Modifier-1 (UFM1); and Ubiquitin-like protein-5 (UBL5), which is known as homologous to ubiquitin-1 [Hub1] in S. pombe. SUMO shares only 18% sequence identity with ubiquitin, but the two proteins contain the same structural fold, called the “ubiquitin fold.” FAT10 and UCRP contain two ubiquitin folds. The compact globular beta-grasp fold is found in ubiquitin, UBLs, and proteins that comprise a ubiquitin-like domain, e.g. the S. cerevisiae spindle pole body duplication protein, Dsk2, and NER protein, Rad23, which contain N-terminal ubiquitin domains.

Five different SUMO genes are found in vertebrates. SUMO1-5, SUMO2 and SUMO3 are almost identical and share 50% identity with SUMO1. All SUMOs use the same E1 and E2 enzymes in the process of SUMOylation and can participate in forming polySUMO or mixed Ubiquitin-SUMO chains. SUMO4 appears to be a pseudogene or it is not processed, while SUMO5 shows tissue-specificity and participates in nuclear body formation. In yeast and Drosophila, there is a single SUMO homolog, Smt3. SUMOylation has been related to transcriptional repression and response to cellular stresses such as DNA damage.

ISG15 is induced by interferons secreted by virus-infected cells and participates in the anti-viral immune response. FAT10 is expressed in immune cells and can also be induced in other cell types by interferon gamma or TNF-alpha (TNF-α). FAT10 can mediate ubiquitin-independent proteasomal degradation. Neither ISG15 nor FAT10 is conserved in lower eukaryotes. Both are composed of two UBL modules, while other UBLs have a single module. The Ubiquitin-Fold Modifier-1, UFM1, is conserved in metazoans and plants. It has a role in erythroid and megakaryocyte development, homeostasis of the endoplasmic reticulum (ER) and vesicle trafficking. ATG8 and ATG12 are involved in the regulation of autophagy. ATG8 is a lipid modifier that is conjugated to phosphatidylethanolamine and participates in autophagosome biogenesis. There are 6 ATG8 orthologs in humans, classified as GABARAP1-2 and MAP1LC3A-B. ATG12 is conjugated to at least one other protein in the outer part of the autophagosome membrane, where this complex acts as the E3 ligase for Atg815. Another UBL, FAU (Finkel-Biskis-Reilly murine sarcoma virus, ubiquitously expressed), is synthesized as a fusion protein with the ribosomal protein S30. In macrophages, FAU exhibits an immunoregulatory role by inhibiting lipopolysaccharide-induced signaling and phagocytosis. The Ub Related Modifier 1, URM1, is conserved in eukaryotes; its structure is similar to that of ancient prokaryotic sulfur carriers. Sulfur donors that are involved in the biosynthetic pathway of thiamine (vitamin B1) and molybdopterin (MPT) in prokaryotes, ThiS and MoaD, have a certain homology with ubiquitin and contain a beta-grasp fold. Thus, URM1 may represent a bridge between the prokaryotic sulfur carriers and eukaryotic UBLs.

These related molecules have novel functions and influence diverse biological processes. There is also cross-regulation between the various conjugation pathways, since some proteins can become modified by more than one UBL, and sometimes even at the same lysine residue. By way of example, SUMO modification often acts antagonistically to that of ubiquitination and serves to stabilize protein substrates. Proteins conjugated to UBLs are typically not targeted for degradation by the proteasome, but rather function in diverse regulatory activities. Attachment of UBLs might alter substrate conformation, affect the affinity for ligands or other interacting molecules, alter substrate localization, and influence protein stability.

UBLs are structurally similar to ubiquitin and are processed, activated, conjugated, and released from conjugates by enzymatic steps that are similar to the corresponding mechanisms for ubiquitin. UBLs are also translated with C-terminal extensions that are processed to expose the invariant C-terminal residues LRGG. These modifiers have their own specific E1 (activating), E2 (conjugating) and E3 (ligating) enzymes that conjugate the UBLs to intracellular targets. These conjugates can be reversed by UBL-specific isopeptidases that have similar mechanisms to that of the deubiquitinating enzymes (DUBs).

### E3 Ubiquitin Ligases

An estimated 500-1000 E3 ligases (alternatively termed “E3,” “E3 ubiquitin ligase,” “E3 ligase,” “E3 enzyme,” “E3 protein”, or “Ubiquitin ligase”) are believed to exist in humans. E3 ligases operate in conjunction with E1 ubiquitin-activating enzyme and E2 ubiquitin-conjugating enzyme, particularly to impart substrate specificity onto the E1 and E2 enzymes. In brief, one major E1 enzyme, shared by all E3 ubiquitin ligases, uses ATP to activate ubiquitin for conjugation and transfers it to an E2 enzyme. The E2 enzyme interacts with a specific E3 ligase partner and transfers the ubiquitin to the target protein. The E3 ligase, which may be a multi-protein complex, is, in general, responsible for targeting ubiquitination to specific substrate proteins.

In particular, E3 ubiquitin ligase is a protein that recruits an E2 ubiquitin-conjugating enzyme that has been loaded with ubiquitin, recognizes a protein substrate, and assists or directly catalyzes the transfer of ubiquitin from the E2 enzyme to the protein substrate. The transferred ubiquitin is attached to a lysine residue on the target protein by an isopeptide bond. E3 ligases interact with both the target protein and the E2 enzyme, and so impart substrate specificity to the E2 enzyme. E3 ligases typically polyubiquitinate their substrate with Lys48-linked chains of ubiquitin, targeting the substrate for destruction by the proteasome. However, a variety of other types of linkages are possible and alter a protein's activity, interactions, or localization. Ubiquitination by E3 ligases regulates diverse areas such as cell trafficking, DNA repair, and signaling and plays a significant role in cell biology. E3 ligases are also key players in cell cycle control, mediating the degradation of cyclins, as well as cyclin-dependent kinase inhibitor proteins. The human genome encodes over 600 putative E3 ligases, thus providing a tremendous diversity in substrates.

The E3 ubiquitin ligases are classified into three major classes based upon the unique catalytic mechanisms that they employ, namely, “really interesting new gene” (RING); “the RING-between-RING” or “RING-BRcat-Rcat” (RBR); and “Homologous to E6AP C-terminus” (HECT E3) ubiquitin ligases. (Y. Wang et al., 2020, J. Cell Science, 33 (7): jcs228072). RING E3 ubiquitin ligases function as scaffolds that bring the E2-ubiquitin complex and substrate into close proximity to facilitate the transfer of ubiquitin onto the substrate protein. In contrast, the RBR and HECT E3 ubiquitin ligases play a more catalytic role by forming a thioester bond with a conserved catalytic cysteine residue in the Rcat domain (for RBRs) or the C-terminal lobe of the HECT domain (for HECTs) prior to the transfer of ubiquitin to its destined substrate. The RING-E3 ligases are the largest family and contain ligases such as the anaphase-promoting complex (APC) and the SCF complex (Skp1-Cullin-F-box protein complex), (K. I. Nakayama et al., May 2006, Nature Reviews. Cancer, 6(5):369-81. doi:10.1038/nrc1881). SCF complexes include four proteins: Rbx1, Cul1, Skp1, which are invariant among SCF complexes, and an F-box protein, which varies. Around 70 human F-box proteins have been identified. F-box proteins contain an F-box, which binds the rest of the SCF complex, and a substrate binding domain, which affords substrate specificity to the E3 ligase.

Non limiting examples of E3 ligases (and their gene sequence reference numbers), in particular, human E3 ligases, include Ubiquitin protein ligase E3A (GenBank Reference No. AAH02582.2); MDM2 (UniProt Ref: Q00987; NM Ref: 001367990); Anaphase-promoting complex (APC), (NCBI Reference Sequence: NP 073153.1); UBR5 (EDD1), (NCBI Reference Sequence: NP 056986.2); LNXp80 (GenBank Reference No.: AAC40075.1); CBX4 (E3 SUMO-protein ligase CBX4, NCBI Reference Sequence: NP 003646.2); CBLL1 (E3 ubiquitin-protein ligase Hakai isoform 1, NCBI Reference Sequence: NP 079090.2); DCAF15 (DDB1- and CUL4-associated factor 15 isoform 1, (UniProt Ref: Q66K64; NM Ref: 138353); DCAF16 (DDB1- and CUL4-associated factor 16, UniProt Ref: Q9NXF7; NM Ref: 001345880); DCAF1 (UniProt Ref: Q9Y4B6; NM Ref: 014703); DCAF11 (UniProt Ref: Q8TEB1; NM Ref: 025230); DCAF6 (UniProt Ref: Q58WW2; NM Ref: 001017977); DCAF8 (UniProt Ref: Q5TAQ9; NM Ref: 015726); DCAF10 (UniProt Ref: Q5QP82; NM Ref: 024345); DCAF5 (UniProt Ref: Q96JK2; NM Ref: 003861); DCAF12 (UniProt Ref: Q5T6F0; NM Ref: 015397); DCAF4

(UniProt Ref: Q8WV16; NM Ref: 015604); DCAF17 (UniProt Ref: Q5H9S7; NM Ref: 025000); HACE1 (E3 ubiquitin-protein ligase HACE1 isoform a, NCBI Reference Sequence: NP 065822.2); HECTD1 (E3 ubiquitin-protein ligase HECTD1, NCBI Reference Sequence: NP 056197.3); HECTD2 (E3 ubiquitin-protein ligase HECTD2 isoform a, NCBI Reference Sequence: NP 877497.4); HECTD3 (E3 ubiquitin-protein ligase HECTD3, NCBI Reference Sequence: NP 078878.3); HECTD4 (E3 ubiquitin-protein ligase HECTD4, NCBI Reference Sequence: NP 001103132.4); HECW1 (E3 ubiquitin-protein ligase HECW1 isoform a, NCBI Reference Sequence: NP 055867.3); HECW2 (E3 ubiquitin-protein ligase HECW2 isoform 1, NCBI Reference Sequence: NP 001335697.1); HERC1 (E3 ubiquitin-protein ligase HERC1, NCBI Reference Sequence: NP 003913.3); HERC2 (GenBank Reference No.: AAD08657.1); HERC3 (E3 ubiquitin-protein ligase HERC3 isoform 1, NCBI Reference Sequence: NP 055421.1); HERC4 (E3 ubiquitin-protein ligase HERC4 isoform a, NCBI Reference Sequence: NP 071362.1); HERC5 (E3 ISG15-protein ligase HERC5, NCBI Reference Sequence: NP 057407.2); HERC6 (E3 ubiquitin-protein ligase HERC6 isoform 1, NCBI Reference Sequence: NP 060382.3); HUWE1 (E3 ubiquitin-protein ligase HUWE1, NCBI Reference Sequence: NP 113584.3); XIAP (E3 ubiquitin-protein ligase XIAP, UniProt Ref: P98170; NM Ref: 001167); ITCH (UniProtKB/Swiss-Prot Reference No.: Q96J02.2), NEDD4 (UniProtKB/Swiss-Prot Reference No.: P46934.4); NEDD4L (GenBank: Reference No. AAH32597.1); PPIL2 (RING-type E3 ubiquitin-protein ligase PPIL2 isoform a, NCBI Reference Sequence: NP 001304925.1); PRPF19 (RING-type E3 ubiquitin transferase PRP19, UniProtKB/Swiss-Prot Reference No.: Q9UMS4.1); PIAS1 (E3 SUMO-protein ligase PIAS1 isoform 1, NCBI Reference Sequence: NP 001307616.1); PIAS2 (E3 SUMO-protein ligase PIAS2 isoform 5, NCBI Reference Sequence: NP 001340967.1); PIAS3 (GenBank Reference No.: CAG33371.1); PIAS4 (RING-type E3 ubiquitin transferase PIAS4, UniProtKB/Swiss-Prot Reference No.: Q8N2W9 and NCBI Reference Sequence: NP_056981.2); RANBP2 (E3 SUMO-protein ligase RanBP2, UniProtKB/Swiss-Prot Reference No.: P49792.2); RNF4 (E3 ubiquitin-protein ligase RNF4 isoform 1, NCBI Reference Sequence: NP 001171938.1); RNF114 (E3 ubiquitin-protein ligase RNF114. NCBI Reference Sequence: NP_061153.1); RBX1 (E3 ubiquitin-protein ligase RBX1, UniProtKB/Swiss-Prot Reference No.: P62877.1); SMURF1 (E3 ubiquitin-protein ligase SMURF1, UniProtKB/Swiss-Prot Reference No.: Q9HCE7.2); SMURF2 (E3 ubiquitin-protein ligase SMURF2, NCBI Reference Sequence: NP_073576.1); STUB1 (STIP1 homology and U-box containing protein 1, GenBank Reference No.: AAH63617.1); TOPORS (E3 ubiquitin-protein ligase Topors, UniProtKB/Swiss-Prot Reference No.: Q9NS56.1); TRIP12 (E3 ubiquitin-protein ligase TRIP12 isoform a, NCBI Reference Sequence: NP_001335244.1); UBE3A (ubiquitin-protein ligase E3A isoform 1, UniProt Ref: Q05086; NM Ref: 000462); UBE3B (UBE3B protein, GenBank Reference No.: AAI08706.1); UBE3C (ubiquitin-protein ligase E3C, NCBI Reference Sequence: NP_055486.2); UBE3D (E3 ubiquitin-protein ligase E3D, UniProtKB/Swiss-Prot Reference No.: Q7Z6J8.2), UBE4A (GenBank Reference No.: AAI11418.1); UBE4B (Ubiquitin conjugation factor E4 B, UniProtKB/Swiss-Prot Reference No.: 095155.1); UBOX5 (RING-type E3 ubiquitin transferase RNF37, UniProtKB/Swiss-Prot Reference No.: 094941.1); UBR5 (E3 ubiquitin-protein ligase UBR5, UniProtKB/Swiss-Prot Reference No.: 095071.2); VHL (von Hippel-Lindau disease tumor suppressor, UniProtKB/Swiss-Prot Reference No.: P40337.2; NM Ref: 000551); WWP1 (NEDD4-like E3 ubiquitin-protein ligase WWP1, NCBI Reference Sequence: NP_008944.1); WWP2 (NEDD4-like E3 ubiquitin-protein ligase WWP2, UniProtKB/Swiss-Prot Reference No.: 000308.2); Parkin (E3 ubiquitin-protein ligase parkin, UniProtKB/Swiss-Prot Reference No.: 060260.2), MKRN1 (E3 ubiquitin-protein ligase makorin-1 isoform 1, NCBI Reference Sequence: NP_038474.2); CRBN (GenBank Reference No.: AAH67811.1); CRBN, isoform 2 (NCBI Reference Sequence: NP_001166953.1); SOCS/BC-box/eloBC/CUL5/RING, MARCH E3 ligase family (H. Lin et al., 2019, Front. Immunol., Vol. 10, Article 1751), e.g., MARCH1 (NCBI Gene ID No. 55061), MARCH5 (NCBI Gene ID No. 54708); KEAP1 (UniProt Ref: Q14145; NM Ref: 203500); ASB9 (UniProt Ref: Q96DX5; NM Ref: 001031739); RNF126 (UniProt Ref: Q9BV68; NM Ref: 194460); ARIH1 (UniProt Ref: Q9Y4X5; NM Ref: 005744); ARIH2 (UniProt Ref: 095376; NM Ref: 001317333); SIAH1 (UniProt Ref: Q8IUQ4; NM Ref: NM_003031); BIRC2 (UniProt Ref: Q13490; NM Ref: 001166); BIRC3 (UniProt Ref: Q13489; NM Ref: 001165); FEM1A (UniProt Ref: Q9BSK4; NM Ref: 018708); FEM1B (UniProt Ref: Q9UK73; NM Ref: 015322); FEM1C (UniProt Ref: Q96JP0; NM Ref: 020177); FBXW11 (UniProt Ref: Q9UKB1; NM Ref: 012300); FBXW7 (UniProt Ref: Q969H0; NM Ref: 033632); TRIM8 (UniProt Ref: Q9BZR9; NM Ref: 030912); VCPIP1 (UniProt Ref: Q96JH7; NM Ref: 025054); SKP2 (UniProt Ref: Q13309; NM Ref: 005983); FBXL2 (UniProt Ref: Q9UKC9; NM Ref: 012157); FBXL3 (UniProt Ref: Q9UKT7; NM Ref: 012158); FBXL4 (UniProt Ref: Q9UKA2; NM Ref: 012160); FBXL5 (UniProt Ref: Q9UKA1; NM Ref: 012161); FBXL6 (UniProt Ref: Q8N531; NM Ref: 012162); FBXL7 (UniProt Ref: Q9UJT9; NM Ref: 012304); FBXL8 (UniProt Ref: Q96CD0; NM Ref: 018378); FBXL12 (UniProt Ref: Q9NXK8; NM Ref: 017703); FBXL13 (UniProt Ref: Q8NEE6; NM Ref: 145032); FBXL14 (UniProt Ref: Q8N1E6; NM Ref: 152441); FBXL15 (UniProt Ref: Q9H469; NM Ref: 024326); FBXL16 (UniProt Ref: Q8N461; NM Ref: 153350); FBXL17 (UniProt Ref: Q9UF56; NM Ref: 001163315); FBXL18 (UniProt Ref: Q96ME1; NM Ref: 024963); FBXL19 (UniProt Ref: Q6PCT2; NM Ref: 001099784); FBXL20 (UniProt Ref: Q96IG2; NM Ref: 032875); FBXL22 (UniProt Ref: Q6P050; NM Ref: 203373); DDB2 (UniProt Ref: Q92466; NM Ref: 000107); AMBRA1 (UniProt Ref: Q9C0C7; NM Ref: 017749); BRWD3 (UniProt Ref: Q6RI45; NM Ref: 153252); TRPC4AP (UniProt Ref: Q8TEL6; NM Ref: 015638); WDTC1 (UniProt Ref: Q8N5D0; NM Ref: 001276252); DTL (UniProt Ref: Q9NZJ0; NM Ref: 016448); PHIP (UniProt Ref: Q8WWQ0; NM Ref: 017934); PWP1 (UniProt Ref: Q13610; NM Ref: 007062); ERCC8 (UniProt Ref: Q13216; NM Ref: 000082); TOR1AIP2 (UniProt Ref: Q9H496; NM Ref: 022347); DET1 (UniProt Ref: Q7L5Y6; NM Ref: 001144074); COP1 (UniProt Ref: Q8NHY2; NM Ref: 022457); and isoforms and homologs thereof, as well as functional portions or fragments thereof. In embodiments, the above E3 ligase proteins encompass proteins having at least 85%, at least 90%, at least 93%, at least 95%, at least 98%, at least 99%, or greater, amino acid sequence identity to the amino acid sequences of the denoted E3 ligase proteins. Other E3 ligases can be found in M. Schapira et al., 2019, Nat Rev Drug Discov. 18, 949-963, which is incorporated herein by reference.

E3 ligases dictate target specificity and typically function as adaptor molecules that recognize substrates through protein-protein interactions and promote ubiquitination by holding those targets proximal to the associated ubiquitination machinery. Of note, a number of E3 ligases are essential in most cancer types (R. M. Meyers et al., 2017, Nat. Genet., 49:1779-1784), thus making E3 ligases and their substrates suitable for the selection and identification approaches described herein.

### Enzymatic Biotinylation

Biotinylation is a process by which biotin is covalently attached to a protein, nucleic acid or other molecule. Biotinylation is rapid, specific and typically does not alter or disturb the natural function of the molecule due to the small size of biotin (MW=244.31 g/mol). In an embodiment, ubiquitin fused to an A3-tag peptide substrate of a non-promiscuous biotin ligase (e.g., BirA) that is fused to an E3 ligase is ligated by the E3 ligase to an E3 substrate and is biotinylated with biotin by the biotin ligase-E3 ligase fusion protein as described herein. In an embodiment, the BirA biotin ligase is a photocaged BirA, e.g., BirA-K183(ONPK). In an embodiment, the BirA biotin ligase is a chemically-caged BirA, e.g., BirA-K183(TOK). Biotin binds to streptavidin and avidin with an extremely high affinity, fast on-rate, and high specificity; these interactions permit the isolation of biotinylated substrate molecules of interest. Biotin-binding to streptavidin and avidin is resistant to extremes of heat, pH and proteolysis, making the selection, isolation, or capture of biotinylated molecules possible using a number of assays. Moreover, multiple biotin molecules can be conjugated to a substrate protein or molecule of interest, which allows binding of multiple streptavidin, avidin, or neutravidin protein molecules and also increases the sensitivity of detection of the substrate protein or molecule of interest. Because of the strong affinity between biotin and streptavidin, the purification of biotinylated protein substrates of an E3 ligase fused to a non-promiscuous BirA is a highly beneficial approach to selecting, identifying and/or isolating ubiquitinylated and biotinylated substrates of the E3 ligase.

Enzymatic biotinylation results in biotinylation of a specific lysine within a certain sequence by a bacterial biotin ligase. Enzymatic biotinylation allows biotin to be linked at precisely one residue present in the protein. As the biotinylation reaction goes to completion, the biotinylated product is generated with high uniformity and can be linked to streptavidin or avidin in a defined orientation. In accordance with the methods described herein, enzymatic biotinylation of ubiquitin proteins attached to E3 ligase substrates is carried out by a non-promiscuous E. coli biotin holoenzyme synthetase, also known as biotin ligase (BirA) described supra. In embodiments, ubiquitin is fused at its N-terminus, C-terminus or at an internal loop to a peptide comprising the amino acid sequence GLNDIFEAQKIEGSG (SEQ ID NO: 22), or the amino acid sequence MGLNDIFEAQKIEGSGGS (SEQ ID NO: 23), termed an Acceptor Peptide (AP), such as the A3-tag peptide described herein. In some embodiments, ubiquitin is fused at its N-terminus, C-terminus or at an internal loop to the amino acid AVI-TAG™ peptide GLNDIFEAQKIEWHE (SEQ ID NO: 7), (D. Beckett et al., 1999, Protein Science, 8(4):921-929, doi:10.1110/ps.8.4.921), or to the peptide MGLNDIFEAQKIEWHEGS (SEQ ID NO: 39). The tagged ubiquitin protein then interacts with BirA biotin ligase fused to E3 ligase, allowing biotinylation to take place in the presence of biotin and ATP. In an embodiment, enzymatic biotinylation occurs in cells of interest through the A3-tag peptide substrate of biotin ligase (or the AVI-TAG™ peptide substrate) fused to ubiquitins that are attached to the substrate of an E3 ligase-biotin ligase fusion protein. In the system, the A3-tagged, ubiquitinated E3 substrate proteins are specifically biotinylated through the activity of the non-promiscuous BirA fused to E3 ligase, which is in proximity to the tagged, ubiquitinated E3 ligase substrate. The natural substrate of BirA is the biotin carboxyl carrier protein (BCCP), and the A3-tag or AVI-TAG™ are each peptides of BCCP containing the site for biotinylation by BirA. In an embodiment, the non-promiscuous BirA fused to E3 ligase is a photocaged BirA, such as BirA-K183(ONPK), which is activated when decaged via light illumination and photolysis. In an embodiment, the non-promiscuous BirA fused to E3 ligase is a chemically-caged BirA, such as BirA-K183(TCOK), which is unblocked and activated by the action of a chemical activator such as dimethyl tetrazine.

Biotin can be used as a capture tag in E3 ligase substrate selection and isolation methods, such as affinity chromatography and precipitation assays using avidin, streptavidin, or neutravidin, attached or bound to a solid support, e.g., a column, beads, wells of a multi-well plate, and the like. Avidin (or streptavidin) is the natural ligand for biotin. The interaction between avidin/streptavidin and biotin may be disrupted using procedures and reagents known in the art, e.g., 6M GuHCl at low pH, e.g., pH 1.5. In an embodiment, the substrate (substrate protein) can be tagged with iminobiotin, a biotin analog that binds strongly to avidin/streptavidin at alkaline pH; however, the affinity is reduced upon lowering the pH. Therefore, an iminobiotin-tagged functional protein can be released from an avidin/streptavidin column or beads by decreasing the pH (to around pH 4). By way of nonlimiting example, an iminobiotin tag may be used if isolation of the tagged E3 ligase substrate protein is desired.

In an embodiment, capture, selection and/or isolation of biotinylated ubiquitinated E3 ligase substrate molecules, e.g., in cell lysates, is carried out using streptavidin- or avidin-conjugated beads. Streptavidin- and avidin-conjugated beads are commercially available and may be particles, microparticles, or nanoparticles composed of, without limitation, latex, carboxylate-modified latex, Sepharose, or agarose. Different types of Streptavidin- or avidin-conjugated beads are known and used by those in the art and are available, for example, from Millipore/SIGMA, St. Louis, MO; ThermoFisher Scientific, Waltham, MA; New England BioLabs, UK). By way of example, the streptavidin-conjugated beads can be magnetic.

Without intending to be limiting, the biotin tagged E3 ligase substrate proteins can also be detected, selected, and identified using anti-biotin antibodies or avidin/streptavidin-tagged detection strategies such as enzyme reporters (e.g., horseradish peroxidase, alkaline phosphatase) or labeled, detectable probes, such as chemiluminescent or fluorescent probes, which can be useful in localization by fluorescent or electron microscopy, ELISA assays, ELISPOT assays, Western blots and other immunoanalytical methods and techniques as known and used in the art. Detection with monovalent streptavidin can avoid clustering or aggregation of the biotinylated target. In addition, high throughput screening (HTS) assays as known and used in the art, may be used in conjunction with the system and methods described herein. HTS assays allow for conducting numerous assays, such as cell-based assays, which are carried out in the wells of a multi-well microtiter plate, e.g., a multi-well, microtiter plate having 96, 192, 384, 1536, 3456, or 6144 wells. By way of nonlimiting example, fluorescence polarization or Homogeneous Time Resolved Fluorescence (HTRF), can be employed to detect the biotinylated substrate. HTS assays may be carried out using either cellular systems (cell extracts or lysates) and/or purified systems (e.g., purified E3 ligase combined with a purified substrate or combined with an extract). In general, HTRF assay technology, which combines standard FRET technology with time-resolved measurement of fluorescence, is used to measure analytes in a homogeneous format, such as for drug screening or target studies and analyses in HTS formats.

Cell-based HTS assays are highly suitable for use with the system and methods described herein. For example, the levels of biotinylated substrates produced via ubiquitin biotinylation by BirA biotin ligase fused to an E3 ligase can be measured using proximity-based detection assays, for example, using streptavidin conjugated donor beads and substrate-specific antibody conjugated acceptor beads (e.g., Amplified Luminescent Proximity Homogenous Assay (Alpha) Screen (ALPHA SCREEN™) technology, as practiced in the art (A. Yasgar et al., 2016, Methods Mol Biol., 1439: 77-98, incorporated by reference herein; and as commercially available from, e.g., Perkin-Elmer, Boston, MA). In brief, ALPHA SCREEN™ is a bead-based, non-radioactive, homogeneous proximity assay performed in multi-well microtiter plate format. Binding of molecules captured on the beads leads to an energy transfer from one bead to the other, ultimately producing a luminescent/fluorescent signal. Upon laser excitation and illumination at 680 mm, a photosensitizer, i.e., phthalocyanine, in a “donor” bead (e.g., a bead (micro- or nanoparticle) conjugated to streptavidin that binds to biotinylated and ubiquitinated E3 ligase substrate) converts ambient oxygen to a more excited, reactive form of O₂, singlet oxygen, which has a 4 μsec half-life. The singlet state oxygen molecules diffuse through solution within the half-life of the singlet O₂ to react with a thioxene derivative in an “acceptor” bead (e.g., a bead (micro- or nanoparticle) conjugated to an antibody that binds to the E3 ligase substrate) that is in proximity of the donor bead. Energy is transferred from the singlet oxygen to the thioxene derivatives within the acceptor bead, which generates chemiluminescence at 370 nm that further activates fluorophores contained in the acceptor bead. The fluorophores subsequently emit light at 520-620 nm. In the absence of a specific biological interaction between a donor bead and an acceptor bead, or in the absence of an acceptor bead in close proximity to the donor bead, the singlet state oxygen produced by the donor bead falls to ground state and no signal is produced. As a result, only a very low background signal is produced. ALPHA SCREEN™ beads are coated with a layer of hydrogel which retains the dyes, minimizes non-specific binding and particle self-aggregation, and provides functional groups for bioconjugation. The size of the beads is optimized and uniform, they are small enough to prevent settling in aqueous suspensions and are easily dispensed using automated liquid handling, yet are large enough to be centrifuged. In addition, because the illumination wavelength is very high at 680 nm, very few biological components and assay compounds interfere in the assay, which is versatile, sensitive, homogeneous, and miniaturizable, making it conducive to higher throughput in HTS assays.

In an embodiment, an HTS assay, i.e., a time-resolved fluorescence resonance energy transfer (TR-FRET) assay, for example, as reported by J. M. Rectenwald et al. (2019, SLAS Discov., 24(6):693-700), can be used in conjunction with the described system and methods.

The non-covalent bond formed between biotin and avidin or streptavidin has a binding affinity that is higher than most antigen and antibody bonds and approaches the strength of a covalent bond. Such tight binding makes biotin-labeled proteins useful for applications such as affinity chromatography using immobilized avidin or streptavidin to separate the biotinylated protein from a mixture of other proteins and biochemicals. In addition, in vivo protein biotinylation allows for the detection and identification of protein-protein interactions as described herein and their proximity in living cells.

### Cereblon (CRBN) E3 Ubiquitin Ligase

Cereblon (CRBN) forms an E3 ubiquitin ligase complex with damaged DNA binding protein 1 (DDB1), Cullin-4A (CUL4A), and regulator of cullins 1 (ROC1), e.g., DDB1-CUL4A-CRBN). (S. Angers et al., 2006, Nature, 443(7111): 590-3. doi:10.1038/nature05175). This complex ubiquitinates a number of other proteins. Through a mechanism not yet completely elucidated, this ubiquitination results in reduced levels of fibroblast growth factor 8 (FGF8) and fibroblast growth factor 10 (FGF10). FGF8, in turn, regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein. Furthermore, CRBN and DDB2 bind to DDB1 in a competitive manner.

CRBN has been reported to bind to immunomodulatory imide drugs (IMiDs) such as lenalidomide, thalidomide and pomalidomide, among others, or other CRBN modulating agents, such as CC-885. By way of example, the drug thalidomide and its analogs, e.g., pomalidomide and lenalidomide, bind to CRBN and alter which substrates can be degraded by CRBN, thus leading to an antiproliferative effect on myeloma cells, by way of nonlimiting example.

### Immunomodulatory Imide Drugs (IMiDs)

Immunomodulatory imide drugs (IMiDs) are a class of immunomodulatory drugs (drugs that affect immune responses) containing an imide group. (R. Knight, 2005, Seminars in Oncology, 32 (4 Suppl 5):S24-S30. doi:10.1053/j.seminoncol.2005.06.018). The IMiD class, which includes, among others, thalidomide and its analogs lenalidomide, pomalidomide, and iberdomide, may also be referred to as ‘Cereblon modulators’. Cereblon (CRBN) is the protein targeted by this class of drugs. The name “IMiD” encompasses both the term “IMD” for “immunomodulatory drug” and the imide, imido-, imid-, and imid forms. IMiDs bind to cereblon (CRBN) to confer differentiated substrate specificity on the CRL4(CRBN) E3 ubiquitin ligase.

Thalidomide and its analogs have anti-angiogenic and anti-inflammatory properties, which may be of beneficial use as therapeutics in the treatment of cancers as well as some inflammatory diseases. While thalidomide is useful for such treatments, the use of is analogs is also encompassed in order to avoid potential problems and adverse events, such as teratogenic side effects, high incidence of other adverse reactions, poor solubility in water and poor absorption from the intestines.

Another cereblon modulator, CC-885, has been reported to have potent anti-tumor activity. (M. Matyskiela et al., 2016, Nature, 535(7611):252-257. doi: 10.1038/nature18611). The anti-tumor activity of CC-885 is mediated through the cereblon-dependent ubiquitination and degradation of the translation termination factor GSPT1, Patient-derived acute myeloid leukemia tumor cells exhibit high sensitivity to CC-885, indicating the clinical potential of this mechanism. While GSPT1 does not have apparent structural, sequence or functional homology to previously known cereblon substrates, the CRBN substrate Ikaros (IKZF1) uses a similar structural feature to bind CRBN, thus suggesting a common motif for substrate recruitment to the CRL4-CRBN E3 ligase.

C-220 represents another example of a CRBN modulator that induces a more potent degradation of IKFZ1 and IKFZ3 than previous IMiDs (M. E. Matyskiela et al., 2018, J. Med Chem., 61:535-542). C-220 has been reported to elicit positive response in patients with systemic lupus erythematosus in a phase 2a study (V. P. Werth et al., Abstract #SAT0255; Annual European Congress of Rheumatology (EULAR) 2017; Jun. 14-17, 2017; Madrid, Spain).

Without wishing to be bound by theory, CRBN-binding IMiDs lack a substrate-targeting chemical moiety. Instead, the target directly interacts with the CRBN-bound phthalimide group via a 0-hairpin that is structurally conserved in all available complex structures, where a critically positioned glycine is in proximity to the phthalimide, producing a unique structural arrangement that is preserved in unrelated IMiD targets such as the zinc-finger proteins IKFZ1 and ZNF692, the kinase CK1a47, and the GTP-binding protein GSPT1. This interaction also seems to also be preserved in the context of heterobifunctional molecules. (M. Schapira et al., 2019, Nat Rev Drug Discov. 18, 949-963).

The primary medical and clinical uses of IMiDs are in the treatment of cancers and autoimmune diseases (including one that is a response to the infection leprosy). Medical indications for the use of these agents as regulatory approved therapeutics include, by way of nonlimiting example, myelodysplastic syndrome, a precursor condition to acute myeloid leukemia, erythema nodosum, a complication of leprosy, and multiple myeloma. Other useful indications include the treatment of Hodgkin's lymphoma, light chain-associated (AL) myeloidosis, primary myelofibrosis (PMF), acute myeloid leukemia, prostate cancer, and metastatic renal cell carcinoma (mRCC).

### Lenalidomide- and Lenalidomide Analog Dependent Mediation of Proteasomal Degradation

The drug thalidomide became infamous in the early 1960s when its use during the first trimester of pregnancy was linked to profound birth defects, most commonly a malformation of the upper limbs known as phocomelia. The discovery of thalidomide's teratogenic property was a major setback for use of the compound; however, thalidomide was later repurposed and today is an FDA-approved therapy for a number of disorders, including erythema nodosum leparum, 5q-myelodysplastic syndrome (MDS), and several mature B-cell malignancies, most notably the plasma cell malignancy multiple myeloma. Thalidomide's success as a treatment for these disorders motivated the synthesis of lenalidomide and pomalidomide, which are more potent derivatives that have largely replaced thalidomide in the clinic today.

It is now understood that these drugs function by mediating efficient proteasomal degradation of several protein targets by the E3 ubiquitin ligase CRL4-CRBN. These targets include the lymphocyte lineage transcription factors Ikaros (IKZF1) and Aiolos (IKZF3), as well as the Wnt pathway regulator Casein Kinase 1 alpha (CSNK1a1). The CRL4-CRBN E3 ubiquitin ligase belongs to the family of cullin-RING ligases and is a multi-subunit complex comprised of Ring Box Protein 1 (RBX1), DNA Damage Binding Protein 1 (DDB1), Cullin 4A (CUL4A), and Cereblon (CRBN). Thalidomide, lenalidomide, and pomalidomide bind specifically to CRBN, the substrate receptor for CRL4-CRBN. In doing so, these drugs increase the affinity of CRBN for Ikaros (IKZF1), Aiolos (IKZF3), and Casein Kinase 1 alpha (CSNK1a1). As a consequence of their increased association with the CRL4-CRBN ubiquitin ligase complex, these factors are efficiently ubiquitinated and degraded by the 26S proteasome. Without wishing to be bound by theory, the degradation of Ikaros (IKZF1) and Aiolos (IKZF3) explains not only the tumoricidal effect on myeloma cells, but also the immunomodulatory properties which have until now defined this class of compounds. Similarly, the degradation of Casein Kinase 1 alpha mediates remission of the malignant stem cell clone in 5q-myelodysplastic syndrome.

Lenalidomide and lenalidomide analogs are effective therapies for a number of diseases or disorders, including 5q-myelodysplastic syndrome (MDS), erythema nodosum leparum, and several mature B-cell malignancies, most notably, the plasma cell malignancy multiple myeloma. Lenalidomide analogs approved for clinical use by the Food and Drug Administration (FDA) include thalidomide and pomalidomide. Lenalidomide is approved by the FDA for treatment of 5q-myelodysplastic syndrome (MDS), erythema nodosum leparum, and multiple myeloma. In some embodiments, lenalidomide and lenalidomide analogs are administered to a subject having 5q-myelodysplastic syndrome (MDS) or plasma cell malignancy multiple myeloma. Lenalidomide or lenalidomide analogs may be also used in the treatment of any other disorders in which Ikaros (IKZF1), Aiolos (IKZF3), Casein Kinase 1 alpha (CSNK1a1), or other targets of lenalidomide may be implicated.

### Targeted Degradation of Substrates Using E3 Ubiquitin Ligase

Recent advances in the understanding of molecular glues and bifunctional PROTAC® molecules, which are reprogrammers of E3 ubiquitin ligase substrate specificity, have brought E3 ligases to the forefront of the therapeutic field and opened up new possibilities as to how E3 ligases can be utilized for therapeutic benefits. The use of molecular glue and PROTAC® molecules can ultimately result in the degradation of E3 substrate molecules in the cell.

Molecular glues or molecular glue degraders induce the interaction of an E3 ubiquitin ligase or another protein degradation-effecting complex with a new protein target.

PROTAC®, i.e., a “proteolysis targeting chimera,” is a heterobifunctional small molecule composed of a ligand that binds to a target protein, a ligand that binds to E3 ubiquitin ligase, and a linker that conjugates or joins these two ligands. By way of nonlimiting example, the linker constitutes about 5-15 carbon or other atoms and covalently interconnects the E3 ligase ligand and the target protein ligand. In an embodiment, a PROTAC® is a chemical knockdown strategy that degrades the target protein through the ubiquitin-proteasome system. In brief, PROTAC®s are composed of two, covalently linked protein-binding molecules, one that is capable of engaging E3 ubiquitin ligase (e.g., thalidomide or a functional analog thereof), and a second that binds to a target protein slated for degradation. Recruitment of the E3 ligase to the target protein results in ubiquitination and subsequent degradation of the target protein by the proteasome. Thus, a PROTAC® induces selective intracellular proteolysis, rather than functioning as a conventional enzyme inhibitor, by recruiting specific E3 ubiquitin ligases to transfer polyubiquitin chains onto target proteins, thereby marking them for degradation by the proteasome in a cell. (See. e.g., M. Schapira et al., 2019, Nat Rev Drug Discov. 18, 949-963 and X. Li et al., 2020, J. Hematol. & Oncol., 13(50); doi.org/10.1186/s13045-020-0085-3). PROTAC®s have the potential to degrade pathogenic target proteins and regulate the related signaling pathways, which cannot be achieved by traditional therapy.

### System and Methods for Specific E3 Ligase Substrate Ubiquitination and Identification

The system and methods utilizing the E3 ubiquitin ligase system and its tagged components as described herein are advantageous for identifying and selecting substrate targets of E3 ubiquitin ligases, both without and with a molecular glue or reprogrammer, such as, for example, an IMiD immunomodulatory agent that redirects CRBN to degrade neo-substrates, and/or or with a PROTAC® bifunctional molecule that artificially induces ligase-substrate interactions.

As noted supra, the new system (or platform) provided herein comprises at least the following two components. One component is ubiquitin (or a ubiquitin-like protein, “UBL”) that is fused to (“tagged with”) a peptide substrate for a biotin ligase. In an embodiment, the peptide substrate is a 15 amino acid peptide, called “A3-tag” herein. In an embodiment, the peptide substrate is a 15 amino acid peptide, called “AVI-TAG™-ubiquitin” (“Avi-tag”) herein. The system (or platform) also comprises an E3 ligase protein (enzyme) that is fused to (“tagged with”) with a non-promiscuous biotin ligase protein (enzyme). In an embodiment, the biotin ligase is a non-promiscuous bacterial biotin ligase. In an embodiment, the biotin ligase is the non-promiscuous E. coli biotin ligase BirA. In an embodiment, the biotin ligase is the non-promiscuous E. coli wild-type biotin ligase BirA. In an embodiment, the BirA biotin ligase fused to an E3 ligase is a photocaged BirA as described supra. In an embodiment, the photocaged BirA biotin ligase is a non-promiscuous BirA biotin ligase. In an embodiment, the photocaged BirA biotin ligase is a wild-type E. coli BirA biotin ligase. In an embodiment, the photocaged BirA biotin ligase is a non-promiscuous, wild-type E. coli BirA biotin ligase. In an embodiment, the photocaged BirA biotin ligase is BirA-K183(ONPK). In an embodiment, the BirA biotin ligase fused to an E3 ligase is a chemically-caged BirA as described supra. In an embodiment, the chemically-caged BirA biotin ligase is a non-promiscuous BirA biotin ligase. In an embodiment, the chemically-caged BirA biotin ligase is a wild-type E. coli BirA biotin ligase. In an embodiment, the chemically-caged BirA biotin ligase is a non-promiscuous, wild-type E. coli BirA biotin ligase in which lysine analog N-((E)-cyclooct-2-en-1-yl)-oxy)carbonyl-L-lysine (TCOK) is incorporated at residue K183. In accordance with the system (or platform) herein, ubiquitin (or a ubiquitin-like protein) is fused to the peptide substrate of biotin ligase (e.g., A3-tag peptide). An E3 ligase is fused to a non-promiscuous biotin ligase (e.g., E. coli wild type BirA) to produce an E3 ligase-biotin ligase fusion protein. In a cell where the interacting proteins are in proximity, the E3 ligase-biotin ligase fusion protein ligates (attaches) the A3-tagged ubiquitins to an E3 substrate. The A3-tagged ubiquitins on the E3 substrate, which is in proximity to the biotin ligase of the E3 ligase-biotin ligase fusion protein, are biotinylated by the biotin ligase fused to the E3 ligase at a specific site within the A3 peptide substrate. (FIG. 1A). In an embodiment, the system and method are active and functional within a cell.

By way of example, in the interaction-specific system described here, the E3 ligase fused to the non-promiscuous, wild type BirA biotin ligase catalyzes the transfer of ubiquitin (or ubiquitin-like) molecules that are tagged with A3-tag-or AVI-TAG™ to a target or substrate (e.g., a target or substrate protein) of the E3 ligase. Because of the proximity of the components in the system, the tagged ubiquitin (or ubiquitin-like) molecules on the E3 ligase substrate are then biotinylated by the BirA ligase that is fused to the E3 ligase. Consequently, the E3 ligase target or substrate protein is ubiquitinated with biotinylated ubiquitin (or ubiquitin-like) molecules, thereby allowing for the selection, identification, enrichment, and/or isolation of the biotinylated, ubiquitinated E3 ligase substrate protein(s), for example, by binding to streptavidin or avidin attached to a solid support using methods known and practiced in the art. Such methods include streptavidin/avidin-based pulldown assays, immunoassays, affinity chromatography, precipitation or immunoprecipitation methods.

In a particular embodiment, ubiquitin (or a ubiquitin like protein) is tagged with the A3-tag peptide substrate of BirA, and the E3 ligase protein is fused to a non-promiscuous, E. coli wild-type BirA biotin ligase. The wild-type biotin ligase (BirA) utilized in the system and methods described herein is optimally a non-promiscuous, BirA, such as non-promiscuous, E. coli wild type BirA. The wild-type biotin ligase (BirA) utilized in the system and methods described herein is distinct from the promiscuous BirA* ligase, for example, as used in BioID, which detrimentally leaks activated biotin, as reported, for example, by R. Sears et al. (2019, Meths. Mol. Biol., 2012:299-313) and in K. J. Roux et al., 2018, Curr. Protoc. Protein Sci., 91:19.23.1-19.23.15).

In the interaction-specific system and methods described herein, the use of a non-promiscuous, wild-type BirA biotin ligase allows only for the biotinylation of E3 ligase target proteins or substrates that are specifically conjugated to ubiquitin or UBLs that are themselves tagged with a peptide substrate for biotinylation, such as A3-tag AVI-TAG™, as described herein. Biotinylation of the tagged ubiquitin or UBLs that decorate the E3 ligase substrate protein is interaction specific, because it is carried out by the non-promiscuous BirA biotin ligase that is fused to the E3 ligase in the system This approach enables the biotinylation of E3 ligase substrates in a directed, ubiquitin-specific, and E3-substrate interaction-specific manner. In an embodiment, the BirA biotin ligase fused to an E3 ligase is a photocaged BirA as described supra. In an embodiment, the photocaged BirA biotin ligase is a non-promiscuous BirA biotin ligase. In an embodiment, the photocaged BirA biotin ligase is a wild-type E. coli BirA biotin ligase. In an embodiment, the photocaged BirA biotin ligase is a non-promiscuous, wild-type E. coli BirA biotin ligase. In an embodiment, the photocaged BirA biotin ligase is BirA-K183(ONPK). In an embodiment, the BirA biotin ligase fused to an E3 ligase is a chemically-caged BirA as described supra. In an embodiment, the chemically-caged BirA biotin ligase is a non-promiscuous BirA biotin ligase. In an embodiment, the chemically-caged BirA biotin ligase is a wild-type E. coli BirA biotin ligase. In an embodiment, the chemically-caged BirA biotin ligase is a non-promiscuous, wild-type E. coli BirA biotin ligase in which lysine analog N-((E)-cyclooct-2-en-1-yl)-oxy)carbonyl-L-lysine (TCOK) is incorporated at residue K183.

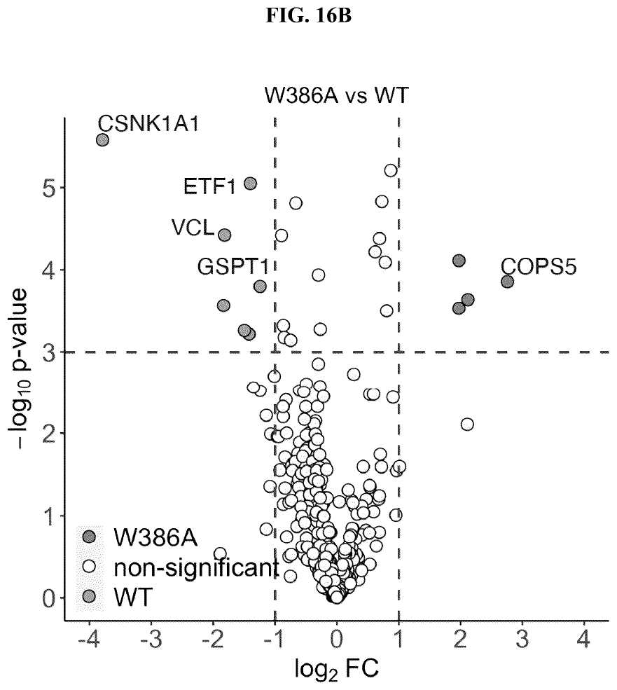

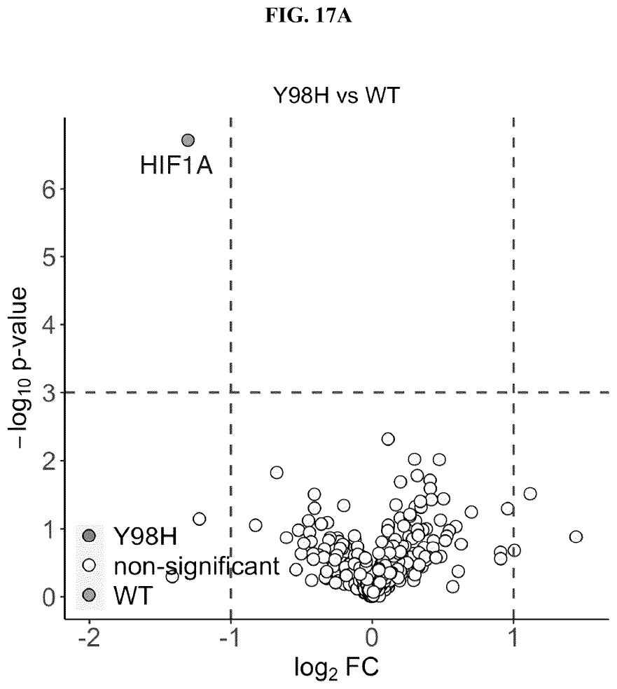

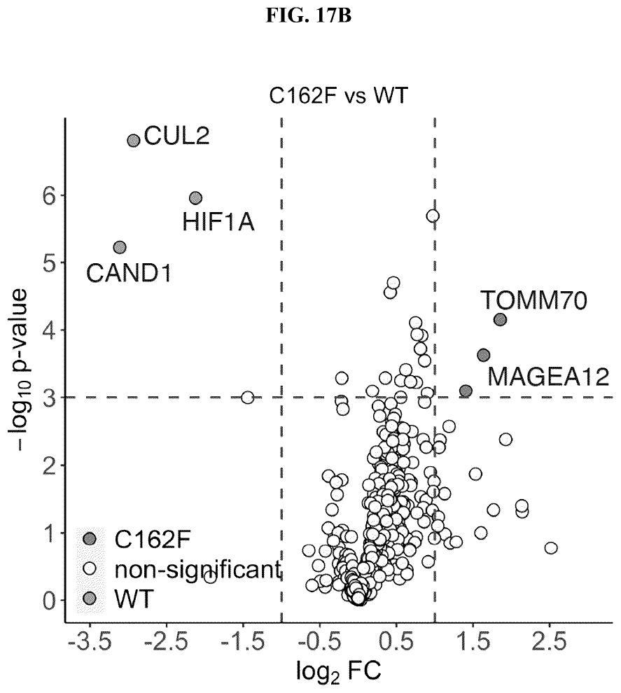

The identification of substrates of an E3 ubiquitin ligase can be achieved using the methods and systems described herein in conjunction with the use of mutants of E3 ubiquitin ligases (e.g., loss-of-function mutants of an E3 ligase) and comparing the ubiquitinated substrate(s) of the loss-of-function mutants with those of the wild-type E3 ligase. Differentially enriched proteins obtained by the use of the methods and systems described herein indicate candidate substrates of the E3 ligase. In an embodiment, the E3 ligase is CRBN or a loss-of-function mutant or variant thereof. (See, e.g., Example 9 and FIGS. 16A and 16B). In an embodiment, the E3 ligase is VHL or a loss-of-function mutant or variant thereof. (See, e.g., Example 10 and FIGS. 17A and 17B).

### Advantages of Biotin Ligase Peptide Substrate Tags, e.g., A3-Tag Peptide, Fused to Ubiquitins

As described supra, an peptide substrate tag, e.g., A3-tag peptide, suitable for use in the systems and methods described herein comprises or consists of amino acid sequence GLNDIFEAQKIE (SEQ ID NO: 2) (in an amino to carboxy terminal orientation). Non-limiting examples of variant A3-tag peptide sequences include the following: GLNDIFEAQKIEWHE (SEQ ID NO: 7), GLNDIFEAQKIEWH (SEQ ID NO: 8), GLNDIFEAQKIEW (SEQ ID NO: 9) GLNDIFEAQKIE (SEQ ID NO: 2), LNDIFEAQKIEWHE (SEQ ID NO: 10), LNDIFEAQKIEWH (SEQ ID NO: 11), LNDIFEAQKIEW (SEQ ID NO: 12), LNDIFEAQKIE (SEQ ID NO: 13), NDIFEAQKIEWHE (SEQ ID NO: 14), NDIFEAQKIEWH (SEQ ID NO: 15), NDIFEAQKIEW (SEQ ID NO: 16), NDIFEAQKIE (SEQ ID NO: 17), DIFEAQKIEWHE (SEQ ID NO: 18), DIFEAQKIEWH (SEQ ID NO: 19), DIFEAQKIEW (SEQ ID NO: 20), DIFEAQKIE (SEQ ID NO: 21). Any of the A3-tag peptide sequences may include an amino terminal initiating methionine (M1) residue and/or may include a carboxy terminal linker peptide or sequence, such as, without limitation, GSGGS (SEQ ID NO: 4), GSGG (SEQ ID NO: 5), GSG, SGGS (SEQ ID NO: 6), GGS, or GS. In an embodiment, the A3-tag peptide sequence comprises GLNDIFEAQKIE (SEQ ID NO: 2) or MGLNDIFEAQKIE (SEQ ID NO: 3), with or without a linker sequence.

In the system and methods herein, the use of ubiquitin (or a ubiquitin like protein) fused to the A3 tag peptide substrate of a non-promiscuous (wild type) BirA fused to E3 ligase, can result in a somewhat lower affinity interaction between the A3 tag peptide substrate and the BirA biotin ligase, compared, for example, with the affinity of BirA for the AVI-TAG™ BirA peptide substrate. By way of non-limiting example, the Kₘ between BirA and Avi-Tag is 25 μM, while that of BirA and A3-Tag is 345 μM, a near 14-fold increase in Kₘ, which reflects a lower reactivity. See. e.g., Hernandez-Suarez et al. (2008, J Am Chem Soc., July 23; 130(29):9251-9253. As such, a somewhat decreased level or amount of biotinylation of ubiquitin (or a ubiquitin-like protein) tagged with the A3 peptide substrate is observed. However, the proximity and interaction-specific nature of the components in the system and method described herein produces a higher local concentration of the components that interact and function together in the system and method, and an adequate and locally high level specificity of biotinylation of A3-tagged ubiquitins conjugated to the E3 ligase substrate is achieved, thus providing advantages and benefits of the described system and methods utilizing A3-tagged ubiquitin.

While a somewhat lower affinity interaction occurs between the non-promiscuous, wild-type E. coli BirA biotin ligase and the A3-peptide tagged ubiquitin, such a dynamic is advantageous in the interaction-specific system and methods described here, in which the local concentrations of E3 ligase substrate ubiquitinated with A3-tagged ubiquitin(s) and non-promiscuous BirA fused to the E3 ligase is enhanced within a cell due to the close proximities of the these components that is required for the biotinylation reaction to occur. Despite a lower total or overall signal produced by biotinylation of E3 substrates ubiquitinated with A3-peptide tagged ubiquitin molecules compared, for example, with biotinylation of E3 substrates ubiquitinated with Avi-tag peptide tagged ubiquitin molecules, an improved and advantageous signal-to-noise ratio is provided by use of the A3-tag. This is due to the interactive design of the tagged components of the system and the close proximities induced by the components and conditions of the system that allow for the functional activities of all of the tagged components to be achieved.

Without intending to be bound by theory, a lower affinity interaction between the A3-tagged ubiquitin and the BirA biotin ligase fused to E3 ligase decreases the extent to which A3-tagged ubiquitin and A3-tagged ubiquitinated proteins are biotinylated by BirA independent of E3 ligase-mediated interactions. This decrease in reactivity between the tag and BirA biotin ligase increases the likelihood that biotinylated A3-tagged ubiquitinated proteins are bona fide substrates of the BirA-fused E3 ligase, because the biotinylation reaction requires additional affinity that is provided by the interaction between the E3 ligase and the substrate to reach half-maximal activity for biotinylation. (FIG. 1A). With the use of the lower affinity A3-tagged ubiquitin molecules ligated to the E3 ligase substrate, the A3-tagged ubiquitinated proteins are preferentially biotinylated only when they are located proximal to the BirA biotin ligase fused to the E3 ligase via interaction with the E3 ligase, thus allowing for specific biotinylation of A3-tagged ubiquitinated substrates to occur. The system and method described herein require such an increased local concentration of the components needed for biotinylation, thereby achieving higher local substrate-enzyme specificity and a biotinylation signal and readout having a low signal-to-noise ratio. Thus, the greater local concentration of the components required by the design of the system and method described herein results in specific biotinylation of the A3-tagged ubiquitin molecules on the E3 ligase substrate by the BirA biotin ligase fused to the E3 ligase and effectively enhances the specificity of the enzyme/substrate biotinylation reaction. Biotinylation of the lower affinity A3-tagged, ubiquitinated E3 ligase substrate by BirA fused to the E3 ligase in the system and method herein successfully results from the greater local concentrations of the components of the system and method, which are brought into close proximity within the environment in which the interactions among the system's components occur, e.g., within a cell. Such interactions are designed to be ubiquitin- and interaction-specific.

In an embodiment, the system and methods described herein are carried out in vivo, e.g., in cells, including, without limitation, eukaryotic cells, mammalian cells, and human cells. In embodiments, the cells may be obtained or derived from a biological sample, a tissue, an organ, a biopsy, and the like. In embodiments, use of the system and methods described herein embrace cells ex vivo, in vivo, and/or in a living subject or organism. In an embodiment, one or more polynucleotides encoding the components of the system and methods, e.g., A3-tag fused to ubiquitin (or UBL molecules) and E3 ligase fused to non-promiscuous and/or wild-type biotin ligase (BirA), or a photocaged BirA biotin ligase, are introduced into cells by methods known and used in the art and as described herein, for example, without limitation, transfection, transduction, electroporation, infection, and the like. In an embodiment in which non-photocaged BirA biotin ligase is used in a cell or cell line, the cell or cell line is grown or cultured in biotin-depleted culture medium to reduce or eliminate the presence of naturally occurring biotin. The cells are cultured in or treated with biotin prior to carrying out an assay or using the cells in conjunction with the systems and methods described herein. In embodiments, the polynucleotides encoding components of the system can be operatively linked to promoters, enhancers, regulatory elements, and the like, and can be harbored in vectors, e.g., expression vectors, such as plasmids or viral vectors, for expression in cells, including mammalian and human cells. In embodiments, cells containing the tagged components of the in vivo biotinylated ubiquitination system described herein are treated with one or more E3 ligase modulatory agents (e.g., drugs, small molecules, and the like), or immunomodulatory imide molecules (IMiDs), as described herein. In embodiments, cells containing the tagged components of the in vivo biotinylated ubiquitination system described herein are treated with one or more molecular glues, reprogramming molecules, or PROTAC®s as described herein. In embodiments, cells containing the tagged components of the in vivo biotinylated ubiquitination system described herein are subjected to specific stress response or environmental stimuli (e.g., UV radiation, hypoxia, reactive oxygen species, nutrient supplementation, or nutrient depletion) for outcome analyses as described herein.

In an embodiment of the controlled, in vivo biotinylation approach as described herein, the short N-terminal biotin peptide epitope fused to ubiquitin, namely, the specific BirA recognition sequence fused to the N terminus of each ubiquitin protein or ubiquitin protein chain, (e.g., A3-tag), is efficiently biotinylated in cells by using a non-promiscuous and/or E. coli wild type BirA enzyme linked to E3 ligase, a photocaged BirA enzyme that is decaged by photolysis to activate the biotin ligase activity of the enzyme, or a chemically-caged BirA enzyme whose ligase activity is unblocked by the addition of a chemical activator such as DM-Tz; and biotinylated ubiquitin is incorporated efficiently into substrates of E3 ligases. In some embodiments, lysis of cells under denaturing conditions allows for the inactivation of UBL isopeptidases (and DUBs) and the high affinity biotin-streptavidin interaction allows stringent washes, to yield pure ubiquitin-modified proteins with almost no background from non-covalent interactors and non-specific contaminants. In an embodiment, the biotinylation of tagged ubiquitin using the system and methods described herein may also be carried out in living organisms.

### Uses of the Systems and Methods Involving Molecularly Tagged E3 Ligase and A3- or Avi-Tagged Ubiquitin Proteins

Without intending to be limiting or wishing to be bound by theory, the systems and methods described herein provide numerous and advantageous uses as described below, e.g., detection, analysis, screening, assessment and selecting, involving a variety of exogenously added or endogenous factors, agents, molecules, effectors treatments, and the like, to determine outcomes.

The methods herein provide useful, specific and directly interactive approaches to selecting, enriching and identifying mammalian (especially human) E3 ubiquitin ligase substrates, e.g., substrates that are targeted for protein degradation. The identification of E3 ligases that target substrates (protein substrates) involved in diseases, disorders and pathologies is of particular importance in the development of therapeutics and treatments for such diseases, disorders and pathologies, including cancers, tumors, neoplasias and a host of others. The system and methods provided herein also embrace the use of reprogramming molecules, such as small molecule molecular glues, IMiDs, and other modulatory agents that redirect E3 ligases, such as CRBN, to interact with and/or degrade new substrates, and PROTAC®s, which are capable of inducing E3 ligase-substrate interactions in cells.

The system and methods described herein are useful for assessing substrates of E3 ligases in cells, or changes induced in the E3 ligase substrates in cells, in different states, for example, cells that have been treated with one or more IMiDs or E3 ligase substrate modulators, versus untreated cells. In cells treated with an IMiD, a change, e.g., an increase or enhancement, in the amount(s) of biotinylated, ubiquitinated substrate species in cells can be detected and the substrate species can be identified, e.g., by Western blot and/or mass spectrometry.

The ubiquitination process, in which ubiquitin and ubiquitin-related proteins post-translationally modify proteins and thereby alter their functions, is involved in various physiological responses, including cell growth, cell death (apoptosis), and DNA damage repair. E3 ligases, the most specific enzymes of the ubiquitination system, bind to their substrates and participate in the turnover of many key regulatory proteins. E3 ligases can play a role in the development of cancer, as they have the ability to regulate the stability and function of proteins and substrates with which they interact. Depending on their modification type, ubiquitinated substrates of E3 ligase participate in different processes, including protein activation, inhibition, and proteasomal or lysosomal degradation. Ubiquitination also regulates protein localization, sorting, and various protein-protein interactions.

Because E3 ligases harbor substrate interaction domains, the compositions, system and methods described herein are useful for identifying key E3 ligase substrates, as well as E3 ligases, that can serve as therapeutic targets, particularly, for those known to be or identified as being associated with diseases and pathologies. The compositions, system and methods as described can be utilized in conjunction with a number of different modulating agents such as molecular glues or IMiDs, which reprogram or redirect E3 ligases, e.g., CRBN or CRL4-CRBN, for the targeted degradation of ubiquitinated neo-substrates, such as those that cause, are associated with, or are involved in a disease or pathology. e.g., a cancer, tumor, or neoplasm, in cells and tissues obtained from subjects. In addition, bifunctional molecules, such as PROTAC®s (proteolysis targeting chimeras), can also be utilized with the described compositions, system and methods in cells and tissues isolated from subjects to artificially induce E3 ligase-substrate interactions and to therefore identify the interacting substrates that are ubiquitinated ubiquitins (or UBLs) fused to an A3 tag, for example, and biotinylated. In embodiments, the cells and tissues are obtained (isolated) from patients having a disease or pathology.

As would be appreciated by the skilled practitioner in the art, E3 ligases recognize mutated and misfolded proteins (endogenous substrate proteins), or proteins that are no longer needed, and tag them with ubiquitin protein molecules, such as A3-tagged ubiquitin proteins of the compositions, system and methods described herein. As such, E3 ligases are critical components of the body's natural protein disposal system, which directs the ubiquitin-tagged target proteins to the proteasome where they are degraded into small peptides. In conjunction with the components of the compositions, system and methods described herein, PROTAC® protein degraders are capable of recruiting a chosen E3 ligase, e.g., an E3 ligase fused to a non-promiscuous biotin ligase, into close proximity with a specific endogenous substrate, namely, a disease-causing substrate protein, that can be tagged with ubiquitin fused to a biotin ligase peptide substrate A3-tag, thus allowing for the identification, selection, and/or therapeutic targeting of the disease-causing substrate and/or the E3 ligase, for example, in cells and/or tissues obtained or isolated from a subject, such as a patient having a disease. PROTAC®s can be used for tissue-specific targeting by recruiting an E3 ligase that is expressed only in a specific cell lineage.

In embodiments, the compositions, system and methods as described can be utilized to determine the activity or function of a mutated or variant E3 ligase fused to a non-promiscuous biotin ligase (BirA) in its interaction with and ubiquitination of a substrate protein compared with that of a non-mutated or non-variant E3 ligase fused to fused to a non-promiscuous biotin ligase (BirA), for example, within a cell or a given cell type.

In embodiments, the compositions, system and methods as described can be utilized to compare substrate-E3 ligase interactions and/or activity or function of an E3 ligase with its target substrate, and/or to identify substrates that may be specific or specifically active in cells and tissues at certain stages in development, e.g., the normal development of cells and tissues in animals, including humans. Nonlimiting examples of cells for use include neuronal cells, neurons, muscle cells, skeletal muscle cells, adipose cells, cells from the liver, heart, kidney, lung, thyroid, pancreas, gall bladder, bladder, stomach, esophagus, eye, brain, testes, ovary, cervix, prostate, and the like.

In embodiments, the compositions, system and methods as described can be utilized to compare substrate-E3 ligase interactions and/or activity or function of an E3 ligase with its target substrate, and/or to identify substrates that may be specific to a disease, in cells associated with a disease state versus normal, healthy, and/or non-diseased cells. By way of example, the components of the compositions and system described herein can be used to investigate and profile the E3 ligase-substrate interactions and/or to select and identify E3 ligase substrates in cancer, tumor, or neoplastic cells, cell lines, or cell cultures versus normal, healthy cells, cell lines, or cell cultures. In such cases, new and different E3 ligase substrates, or substrates harboring different post-translational modifications, may be detected and identified in cancer cells compared with normal, healthy cells using the system and compositions described herein. In embodiments, the cells are primary cells, cells isolated from tissues, cells isolated from tumors or cancers or neoplasms, cell lines, or cultured cells or cell lines.

In embodiments, the compositions, system and methods as described can be utilized to compare substrate-E3 ligase interactions and/or activity or function of an E3 ligase with its target substrate under different physiological or environmental conditions, e.g., normal versus aberrant, abnormal, or different from normal conditions, particularly in various developmental and stress response pathways. The conditions may be induced or found naturally. By way of non-limiting example, the substrate-binding E3 ligase von Hippel Landau (VHL) protein is a tumor suppressor that functions as a master regulator of the activity of Hypoxia-Inducible Factors (HIFs), which are heterodimeric oxygen-sensitive basic helix-loop-helix transcription factors that play roles in cellular adaptation to low oxygen environments. VHL regulates HIF activity by targeting the hydroxylated HIF-α subunit for ubiquitination and rapid proteasomal degradation under normoxic conditions. Mutations in VHL can be found in familial and sporadic hemangioblastomas, clear cell carcinomas of the kidney, pheochromocytomas and inherited forms of erythrocytosis and evidence the importance of disrupted molecular oxygen sensing in the pathogenesis of these diseases. VHL recognizes substrate and ubiquitinates and targets HIFα subunit for oxygen-dependent proteolysis. Therefore, either loss of VHL gene expression as a result of gene deletion or promoter hypermethylation, or mutations in VHL that affect its ability to capture and/or ubiquitinate HIF-α, results in constitutive HIF stabilization and activation of HIF controlled transcriptional programs irrespective of oxygen levels. The VHL/HIF-α interaction is highly conserved between species, underscoring its importance in molecular oxygen sensing. (V. Hasse, 2009, Curr Pharm Des, 15(33):3895-3903). In particular, VHL fused to non-promiscuous biotin ligase BirA may be constitutively or inducibly expressed in cells with ubiquitins fused to the A3 peptide substrate of BirA in cells which are placed under normal oxygen (O₂) conditions or in cells which are placed under hypoxic conditions to determine and identify endogenous VHL substrate(s), for example, HIF1A and HIF2A, that are ubiquitinated in cells under normal O₂ conditions compared with substrates that are ubiquitinated in cells under hypoxic conditions. The results of such analyses allow for the potential development of therapeutics and therapeutic treatment and/or intervention.

In embodiments, the compositions, system and methods as described can be utilized to investigate and identify new/neo substrates, or mutated or variant substrates of many different E3 ligases. Any E3 ligase of interest can be fused to a non-promiscuous biotin ligase, such as the non-promiscuous E coli wild type BirA biotin ligase described herein, and used as a component in the system and methods described herein to biotinylate those E3 ligase substrates that are ubiquitinated with ubiquitins fused to the biotin ligase peptide substrate (e.g., A3-tagged ubiquitins) and to enrich for, select and identify the substrates.

In embodiments, the compositions, system and methods as described can be utilized to investigate and identify E3 ligase substrates in cells at different developmental stages, in cells derived or obtained from an organism at different stages of development, in cells derived or obtained from a subject with a genetic disorder, in cells derived or obtained from a subject at different stages of disease, or in cells derived or obtained from a subject at different stages of treatment or therapy of a disease. By way of nonlimiting example, the systems and methods described herein can be used to monitor the status, levels, and/or presence of biotinylated and tagged ubiquitinated substrates of various E3 ligases in cells (or tissues) obtained from a subject (a patient or individual or organism) at a first timepoint compared with one or more later (or different) timepoints. Such timepoints may embrace times prior to or at the start of a treatment or therapy of the subject for a certain disease or disorder, and at different times during the course of the treatment or therapy for the disease or disorder to determine the progress or resolution of the treatment or therapy. Such timepoints may embrace one or more times at an early stage of development and at different times during the course of development or at different developmental stages of a subject or organism. In embodiments of the foregoing, cells and tissues obtained or isolated from a subject, such as a patient having a disease or a condition, are used in conjunction with the systems and methods described herein.

In embodiments, the compositions, system and methods as described can be utilized to identify new molecular glues, such as other molecules (e.g., proteins, peptides, small molecules) that create or modulate an interaction between a ligase and a substrate. In an embodiment, the ligase is E2 ligase. In an embodiment, the ligase is E3 ligase. In other embodiments, the compositions, system and methods as described can be utilized as a profiling assay for determining the specificity of agents, molecules, or drugs that alter ligase function (e.g., inhibitors, glues, PROTAC®s). By way of example, panels of BirA-ligase fusions can be used. For identifying agents that modulate ubiquitination activities of a ligase toward a substrate, almost any proximity-based detection method that can detect the presence of biotin, e.g., using streptavidin or an anti-biotin antibody, linked to the substrate of interest, such as an antibody that recognizes the substrate directly or a tag on the substrate, is suitable for use. Nonlimiting examples of assays that may be used include time-resolved fluorescence resonance energy transfer (TR-FRET) and ALPHAScreen. Ligase activities can be profiled either under endogenous conditions or in the presence of a ligase modifying agent or a specificity-modulating agent, e.g., CRBN and VHL, as well as other molecules described herein. Such a ligase panel could expand to tens or hundreds of ubiquitin and ubiquitin-like protein ligases.

### Use of the Described System and Methods to Identify Target Molecules of PROTAC®s Containing IMiDs or Other Ligase Modifying Agents Linked to Protein-Targeting Agents, Such as Kinase Inhibitors or Degraders

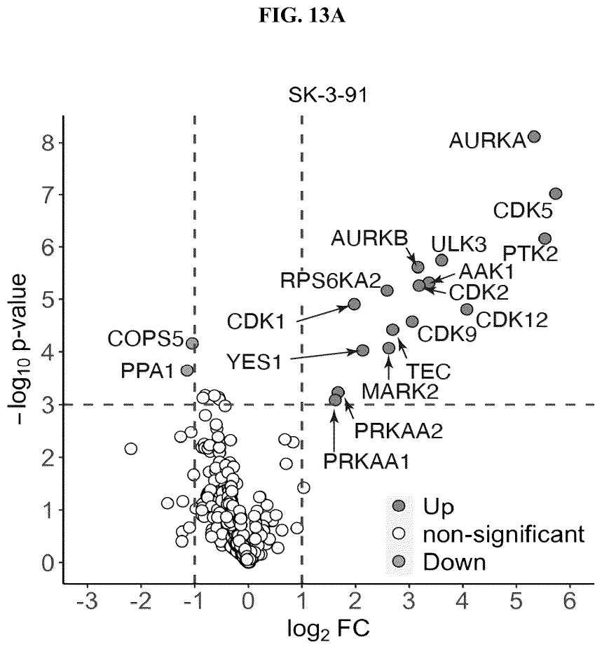

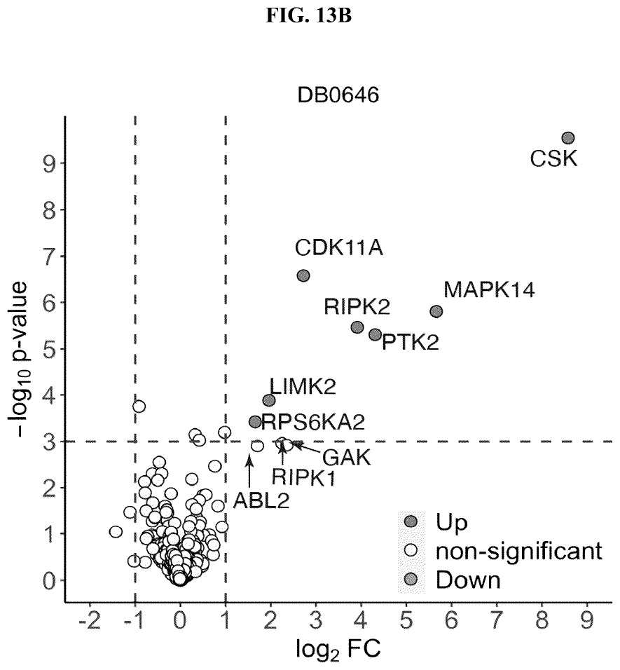

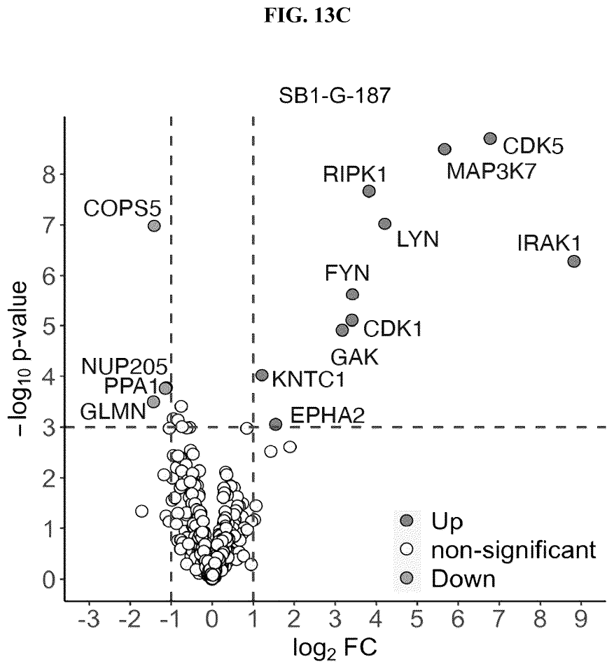

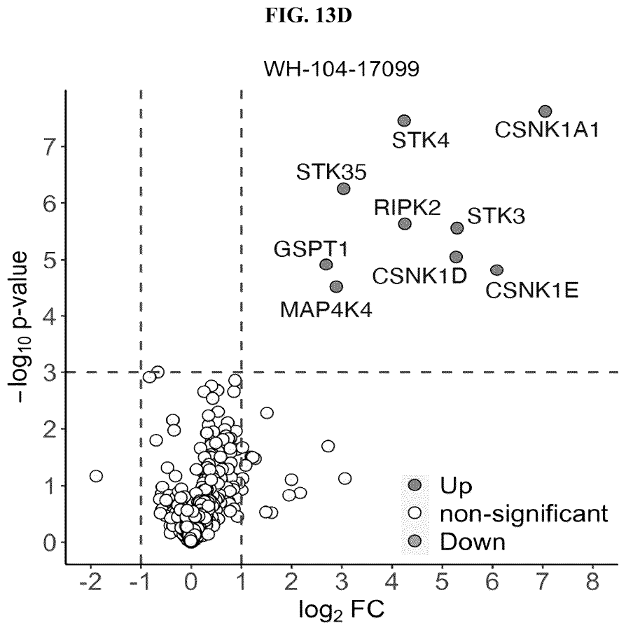

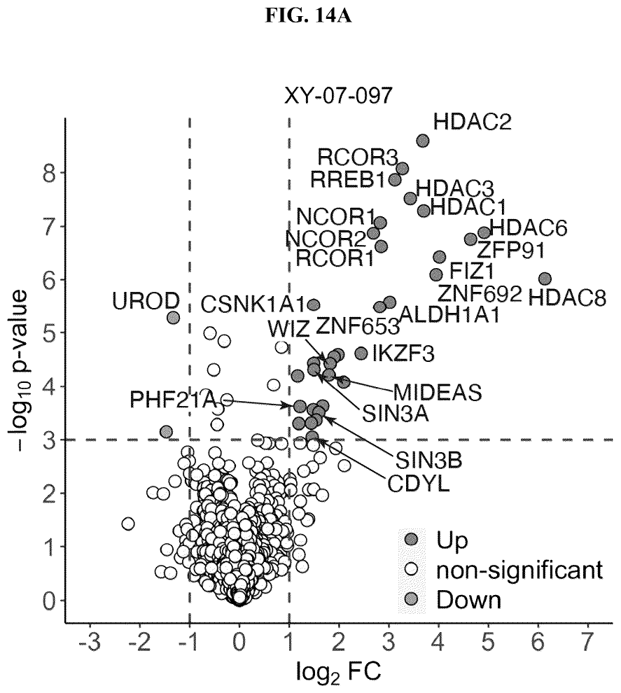

To study proteins that are downregulated by PROTAC®s (proteolysis targeting chimeras), total proteomics are typically carried out. However, it is currently difficult to directly identify ubiquitinated targets of E3 ligases. The presently described systems and methods afford the ability to directly screen for and identify ubiquitinated targets of E3 ligases. In an aspect for this purpose, and by way of example, kinase-targeting PROTAC® molecules were created by linking kinase inhibitors or degraders with IMiDs that modulate E3 ligases. (FIG. 12). Inhibitors for different kinases, e.g., tyrosine kinases, serine/threonine kinases, transcriptional kinases, kinases associated with various cancers and diseases, for use in the systems and methods are not intended to be limiting, nor are the E3 ligases and IMiDs that may be used. By way of example, numerous kinases (and potential degradable kinases) are known. (See. e.g., K. A. Donovan et al., 2020, Cell, 183(6):1714-1731.e10). The system and methods as described herein were used to profile the ubiquitinated targets of four CRBN-recruiting multi-kinase PROTAC®s, which were generated to contain the following multikinase degraders: SK-3-91, DB0646, SB1-G-187 and WH10417-099. These four degraders can collectively induce degradation of a large number of distinct kinases (>125 distinct kinases), (Ibid., at page 1718). In embodiments, each of these four multikinase degraders was linked to an IMiD analog, e.g., the IMiD analog pomalidomide, in the system and methods described herein to identify targets of the kinase inhibitors or kinase degraders. (FIG. 12 and FIGS. 13A-13D).

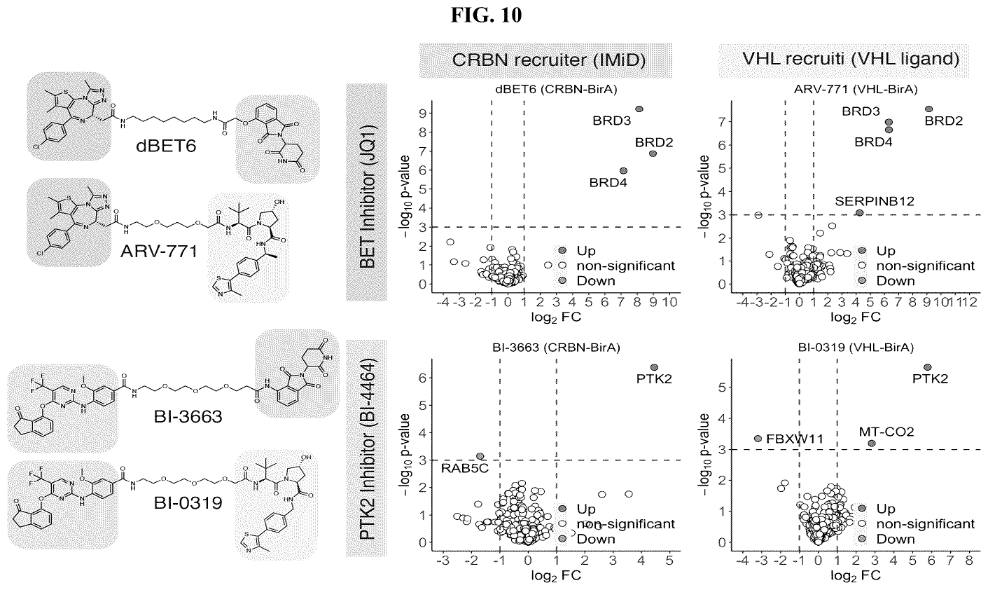

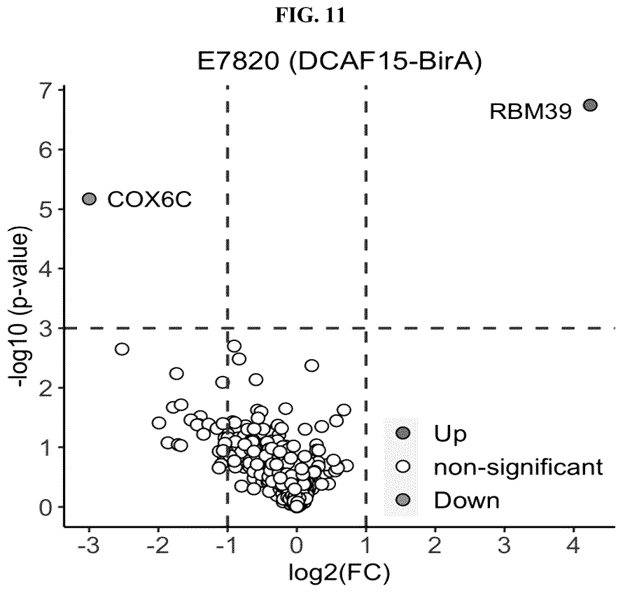

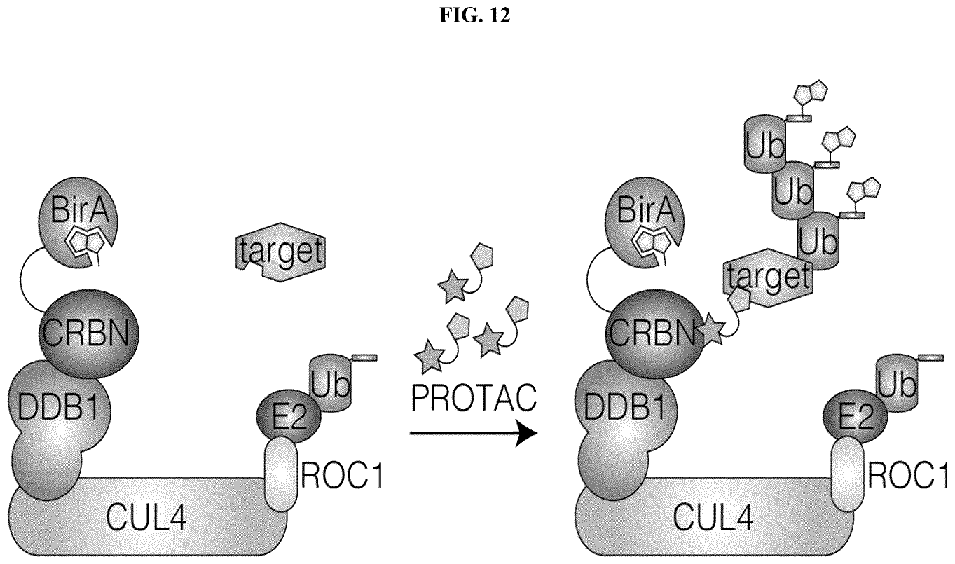

In addition to multikinase degraders, the systems and methods described herein were also found to be effective in profiling, detecting and identifying the ubiquitinated targets of PROTAC®s, such as, for example, BET bromodomain degraders and PTK2 degraders, that recruit not only CRBN E3 ubiquitin ligase, but also other E3 ligases, for example, VHL E3 ubiquitin ligase, whose activities generate identifiable ubiquitinated products (substrates). The ubiquitinated substrates of different E3 ubiquitin ligases were detected and identified using the system and method as described. (See, Example 6 and FIG. 10). In addition, the systems and methods described herein were also found to be effective in profiling, detecting and identifying the ubiquitinated targets of E3 ligases using non-CRBN-related protein degraders, such as molecular glues. By way of nonlimiting example, an aryl sulfonamide may function as a molecular glue, by virtue of its activity in altering the substrate repertoire of the CRL4-DCAF15 E3 ubiquitin ligase to induce ubiquitination and proteasome degradation of the splicing factor RNA binding motif protein 39 (RMB39), (Han, T. et al., 2017, Science, 356(6336); Uehara, T. et al., 2017, Nat Chem Biol., 13(6):675-680), (See, Example 7 and FIG. 11).

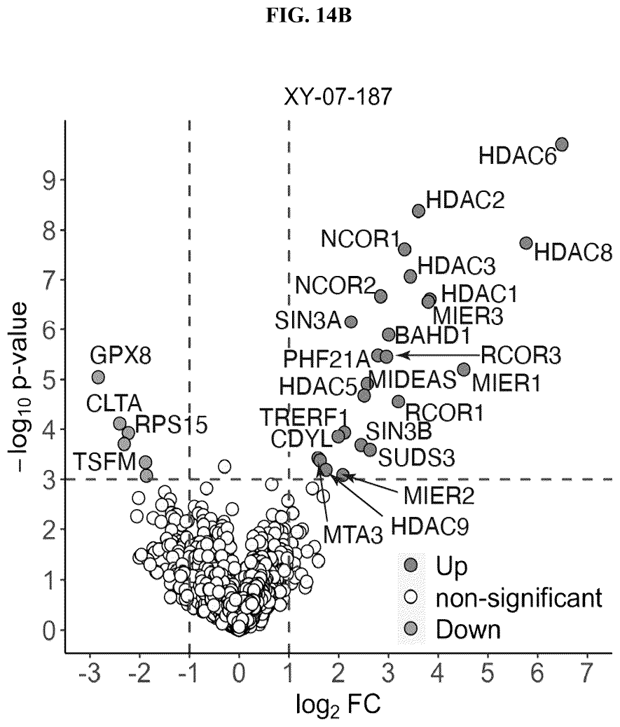

The system and method described herein were found to be effective in profiling, detecting and identifying the ubiquitinated targets of E3 ligases using other PROTAC®s, namely, Histone Deacetylase (HDAC) inhibitors (degraders), for example, as described in Example 8 and FIGS. 14A and 14B.

### Use of Inhibitors of E3 Ligases to Identify E3 Ligase Substrates

The methods and systems described and featured herein are useful in the identification of substrates for E3 ligases (or cullin ring E3 ubiquitin ligases (CRLs)) when performed and used in conjunction with a specific inhibitor of a particular E3 ubiquitin ligase or of an E3 ubiquitin ligase undergoing study. In an embodiment, the inhibitor is an enzymatic inhibitor of the E3 ligase. In an embodiment, the inhibitor is an inhibitor of a cullin-RING E3 ubiquitin ligase (CRL). CRLs constitute the largest superfamily of E3 ubiquitin ligases, with over 400 members known in mammals. They constitute modular complexes that are tightly regulated in the cell. (See, e.g., Nguyen, H. C. et al., 2017, Subcell Biochem., 83:323-347).

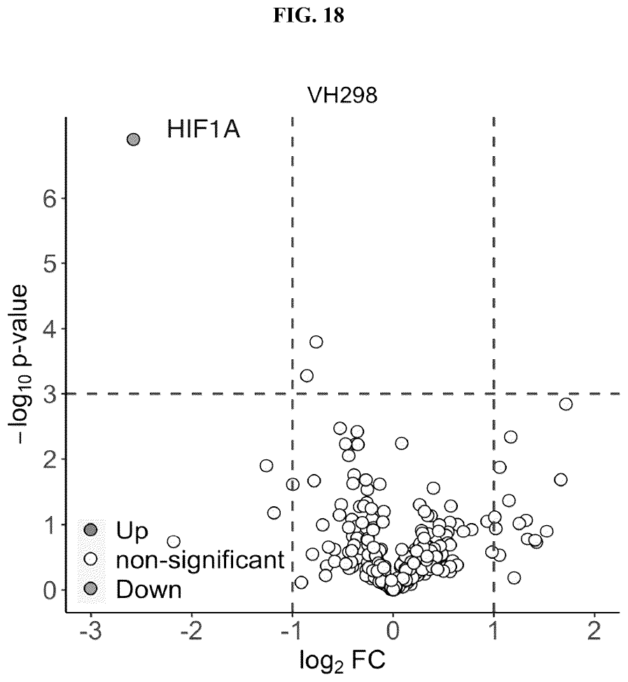

In an aspect, mass spectrometry experiments were carried out in which protein substrates of a representative E3 ligase, i.e., VHL E3 ligase, were identified using a VHL inhibitor. By way of nonlimiting example, the inhibitor is VH298, a cell-permeable ligand that competes with its endogenous substrates for VHL binding. (See, Example 11 and FIG. 18). In particular, VH 298 is a high-affinity inhibitor of VHL E3 ubiquitin ligase (K_{d}=80-90 nM), which blocks the interaction between VHL and HIF-α downstream of HIF-α hydroxylation, thereby initiating an hypoxic response. This activity results in a time- and concentration-dependent accumulation of hydroxylated HIF-α, and upregulates mRNA and protein levels of HIF target genes, showing a transcriptional profile similar to that of hypoxia. The products resulting from the performance of the methods described herein are analyzed via mass spectrophotometry to allow for statistical analysis of differentially enriched proteins, represented by log 2-fold-change between cells treated with the E3 ligase inhibitor and untreated cells containing wild-type E3 ligase. The ubiquitinated substrates of the E3 ligase are assessed and identified following mass spectrometry analysis, which reveals a decrease in biotinylation as a result of treatment of the cells with the specific E3 ubiquitin ligase. Thus, E3 ligase substrates can be discovered, detected, and identified using an inhibitor of the E3 ligase of interest in the methods and systems described herein.

### Inhibition of Overall E3 Ligase Activity in Cells Prior to the Identification of Ubiquitinated Substrates of E3 Ubiquitin Ligases Using the Described Methods and Systems

In an aspect related to the methods and systems described herein, cells used in the methods can be treated, e.g., pre-treated, with an agent, compound, drug, or other means to inhibit, block, or stall the ubiquitin conjugating process in cells, for example, a pan-cullin RING ligase (CRL) inhibitor or a CRL inhibitor, followed by a release or termination of the inhibition, blocking, or stalling at the time of carrying out the methods as described herein to identify ubiquitinated substrates of E3 ligases, including cullin-RING ligases (CRLs), (Petroski M. et al., 2005, Nature Reviews Molecular Cell Biology, Vol. 6, pp. 9-20). The agent, compound, drug, or other means is not particularly limited, except that it must act to inhibit, block, or stall the ubiquitin conjugating and proteasome systems in cells for a predetermined time prior to its being removed from the cell culture or environment, for example, by a washout, followed by replacement with normal cell culture medium. In embodiments, pre-treatment or treatment of the cells with the ubiquitin conjugating system inhibiting agent, small molecular weight compound, drug constitutes a time period of 0.2, 0.25, 0.3, 0.4, 0.5, 0.6, 0.7, 0.8, 0.9, 1.0, 1.25, 1.5, 1.75, 2.0. 2.25, 2.5 hours, and time periods therebetween, prior to washout.

In an embodiment, the cullin ubiquitin conjugating system inhibiting agent, compound, or drug utilized in the pre-treatment is MLN4924 (Pevonedistat), a potent and selective small molecule NEDD8-activating enzyme (NAE) inhibitor (Brownell, J. E. et al., 2010, Mol Cell, 37(1): 102-111; Soucy, T. A. et al., 2009, Clin Cancer Res, 15(12):3912-3916; Soucy, T. A. et al., 2009, Nature, 458(7239):732-736). NAE plays an important role in regulating the activity of CRLs, a subset of the E3 ubiquitin ligases, which regulate the destruction of many intracellular proteins. (See, Example 10 herein). In other embodiments, the CRL inhibitor is selected from 33-11 (7,8-dihydroxy-3-(1-phenyl-1H-pyrazole-4-yl)-2-(trifluoromethyl)-4H-chromen-4-one (ChemBridge) or KH-4-43 (Wu, K. et al., 2021, Proc. Natl. Acad. Sci. USA, 118(8), pp 1-11). By way of example, for VHL E3 ligase substrate identification, the NEDD8 activating enzyme inhibitor MLN4924 (Pevonedistat) was used to pre-block cullin-RING ligase (CRL) activities (see, e.g., FIGS. 17A, 17B and FIG. 18).

The pretreatment or contacting of the cells used in the method with the E3 ligase (e.g., CRL ligase) inhibitor, e.g., MLN4924, serves to inhibit the activity of all CRLs, which constitute a subset of ubiquitin ligases, thereby allowing stabilized E3 ligase or CRL substrates to accumulate in the cell. Without intending to be limiting, in embodiments, the pre-treatment period may be 15 minutes to 30 minutes, 30 minutes to 1 hour, 1 hour to 1.5 hours, 1.5 hours to 2 hours, or 1.5 hours to 3 hours. This pretreatment is followed by a washout of the inhibitor, which releases the ligase activity from being blocked, so that the E3 ligase or CRL substrates are re-ubiquitinated. During the washout time period, biotin labeling of ubiquitinated substrates (proteins, peptides, products) is carried out by performing the cellular biotin labeling method as described herein. In embodiment, one washout is performed. Without intending to be limiting, in embodiments, the time period of the washout can range from 15 minutes to 30 minutes, or from 15 minutes to 1 hour, during which time period biotin labeling is performed in the last 15 minutes. The biotinylated and ubiquitinated reaction products of the method are analyzed and identified, e.g., by mass spectrometry, as described. (See, e.g., Example 11, FIG. 17).

Similarly, experiments similar to those described in Examples 10 and 11 can be performed in which a ubiquitin activating enzyme inhibitor that inhibits all ubiquitin ligase activities can be used. For the identification of substrates of non-cullin-RING E3 ligases, such as RING E3 ligases and HECT E3 ligases, other E3 ligase inhibitors may be employed. In an embodiment, such a ubiquitin activating enzyme inhibitor is MLN7243 (TAK243) (MedChemExpress LLC, Monmouth, NJ), having the below structure, can be used.

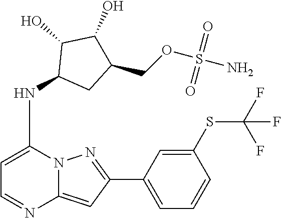 [SMILES: NS(=O)(=O)OC[C@H]1C[C@@H](Nc2ccnc3cc(-c4cccc(SC(F)(F)F)c4)nn23)[C@H](O)[C@@H]1O]

MLN7243 (TAK-243) is a selective ubiquitin activating enzyme (UAE), (UBA1), inhibitor (IC₅₀=1 nM), which blocks ubiquitin conjugation, thereby disrupting mono-ubiquitin signaling as well as global protein ubiquitination. Other activities of TAK-243 (MLN7243) include inducing endoplasmic reticulum (ER) stress, abrogating NF-κB pathway activation and promoting apoptosis. Accordingly, for studies and experiments involving the methods and systems described herein in which inhibition of the activity of a subset of ubiquitin ligases, such as all CRLs, is desired or required, the inhibitor MLN4924, which inhibits all CRL activities, can be used. In addition, for studies and experiments in which inhibition of all ubiquitin ligases is desired or required, the inhibitor MLN7243, which inhibits all E3 ubiquitin ligase activities, can be used. In an embodiment, inhibitors such as MLN7243 can be used to identify substrates of non-cullin-RING ligases, such as RING ligases and HECT ligases.

### Cloning and Recombinant Protein Expression and Purification

In embodiments, assays and compositions for protein engineering are used to construct the tagged/fused components of the system and compositions described herein. To obtain high level expression of a cloned gene, a nucleic acid is subcloned into an expression vector that contains a promoter to direct transcription, a transcription translation terminator, and for a nucleic acid encoding a protein, a ribosome binding site for translational initiation. Suitable bacterial promoters are well known and used in the art and are described, for example, in “Molecular Cloning: A Laboratory Manual”, second edition (Sambrook et al, 1989), and Ausubel et al., Eds., Current Protocols in Molecular Biology, 1995 supplement (and more recent editions). Bacterial expression systems for expressing proteins in e.g., E. coli, Bacillus sp., and Salmonella are reported by Palva et al., (Gene 22:229-235 (1983)) and Mosbach et al., Nature 302:543-545 (1983)). Kits for such expression systems, as well as retroviral expression systems, are commercially available. Eukaryotic expression systems for mammalian cells, yeast, and insect cells are well known in the art and are also commercially available.

Polynucleotide Vectors and Methods of Introducing or Delivering Polynucleotides and Polypeptides into Cells

Delivery or introduction of one or more polynucleotides encoding component fusion proteins of the system and methods described herein and their expression in a cell may be carried out by a variety of methods as known and practiced in the art. A number of vectors may be used to introduce polynucleotides encoding proteins that are expressed and have functional activity in a cell. Typically, recombinant polypeptides are produced by transfection or transformation of a suitable host cell with all or part of a polypeptide-encoding nucleic acid molecule or fragment thereof in a suitable expression vehicle. In some embodiments, cells are transfected for stable expression of a recombinant or genetically engineered protein (stable expression). In some embodiments, cells are transfected for transient expression of a recombinant or genetically engineered protein (transient transfection/expression).

By way of example, transducing viral (e.g., retroviral, lentiviral, adenoviral, and adeno-associated viral) vectors can be used, as they have high efficiency of infection and stable integration and expression (see, e.g., Cayouette et al., Human Gene Therapy, 8:423-430, 1997; Kido et al., Current Eye Research, 15:833-844, 1996; Bloomer et al., Journal of Virology, 71:6641-6649, 1997; Naldini et al., Science, 272:263-267, 1996; and Miyoshi et al., Proc. Natl. Acad. Sci. U.S.A., 94:10319, 1997). For example, a polynucleotide encoding ubiquitin fused to the peptide substrate of a non-promiscuous biotin ligase (e.g., the A3-tag peptide substrate of non-promiscuous BirA biotin ligase as described herein), and a polynucleotide encoding an E3 ligase fused to a non-promiscuous biotin ligase enzyme (e.g., E. coli wild type BirA) can be cloned into one or more retroviral vectors, and expression can be driven from an endogenous promoter, from an inducible promoter, from the retroviral long terminal repeat, or from a promoter specific for a target cell type of interest. By way of example, an inducible promoter may be used to induce expression of the E3 ligase fused to the non-promiscuous biotin ligase in a particular cell type or under certain culture or physiological conditions.

Expression of proteins from eukaryotic vectors can be regulated using inducible promoters. With inducible promoters, expression levels are tied to the concentration of inducing agents, such as, for example, tetracycline, doxycycline, or tamoxifen, by the incorporation of response elements for these agents into the promoter. Generally, high level expression is obtained from inducible promoters only in the presence of the inducing agent; basal expression levels are minimal. In addition, vectors can have a regulatable promoter, e.g., tet-regulated systems and the RU-486 system (see. e.g., Gossen and Bujard, 1992, PNAS USA 89:5547; Oligino et al., 1998, Gene Ther. 5:491-496; Wang et al., 1997, Gene Ther. 4:432-441; Neering et al., 1996, Blood 88: 1147-1155; and Rendahl et al., 1998, Nat. Biotechnol. 16:757-761). The use of such promoters imparts small molecule control on the expression of the candidate target nucleic acids, which can be used to determine that a desired phenotype results from a transfected cDNA rather than a somatic mutation. In an embodiment, a tetracycline/doxycycline-inducible promoter/expression (e.g., viral expression) system is used. Such systems are commercially available, e.g., Tet-One systems or Tet-On 3G systems (Takara Bio USA, Inc., San Jose, CA); or pLIX402; pINDUCER20 and pINDUCER21 (Addgene, Watertown, MA).

Some expression systems have markers that provide gene amplification such as thymidine kinase and dihydrofolate reductase. Alternatively, high yield expression systems not involving gene amplification are also suitable, such as using a baculovirus vector in insect cells, with a sequence of choice under the direction of the polyhedrin promoter or other strong baculovirus promoters. Other elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, as any of the many resistance genes known in the art are suitable. The prokaryotic sequences are preferably chosen such that they do not interfere with the replication of the DNA in eukaryotic cells, if necessary.

Other viral vectors that can be used include, for example, lentivirus, vaccinia virus, bovine papilloma virus, or herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cometta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and L. G. Johnson, Chest, 107:77S-83S, 1995.

Non-viral approaches can also be employed for the introduction of a nucleic acid molecule (polynucleotide) into a cell. In some embodiments, the polynucleotide is DNA, RNA, mRNA, cDNA, and the like. For example, a nucleic acid molecule (polynucleotide) can be introduced into a cell by administering the nucleic acid in the presence of lipofection (Feigner et al., Proc. Natl. Acad. Sci. U.S.A., 84:7413, 1987; Ono et al., Neuroscience Letters, 17:259, 1990; Brigham et al., Am. J. Med. Sci., 298:278, 1989; Staubinger et al., Methods in Enzymology, 101:512, 1983), asialoorosomucoid-polylysine conjugation (Wu et al., Journal of Biological Chemistry, 263:14621, 1988; Wu et al., Journal of Biological Chemistry, 264:16985, 1989), micro-injection under surgical conditions (Wolff et al., Science, 247:1465, 1990), or by nanoparticles and the like. In some embodiments, the nucleic acids are administered in combination with a liposome and protamine.

The delivery or introduction of a nucleic acid molecule (polynucleotide or cloned genomic DNA, cDNA, synthetic DNA or other foreign genetic material) into cells can also be achieved using non-viral means involving transfection in vitro. Such methods include, without limitation, the use of calcium phosphate, polybrene, DEAE dextran, electroporation, protoplast fusion, biolistics, microinjection, and any of the other well-known methods practiced in the art. Liposomes or lipid particles (lipid nanoparticles) can also used for delivery of DNA into a cell, such as a 293T cell. In some embodiments, electroporation (electro-permeabilization) methods can be used to introduce polynucleotides encoding the proteins or proteins (recombinant proteins) into cells, such as mammalian cells. As will be appreciated by those having skill in the art, electroporation is a technique in which an electrical field is applied to cells in order to increase the permeability of the cell membrane, allowing polynucleotides (DNA), chemicals, drugs, or electrode arrays to be introduced into the cells (also called electrotransfer). It is only necessary that the particular genetic engineering procedure used is capable of successfully introducing at least one gene into a host cell that is capable of expressing proteins and encoding nucleic acids as described herein.

cDNA expression for use in polynucleotide expression methods can be directed from any suitable promoter (e.g., the human cytomegalovirus (CMV), simian virus 40 (SV40), or metallothionein promoters), and regulated by any appropriate mammalian regulatory element. The selection of a promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. Heterologous refers to portions of a nucleic acid and indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature, e.g., a fusion protein.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence encoding the nucleic acid of choice and signals required for efficient polyadenylation of the transcript, ribosome binding sites, and translation termination. Additional elements of the cassette can include enhancers and, if genomic DNA is used as the structural gene, introns with functional splice donor and acceptor sites. In addition to a promoter sequence, an expression cassette should also contain a transcription termination region downstream of the structural gene to provide for efficient termination. The termination region can be obtained from the same gene as the promoter sequence or can be obtained from different genes.

If desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a construct, regulation can be mediated by the cognate regulatory sequences or, if desired, by regulatory sequences derived from a heterologous source, including any of the promoters or regulatory elements described above.

The particular expression vector used to transport the genetic information into the cell is not particularly critical. Any of the conventional vectors used for expression in eukaryotic or prokaryotic cells can be used. Standard bacterial expression vectors include plasmids such as pBR322 based plasmids, pSKF, pET23D, PGEX6P-1, pRSFDuet, and fusion expression systems such as MBP, GST, and LacZ. Epitope tags can also be added to recombinant proteins to provide convenient methods of isolation, e.g., c-myc, HA, and the like. Sequence tags can be included in an expression cassette for nucleic acid rescue. Markers such as fluorescent proteins, green or red fluorescent protein, 13-gal, CAT, and the like can be included in the vectors as markers for vector transduction.

As will be appreciated by the skilled person in the art, any of a wide variety of expression systems may be used to provide the recombinant protein. A polypeptide may be expressed and produced in a eukaryotic host cell, e.g., mammalian cells, primary cells, cultured cell lines, or transformed cells. Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include, without limitation, pMSG, pAV009/A+, pMT010/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters that are effective for expression in eukaryotic cells.

Cells may be obtained from a number of sources, for example, from a biological sample, e.g., blood, plasma, serum, saliva, sputum, tissue or organ preparation, biopsy, tumor neoplasm biopsy, and the like. Cell cultures or a variety of cell lines can be used. Non-limiting examples of suitable cells for use in expressing the components of the system and methods described herein include 293T cells (i.e., human embryonic kidney (HEK) 293T cells, also often referred to as HEK293T or 293T cells), MOLT4 cells (i.e., a hypertetraploid human T cell line originally derived from a patient with T-cell acute lymphoblastic leukemia in relapse), NIH 3T3, HeLa cells, COS cells, etc. In a particular embodiment, HEK293T cells are used. By way of example, HEK293 cells are derived from a primary embryonic human kidney cell line transformed with sheared human adenovirus type 5 DNA. The E1A adenovirus gene is expressed in these cells and participates in transactivation of some viral promoters, allowing the cells to produce very high levels of protein. HEK293T is a variant of HEK293 that expresses a temperature-sensitive allele of the SV40 T antigen, which allows for the amplification of vectors containing the SV40 ori and thus increases the protein expression levels during transient transfection. Other cell types that may be used include, without limitation, vertebrate cells, insect cells, chicken cells, and mouse cells. Such cells are available from a wide range of sources (e.g., the American Type Culture Collection, Rockland, Md.; also, see, e.g., Ausubel et al., Current Protocol in Molecular Biology, New York: John Wiley and Sons, 1997 and later editions). The method of transformation or transfection and the choice of expression vehicle will depend on the host cell and system selected, as known by those skilled in the art. Transformation and transfection methods are routinely practiced in the art and are described, e.g., in Ausubel et al. (supra); expression vehicles may be chosen from those provided, e.g., in Cloning Vectors: A Laboratory Manual (P. H. Pouwels et al., 1985, Supp. 1987).

After the expression vector is introduced into the cells, the transfected cells are cultured under conditions favoring expression of the introduced proteins, which may be recovered from the culture using standard techniques known in the art. See. e.g., Freshney et al., 1994, Culture of Animal Cells, A Manual of Basic Technique (3rd ed.), and the references cited therein. In general, the cell culture environment includes consideration of such factors as the substrate for cell growth, cell density and cell contract, the gas phase, the medium, and temperature. Incubation of cells is generally performed under conditions known to be optimal for cell survival. Plastic dishes, tissue culture plates, flasks, or roller bottles, or culture vessels including, for example, multi-well plates, Petri dishes, tissue culture tubes, and the like, may be used to culture cells. In an embodiment of the described methods, cells or cell lines are grown and cultured in culture medium that does not include biotin prior to carrying out the assay methods, at which time biotin is added to the medium. In an embodiment of the methods in which a photocaged BirA biotin ligase (e.g., BirA-K183(ONPK) is utilized, the cells or cell lines may be grown and cultured in culture medium containing biotin, as the photocaged BirA is not active until the enzyme (in the cells or cell lines) is photo-illuminated and becomes decaged and its biotin ligase activity is restored. Similarly, in an embodiment of the methods in which a chemically-caged BirA biotin ligase (e.g., BirA-K183(TCOK) is utilized, biotin may be present, as the chemically-caged BirA is not active until a chemical trigger such as dimethyl tetrazine is added (in cells, cell lines) or administered to animals, and the biotin ligase activity of the BirA enzyme is restored.

Cells are grown at optimal densities that are determined empirically based on the cell type. Cultured cells are normally grown in an incubator that provides a suitable temperature, e.g., physiological body temperature. In general, 37° C. is typically the temperature used for cell culture in incubators that are humidified to approximately atmospheric conditions.

Defined cell media are available as packaged, premixed powders or presterilized solutions. Examples of commonly used media include YT, MEM-α, DME, RPMI 1640, DMEM, Iscove's complete media, or McCoy's Medium (see, e.g., GibcoBRL/Life Technologies Catalogue and Reference Guide, Sigma Catalogue). Defined cell culture media are often supplemented with 5-20% serum, e.g., bovine calf serum, typically heat inactivated. Cell culture can be further supplemented with selection compounds, including, without limitation, ampicillin. The culture medium is usually buffered to maintain the cells at a pH preferably from about 7.2 to about 7.4. Other media supplements may include, e.g., antibiotics, amino acids, and sugars, and growth factors.

Proteins in cells can be purified to substantial purity by using standard techniques, including selective precipitation with such substances as ammonium sulfate, column chromatography, immuno-purification methods, and the like (see, e.g., Ausubel et al., supra; and Sambrook et al., supra). A number of procedures can be used to purify recombinant proteins and proteins associated therewith. For example, biotinylated proteins (E3 ligase substrates) can be selectively and reversibly adsorbed to a streptavidin or avidin-coupled solid support or resin, such as a purification column, beads (Sepharose, agarose, magnetic) and the like, and eluted from the support or resin using elution solutions/buffers containing reagents as known in the art, e.g., 0.5% formic acid in 30% acetonitrile; SDS-containing buffer, or other appropriate elution methods. In some cases, for example, to prepare samples for Western blotting analysis, the streptavidin beads were boiled in biotin-containing sample buffer due to the strong binding affinity between biotin and streptavidin. In some cases, for example, mass spectrometry-based analysis, biotinylated proteins were not eluted, but directly digested by LysC and trypsin proteases on beads. Recombinant protein can be purified from any suitable source, including yeast, insect, bacterial, and mammalian cells.

### Ubiquitination Assays

The rate or extent of ubiquitination of a target substrate can be determined and/or measured in a variety of methods as practiced in the art. In an embodiment, following ubiquitination of protein substrates in a cell, the proteins in the lysate, medium, or reaction mixture can be separated by electrophoresis. The separated proteins are transferred to a substrate, e.g., Western blotting; the blot is probed with antibodies directed against the substrate, and changes in mobility of the substrate that reflect attachment of ubiquitin, or a ubiquitin-like protein, to the substrate are detected. Other methods of measuring ubiquitination can be used, for example, without limitation, immunological assays (ELISA, immunoprecipitation), mass spectrometry, electromagnetic spectrum spectroscopic methods, chromatographic methods, ubiquitination using detectably-labeled, such as biotinylated or biotin-tagged, ubiquitin or a detectably labeled ubiquitin-like protein. Other methods involved in the detection of ubiquitinated proteins (E3 ligase substrates) include plate assays, (e.g., biotin-tagged ubiquitin or a ubiquitin-like protein can be detected using avidin, streptavidin, or labeled forms thereof), and epitope-tagged proteins, such as epitope-tagged ubiquitin or an epitope-tagged ubiquitin-like protein can be detected in immunoassays using antibodies directed against the selected epitope-tag.

### Compositions

Compositions containing the components of the system and methods described herein can also include buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes, suspending agents, thickening agents and/or preservatives. A wide variety of formulations of compositions, including pharmaceutical compositions, are known an available to practitioners in the art. (e.g., Remington's Pharmaceutical Sciences, 17th ed., 1989 and later editions).

### Kits

Kits for practicing and carrying out the methods and assays described herein are provided. In one aspect, kits are provided for performing the E3 ligase substrate detection and identification methods described herein. The kits can be used to detect, select, identify, and/or isolate substrates of E3 ligases, or modified substrates of E3 ligases as described herein and those that may be identified using the systema and methods herein.

In various embodiments, the kit includes one or more vectors (or plasmids) harboring the components of the E3 ligase substrate identification methods described herein. In particular, such vectors include one or more vectors comprising polynucleotide(s) encoding ubiquitin (or a UBL) fused to an A3-tag peptide and polynucleotide(s) encoding the E3 ligase fused to a non-promiscuous BirA biotin ligase. In various embodiments, the kit includes one or more polynucleotide(s) encoding ubiquitin (or a UBL) fused to an A3-tag peptide and polynucleotide(s) encoding the E3 ligase fused to a non-promiscuous BirA biotin ligase. In other embodiments, the kit includes one or more polynucleotide(s) encoding ubiquitin (or a UBL) fused to an AVI™-tag peptide and polynucleotide(s) encoding the E3 ligase fused to a non-promiscuous BirA biotin ligase. In an embodiment, the non-promiscuous BirA is a photocaged BirA, such as BirA-K183(ONPK). In an embodiment, the non-promiscuous BirA is a chemically-caged BirA, such as BirA-K183(TCOK). In other embodiments, the kit can include a sterile container which contains the polynucleotides; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids.

The kit may also include instructions for carrying out the methods, reagents, testing equipment (test tubes, reaction vessels, needles, syringes, etc.), standards for calibrating the methods, and/or equipment provided or used to conduct the methods. The instructions provided in the kit may be directed to suitable operational parameters in the form of a label or a separate insert. The instructions will generally include information about the use of the polynucleotides described herein and their use in detecting and identifying substrates of E3 ligase in a cell, e.g., a cell line or an isolated primary cell or a cell obtained or derived from a tumor, neoplasia, or cancer, e.g., without limitation, a breast, ovarian, cervical, prostate, testis, lung, liver, bladder, lymphocytic, brain, nervous system, or immune system, cardiac, colon, kidney, gall bladder, pancreas cancer, tumor, or neoplasia. The kit can further comprise reagents used in carrying out the methods as described herein. In other embodiments, the instructions include at least one of the following: description of the polynucleotides and other reagents; methods for using the enclosed materials for detecting, selecting, and identifying substrates of E3 ligase; precautions; warnings; indications; research studies; and/or references. The instructions may be printed directly on the container (when present), or as a label applied to the container, or as a separate sheet, pamphlet, card, or folder supplied in or with the container.

The practice of the present disclosure and its aspects and embodiments employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology;” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides as described herein, and, as such, may be considered in making and practicing the aspects and embodiments described in the present disclosure. Particularly useful techniques for particular embodiments will be discussed in the sections that follow.

The following examples are put forth so as to provide those of ordinary skill in the art with a complete disclosure and description of how to make and use the aspects and embodiments as described herein and are not intended to be limiting in scope.

### EXAMPLES

### Example 1: CRBN-BirA and BirA-CRBN Exhibit E3 Ligase Functions

Experiments were performed using the system and method described herein. In the experiments, the CRBN E3 ligase was fused to BirA at either the amino (NH₂) terminus (“BirA-CRBN”) or the carboxy (COOH) terminus (“CRBN-BirA”) of CRBN.

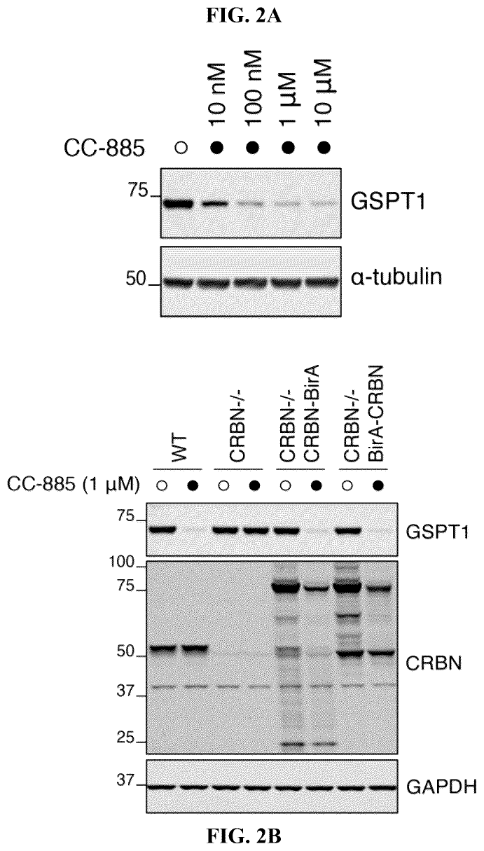

In a first experiment to validate reagents and evaluate HEK293T cells as an appropriate cellular model, HEK293T cells were seeded into a 6-well tissue culture plate and were treated with either no CC-885 (DMSO control) or different doses of CC-885 (10 nM, 100 nM, 1 μM, or 10 μM) for 3.5 hours. CC-885 is a small molecule IMiD analogue that can induce the degradation of neo-substrates such as GSPT1 by promoting the interaction between the neo-substrate and the E3 ligase CRBN. Thereafter, the cells were washed and lysed using RIPA buffer (50 mM Tris-HCl, 150 mM NaCl, 1% NP-40, 0.5% sodium deoxycholate, and 0.1% sodium dodecyl sulfate, pH 7.4) supplemented with COMPLETE™ EDTA-free protease inhibitor cocktail (Roche; 04693159001), 1 tablet per 10 ml of buffer), and benzonase (Millipore; E1014), 1 μl per 10 ml of buffer, as described infra, and the cell lysate was subjected to Western Blotting analysis. A Western blot showing the results is presented in FIG. 2A. As observed in the Western blot, the GSPT1 substrate was degraded in cells in a dose-dependent matter, consistent with the activity of CC-885 as a GSPT1 degrader. Alpha-tubulin (α-tubulin) was used as a protein loading control to show that each lane contained the same amount of protein.

A second experiment was performed to assess the activity of the E3 ligase CRBN to which E. coli wild-type BirA biotin ligase was attached at either the amino (NH₂) terminus (“BirA-CRBN”) or the carboxy (COOH) terminus (“CRBN-BirA”). In particular, the experiment was designed to evaluate the functional activity of CRBN-BirA and BirA-CRBN in the degradation of the CC-885 (an IMiD)-recruited GSPT1 substrate endogenously expressed in 293T cells. The following protocol was used. HEK293T cells with homozygous CRBN knockout (293T^{CRBN-/-}) were transduced with pLX307 vector that expressed CRBN fused to E. coli wild-type BirA biotin ligase (pLX307-CRBN-3F-BirA or pLX307-BirA-3F-CRBN). pLX307 is a lentiviral vector containing an EF-1α promoter for the constitutive expression of a transgene and an SV40 promoter for the expression of the puromycin resistance gene for selection. 1.5×10⁶ cells of wild-type 293T cells, 293T^{CRBN-/-}, and 293T^{CRBN-/-} cells stably expressing either BirA-CRBN or CRBN-BirA were seeded in 6-well tissue culture plates on Day 1. On Day 2, cells were subjected to either DMSO (untreated) or CC-885 (1 μM) for 4 hours. After the 4 hour treatment, the cells were washed and lysed, and the lysate was subjected to Western blotting analysis. The Western blots in FIG. 2B show that the GSPT1 substrate was not degraded in 293T^{CRBN-/-} cells, but was degraded in the 293T^{CRBN-/-} cells expressing either the BirA-CRBN or the CRBN-BirA tagged E3 ligases (as indicated by the faint GSPT1 bands in the lanes in which the cells had been treated with CC-885), suggesting that both BirA-CRBN and CRBN-BirA are functional E3 ligases capable of reconstituting the activity of endogenous CRBN. While both BirA fusions of E3 ligase CRBN are functional, only CRBN-BirA was used in following Examples. The Western blots also show that CRBN was present or absent in the cells as expected. GAPDH levels are shown as a loading control.

### Example 2: Ubiquitinated Substrates are Biotinylated by CRBN-BirA and Captured by Streptavidin Beads

### VS-IKZF1 as an Exogenous, Pomalidomide-Induced Substrate of CRBN in HEK293T Cells

Experiments were performed using the system and method described herein (also referred to as an interaction-specific “E3 substrate tagging system”), constituting E. coli wild type BirA biotin ligase fused to the E3 ligase CRBN, and ubiquitin fused to the peptide substrate of BirA, i.e., AVI-TAG™ (“Avi-Ub”), for directed and specific biotinylation of “tagged” ubiquitins on CRBN substrate by the BirA enzyme fused to CRBN. In an embodiment, ubiquitin was fused to the A3-tag comprising the peptide substrate of the BirA biotin ligase.

In an experiment, 2.2×10⁶ 293T^{CRBN-/-} cells lentivrially engineered to express CRBN-BirA were cultured in 60 mm cell culture dishes containing biotin-free medium for 1 day, followed by transfection with pRK5 vectors containing a polynucleotide encoding ubiquitin fused to (tagged with) the peptide substrate of the BirA biotin ligase, i.e., an AVI-TAG™ peptide, and also a hemagglutinin (HA) tag, (“Avi-HA-Ub); and V5-tagged IKZF1, a neo-substrate of CRBN that is not endogenously expressed in 293T cells. pRK5 is a mammalian expression vector that uses the CMV promoter to drive transgene expression. The V5 tag is derived from a small epitope (Pk) of the P and V protein of the simian virus 5 (SV5, a paramyxovirus), namely, amino acid residues 95 to 108 of RNA polymerase alpha subunit. (T. Hanke et al, 1992, J. General Virology, 73: 653-660). The V5 tag generally contains all 14 amino acids (GKPIPNPLLGLDST (SEQ ID NO: 40)), but a shorter 9-amino acid (IPNPLLGLD (SEQ ID NO: 41)) sequence may be used. The polynucleotide sequence encoding the 14 amino acid V5 tag (for expression in mammalian and insect cells) comprises GGT AAG CCT ATC CCT AAC CCT CTC CTC GGT CTC GAT TCT ACG (SEQ ID NO: 42). The V5 peptide tag can be fused to/cloned into a recombinant protein and detected by assays such as ELISA, flow cytometry, immunoprecipitation, immunofluorescence, and Western blotting using cognate antibodies or nanobodies.

In some cases as described in the Examples below, a vector containing a polynucleotide encoding ubiquitin fused to (tagged with) an A3-tag peptide substrate of the BirA biotin ligase, (“A3-Ub”), was used instead of Avi-Ub. The Avi-Ub or A3-Ub peptide-tagged ubiquitin expressed in the cells was utilized in the ubiquitination of CRBN substrate by the BirA biotin ligase-tagged E3 ligase CRBN, resulting in a ubiquitinated substrate decorated with ubiquitin molecules that were able to be biotinylated by BirA fused to the CRBN E3 ligase.

One day (approximately 24 hours) after the transfection, DMSO (untreated) or carfilzomib (0.4 μM), (“carf”), was added to the cells for 1 hour. Carfilzomib (sold under the brand name Kyprolis) is a selective proteasome inhibitor. Chemically, it is a tetrapeptide epoxyketone and an analog of epoxomicin. Carfilzomib covalently and irreversibly binds to and inhibits the chymotrypsin-like activity of the 20S proteasome, an enzyme that degrades unwanted cellular proteins. Carfilzomib interacts minimally with non-proteasomal targets. The inhibition of proteasome-mediated proteolysis by carfilzomib results in an accumulation of polyubiquitinated proteins in the cell. Because the accumulation of polyubiquitinated proteins in the cell may cause cell cycle arrest, apoptosis and inhibition of tumor growth, carfilzomib has been used as a therapeutic in patients with relapsed and refractory multiple myeloma.

An hour after the addition of carfilzomib, either DMSO (untreated) or an IMiD agent pomalidomide, was added to the cells for 1 hour, after which time biotin (50 μM) was added for 15 minutes. Pomalidomide is an IMiD analog that recruits the transcription factor IKZF1 to the E3 ubiquitin ligase CRBN (CRL4-CRBN). V5-IKZF1, which is introduced to the 293T cells in this experiment, binds to the CRBN fused to BirA in the cells, is ubiquitinated with Avi-tagged or A3-tagged ubiquitins, which are biotinylated by the BirA fused to CRBN.

After the 15-minute biotin treatment, the cells were washed and lysed, and the lysate was subjected to overnight precipitation (“pulldown”) at 4° C. with streptavidin attached to a solid substrate, namely, streptavidin-coated beads (CYTIVA™ Streptavidin Sepharose High Performance (17511301)), that bound to the ubiquitinated GSPT1 substrate protein that had been biotinylated by the BirA biotin ligase fused to the CRBN E3 ligase. A portion of the lysates (referred to as inputs) were saved for Western blotting analyses. Following the pulldown, the biotinylated cellular proteins bound to the streptavidin beads were washed once with 2% SDS washing buffer and twice with RIPA cell lysis buffer. The biotinylated substrate proteins resulting from the pulldowns were eluted from the beads by boiling for 10 minutes in 1× sample buffer (Invitrogen NUPAGE™ LDS Sample Buffer (4×)) containing 50 mM DTT and 2 mM biotin. The eluates were analyzed by SDS-PAGE gel electrophoresis and Western blotting analyses.

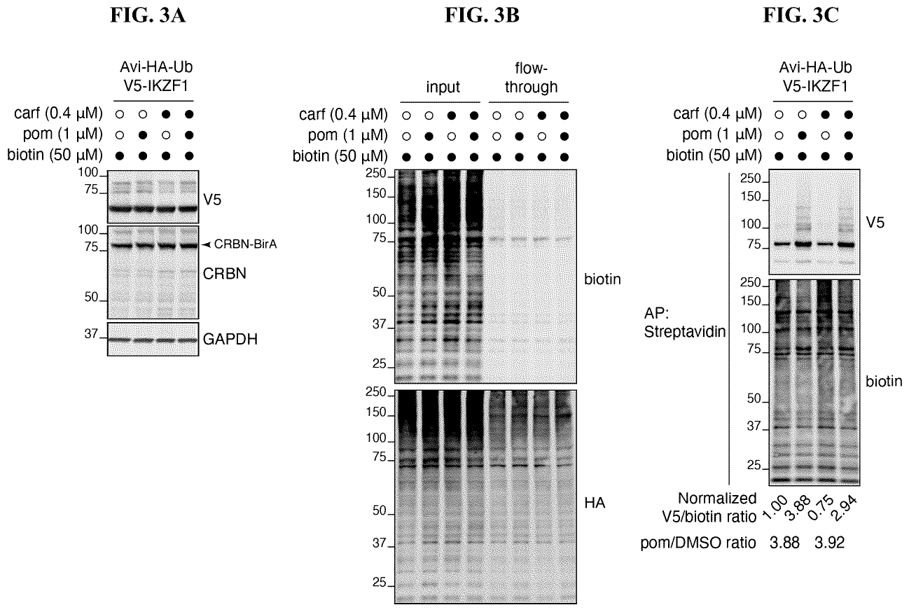

FIGS. 3A and 3B show the analyses of input lysate samples, whereas FIG. 3C shows the analyses of streptavidin pulldown samples. FIG. 3A shows a Western blot probed using an anti-V5 tag antibody (upper portion of the blot), an anti-CRBN antibody (middle portion of the blot) and anti-GAPDH antibody (lower portion of the blot), used as an endogenous control for the input in each well. The results demonstrate that CRBN-BirA and V5-IKZF1 are present in cells and are equal across the conditions. FIG. 3B shows a Western blot analysis of the input and flow through probed with ALEXA FLUOR™ 680 conjugated streptavidin to detect biotinylated species (upper portion of the blot) and an anti-HA tag antibody (lower portion of the blot). The results demonstrate that equal amounts of Avi-HA-Ub were present across the conditions used; that Avi-HA-Ub is biotinylated in cells; and that the streptavidin beads successfully captured the biotinylated species and depleted them from the flow-through. FIG. 3C shows a Western blot analysis probed with an anti-V5 tag antibody (upper portion of the blot) and ALEXA FLUOR™ 680 conjugated streptavidin (lower portion of the blot). The results demonstrate that the streptavidin beads successfully captured biotinylated Avi-tagged and ubiquitinated species in the cells, including V5-IKZF1. Furthermore, in conditions where pomalidomide was present, an increased amount of mono- and poly-ubiquitinated V5-IKZF1 was captured by the streptavidin beads, suggesting that the method described herein can identify pomalidomide-induced ubiquitination of IKZF1 by E3 ligase CRBN.

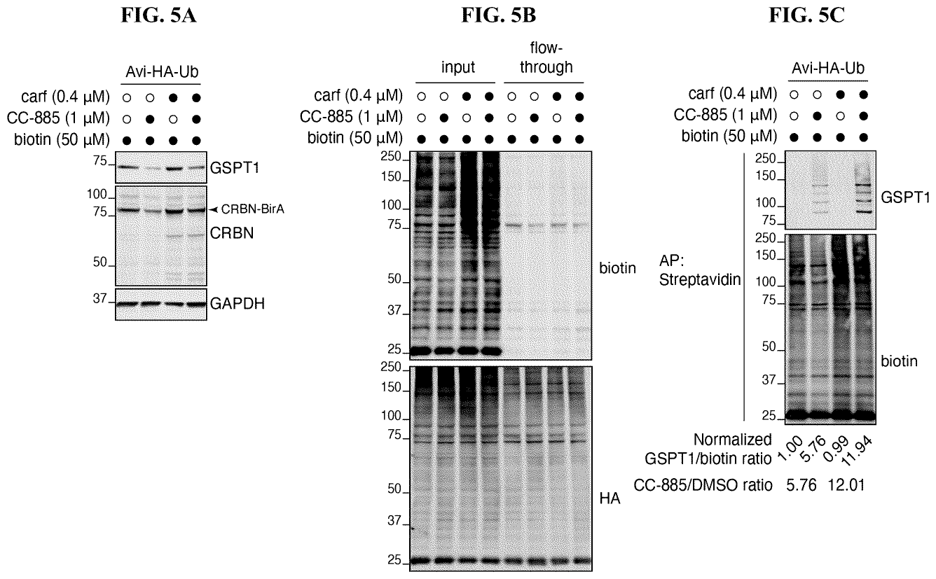

GSPT1 as an Endogenous, CC-885-Induced Substrate of CRBN in HEK293T Cells FIGS. 5A-5C show the results of experiments similar to the above-described experiments in this Example, using 293T^{CRBN-/-} cells lentivirally engineered to express CRBN-BirA and transiently expressing Avi-HA-Ub. However, instead of assessing transiently-expressed V5-tagged IKZF1, an endogenous neo-substrate of CRBN, namely, GSPT1, was assessed. The 293T^{CRBN-/-} cells were either treated or untreated with carf (0.4 μM) for 1 hour and were either treated or untreated with the IMiD analog CC-885 (1 μM) for 2 hours. Subsequently, biotin (50 μM) was added for 15 minutes. Following incubation with the components and added agents, the cells were lysed, and streptavidin-coated beads were used to capture from the cell lysate biotinylated, ubiquitinated proteins, e.g., the endogenous GSPT1 substrate. The biotinylated proteins eluted from the beads were subjected to Western blot analyses. FIGS. 5A and 5B show Western blot analyses of the cell lysates and FIG. 5C shows the eluates following streptavidin pulldowns. Specifically, FIG. 5A shows a Western blot probed using an anti-GSPT1 antibody (upper portion of the blot), an anti-CRBN antibody (middle portion of the blot), and an anti-GAPDH antibody (lower portion of the blot), used as an endogenous control for the input in each well. The results demonstrate that GSPT1 is degraded in the presence of CC-885, as expected, and the degradation is partially rescued by inhibiting the proteasome using carfilzomib. FIG. 5B shows a Western blot of input and flow-through probed with ALEXA FLUOR™ 680 conjugated streptavidin (upper portion of the blot) and an anti-HA tag antibody (lower portion of the blot). The results demonstrate that equal amounts of Avi-HA-Ub were present across conditions; that Avi-HA-Ub was biotinylated in cells; and that the streptavidin beads successfully captured the biotinylated species and depleted them from the flow-through. FIG. 5C shows a Western blot probed with an anti-GSPT1 antibody (upper portion of the blot) and ALEXA FLUOR™ 680 conjugated streptavidin (lower portion of the blot). The results demonstrate that the streptavidin beads successfully captured biotinylated Avi-tag-ubiquitinated species in the cells, including endogenous GSPT1. Furthermore, mono- and poly-ubiquitinated GSPT1 captured by the streptavidin beads were only observed in conditions where CC-885 was present, as can be specifically observed by comparing the first and second and the third and fourth lanes in the upper portion of the Western blot in FIG. 5C, demonstrating that the method described herein identifies CC-885-induced ubiquitination of GSPT1 by E3 ligase CRBN.

### Example 3: A3-Tagged Ubiquitin Significantly Reduces Background Compared to AVI-TAG™ Ubiquitin in Analyses of Biotinylated Substrate Proteins

V5-IKZF1 as an Exogenous. Pomalidomide-Induced Substrate of CRBN in HEK293T Cells

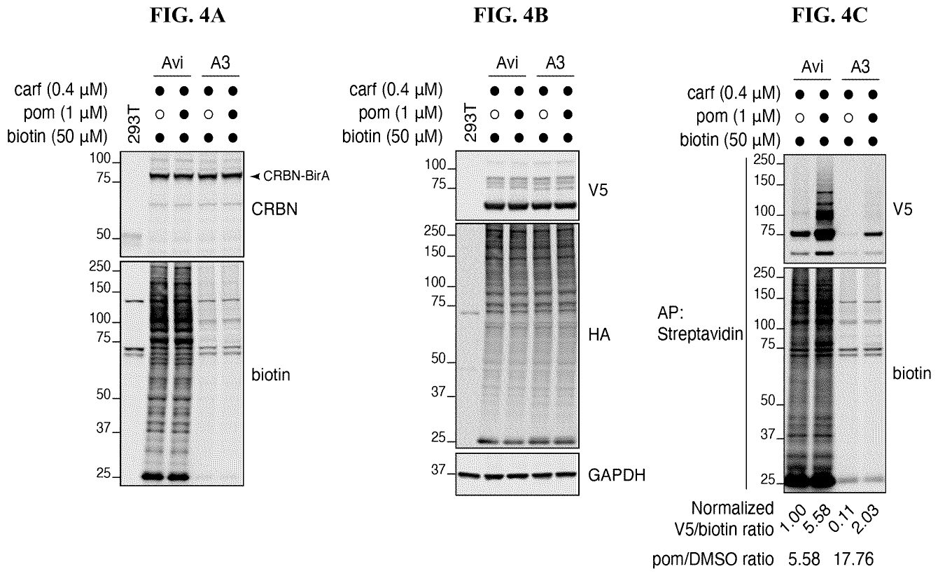

Experiments similar to those described in Example 2 were performed using HEK293T^{CRBN-/-} cells cultured in biotin-free medium, except that ubiquitin fused to either the AVI-TAG™ or the A3-tag peptide substrate of BirA biotin ligase was used. The cells were either treated or untreated with carfilzomib (carf), (0.4 μM) and were either treated or untreated with the IMiD agent pomalidomide (1 μM). Biotin (50 μM) was added to the cells. FIGS. 4A-4C show Western blot analyses of biotinylated proteins from cell lysates (FIGS. 4A and 4B) or the eluates following streptavidin/avidin pulldowns (FIG. 4C) in the experiments using either an AVI-TAG™ or an A3-tag biotin ligase peptide substrate in the interaction-specific ubiquitination system described herein. FIG. 4A shows a Western blot of lysate inputs probed using an anti-CRBN antibody (upper portion of the blot) and ALEXA FLUOR™ 680 conjugated streptavidin to detect biotinylated species (lower portion of the blot). The results demonstrate that CRBN-BirA was present and equally expressed in the experimental conditions, and that the total biotinylation levels that occurred in cells were significantly lower for A3-Ub than Avi-Ub, suggesting less background when A3-Ub was used (i.e., fewer nonspecific biotinylated proteins were pulled down and reacted on the blots). FIG. 4B shows a Western blot analysis using an anti-V5 tag antibody (middle portion of the blot), an anti-HA tag antibody (middle portion of the blot), and anti-GAPDH antibody (lower portion of the blot), used as an endogenous control for the input in each well. The results demonstrate that the components of the method described herein are present and equally expressed across conditions. FIG. 4C shows a Western blot analysis of streptavidin bead eluates probed with an anti-V5 tag antibody (upper portion of the blot) and ALEXA FLUOR™ 680 conjugated streptavidin (lower portion of the blot). The results demonstrate that the background biotinylation level was indeed much lower for A3-Ub than Avi-Ub, while both substrate peptide-tagged ubiquitin variants showed pomalidomide-induced ubiquitination of IKZF1 by E3 ligase CRBN.

### GSPT1 as an Endogenous, CC-885-Induced Substrate of CRBN in HEK293T Cells

In related experiments like those described in Example 2, the engineered HEK293T cells were transfected to express (or overexpress) ubiquitin fused either to the AVI-TAG™ or the A3-tag peptide substrate of BirA biotin ligase. The cells were either treated or untreated with carfilzomib (0.4 μM) for 1 hour and were either treated or untreated with the CRBN-IMiD modifying agent CC-885 (1 μM) for another hour. Biotin (50 μM) was added exogenously to the cells for 15 minutes. As discussed above in Example 1, the GSPT1 substrate of BirA-tagged CRBN is expressed endogenously in 293T cells.

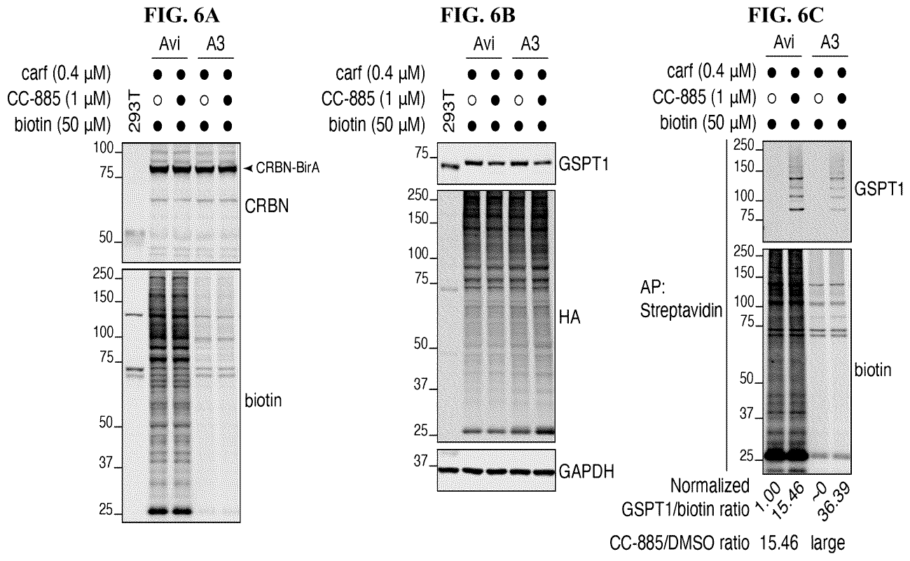

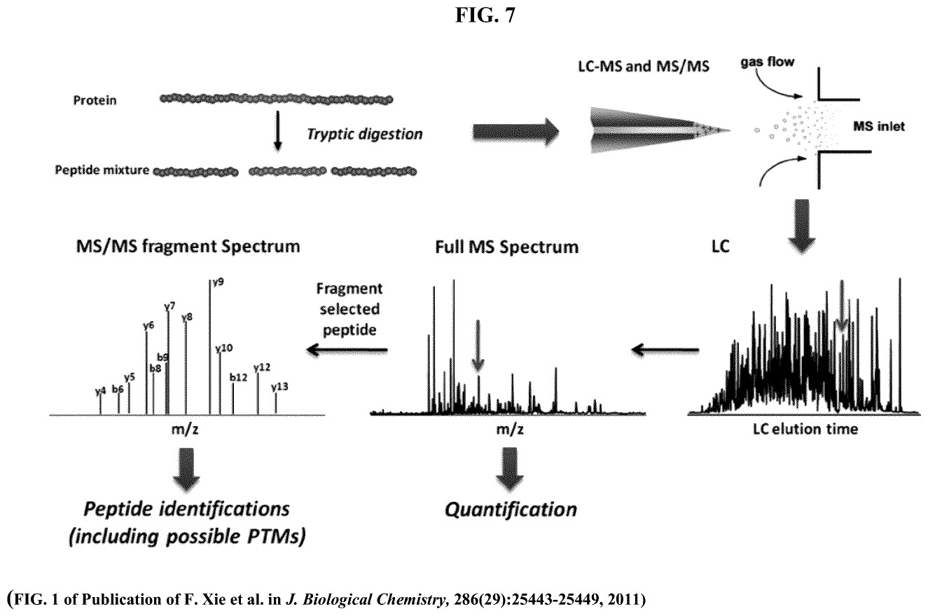

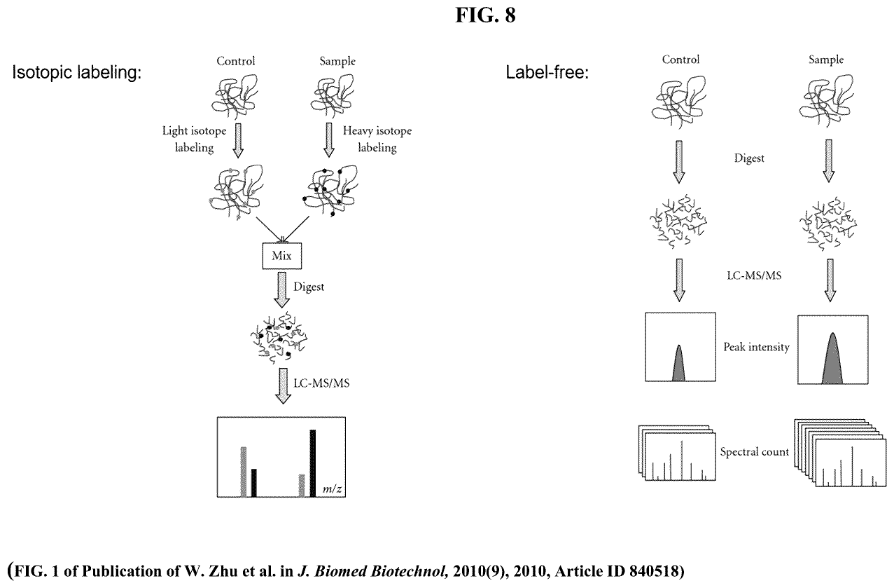

FIGS. 6A-6C show Western blot analyses of input lysates (FIGS. 6A and 6B) or the eluates following streptavidin pulldowns (FIG. 6C) in the experiments using either an AVI-TAG™ or an A3-tag peptide substrate in the interaction-specific ubiquitination system described herein. FIG. 6A shows a Western blot probed with an anti-CRBN antibody (upper portion of the blot) and ALEXA FLUOR™ 680 conjugated streptavidin (lower portion of the blot). The results demonstrate that CRBN-BirA was present and equally expressed in the experimental conditions described, and that the total biotinylation levels that occurred in cells were significantly lower for A3-Ub than Avi-Ub, demonstrating that less background occurred when A3-Ub was used. FIG. 6B shows a Western blot probed using an anti-GSPT1 antibody (upper portion of the blot), an anti-HA tag antibody (middle portion of the blot), and anti-GAPDH antibody (lower portion of the blot), used as an endogenous control for the input in each well. The results demonstrate that the components of the method described herein were present and equally expressed across the described conditions. FIG. 6C shows a Western blot probed with an anti-GSPT1 antibody (upper portion of the blot), which detected biotinylated, ubiquitinated endogenous GSPT1, and with ALEXA FLUOR™ 680 conjugated streptavidin (lower portion of the blot). As described for the experiments above (e.g., FIGS. 4A-4C), the use of ubiquitin fused to the A3-tag BirA peptide substrate advantageously demonstrated considerably less background (i.e., fewer nonspecific biotinylated proteins were precipitated and reacted on the blots) compared with the use of ubiquitin fused to the AVI-TAG™ BirA peptide substrate. This can be observed, for example, by comparing the “Avi” and the “A3” lanes of the lower portions of the Western blots probed with ALEXA FLUOR™ 680 conjugated streptavidin (“biotin”) shown in FIG. 6A and FIG. 6C. The stronger background of biotinylation observed using AVI-TAG™ compared to that using A3-tag indicates that the A3-tag has a better signal-to-background ratio.

### Example 4: Mass Spectrometry Analyses of Biotinylated, Ubiquitinated E3 Substrates

The biotinylated ubiquitinated proteins produced as described in the above Examples, specifically Example 3 (CC-885 and GSPT1), were analyzed and identified by mass spectrometry (MS) analysis.

For such MS analyses, 50 μl of resuspended streptavidin beads were incubated with 1 ml of cell lysates (3 mg/ml) at 4° C. overnight. On the next day, the beads were pelleted (400×g, 1 minute), washed two times with 2% SDS (2% SDS, 25 mM Tris-HCl pH 7.5), and then washed three times with 6M urea (6M urea, 50 mM Tris-HCl pH 8). Each wash was performed by rotating the samples at room temperature for at least five minutes. Following the washes, the beads were resuspended in 90 μl of 2M urea, to which 10 μl of 0.1M TCEP (tris(2-carboxyethyl)phosphine, reducing agent) was added for a final concentration of 10 mM TCEP. The samples were agitated at 37° C. for 30 minutes to allow for protein reduction. Next, 10 μl of 0.165 M IAA, (iodoacetamide, alkylating agent) was added to a final concentration of 15 mM IAA. The samples were agitated in the dark at room temperature for 45 minutes. Thereafter, 1 μg of trypsin/LysC (Promega, Trypsin/Lys-C Mix, Mass Spec Grade, V5071) was added to each sample, and the samples were agitated for 3-4 hours at 37° C. Subsequently, 0.5 μg of trypsin/LysC (Promega, Trypsin/Lys-C Mix, Mass Spec Grade, V5071) was added to each sample, and the samples were agitated overnight at 37° C. On the next day, the supernatant containing the digested peptides was collected. The beads were washed two times with HPLC-grade water and were combined with the previously collected supernatant. Samples were centrifuged at 21,000×g for 10 minutes, and the clarified supernatant at the top was saved. Samples were acidified by the addition of 50% formic acid to a final concentration of 2%. Samples were vacuum dried (Speedvac) overnight and kept at −80° C. until ready for analysis by MS.

A synopsis of the MS analysis results of the biotinylated ubiquitinated substrate proteins identified from cells treated with either DMSO (control) or the CRBN modulating agent CC-885 and expressing either ubiquitins fused to the AVI-TAG™ peptide substrate of biotin ligase BirA or ubiquitins fused to the A3-tag peptide substrate of BirA, as well as the other components of the E3 substrate tagging system described and exemplified herein (e.g., CRBN E3 ligase fused to non-promiscuous E. coli wild type BirA) is presented in Table 1 below.

TABLE 1
Protein Identification by MS

| | Avi- | Avi- | A3- | A3- |
|---|---|---|---|---|
| | DMSO | CC-885 | DMSO | CC-85 |
| No. of proteins identified | 1006 | 674 | 148 | 114 |
| Contaminants removed | 990 | 660 | 138 | 101 |
| PSM <3 removed | 287 | 194 | 47 | 41 |

Table 1 shows that the “total number of proteins identified,” “contaminants removed” and “PSM<3 removed” using A3-tagged ubiquitins for ubiquitination of CRBN E3 ligase substrates in the cells were significantly reduced compared with AVI-TAG™-tagged ubiquitins, demonstrating the decreased background signals achieved by the use of the A3-tag for selecting and identifying biotinylated, ubiquitinated substrates of E3 ligase in pulldowns of cell lysates using a streptavidin-coated solid substrate.

Table 2 below shows the results of MS analysis of streptavidin pulldown of biotinylated proteins in 293T cells that expressed Avi-tagged ubiquitin (Avi-Ub), i.e., ubiquitin fused to AVI-TAG™, and E3 ligase fused to non-promiscuous, E. coli wild type BirA biotin ligase, and that were treated with DMSO (positive control) or with the CRBN modulator CC-885, which recruits the substrate GSPTI (eRF3a) to the CRL4-CRBN E3 ubiquitin ligase. Table 2 presents the most enriched proteins from the streptavidin pulldown, rank ordered based on peptide spectrum match (PSM) counts in the CC-885 treated condition. In particular, endogenously biotinylated carboxylase enzymes, shown in bold font in Table 2 (PC, ACACA, PCCA and MCCC1) are present in high abundance, suggesting that the streptavidin pulldown performed as expected. Many E1, E2 or E3 enzymes (underlined) were also among the most enriched proteins. This result is consistent with the fact that E1, E2 and E3 enzymes are often ubiquitinated due to their functions. As all ubiquitinated proteins in the cells can potentially be biotinylated by BirA-fused to CRBN, their enrichment in streptavidin pulldown is reasonable and represents part of the background.

The biotinylated ubiquitinated endogenous proteins resulting from the analysis include the following: PC (Pyruvate Carboxylase); VCL (vinculin); ACACA (acetyl-CoA carboxylase 1); PCCA (Propionyl-CoA carboxylase alpha chain), UBA6 (ubiquitin-like modifier-activating enzyme 6 (ubiquitin E1 ligase)); BIRC6 (Baculoviral IAP repeat-containing protein 6); UBA1 (ubiquitin-like modifier-activating enzyme 1 (ubiquitin E1 ligase)), HUWE1 (HECT, UBA And WWE Domain Containing E3 Ubiquitin Protein Ligase 1), and HECTD1 (HECT Domain E3 Ubiquitin Protein Ligase 1). MCCC1 (Methylcrotonyl-CoA Carboxylase Subunit 1) is a biotin-requiring enzyme located in the mitochondria and is involved in the processing of the amino acid leucine in cells. GSPT1 and GSPT2, shown in bold italics in Table 2, are not the most enriched proteins by absolute PSM counts, yet are still among the top hits. Of note, when comparing the PSM counts between the two conditions (DMSO versus CC-885), GSPT1/2 peptides were only present in the latter condition (0 versus 25 and 0 versus 13, respectively), supporting the fact that GSPT1/2 are CC-885-induced ubiquitinated substrates of CRBN. (See, also, FIGS. 5C and 6C).

Additionally, enriched biotinylated E1, E2 and E3 protein-associated substrate proteins using the system described herein are underlined as shown in the tables, e.g., BIRC6 (Baculoviral IAP Repeat Containing 6), HUWE1 (HECT, UBA And WWE Domain Containing E3 Ubiquitin Protein Ligase 1), HECTD1 (HECT Domain E3 Ubiquitin Protein Ligase 1), CRBN (Cereblon E3 ligase), MYCBP2 ((MYC Binding Protein 2), DDB1 (Damage Specific DNA Binding Protein 1) and TRIP12 (Thyroid Hormone Receptor Interactor 12), (dotted underlining) and to a lesser extent, UBA6 (Ubiquitin Like Modifier Activating Enzyme 6), UBA1 (Ubiquitin Like Modifier Activating Enzyme 1), UBE20 (Ubiquitin Conjugating Enzyme E2 O), UBE2K (Ubiquitin Conjugating Enzyme E2 K) and UBE2N (Ubiquitin Conjugating Enzyme E2 N), (single underlining).

In Table 2 as well as in Table 3 below, “# of PSMs” associated with mass spectrometry results refers to the number of peptide spectrum matches (PSMs); the number of PSMs is the total number of identified peptide spectra matched for the protein.

The peptide-spectrum match (PSM) scoring function assigns a numerical value to a peptide-spectrum pair (P,S) expressing the likelihood that the fragmentation of a peptide with sequence P is recorded in the experimental mass spectrum S. A description of the derivation of PSM values is described below.

Table 3 shows the results of MS analysis of streptavidin/avidin pulldown of biotinylated and ubiquitinated proteins in cells expressing ubiquitin linked to the A3-peptide substrate of BirA, A3-Ub, rather than ubiquitin linked to the AVI-TAG™-peptide substrate (Avi-Ub), and CRBN E3 ligase fused to non-promiscuous, E. coli wild type BirA biotin ligase and treated with either DMSO (“A3-DMSO” control) or the CRBN modulator CC-885 (“A3-CC885”), which induces ubiquitination of the GSPT1 and GSPT2 substrates of CRBN E3 ligase linked to BirA (E. coli wild type BirA) and biotinylation of A3-Ub on the substrates by the E3 ligase-linked BirA. The top proteins identified in this experiment are shown and are rank ordered based on peptide spectrum match (PSM) counts in the CC-885 treated condition. Endogenously biotinylated carboxylase enzymes are still among the most abundant hits. Interestingly, GSPT1 and GSPT2 were the most abundant hits, excluding carboxylase enzymes, and many E1, E2 and E3 enzymes that were abundant in the Avi-Ub group (Table 2) were no longer abundant or even present, indicating a reduced background. Therefore, the greater specificity afforded by the use of A3-peptide tagged ubiquitin (compared with AVI-TAG™-tagged ubiquitin) for ubiquinating the endogenous GSPT1 and GSPT2 substrates, which were biotinylated by the BirA fused to CRBN E3 ligase in accordance with the functional system described herein, was demonstrated. Furthermore, greater amounts of biotinylated, ubiquitinated, endogenous GSPT1 and GSPT2 substrates (bold, italicized in Table 3) were pulled down and enriched for by streptavidin-coated beads from the A3-CC-885-treated cells compared with control cells treated with DMSO (A3-DMSO) cells (30 versus 0, and 20 versus 0, respectively), supporting the fact that GSPT1/2 are CC-885-induced ubiquitinated substrates of CRBN.

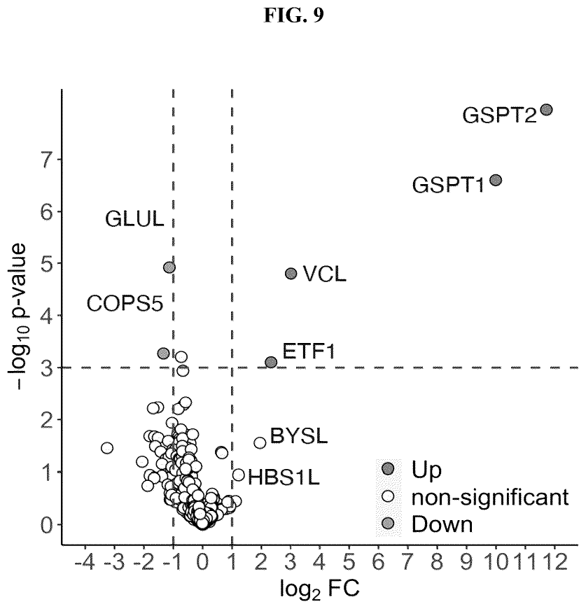

FIG. 9 shows the results of an experiment whose results are shown in Table 3; in this experiment, three replicate samples were prepared for each condition (i.e., 293TCRBN−/− cells stably expressing CRBN-BirA, transiently expressing A3-Ub, and treated with DMSO vs CC-885). Triplicate samples allowed for statistical analysis of differentially enriched proteins, represented by log 2-fold-change (FC) between the CC-885-treated group and the DMSO-treated group on the x-axis, and significance (p-value estimated by limma) on the y-axis in FIG. 9. As demonstrated in FIG. 9, GSPT1 and GSPT2 are observed to be the most upregulated proteins, both by amount and by statistical significance, in the streptavidin pulldown samples treated with CC-885. The result is consistent with the fact that CC-885 induces the ubiquitination of GSPT1/2 via hijacking CRBN. Critically, the results demonstrate that known substrates of E3 ligase (e.g., CRBN) can be identified without bias using the system and methods described herein, in conjunction with mass spectrometry-based identification. Other top hit proteins observed following MS analysis include VCL—vinculin; ETF1—Eukaryotic peptide chain release factor subunit 1 (termination of nascent peptide synthesis); HBS1L—HBS1-like protein (co-translational quality control); BYSL—Bystin (processing of 20S pre-rRNA precursor and biogenesis of 40S ribosomal subunit); GLUL—glutamine synthetase.

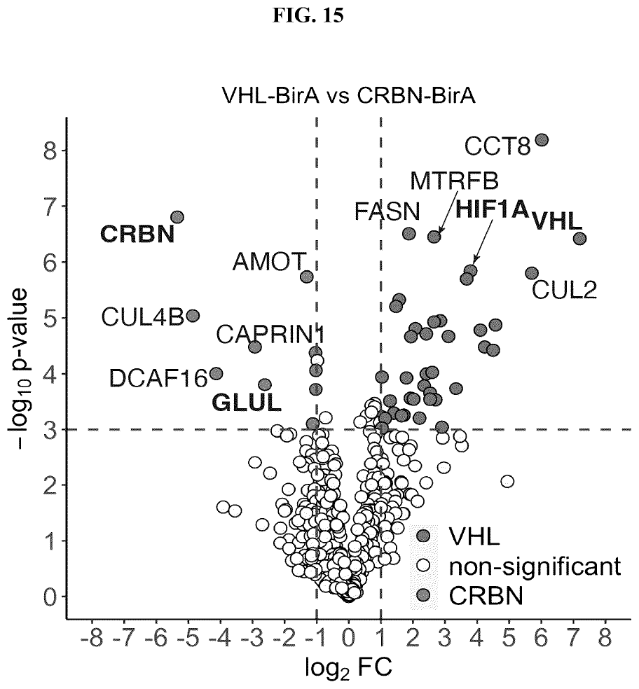

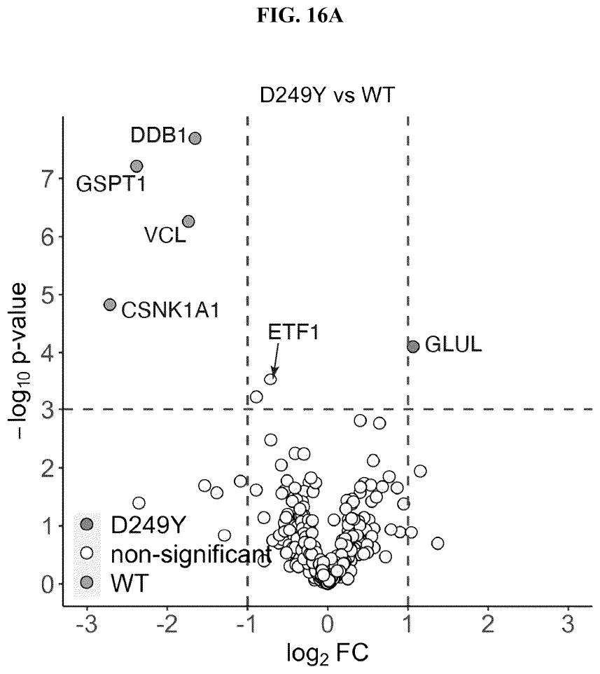

FIG. 15 illustrates the log 2-transformed ratio of proteins biotinylated by the E3 ligase VHL fused to non-promiscuous wild type E. coli BirA over those biotinylated by the E3 ligase CRBN fused to non-promiscuous wild type E. coli BirA on the x-axis and the statistical significance associated with the observed differential enrichment (−log₁₀ p value) as calculated using limma on the y-axis. Specifically, the biotinylated ubiquinone of HEK293T cells stably expressing VHL-BirA (triplicate) was compared with that of HEK293T CRBN−/− cells stably expressing CRBN-BirA (triplicate). The results of the transient expression of A3-tagged ubiquitin are shown. To perform these experiments, the cells were treated with carfilzomib (0.4 μM) for 2 hours, followed by 15-minute treatment with biotin (50 μM). As shown in FIG. 15, VHL and CRBN are shown as differentially biotinylated proteins, which is consistent with the fact that they serve as ‘baits’ in the system and that E3 ligases are often ubiquitinated themselves. One of the best known VHL substrates, HIF1A was among the hits of differentially biotinylated proteins by VHL-BirA, suggesting that the system and methods described herein can identify E3 ligase substrates. Another VHL substrate, HIF2A, was not detected, likely because HIF2A is poorly expressed in HEK293T cells. Other identified proteins included GLUL, which is a reported a CRBN substrate.

TABLE 2

| | # of PSMs | | | # of PSMs | |
|---|---|---|---|---|---|
| Gene | Avi- | Avi- | Gene | Avi- | Avi- |
| Symbol | DMSO | CC885 | Symbol | DMSO | CC885 |
| PC | 116 | 104 | UBE2K | 19 | 17 |
| VCL | 10 | 56 | MCCC1 | 20 | 16 |
| ACACA | 60 | 52 | [symbol] | 22 | 15 |
| PCCA | 45 | 41 | HSP90AB1 | 22 | 15 |
| UBA6 | 39 | 37 | CTNNB1 | 20 | 15 |
| [symbol] | 39 | 33 | H2AFV | 14 | 14 |
| UBA1 | 34 | 31 | [symbol] | 22 | 14 |
| [symbol] | 34 | 30 | UBE2N | 20 | 14 |
| [symbol] | 36 | 29 | DSP | 21 | 14 |
| GSPT1 | 0 | 25 | HSP90AA1 | 24 | 13 |
| UBE2O | 37 | 23 | [symbol] | 21 | 13 |
| RPS3 | 17 | 21 | DYNC1H1 | 17 | 13 |
| USP9X | 32 | 21 | GSPT2 | 0 | 13 |
| AMOT | 23 | 19 | EEF2 | 22 | 13 |
| PRPF8 | 20 | 19 | SMC1A | 12 | 13 |
| HSPA1B; | 18 | 18 | HSPA8 | 20 | 13 |
| HSPA1A | | | | | |
| [symbol] | 16 | 18 | CTNNA1 | 15 | 12 |
| HIST2H2AC | 19 | 18 | VCP | 20 | 11 |
| PRKDC | 24 | 17 | PSMD4 | 10 | 11 |
| FASN | 28 | 17 | POLR2A | 9 | 11 |

TABLE 3

| | # of PSMs | | | # of PSMs | |
|---|---|---|---|---|---|
| Gene | Avi- | Avi- | Gene | Avi- | Avi- |
| Symbol | DMSO | CC885 | Symbol | DMSO | CC885 |
| PC | 155 | 167 | HSP90AB1 | 9 | 9 |
| PCCA | 106 | 103 | VCL | 0 | 9 |
| ACACA | 71 | 75 | HIST1H4A . . . | 9 | 9 |
| GSPT1 | 0 | 30 | THAP5 | 6 | 7 |
| MCCC1 | 26 | 24 | EEF1A1 | 7 | 6 |
| GSPT2 | 0 | 20 | HSPA1B; | 15 | 6 |
| | | | HSPA1A | | |
| [symbol] | 23 | 17 | ENO1 | 7 | 6 |
| ACACB | 0 | 17 | HIST2H2BE | 6 | 6 |
| CCT8 | 7 | 14 | UBE2N | 7 | 6 |
| HSP90AA1 | 14 | 12 | [symbol] | 6 | 6 |
| [symbol] | 10 | 11 | HIST1H1D | 0 | 5 |
| CCDC144A | 3 | 10 | DDX5 | 3 | 5 |
| HSPA8 | 15 | 10 | EEF2 | 7 | 5 |
| SPEN | 5 | 9 | HIST2H2BF | 5 | 5 |

### Example 5: Profiling Ubiquitinated Substrates of Kinase-Targeting PROTAC®s Containing IMiDs Linked to Kinase Inhibitors or Kinase Degraders

In this example, PROTAC® (proteolysis-targeting chimera) molecules that target kinases were created by linking certain kinase degraders with IMiDs analogs. (FIG. 12). PROTAC®s recruit a target protein to be in proximity to an E3 ubiquitin ligase to trigger protein degradation. The ubiquitinated targets of four CRBN-recruiting multi-kinase PROTAC®s were generated to contain the following multikinase degraders: SK-3-91, DB0646, SB1-G-187 and WH-10417-099. These four degraders can collectively induce degradation of a large number of distinct kinases (>125 distinct kinases). FIGS. 13A-13D show the results of a mass spectrometry experiment in which protein substrates susceptible to induced ubiquitination by the abovementioned four multikinase degraders were identified, respectively. These experiments were performed in HEK293T^{CRBN-/-} cells lentivirally engineered to express CRBN-BirA and transiently expressing A3-HA-Ub. While cultured in biotin-free medium, the cells were pre-treated with carfilzomib (carf; 0.4 μM) for 1 hour, and then were either treated or untreated with the kinase-targeting PROTAC®s (1 μM) for another 45 minutes. Subsequently, biotin (50 μM) was added to the cells for 15 minutes, after which time the cells were lysed and maintained on ice to terminate the reaction. Enrichment of biotinylated species by streptavidin-coated beads, protein digestion, and protein identification and quantification by mass spectrometry were performed exactly as described in Example 3 and 4. Triplicate samples allowed for statistical analysis of differentially enriched proteins, represented by log 2-fold-change (FC) between the PROTAC®-treated group and the DMSO-treated group on the x-axis, and significance (p-value estimated by limma) on the y-axis in the figures. FIGS. 13A-13D present the induced ubiquitinated substrates for SK-3-91, DB0646, SB1-G-187 and WH-10417-099, respectively, shown and labeled in the upper right quadrant of the plot. Consistent with the expected actions of these multikinase degraders, the majority of the induced ubiquitinated proteins detected were kinase targets previously described for these PROTAC®s based on binding activity or protein degradation. The data suggest that the system and methods described herein can detect induced ubiquitination targets induced by E3-ligase modulating agents such as PROTAC®s.

### Example 6: Profiling Ubiquitinated Substrates of PROTAC®s Containing BET Bromodomain- or PTK2-Inhibitors to Recruit Other E3 Ubiquitin Ligases

Experiments were performed using the methods and systems described herein to characterize other protein degraders (PROTAC®s) that recruit not only CRBN E3 ubiquitin ligase, but also other E3 ubiquitin ligases, such as Von Hippel-Lindau (VHL) E3 ligase. In these studies, Bromo- and Extra-terminal (BET) bromodomain degraders and PTK2 (Protein Tyrosine Kinase 2) degraders were used as PROTAC®s. (FIG. 10). BET bromodomain-containing proteins, such as BRD2, BRD3, and BRD4, play important roles in transcriptional regulation, epigenetics, and cancer. BET proteins are the targets of the pan-BET selective bromodomain inhibitor JQ1 (Filippakopoulos, P. et al., 2010, Nature, 468, pp. 1067-1073). PROTAC®s have been designed that tether JQ1 to a ligand for VHL E3 ligase, for triggering the intracellular destruction of BET proteins. The BET family of proteins, including the ubiquitously expressed BRD2, BRD3, and BRD4, recruit transcriptional regulatory complexes to acetylated chromatin, resulting in the control of specific networks of genes involved in cellular proliferation and cell cycle progression. (Zengerle, M. et al., 2015, ACS Chem. Biol., 10(8):1770-1777). Deregulation of BET protein activity, in particular, BRD4, has been linked to cancer and inflammatory diseases, making BET proteins attractive drug targets. Two highly homologous bromodomains are present in the amino-terminal regions of BET proteins and direct recruitment to nucleosomes by binding to specific acetylated lysines (K_{Ac}) within histone tails. Small molecule BET inhibitors, including the triazolodiazepine-based JQ1, among others, bind to the K_{Ac}-binding pocket of the bromodomains and disrupt their interaction with histones, thereby displacing BET proteins and their associated transcriptional regulatory complexes from chromatin. BET inhibitors are highly potent (K_{d}˜100 nM) and cell-penetrant (Zengerle, M. et al., Ibid.).

As shown in FIG. 10, the protein degrader dBET6 is composed of a BET antagonist (+)-JQ1 composed of a BRD4-binding moiety conjugated to a ligand for CRBN E3 ubiquitin ligase. (Bio-techne/Tocris, Minneapolis, MN). dBET6 is a potent, selective and cell-permeable degrader of BET bromodomains (1C50=˜10 nM). ARV-771 is a potent BET bromodomain PROTAC®, which is composed of a BRD4-binding moiety linked to a ligand for VHL E3 ligase. (Bio-techne/Tocris, Minneapolis, MN).

PTK2 (focal adhesion tyrosine kinase 2) is overexpressed in cancer cells, such as human hepatocellular carcinoma (HCC) cells. PROTAC® degraders that target PTK2 have been described by Popow, J. et al., 2019, J. Med. Chem., 62(5):2508-2520. Selective PTK degraders were developed that are composed of a highly selective PTK2 inhibitor conjugated to by linkers (e.g., polyethylene glycol linkers) either a ligand of a CRBN or a VHL E3 ubiquitin ligase. By way of nonlimiting example and as shown in FIG. 10, BI-3663 (Boehringer Ingelheim) is a low molecular weight PROTAC® degrader that tethers a selective PTK2/FAK (Focal adhesion tyrosine kinase) kinase inhibitor, i.e., BI-4464, to a ligand of CRBN (CRL4) E3 ubiquitin ligase. More specifically, BI-3663 is composed of BI-4464 (a highly selective ATP competitive inhibitor of PTK2/FKA) linked to pomalidomide. BI-3663 inhibits PTK2 (IC50+18 nM) and triggers the intracellular destruction/degradation of the PTK2 protein. BI-0319 (Boehringer Ingelheim) is a low molecular weight PROTAC® degrader that tethers a selective PTK2/FAK (Focal adhesion tyrosine kinase) kinase inhibitor, i.e., BI-4464, to a ligand of VHL (CRL2) E3 ubiquitin ligase. More specifically, BI-3663 is composed of BI-4464 (a highly selective ATP competitive inhibitor of PTK2/FKA) linked to pomalidomide.

FIG. 10 shows the results of mass spectrometry experiments in which protein substrates susceptible to induced ubiquitination by the abovementioned PROTAC® degraders (BET bromodomain degrader dBET6 (CRBN ligand); BET bromodomain degrader ARV-771 (VHL ligand); PTK2 degrader BI-3663 (CRBN ligand); and PTK2 degrader BI-0319 (VHL ligand) were identified, respectively. The experiments involving CRBN degraders were performed in HEK293T^{CRBN-/-} (CRBNnull cell lines lentivirally engineered to express CRBN-BirA and transiently expressing A3-HA-Ub. The experiments involving VHL degraders were performed in HEK293T cell lines lentivirally engineered to express VHL-BirA and transiently expressing A3-HA-Ub. While cultured in biotin-free medium, the cells were pre-treated with carfilzomib (carf; 0.4 μM) for 1 hour, and then were either treated or untreated with the specified PROTAC®s (1 μM) for another hour. Subsequently, biotin (50 μM) was added to the cells for 15 minutes, after which time the cells were lysed and maintained on ice to terminate the reaction. Enrichment of biotinylated species by streptavidin-coated beads, protein digestion, and protein identification and quantification by mass spectrometry were performed exactly as described in Examples 3 and 4. Triplicate samples allowed for statistical analysis of differentially enriched proteins, represented by log 2-fold-change (FC) between the PROTAC®-treated group and the DMSO-treated group on the x-axis, and significance (p-value estimated by limma) on the y-axis in the figures. FIG. 10 presents the induced ubiquitinated substrates related to the use of the BET inhibitor (JQ1) and dBET6 PROTAC® recruiter for CRBN E3 ligase (CRBN-BirA; ubiquitinated substrates BRD2, BRD3, BRD4 observed in the upper right portion of the top left volcano plot in FIG. 10); the use of the BET inhibitor (JQ1) and ARV-771 PROTAC® recruiter for VHL E3 ligase (VHL-BirA; ubiquitinated substrates BRD2, BRD3, BRD4 observed in the upper right portion of the top right volcano plot in FIG. 10); the use of the PTK2 inhibitor (BI-4464) and PTK2 PROTAC® recruiter BI-3663 for CRBN E3 ligase (CRBN-BirA; ubiquitinated substrate PTK2 observed in the upper right portion of the lower left volcano plot in FIG. 10); and the use of the PTK2 inhibitor (BI-4464) and BI-0319 PROTAC® recruiter for VHL E3 ligase (VHL-BirA; ubiquitinated substrate PTK2 observed in the upper right portion of the lower right volcano plot in FIG. 10). Consistent with the expected actions of these degraders, the most induced ubiquitinated proteins detected were the expected targets previously described for these PROTAC®s based on binding activity or protein degradation. The data suggest that the system and methods described herein can detect induced ubiquitination targets induced by E3-ligase modulating agents such as PROTAC®s specific for CRBN and other E3 ubiquitin ligases, such as VHL.

### Example 7: Profiling Ubiquitinated Substrates of a Sulfonamide Molecular Glue Stabilizer of a CAPERα-DCAF15 Complex Associated with an CRL4 E3 Ligase

A study similar to that described above was conducted to demonstrate that the methods and systems described herein reliably characterize protein degraders (PROTAC®s or molecular glues) other than those based on CRBN or VHL E3 ligases and their respective ligands. In this experiment, the compound used was E7820, an aromatic sulfonamide molecular glue that degrades RBM39 (RNA-Binding Motif Protein 39), /CAPERα (Co-activator of Activating Protein 1 and Estrogen Receptors), which is a target of sulfonamides. RBM39 and CAPERα are alternative names for the same protein (See. Han, T., et al., 2017, Science, 356(6336 and Uehara, T. et al., 2017, Nat Chem Biol., 13(6):675-680), and are designated “RBM39/CAPERα” herein. E7820, which has protein-protein interaction stabilizing activity, acts as a molecular glue by stabilizing the formation of a complex between RBM39/CAPERα and DCAF15 (DDB-1 and Cullin-4 Associated Factor 15), resulting in the increased proteasomal degradation of RBM39/CAPERα. In this process. DCAF15 forms a complex with CUL4A or CUL4B having E3 ubiquitin ligase activity and serves as an adaptor conferring substrate specificity to CRL4 (cullin-RING ubiquitin ligase 4). (Halskamp, M. D. et al., 2021, BMC Cancer, 21(571), pp. 1-12). RBM39/CAPERα is upregulated in many cancers and is a key factor in tumor-targeted mRNA and protein expression: it regulates transcription of several tumor-related genes. The characteristics of the RBM39/CAPERα protein are described in a review by Xu, C. et al., 2021, Cell Death Discovery, 7(214).

The protocol used in this experiment is the same as that described in Example 6 above, except that the system was evaluated in a HEK293T cell line lentivirally engineered to express DCAF15-BirA and transiently expressing A3-HA-Ub. While cultured in biotin-free medium, the cells were pre-treated with carfilzomib (carf; 0.4 μM) for 1 hour, and then were either treated or untreated with E7820 (10 μM) for another hour. Subsequently, biotin (50 μM) was added to the cells for 15 minutes, after which time the cells were lysed and maintained on ice to terminate the reaction. Enrichment of biotinylated species by streptavidin-coated beads, protein digestion, and protein identification and quantification by mass spectrometry were performed exactly as described in Examples 3 and 4. Triplicate samples allowed for statistical analysis of differentially enriched proteins, represented by log 2-fold-change (FC) between the E7820-treated group and the DMSO-treated group on the x-axis, and significance (p-value estimated by limma) on the y-axis in the figures. FIG. 11 shows that the induced ubiquitinated substrate RBM39/CAPERα was produced as a result of the use of the E7820 molecular glue in conjunction with DCAF15-BirA and associated CRL4 E3 ligase. Consistent with the expected action of the E7820 glue in the system, the induced ubiquitinated protein RBM39/CAPERα was detected as the target of the E7820 sulfonamide, based on binding activity or protein degradation. The data suggest that the system and methods described herein can detect ubiquitination targets induced by E3-ligase modulating agents such as a molecular glue that is specific for a DCAF15 E3 ubiquitin ligase associated with CRL4.

### Example 8: Profiling Ubiquitinated Substrates of E3 Ubiquitin Ligases Using HDAC Degrading PROTAC®s

Experiments were conducted using the methods and systems described herein to characterize the activity of histone deacetylase (HDAC) protein inhibitors (degraders) (PROTAC®s) that include ligands for CRBN (e.g., pomalidomide) or VHL (VH032, a VHL/HIF-1α, interaction inhibitor) E3 ligases to target HDACs, such as Class 1 HDAC complexes, in cells.

Class I histone deacetylase (HDAC) enzymes include eleven zinc-dependent HDAC proteins that catalyze the hydrolysis of acetyl groups in N-ε-acetyl-L-lysine residues in histones and nonhistone proteins. HDAC1, 2, 3, and 8 are four members of the class 1 HDAC family. HDAC1/2 shares over 80% sequence homology, is localized in the nucleus, and exists in several multiprotein corepressor complexes including Sin3 (e.g., SIN3A), CoREST, MiDAC, and NuRD. HDAC3 shares approximately 50% sequence homology with HDAC1/2, is also predominantly localized in the nucleus, and exists exclusively in the SMRT/NCoR corepressor complex. Because each of the individual corepressor complexes that incorporate HDAC1/2 and 3 has a distinct cellular function, and therefore, the selective targeting of individual complexes may have potential therapeutic benefits in differing clinical applications. (Smalley, J. P. et al., 2022, J. Med. Chem., 65(7):5642-5659).

Heterobifunctional benzamide-based VHL E3 ligase or CRBN E3 ligase proteolysis targeting chimeras (PROTAC®s) were assessed for their ability to degrade, HDAC enzyme activity. PROTAC® XY-07-097 containing a ligand for CRBN E3 ligase linked by a linker to an HDAC inhibitor (dacinostat) that has the following structure:

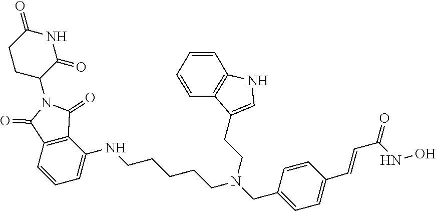 [SMILES: O=C(/C=C/c1ccc(CN(CCCCCNc2cccc3c2C(=O)N(C2CCC(=O)NC2=O)C3=O)CCc2c[nH]c3ccccc23)cc1)NO]

### (Xiong, Y. et al., 2021, Cell Chemical Biology, 28:1514-1527).

PROTEC XY-07-187 containing a ligand for VHL2 E3 ligase linked by a linker to an HDAC inhibitor (dacinostat) that has the following structure:

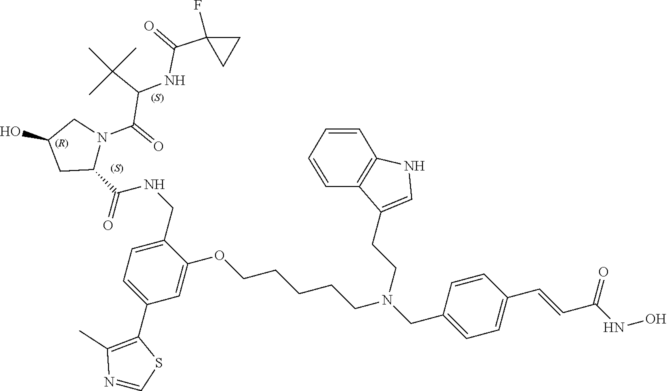

### (Xiong, Y. et al., 2021, Cell Chemical Biology, 28:1514-1527).

FIG. 14A and FIG. 14B show the results of a mass spectrometry experiment in which protein substrates susceptible to induced ubiquitination by HDAC degraders were identified in conjunction with CRBN E3 ligase (FIG. 14A) or VHL E3 ligase (FIG. 14B). The experiments involving CRBN E3 ligase were performed in HEK293T^{CRBN-/-} cells lentivirally engineered to express CRBN-BirA and transiently expressing A3-HA-Ub. The HDAC degrader XY-07-097 was used in the experiments using the CRBN E3 ligase. The experiments involving VHL E3 ligase were performed in HEK293T cells lentivirally engineered to express VHL-BirA and transiently expressing A3-HA-Ub. The HDAC degrader XY-07-187 was used in the experiments using the VHL E3 ligase. While cultured in biotin-free medium, the cells were pre-treated with carfilzomib (carf; 0.4 μM) for 1 hour, and then were either treated or untreated with the HDAC-targeting PROTAC®s (1 μM) for another 45 minutes. Subsequently, biotin (50 μM) was added to the cells for 15 minutes, after which time the cells were lysed and maintained on ice to terminate the reaction. Enrichment of biotinylated species by streptavidin-coated beads, protein digestion, and protein identification and quantification by mass spectrometry were performed exactly as described in Example 3 and 4. Triplicate samples allowed for statistical analysis of differentially enriched proteins, represented by log 2-fold-change (FC) between the PROTAC®-treated group and the DMSO-treated group on the x-axis, and significance (p-value estimated by limma) on the y-axis in the figures. FIG. 14A demonstrates that HDAC induced ubiquitinated substrates, e.g., HDAC2, HDAC6, and HDAC8 were found, as expected, when the CRBN- and HDAC-recruiting PROTAC® XY-07-097 was used in 293T-CRBN^{-/-} CRBN-BirA cells. FIG. 14B demonstrates that HDAC induced ubiquitinated substrates, e.g., HDAC1, HDAC2, HDAC6, and HDAC8 were found, as expected, when the VHL- and HDAC-recruiting PROTAC® XY-07-187 was used in 293T-VHL BirA cells. The results also showed that NCOR1/2, SIN3A/B, RCOR1/3, MIER1/2/3, BAHD1, PHF21A, MIDEAS, MTA3, SUDS3, TRERF1, and CDYL, which are corepressor complex members associated with the HDAC proteins, were also detected, suggesting potential collateral ubiquitination detected in the system. The data suggest that the system and methods described herein can detect ubiquitination targets induced by other types of CRBN and VHL E3-ligase modulating agents such as HDAC degrading PROTAC®s.

### Example 9: Use of CRBN E3 Ligase Mutants to Identify E3 Ligase Substrates

Experiments were performed to identify substrates of an E3 ligase using CRBN as a representative E3 ligase. The experiments compared loss-of-function mutants of an E3 ligase (e.g., CRBN E3 ligase) to the wildtype (WT) ligase. FIGS. 16A and 16B show the results of a mass spectrometry experiment in which protein substrates susceptible to CC-885 induced ubiquitination were identified. These experiments were performed in HEK293T^{CRBN-/-} cells transiently expressing A3-HA-Ub and either WT CRBN-BirA or CRBN-BirA mutant D249Y (FIG. 16A) or CRBN-BirA mutant W386A (FIG. 16B). Compared with CRBN(WT), CRBN(D249Y) had decreased association with the CRL4 complex, while CRBN(W386A) had decreased affinity for CC-885. Both mutants were expected to have a diminished ability to ubiquitinate CC-885-induced substrates. While cultured in biotin-free medium, the cells were pre-treated with carfilzomib (carf; 0.4 μM) for 1 hour, and then were treated with the IMiD derivative CC-885 (1 μM) for another hour. Subsequently, biotin (50 μM) was added to the cells for 15 minutes, after which time the cells were lysed and kept on ice to terminate the reaction. Enrichment of biotinylated species by streptavidin-coated beads, protein digestion, and protein identification and quantification by mass spectrometry were performed exactly as described in Example 3 and 4. Triplicate samples allowed for statistical analysis of differentially enriched proteins, represented by log 2-fold-change between the mutant ligase group and the wild-type ligase group on the x-axis, and significance (p-value estimated by limma) on the y-axis in the figures. FIG. 16A (for the D249Y CRBN mutant) and FIG. 16B (for the W386A CRBN mutant) present the induced ubiquitinated substrates for CC-885 in the upper left quadrant, as revealed by a decrease in biotinylation by the loss-of-function CRBN-BirA mutants. Consistent with the expected actions of CC-885, substrates GSPT1, GSPT2, VCL and CSNK1A1 were identified. Differentially enriched proteins are candidate substrates. The data suggest that E3 ligase substrates can be discovered by the use of the methods and systems described herein, and loss-of-function E3 ligase mutants as reference controls.

### Example 10: Use of VHL E3 Ligase Mutants to Identify E3 Ligase Substrates

Experiments were performed to identify substrates of an E3 ligase using VHL as a representative E3 ligase. The experiments compared loss-of-function mutants of an E3 ligase (e.g., VHL E3 ligase) to the wildtype (WT) ligase. FIGS. 17A and 17B show the results of a mass spectrometry experiment in which protein substrates of VHL were identified. These experiments were performed in A375 cells transiently expressing A3-HA-Ub and either WT VHL-BirA or a VHL-BirA mutant Y98H (FIG. 17A) or C162F (FIG. 17B). Compared with VHL(WT), VHL(C162F) had decreased association with the CRL2 complex, while VHL(Y98H) is a mutation found in von Hippel-Lindau syndrome. Both mutants were expected to have a diminished ability to ubiquitinate VHL substrates. While cultured in biotin-free medium, the cells were pre-treated with MLN4924 (1 μM) for 2 hours. MLN4924 (clinical name: Pevonedistat) is a small molecule inhibitor of the NEDD8 activating enzyme NAE that blocks cullin neddylation to inactivate cullin-RING ligases (CRL) or E3 ligases. The chemical structure of Pevonedistat ([(1S,2S,4R)-4-[4-[[(1S)-2,3-Dihydro-1H-inden-1-yl]amino]-7H-pyrrolo[2,3-d]pyrimidin-7-yl]-2-hydroxycyclopentyl]methyl sulfamic acid ester) is as follows.

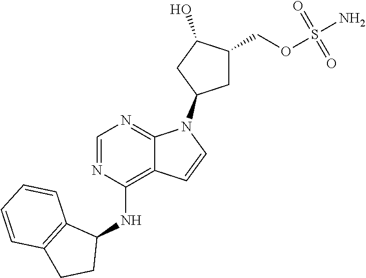 [SMILES: NS(=O)(=O)OC[C@@H]1C[C@@H](n2ccc3c(N[C@H]4CCc5ccccc54)ncnc32)C[C@@H]1O]

“NEDD8” is the acronym for “neuronal precursor cell-expressed developmentally downregulated protein 8.” MLN4924 blocks/inhibits neddylation of Cullin family members and inactivates Cullin-Ring ligases (CRLs). This, in turn, triggers cell-cycle arrest, apoptosis, senescence and autophagy in many cancer cells. Neddylation, a process that is analogous to ubiquitination, refers to the post-translational addition of Nedd8, a ubiquitin-like molecule, to target proteins. Substrate-specific E3 ligases are involved in the neddylation process.

Following the pre-treatment, MLN4924 was washed out of the cell culture with one PBS wash and was replaced with biotin-free medium. Subsequently, biotin (50 μM) was added to the cells for 15 minutes, after which time the cells were lysed and kept on ice to terminate the reaction. Enrichment of biotinylated species by streptavidin-coated beads, protein digestion, and protein identification and quantification by mass spectrometry were performed exactly as described in Example 3 and 4. Triplicate samples allowed for statistical analysis of differentially enriched proteins, represented by log 2-fold-change (FC) between the mutant ligase group and the wild-type ligase group on the x-axis, and significance (p-value estimated by limma) on the y-axis in the figures. FIGS. 17A and 17B present the ubiquitinated substrates of VHL in the upper left quadrant, as revealed by a decrease in biotinylation by the loss-of-function VHL-BirA mutants. Consistent with the expected actions of VHL, HIF1A was identified as a substrate. The data suggest that E3 ligase substrates can be discovered by the use of the methods and systems as described herein, and loss-of-function E3 ligase mutants as reference controls.

### Example 11: Use of Inhibitors of E3 Ligases to Identify E3 Ligase Substrates

Experiments were conducted using the methods and systems described herein in conjunction with an inhibitor of an E3 ligase utilized in the experiment. FIG. 18 shows the results of a mass spectrometry experiment in which protein substrates of a representative E3 ligase, i.e., VHL E3 ligase (“VHL”), were identified using a VHL inhibitor. The experiments were performed in A375 cells that transiently expressed A3-HA-Ub and VHL-BirA. While cultured in biotin-free medium, the cells were pre-treated with MLN4924 (1 μM) for 2 hours. Thereafter, MLN4924 was washed out of the cell culture with one PBS wash and was replaced with biotin-free medium. Subsequently, the cells were treated for 30 minutes with the compound VH298 ((Bio-techne/Tocris, Minneapolis, MN), a ligand that competes with its endogenous substrates for VHL binding. In particular, VH298, having the structure.

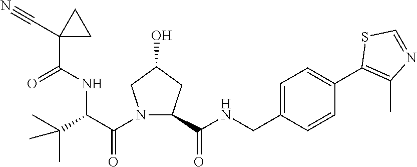 [SMILES: Cc1ncsc1-c1ccc(CNC(=O)[C@@H]2C[C@@H](O)CN2C(=O)[C@@H](NC(=O)C2(C#N)CC2)C(C)(C)C)cc1]

is a potent inhibitor of the interaction of VHL:with its substrate HIF-α. In an embodiment, VH298 inhibitory activity is associated with a K_{d} value of 80 to 90 nM).

During the treatment with the VHL E3 ligase inhibitor, biotin (50 μM) was added to the cells for the last 15 minutes of the 30 minute time period of VH298 treatment. Next, the cells were lysed and kept on ice to terminate the reaction. Enrichment of biotinylated species by streptavidin-coated beads, protein digestion, and protein identification and quantification by mass spectrometry were performed exactly as described in Examples 3 and 4. The samples were analyzed in triplicate to allow for statistical analysis of differentially enriched proteins, represented by log 2-fold-change between the VH298-treated E3 ligase group and the wild-type E3 ligase group on the y-axis, and significance (p-value estimated by limma) on the x-axis in the figures. FIG. 18 shows the ubiquitinated substrates of VHL in the upper left quadrant. A decrease in biotinylation owing to the inhibitor treatment was observed. Consistent with the expected actions of VHL, HIF1A was identified as a substrate. The results suggest that E3 ligase substrates can be discovered using an inhibitor of the E3 ligase of interest in the methods and systems described herein.

### Example 12: Materials and Methods

Materials and methods used in the above-described Examples are provided below.

### Cell Lines

HEK293T cells were authenticated using short tandem repeat (STR) profiling. Cell lines were routinely examined to ensure that they were free of mycoplasma using the MycoAlert mycoplasma detection kit (Lonza, LT07-318). HEK293T cells and their genetically engineered derivatives described herein were maintained in DMEM medium (Gibco; 10569-010) supplemented with 10% feral bovine serum (FBS) (SAFC; 12306C). All cell lines were cultured at 37° C. in a humidified chamber in the presence of 5% CO₂.

### Vectors

Ubiquitin (pRK5-HA-Ubiquitin-WT; Addgene #17608) and BirA (pJW1512; Addgene #62366) sequences were acquired from Addgene, whereas CRBN and VHL sequences were provided by the Genetic Perturbation Platform at the Broad Institute. By applying site-directed mutagenesis, long homology-based cloning and Gateway cloning to these DNA templates, Avi-HA-ubiquitin, A3-HA-ubiquitin, CRBN-3F-BirA and VHL-3F-BirA were created and inserted into the appropriate expression vectors. For transient expression of recombinant proteins, such as Avi-Tag ubiquitin and A3-tag ubiquitin, the pRK5 vector (CMV promoter), kindly provided by Dr. Douglas B. Wheeler, was used. Lentiviral vectors pLX307 (EF1α promoter and puromycin resistance) and pLX305 (hPGK promoter and puromycin resistance), kindly provided by the Genetic Perturbation Platform at the Broad Institute, were used to create stable cell lines expressing genes of interest, such as CRBN-3F-BirA.

The CRBN gene-editing vector (PX330_sgCRBN-2) was generated by ligating annealed oligonucleotides containing CRBN-targeting sequences with BbsI-digested PX330 vector (acquired from Addgene; #42230). The CRBN-targeting oligonucleotide pair was sgCRBN-2F: 5′-CACCGTAAACAGACATGGCCGGCGA-3′ (SEQ ID NO: 43) and sgCRBN-2R: 5′-AAACTCGCCGGCCATGTCTGTITAC-3′ (SEQ ID NO: 44).

### Transfection

One day prior to transfection, an appropriate number of HEK293T cells (˜0.8 million cells/cm² of surface area) was seeded into tissue culture plates. The next day, 0.2 μg of plasmids for every cm² of surface area were mixed with 100 μl of serum-free DMEM or Opti-MEM for every μg of plasmids used. Thereafter, 3 μL of 1 mg/ml linear polyethyleneimine (Polysciences; 23966-1) per μg of plasmids was added to the mixture. The mixture was briefly vortexed and centrifuged. After 15-20 minutes of incubation at room temperature, the DNA-PEI solution was added dropwise to the HEK293T cells. The cells were then used in experiments after at least 24 hours.

### Lentiviral Transduction

Lentiviruses were generated by transfecting 1.5-2 million HEK293T cells seeded in a T25 flask one day prior. Specifically, 5 mg of total plasmids including lentiviral transfer plasmid, packaging plasmid (pMD2.G), and packaging plasmid (psPAX2) in a molar ratio of 4:1:4 were used. The plasmids were mixed in 500 μl of serum-free DMEM or Opti-MEM, and 15 μL of 1 mg/ml linear polyethyleneimine (Polysciences; 23966-1) was added. After 15-20 minutes of incubation at room temperature, the DNA-PEI solution was added dropwise to the HEK293T cells. Viral supernatant was collected at 48 hours and 72 hours after transfection, filtered through 0.45 μm membrane, and added to target cells in the presence of 8 μg/ml polybrene (Millipore, Billerica, MA). Cells were selected with antibiotics starting 48 hours after the initial infection. HEK293T cells were selected and maintained in medium containing 1 μg/mL of puromycin.

### Generation of CRBN-Knockout HEK293T Cells

HEK293T cells were co-transfected with PX330_sgCRBN-2 and pRK5-puroR on Day 0 and were treated with puromycin (1 μg/mL) from Day 2 to Day 4. On Day 7, these cells were plated in 96-well plates at an estimated concentration of 0.5 cells/well. After 2-3 weeks, cell clones that grew up from each individual well were expanded. CRBN-knockout clones were validated by both Western blotting and PCR genotyping. The PCR primers used for amplifying the targeted CRBN locus were CRBN-Ex1-F: 5′-GGCCTGTAATTGTCCCTC-3′ (SEQ ID NO: 45) and CRBN-Ex1-R: 5′-GTAACCGCTGTGAATCTG-3′ (SEQ ID NO: 46).

### Cell Lysis

Cells were rinsed one time with ice-cold PBS and lysed directly in tissue culture plates on ice for >5 minutes with RIPA buffer (50 mM Tris-HCl, 150 mM NaCl, 1% NP-40, 0.5% sodium deoxycholate, and 0.1% sodium dodecyl sulfate, pH 7.4) supplemented with COMPLETE™ EDTA-free protease inhibitor cocktail (Roche; 04693159001), 1 tablet per 10 ml of buffer), and benzonase (Millipore; E1014), 1 μl per 10 ml of buffer. When immunoprecipitation was needed, IP lysis buffer (25 mM Tris-HCl, 150 mM NaCl, 1 mM EDTA, 1% NP-40, and 5% glycerol, pH 7.4) was used instead of RIPA buffer. Whole cell lysate was rotated at 4° C. for at least 15 minutes and clarified by centrifugation at 21,000×g at 4° C. for 15 minutes. Protein concentrations were estimated using a Pierce BCA protein assay (Thermo Scientific; 23225), and lysate volumes were adjusted to normalize protein concentrations across different samples.

### Western Blotting

To prepare samples for SDS-PAGE, lysates were mixed with NUPAGE™ LDS Sample Buffer (Invitrogen; NP0007), to 1×, and with 1M DTT (Sigma-Aldrich; D0632), to 50 mM, and then were heated at 70° C. for 10 minutes. Equal amounts of proteins (20-40 mg) were resolved using NUPAGE™ 4-12%, Bis-Tris, 1.5 mm, on mini protein gels (Invitrogen; NP0336BOX). Proteins were transferred onto 0.45 μm nitrocellulose membranes (Bio-Rad; 1620115) in Tris-Glycine transfer buffer (25 mM Tris, 192 mM glycine, 20% methanol) using the XCell II Blot Module (Invitrogen; EI9051) system. Following transfer, the membranes were blocked with 5% non-fat milk in TBS-T (TBS with 0.2% Tween-20), followed by incubation with primary antibodies at 4° C. overnight. On the next day, after washing with TBS-T, the membranes were incubated with fluorophore-conjugated secondary antibodies for 1 hour at room temperature. The membranes were then washed and scanned with an Odyssey Infrared scanner (Li-Cor Biosciences, Lincoln, NE).

### Antibodies

Primary antibodies used in the above-described Examples include anti-GSPT1, anti-CRBN (abcam; ab244223), anti-GAPDH (Cell Signaling; 5174S), anti-HA (Sigma Aldrich; 11583816001), anti-α-tubulin (Cell Signaling; 3873S) and anti-V5 (Absolute Antibody; Ab00136-1.1). To detect biotin, ALEXA FLUOR™ 680 conjugated streptavidin (Invitrogen; S21378) was used. The secondary antibodies included IRDye680-conjugated goat anti-rabbit IgG (LI-COR Biosciences; 926-68071) and IRDye800-conjugated goat anti-mouse IgG (LI-COR Biosciences; 926-32210).

### Streptavidin Affinity Enrichment

The preparation of cellular lysate was as described above for Cell Lysis, except that the RIPA buffer was supplemented with COMPLETE™ EDTA-free protease inhibitor cocktail (Roche; 04693159001; 1 tablet per 10 ml of buffer), benzonase (Millipore; E1014; 1 μl per 10 ml of buffer), 1.5 mM MgCl₂, 1 mM EGTA, and 10 mM NEM (N-ethylmaleimide). For each sample, 1 ml of 3 mg/ml lysate was rotated overnight at 4° C. with 50 μl of resuspended streptavidin Sepharose beads (CYTIVA™; 17511301), which were pre-washed with RIPA lysis buffer 3 times. For all steps that required pelleting of beads, centrifugation was performed at 400×g for 1 minute. On the next day, streptavidin beads were pelleted, and the supernatant (a.k.a. flow-through) was either saved for analysis by Western blotting or was discarded. Streptavidin beads were then washed two times with 1 ml of 2% SDS wash buffer (2% SDS, 25 mM Tris-HCl, pH 7.5), and three times with 0.5-1 ml of 2M urea wash buffer (2M urea, 50 mM Tris-HCl, pH 8). Between each wash, the beads were rotated at room temperature for at least 5 minutes. During the last wash, the beads and the washing buffer were transferred to a new microfuge tube. For Western blotting, the beads were heated at 70° C. for 10 minutes in sample buffer (RIPA lysis buffer containing 1× NUPAGETH LDS Sample Buffer, (Invitrogen; NP0007), 50 mM DTT (Sigma-Aldrich; D0632), and 2 mM biotin). For mass spectrometry-based analysis, the procedures described in On-bead Digestion below were used.

### On-Bead Digestion

After affinity enrichment and washing (as described for Streptavidin Affinity Enrichment), Streptavidin beads were resuspended in 2M urea buffer (2M urea, 50 mM Tris-HCl, pH 8). Next, TCEP was added to each sample to a final concentration of 10 mM, and the samples were incubated on a thermomixer at 37° C. for 30 minutes. Thereafter, DTT was added to the samples to a final concentration of 15 mM, and the samples were incubated on a mixer at room temperature for 45 minutes. 0.5 μg of Trypsin/Lys-C (Promega; V5073) were added to the samples, which were then incubated at 37° C. for 3-4 hours for protein digestion. Next, the samples were diluted with Tris buffer (50 mM Tris-HCl, pH 8) to <1M urea, and an additional 1 μg of Trypsin/Lys-C was added for further digestion at 37° C. overnight. On the following day, the beads were pelleted and the supernatant was collected in a microfuge tube. The beads were washed two times with 30 μl of HPLC-grade water, the washes were combined with the supernatant collected previously. The sample was centrifuged at 21,000×g for 10 minutes, and the clarified supernatant was transferred to a new microfuge tube and acidified with 50% formic acid to a final concentration of 2%. Digested peptides were dried in a centrifugal evaporator. The dried peptides were stored in −80° C. until further analysis by mass spectrometry.

### Substrate Identification by Ubiquitin Biotinylation

HEK293T cells stably expressing an E3 ligase of interest fused to biotin ligase BirA were seeded in biotin-free DMEM media. Biotin-free DMEM medium was prepared by incubating FBS (SAFC; 12306C) with Strep-Tactin Sepharose resin (IBA Lifesciences; 2-1201-002) overnight, followed by the addition of biotin-depleted FBS to DMEM to a final concentration of 10% and sterile filtering. On the next day, the cells were transfected using pRK5-Avi-HA-ubiquitin or pRK5-A3-HA-ubiquitin in serum-free DMEM. After twenty-four hours, the cells were treated with carfilzomib (0.4 μM) for 1-2 hours, followed by an optional treatment with an inducer of ubiquitination (e.g., CC-885) for 1-2 hours, followed by the addition of biotin (50 μM). After a period of biotin labeling (2-15 minutes), the cells were lysed as previously described and analyzed by Western blotting or mass spectrometry.

Liquid Chromatography-Mass Spectrometry (LC-MS) with Orbitrap Exploris 480

Protein digests were resuspended in 100 uL 1% formic acid, and further acidified with addition of 1% formic acid to a pH of 2-3 prior to desalting using C18 solid phase extraction plates (SOLA, Thermo Fisher Scientific).

Desalted peptides were dried in a vacuum-centrifuged and reconstituted in 1.0% formic acid for LC-MS analysis. Data were collected using an Orbitrap Exploris 480 mass spectrometer (Thermo Fisher Scientific) coupled with a UltiMate 3000 RSLCnano System. Peptides were separated on an Aurora 25 cm×75 μm inner diameter microcapillary column (IonOpticks), and using a 60 min gradient of 5-25% acetonitrile in 1.0% formic acid with a flow rate of 250 nL/min. Each analysis used a TopN data-dependent method. The data were acquired using a mass range of m/z 350-1200, resolution 60,000, AGC target 3×10⁶, auto maximum injection time, dynamic exclusion of 15 sec, and charge states of 2-6. TopN 15 data-dependent MS2 spectra were acquired with a scan range starting at m/z 110, resolution 15,000, isolation window of 1.4 m/z, normalized collision energy (NCE) set at 30%, AGC target 1×10⁵ and the automatic maximum injection time.

LC-MS with Orbitrap Exploris 480 was used to acquire all data as described in the above examples and corresponding figures, except for Example 8 (FIGS. 14A and 14B), in which the data were acquired using LC-MS with Bruker Tims TOP Pro2, described below.

Liquid Chromatography-Mass Spectrometry (LC-MS) with Bruker TimsTOF Pro 2

Protein digests were resuspended in 100 uL 1% formic acid, and further acidified with addition of 1% formic acid to a pH of 2-3 prior to desalting using C18 solid phase extraction plates (SOLA, Thermo Fisher Scientific).

Desalted peptides were dried in a vacuum-centrifuged and reconstituted in 0.1% formic acid for LC-MS analysis. Data were collected using a TimsTOF Pro2 (Bruker Daltonics, Bremen, Germany) coupled to a nanoElute LC pump (Bruker Daltonics, Bremen, Germany) via a CaptiveSpray nano-electrospray source. Peptides were separated on a reversed-phase C18 column (25 cm×75 μm ID, 1.6 μM, IonOpticks, Australia) containing an integrated captive spray emitter. Peptides were separated using a 50 min gradient of 2-30% buffer B (acetonitrile in 0.1% formic acid) with a flow rate of 250 nL/min and column temperature maintained at 50° C.

DDA was performed in Parallel Accumulation-Serial Fragmentation (PASEF) mode to determine effective ion mobility windows for downstream diaPASEF data collection (Meier et al., 2020). The ddaPASEF parameters included: 100% duty cycle using accumulation and ramp times of 50 ms each, 1 TIMS-MS scan and 10 PASEF ramps per acquisition cycle. The TIMS-MS survey scan was acquired between 100-1700 m/z and 1/k0 of 0.7-1.3 V·s/cm2. Precursors with 1-5 charges were selected and those that reached an intensity threshold of 20,000 arbitrary units were actively excluded for 0.4 min. The quadrupole isolation width was set to 2 m/z for m/z<700 and 3 m/z for m/z>800, with the m/z between 700-800 m/z being interpolated linearly. The TIMS elution voltages were calibrated linearly with three points (Agilent ESI-L Tuning Mix Ions; 622, 922, 1,222 m/z) to determine the reduced ion mobility coefficients (1/K0). To perform diaPASEF, the precursor distribution in the DDA m/z-ion mobility plane was used to design an acquisition scheme for DIA data collection which included two windows in each 50 ms diaPASEF scan. Data was acquired using sixteen of these 25 Da precursor double window scans (creating 32 windows) which covered the diagonal scan line for doubly and triply charged precursors, with singly charged precursors able to be excluded by their position in the m/z-ion mobility plane. These precursor isolation windows were defined between 400-1200 m/z and 1/k0 of 0.7-1.3 V·s/cm2.

The data and results described in Example 8 (FIGS. 14A and 14B) were acquired using LC-MS with Bruker Tims TOP Pro2,

### LC-MS Data Analysis

Proteome Discoverer 2.4 (Thermo Fisher Scientific) was used for .RAW file processing and controlling peptide and protein level false discovery rates, assembling proteins from peptides, and protein quantification from peptides. MS/MS spectra were searched against a Swissprot human database (January 2021) with both the forward and reverse sequences, as well as known contaminants such as human keratins. Database search criteria were as follows: tryptic with two missed cleavages, a precursor mass tolerance of 10 ppm, fragment ion mass tolerance of 0.6 Da, static alkylation of cysteine (57.02146 Da), and variable oxidation of methionine (15.99491 Da). Peptides were quantified using the MS1 area under the curve (AUC), and peptide abundance values were summed to yield the protein abundance values.

The resulting data were filtered to include only proteins that had a minimum of 2 unique peptides and 2 abundance counts per protein in at least 2 replicates. Abundances were normalized and scaled using in-house scripts in the R framework (R Development Core Team, 2014). Proteins with missing values were imputed by random selection from a gaussian distribution either with a mean of the non-missing values for that treatment group or with a mean equal to the median of the background (in cases when all values for a treatment group are missing). Significant changes comparing the relative protein abundance of treatment samples to the DMSO control treatments were assessed by moderated t test as implemented in the limma package within the R framework (M. E. Ritchie et al., 2015, Nucleic Acids Res, 43(7):e47). A protein was considered a ‘hit’ if it met the pre-determined ‘hit’ threshold of P-value<0.01 and fold change>2.

The diaPASEF raw file processing and controlling peptide and protein level false discovery rates, assembling proteins from peptides, and protein quantification from peptides were performed using library free analysis in DIA-NN 1.8 (Demichev, V. et al., 2020, Nature Methods, 17, pp. 41-44; DOI 10.1038/s41592-019-0638-x). Library free mode performs an in silico digestion of a given protein sequence database alongside deep learning-based predictions to extract the DIA precursor data into a collection of MS2 spectra. The search results are then used to generate a spectral library that is then employed for the targeted analysis of the DIA data searched against a Swissprot human database (January 2021). Database search criteria largely followed the default settings for directDIA, including tryptic with two missed cleavages, carbomidomethylation of cysteine as a fixed modification, and oxidation of methionine as a variable modification and precursor Q-value (FDR) cut-off of 0.01. Precursor quantification strategy was set to Robust LC (high accuracy) with RT-dependent cross run normalization. Proteins with missing values were imputed by random selection from a gaussian distribution either with a mean of the non-missing values for that treatment group or with a mean equal to the median of the background (in cases when all values for a treatment group are missing). Protein abundances were scaled using in-house scripts in the R framework (R Development Core Team, 2014). The resulting data comparisons (treatment vs control groups) were filtered to include only proteins that had a minimum of 2 abundance counts per protein in at least 2 replicates followed by statistical analysis using the limma package within the R framework (Ritchie et al., 2015, Ibid.).

### Cell Transfection and Incorporation of ONPK

Chemical synthesis of ONPK 1.41 g 4-nitrophenyl carbonochloridate is dissolved in 20 mL DCM and is added dropwise to 20 mL DCM containing 1.24 g 1-(6-nitrobenzo[d][1,3]dioxol-5-yl)ethanol. (See. e.g., Y. Liu et al., 2021, PNAS USA, Vol. 118, No. 25). Thereafter, 2 mL TEA is added dropwise into the mixture and stirred at room temperature overnight. The reaction solution is evaporated to remove the solvent and redissolved in 30 mL THF. 1.20 g Na-Boc-lysine is dissolved in saturated NaHCO₃ solution. The THF solution is added dropwise to the solution in an ice bath. After incubation of the reaction overnight, the reaction solution is extracted with ethylacetate and purified by column chromatography to yield Na-Boc-ONPK. Deprotection is carried out by TFA in DCM solution at room temperature for 1 hour. The reaction product is subsequently evaporated and redissolved in MeOH (5 mL) and precipitated into Et₂O (250 mL) to obtain ONPK.

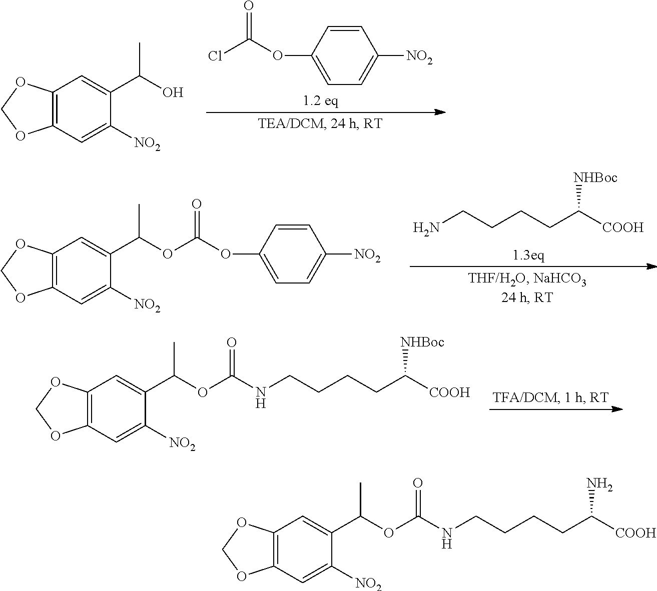 [SMILES: CC(O)c1cc2c(cc1[N+](=O)[O-])OCO2 ; O=C(Cl)Oc1ccc([N+](=O)[O-])cc1 ; CC(OC(=O)Oc1ccc([N+](=O)[O-])cc1)c1cc2c(cc1[N+](=O)[O-])OCO2 ; CC(C)(C)OC(=O)N[C@@H](CCCCN)C(=O)O ; CC(OC(=O)NCCCC[C@H](NC(=O)OC(C)(C)C)C(=O)O)c1cc2c(cc1[N+](=O)[O-])OCO2 ; CC(OC(=O)NCCCC[C@H](N)C(=O)O)c1cc2c(cc1[N+](=O)[O-])OCO2]

### Cell Transfection and ONPK Incorporation

Methods for cell transfection and incorporation of ONPK in BirA are performed as described in Y. Liu et al., 2021, PNAS USA, Vol. 118, No. 25. For the methods, plasmid encoding BirA-K183(ONPK) (1 μg) and 1 μg of the plasmid encoding 4×tRNA^{Pyl}-ONPK-RS (CUA, the anticodon of the amber codon; Pyl, pyrrolysine) pair, are diluted in 50 μL Opti-MEM, and then 4 μL P3000 regent are added. Next, the diluted DNA are added into 50 μL Opti-MEM with 4 μL Lipofectamine 3000 reagent and incubated for 15 minutes. Thereafter, the DNA-lipid complexes are added into 1 mL DMEM with 10% FBS containing 100 μM ONPK. Subsequently, this mixture is added into HEK293T cultured in 12-well plates with 60-70% confluence and cultured for another 24 hours before carrying out the experimental methods.

Stable cell line construction. For preparation lentivirus for transfection, plasmid vector encoding BirA-ONPK (0.5 μg) are diluted in 50 μL Opti-MEM. 3 μL P3000 regent are added, followed by addition into 50 μL Opti-MEM with 3 μL Lipofectamine 3000 reagent and incubation for 15 min. The plasmid-lipid complexes are added into HEK293T cells cultured in 12-well plates at 60-70% confluence. The culture medium containing prepared lentivirus is collected and filtered through a 0.45 μm filter after 48 h. Thereafter, 0.4 mL of the culture medium is collected and added to fresh HEK293T cells at 70-80% confluence in 12-well plates for another 48 h incubation, followed by replacement with fresh medium containing 5 μg ml⁻¹ blasticidin (Selleck, S7419) every day for selection. After a week, cells expressing ONPK-RS are sorted by flow cytometry.

Photo-activation of BirA-K183(ONPK) and biotin labeling in living mammalian cells. Before activation, 100 mM biotin DMSO stock is diluted directly into cell culture medium to

a final concentration of 100 μM (HEK293T). Cells expressing BirA-K183(ONPK) are subjected to UV irradiation in the cell culture medium. The cell culture plate or dish is placed on the ChemiDoc XRS+ and irradiated from the bottom by UV light at intensity of 0.5 milliwatt per cm² for 5 min. Next, HEK293T cells are incubated at 37° C. for 10 minutes. Labeling is stopped by exchanging the cell culture medium to ice-cold PBS. For negative control, the UV irradiation was not used.

Activation of ChemoBirA in Animals (Mice). HEK293T cells are cultured in 10 cm dishes, cotransfected with a vector containing a polynucleotide encoding BirA biotin ligase and TCOK-RS in DMEM/10% FBS supplemented with 200 μM TCOK. After 18 hours of further culture, the cells are lysed, centrifuged, and resuspended in PBS. The cells (˜1×10⁷ cells/50 μL) are then injected subcutaneously (SC) into the hind legs of mice (Nu-Nu nude mice (male, 5-6 weeks). 50 μL DM-Tz (300 mM concentration; equal to 66 mg/kg body weight for an ˜25 g mouse) are then intravenously (IV) injected into mice via the tail vein. 2 h after the injection of cells, biotin (100 μL, 200 mM) is injected intraperitoneally (IP). After 4 hours, the cells are extracted, followed by analysis of biotinylation level by Western blotting or enrichment for MS.

### OTHER EMBODIMENTS

From the foregoing description, it will be apparent that variations and modifications may be made to the embodiments described herein for adoption to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of identifying a substrate of an E3 ligase, the method comprising:
contacting a ubiquitinated substrate of an E3 ligase with a biotin ligase fused to the E3 ligase, wherein the E3 ligase substrate is ubiquitinated with one or more ubiquitin or ubiquitin-like molecules, each of which is fused to a biotin ligase peptide substrate comprising a biotinylation site of the biotin ligase, wherein the biotin ligase fused to the E3 ligase biotinylates the tagged ubiquitinated E3 ligase substrate when in proximity to the tag; and
identifying the substrate of the E3 ligase by detecting and/or selecting the biotinylated ubiquitinated E3 ligase substrate.

2. The method of claim 1, wherein the E3 ligase is fused at its amino (NH₂)- or carboxy (COOH)-terminus to the biotin ligase.

3. A method of identifying a substrate of an E3 ligase, the method comprising:
(i) expressing in a cell one or more ubiquitin or ubiquitin-like molecules fused to a biotin ligase peptide substrate which comprises a biotinylation site of the biotin ligase;
(ii) expressing in the cell an E3 ligase fused at its amino (NH₂)- or carboxy (COOH)-terminus to a non-promiscuous biotin ligase, wherein the E3 ligase catalyzes the ubiquitination of an E3 ligase substrate bound thereto with the one or more ubiquitin or ubiquitin-like molecules of (i), which are biotinylated by the non-promiscuous biotin ligase fused to the E3 ligase; and
(iii) identifying the substrate of the E3 ligase by detecting and/or selecting the biotinylated and ubiquitinated substrate bound to the E3 ligase.

4. The method of claim 2, wherein the biotin ligase fused to the E3 ligase comprises a non-promiscuous biotin ligase.

5. The method of claim 6, wherein the biotin ligase is BirA.

6. The method of claim 1, wherein the biotin ligase peptide substrate comprises one or more of the following:
an amino acid sequence (X)DIFEAQKIE(Y) (SEQ ID NO: 1), wherein X is selected from no amino acid; amino acids G, L, and N; amino acids L and N; or amino acid N; and
Y is selected from no amino acid; amino acids W, H, and E; amino acids W and H; or amino acid W;
an amino terminus initial methionine (M) residue.
an amino acid sequence having at least 85% sequence identity to amino acid sequence GLNDIFEAQKIE (SEQ ID NO: 2) or MGLNDIFEAQKIE (SEQ ID NO: 3).
a carboxy terminal linker sequence.

7. The method of claim 2, wherein the biotin ligase fused to the E3 ligase is a biotin ligase enzyme genetically or recombinantly substituted with an unnatural amino acid residue.

8. The method of claim 7, wherein the unnatural amino acid residue comprises a photocaged lysine (K) analog at position 183 (K183) in a non-promiscuous BirA biotin ligase enzyme derived from wild-type E. coli, or a photocaged K analog at an analogous position in another biotin ligase enzyme.

9. The method of claim 8, wherein the photocaged lysine analog comprises N^{ε}-[1-(6-Nitrobenzo[d][1,3]dioxol-5yl)ethoxy)carbonyl]-L-lysine (ONPK) at position 183 in the BirA biotin ligase enzyme or at an analogous position in another biotin ligase enzyme.

10. The method of claim 8, further comprising expressing in the cells pyrrolysine-amino acyl tRNA synthase (PylRS) for ONPK incorporation in the BirA lysine analog; and subjecting the cell to photo-illumination prior to step (iii) to restore activity of the photocaged BirA biotin ligase enzyme.

11. The method of claim 1, wherein the ubiquitin-like molecule is selected from the group consisting of NEDD8, SUMO, ISG15, ATG8, ATG12, FAT10 and functional equivalents thereof.

12. The method of claim 1, wherein the E3 ligase is selected from the group consisting of an RBR, HECT, RING, or cullin-RING, class of E3 ubiquitin ligases, E3A, MDM2, Anaphase-promoting complex (APC), UBR5 (EDD1), SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HERC5, HERC6. HUWE1, ITCH, NEDD4, NEDD4L, PPIL2, PRPF19, PIAS1, PIAS2, PIAS3, PIAS4, RANBP2, RNF4, RNF13, RNF38, RNF139, RNFx2, RNF126, RBX1, SMURF1, SMURF2, STUB1, TEB4, TOPORS, TRIP12, UBE3A, UBE3B, UBE3C, UBE3D, UBE4A, UBE4B, UBOX5, UBR5, VHL, WWP1, WWP2, Parkin, MKRN1, CRBN, CRL4-CRBN, CRBN, XIAP, cIAPl, gp78, DoalO, Hrdl, MARCH1 MARCH5, DCAF15, and multimeric complexes thereof.

13. A system or composition comprising:
(i) one or more ubiquitin or ubiquitin-like molecules fused to a biotin ligase peptide substrate which comprises a biotinylation site of the biotin ligase, or one or more polynucleotides encoding said one or more ubiquitin or ubiquitin-like molecules; and
(ii) an E3 ligase fused to a biotin ligase, or a polynucleotide encoding said E3 ligase; wherein the E3 ligase catalyzes the ubiquitination of an E3 ligase substrate bound thereto with the one or more ubiquitin or ubiquitin-like molecules of (i), and wherein the one or more ubiquitin or ubiquitin-like molecules are biotinylated by the biotin ligase fused to the E3 ligase.

14. A kit or cell comprising composition of claim 1.

15. An in vitro or ex vivo method comprising:
contacting a cell with the system of claim 13, wherein the contacting results in the expression in the cell of the one or more ubiquitin or ubiquitin-like molecules fused to a biotin ligase peptide substrate or one or more polynucleotides encoding said one or more ubiquitin or ubiquitin-like molecules; and the E3 ligase fused at its amino (NH₂)- or carboxy (COOH)-terminus to a non-promiscuous biotin ligase, or a polynucleotide encoding said E3 ligase.

16. A system or composition comprising:
(i) one or more ubiquitin proteins fused to a biotin ligase peptide substrate tag which comprises a biotinylation site of the biotin ligase; or one or more polynucleotides encoding said one or more ubiquitin proteins;
(ii) an E3 ligase fused at its amino (NH₂)- or carboxy (COOH)-terminus to a biotin ligase, or a polynucleotide encoding said E3 ligase; and
(iii) an agent selected from an IMiD, a molecular glue, a reprogramming molecule, or a proteolysis targeting chimera (PROTAC®), that facilitates an association of a target protein or a target molecule with the E3 ligase, whereby the target protein or target molecule is ubiquitinated with the one or more tagged ubiquitin proteins of (i), which are biotinylated by the biotin ligase fused to the E3 ligase.

17. A method for identifying a target protein, the method comprising:
(i) providing one or more ubiquitin proteins fused to a biotin ligase peptide substrate tag which comprises a biotinylation site of the biotin ligase; or one or more polynucleotides encoding said one or more ubiquitin proteins;
(ii) providing an E3 ligase fused at its amino (NH₂)- or carboxy (COOH)-terminus to a biotin ligase, or a polynucleotide encoding said E3 ligase;
(iii) providing an agent selected from an IMiD, a molecular glue, a reprogramming molecule, or a proteolysis targeting chimera (PROTAC®), that facilitates an association of the target protein with the E3 ligase, whereby the target protein is ubiquitinated with the one or more tagged ubiquitin proteins of (i), which are biotinylated by the biotin ligase fused to the E3 ligase; and
(iv) detecting and/or selecting the biotinylated and ubiquitinated target protein associated with the E3 ligase, thereby identifying the target protein.

18. A high-throughput screening method for screening for target proteins, the method comprising:
(a) contacting a cell with the system of claim 13, wherein the contacting results in the delivery into the cell of the one or more ubiquitin or ubiquitin-like molecules fused to a biotin ligase peptide substrate, or one or more polynucleotides encoding said one or more ubiquitin or ubiquitin-like molecules; and the E3 ligase fused at its amino (NH₂)- or carboxy (COOH)-terminus to a non-promiscuous biotin ligase, or a polynucleotide encoding said E3 ligase;
(b) contacting a cell lysate or extract produced from the cell of step (a) with donor beads conjugated to streptavidin and acceptor beads conjugated to an antibody specifically directed to the substrate of the E3 ligase; and
(c) measuring levels of biotinylated and ubiquitinated substrate produced following ubiquitin biotinylation by the non-promiscuous biotin ligase fused to the E3 ligase.

19. A method of identifying a substrate of an E3 ubiquitin ligase, the method comprising:
introducing into a cell a polynucleotide encoding a mutant E3 ubiquitin ligase fused to a biotin ligase and expressing the mutant E3 ubiquitin ligase in the cell; wherein the cell expresses one or more ubiquitin or ubiquitin-like molecules fused to a biotin ligase peptide substrate which comprises a biotinylation site of the biotin ligase;
culturing the cell in biotin-containing medium for a time sufficient for biotinylation of ubiquitinated protein substrates of the E3 ubiquitin ligase;
enriching the biotinylated protein substrates with streptavidin; and
detecting the levels of biotinylated protein substrates ubiquitinated by the mutant E3 ubiquitin ligase in lysates of the cells compared with the levels of biotinylated protein substrates ubiquitinated by a wildtype E3 ubiquitin ligase to identify substrate proteins differentially enriched by the mutant E3 ubiquitin ligase versus the wildtype E3 ubiquitin ligase.

20. A method of identifying a substrate of an E3 ligase, the method comprising:
contacting a cell of claim 14 with an inhibitor of all cullin-RING ligase (CRL) activity or an inhibitor of all ubiquitin ligase activity in a cell for a time sufficient to block E3 ligase activity and accumulate E3 ligase substrate in the cell; wherein the cell is cultured in biotin-free medium;
removing the inhibitor from the culture to restore E3 ligase activity and culturing the cell in biotin-free medium prior to supplementing the culture medium with biotin, wherein ubiquitinated substrates of the E3 ligase are biotinylated in the cell;
enriching the biotinylated protein substrates with streptavidin following cell lysis; and
detecting and identifying the ubiquitinated substrate of the E3 ligase in the cell lysate.

21. A method of identifying a substrate of an E3 ligase, the method comprising:
(a) contacting a cell of claim 14 with a first E3 ligase inhibitor for a time sufficient to block E3 ligase and cullin ring ligase (CRL) activity in the cell; wherein the cell is cultured in biotin-free medium;
(b) removing the first inhibitor from the culture to restore E3 ligase and CRL activity and culturing the cell in biotin-free medium prior to supplementing the culture medium with biotin and a second inhibitor that blocks or inhibits E3 ligase activity;
(c) lysing the cell and enriching the biotinylated ubiquitinated protein substrates of the E3 ligase from the cell lysate with streptavidin; and
(d) detecting and identifying the enriched biotinylated and ubiquitinated protein substrates having decreased biotinylation due to inhibition of E3 ligase activity by the first and second E3 ligase inhibitors.
